(19) United States
(12) Patent Application Publication
(10) Pub. No.: US 2024/0254208 A2
(43) Pub. Date: Aug. 1, 2024

(54) COMPOSITIONS AND METHODS FOR USING BISPECIFIC ANTIBODIES TO BIND COMPLEMENT AND A TARGET ANTIGEN

(71) Applicant: Regeneron Pharmaceuticals, Inc., Tarrytown, NY (US)

(72) Inventors: Christos Kyratsous, Irvington, NY (US); Chia-Yang Lin, Scarsdale, NY (US); Andrew Murphy, Croton-on-Hudson, NY (US); Brinda Prasad, Princeton, NJ (US); Neil Stahl, Carmel, NY (US)

(21) Appl. No.: 17/430,117

(22) PCT Filed: Feb. 12, 2020
(86) PCT No.: PCT/US2020/017889

§ 371 Date: Aug. 11, 2021

Related U.S. Application Data
(60) Provisional application No. 62/804,358, filed on Feb. 2, 2019.

(51) Int. Cl. C07K 16/12; A61P 31/04; A61P 31/16; C07K 16/10; C07K 16/28

(52) U.S. Cl. CPC C07K 16/1271; A61P 31/04; A61P 31/16; C07K 16/1018; C07K 16/1214; C07K 16/2878; C07K 16/2887; A61K 2039/505

(57) ABSTRACT

According to certain embodiments, the present disclosure provides bispecific antigen-binding molecules comprising a first antigen-binding domain that specifically binds a target antigen and a second antigen binding domain that binds a complement component. In certain embodiments, the bispecific antigen-binding molecules of the present disclosure are capable of binding to the target antigen with an EC₅₀ of about 10 nM or less, and/or are capable of promoting complement deposition on the target antigen with an EC₅₀ of about 10 nM. In certain embodiments, the bispecific antigen-binding molecules of the disclosure are useful for treating diseases in which inhibition or reduction of the growth of an infectious agent or cancer cell is desired and/or therapeutically beneficial.

### CROSS-REFERENCE TO RELATED APPLICATIONS

This application is a U.S. National Phase Application filed under 35 U.S.C. § 371 of PCT Application No. PCT/US2020/017889 filed Feb. 12, 2020, which claims priority to U.S. Provisional Application Ser. No. 62/804,358, filed Feb. 12, 2019, each of the applications of which are incorporated by reference in their entireties.

### GOVERNMENT LICENSE RIGHTS

This invention was made with Government support under Agreement HHSO100201700020C, awarded by the U.S. Department of Health and Human Services. The Government has certain rights in the invention.

### FIELD OF THE DISCLOSURE

The present disclosure relates to bispecific antigen binding molecules that bind to a target antigen and a complement component, and uses thereof.

### BACKGROUND

Therapeutics that direct powerful effector functions of the immune system towards target cells hold great promise in settings of therapeutic resistance of infectious diseases and cancer. The complement system is an ancient and well-conserved branch of innate immunity which recognizes and clears pathogens by direct lysis, opsonization, and recruitment of effector cells. Targeting these activities to target organisms and/or cells associated with disease can provide a novel modality to add to the therapeutic arsenal.

Classical complement activation is initiated by the C1 complex, composed of C1q, C1r, and CIs proteins. C1q is a hexameric protein comprised of one stalk domain and six head domains which recognize molecular patterns on cell surfaces, as well as antigen-antibody complexes via binding to IgG1 or IgG3 Fc domains. C1q binds to individual Fc domains with low affinity, but antigen-induced multimerization of Fc domains results in avidity-driven enhancement of C1q binding. The ensuing serine protease cascade results in sequential recruitment and cleavage of complement components that culminates in the insertion of the lytic membrane attack complex (MAC, C5b9) pore causing rapid loss of cell viability. Cell-surface bound C1q can also be directly recognized by C1q binding proteins or a C1q receptor on a variety of effector cells leading to phagocytosis or cytotoxicity.

Directed complement recruitment has not been effectively harnessed for broad therapeutic application. To date, therapeutic antibodies engaging complement do so via their IgG1 Fc domains and therefore potency is dependent on the density and geometry of antibody binding to the target antigen. For example, anti-CD20 IgG1 mAbs, such as rituximab, induce complement dependent cytotoxicity (CDC) [e.g. MAC-mediated lysis] of CD20-expressing B cells in vitro, with potency related to CD20 density. However in vivo, the relative contribution of CDC versus effector cell-mediated death such as antibody-dependent cell-mediated cytotoxicity (ADCC) is unknown.

Thus, there is a need to develop compositions and methods for enhancing complement deposition and mediating cytotoxicity of bacterial, mammalian and virally-infected target cells.

### BRIEF SUMMARY OF THE DISCLOSURE

According to a first aspect, the present disclosure provides bispecific antigen-binding molecules comprising a first antigen-binding domain (D1) that binds a target antigen; and a second antigen-binding domain (D2) that binds a complement component. Classical complement components comprise C1q, C1r, C1s, C2, C3, C4, C5, C6, C7, C8, C9, or any fragments thereof (including proteolytic fragments generated by the complement cascade). In embodiments, the complement component is C1q. In yet other embodiments the C1q is a human C1q.

In some embodiments, the target antigen is: (a) expressed on the surface of a cell or organism; or (b) a soluble antigen. In some embodiments, the target antigen is expressed on the surface of a cell (e.g., a eukaryotic cell, e.g., a human cell) or an organism. In some embodiments, the target antigen is expressed on the surface of the cell, such as but not limited to an immune cell, a blood or hematopoietic cell, a tissue or organ cell, a stem cell, or a precursor cell. In some embodiments, the cell is a diseased cell (e.g., a cancer cell or a bacterially- or virally-infected cell). In some embodiments, the cell is a non-diseased cell.

In embodiments, the target antigen is on the surface of a cell or organism; wherein the target antigen is a marker of a disease state of a subject. In embodiments, the target antigen comprises an antigen found on the surface of a bacteria, on the surface of a virus, on the surface of a bacterial or virus infected cell, on the surface of a cancer cell, or on the surface of an immune cell. In some embodiments, the disease state of the subject comprises a bacterial infection, a viral infection, cancer or an immune dysfunction.

In some embodiments, the target antigen is a soluble antigen that is not bound to a cell, bacteria, or virus, e.g., a soluble multimer.

In embodiments, the bispecific antigen-binding molecule binds to the target antigen with an EC₅₀ of about 10 nM or less. In further embodiments, the bispecific antigen-binding molecule promotes deposition of a complement component on the target antigen with an EC₅₀ of about 10 nM or less.

In embodiments, the target antigen comprises a Staphylococcus species target antigen, a Pseudomonas species target antigen, an Influenza strain target antigen, a B cell antigen, a cancer cell antigen, or a T cell antigen.

In some embodiments, the bispecific antigen-binding molecule binds to a bacterial target antigen. In embodiments, the bacterial target antigen is from a species of bacteria other than a Staphylococcus species. In embodiments, the bacterial target antigen is a Gram-positive bacterial target antigen. In embodiments, the bacterial target antigen is a Gram-positive bacterial target antigen from a species of bacteria other than a Staphylococcus species. In embodiments, the bacterial target antigen is a Gram-negative bacterial target antigen.

In some embodiments, the target antigen comprises a Staphylococcus species target antigen. In embodiments, a Staphylococcus species is Staphylococcus aureus or Staphylococcus epidermidis. In embodiments, the Staphylococcus species target antigen comprises capsular polysaccharide type 5, capsular polysaccharide type 8, protein A, IsdB, IsdA, IsdH, lipoteichoic acid, wall teichoic acid, lipase, V8 lipase, fatty acid modifying enzyme, microbial surface components recognizing adhesive matrix molecules (e.g. adhesins, fibrinogen binding molecules, fibronectin binding protein A, fibronectin binding protein B), clumping factor A (ClfA), poly-N-acetyl glucosamine (PNAG), or combinations thereof. In embodiments, the Staphylococcus species target antigen is iron regulated surface determinant B (IsdB). In embodiments, the Staphylococcus species target antigen is iron regulated surface determinant A (IsdA). In embodiments, the Staphylococcus species target antigen is protein A. In embodiments, the Staphylococcus species target antigen is poly-N-acetyl glucosamine (PNAG). In embodiments, the Staphylococcus species target antigen is clumping factor A (ClfA).

According to certain exemplary embodiments, the bispecific antigen-binding molecules bind to the Staphylococcus species target antigen iron regulated surface determinant B (IsdB) and to the complement component C1q, e.g., human C1q; such bispecific antigen-binding molecules are also referred to herein as “anti-IsdB×anti-C1q bispecific molecules.” The anti-IsdB antigen binding domain of the anti-IsdB×anti-C1q bispecific molecule is useful for targeting microbes that express IsdB (e.g., Staphylococcus aureus), and the anti-C1q antigen binding domain of the bispecific molecule is useful for promoting deposition of C1q on the Staphylococcus species. The simultaneous binding of antigen binding molecules to a target antigen, such as IsdB, on a microbe and to human C1q facilitates directed killing (cell lysis) of the targeted microbe. The anti-IsdB×anti-C1q bispecific antigen-binding molecules of the disclosure are therefore useful, inter alia, for treating diseases and disorders related to or caused by microbes (e.g., infection, sepsis, and the like). Exemplary anti-IsdB antibodies are described in U.S. Ser. No. 16/143,901 and PCT/US2018/053064.

According to certain exemplary embodiments, the bispecific antigen-binding molecules bind to the Staphylococcus species target antigen protein A and to the complement component C1q, e.g., human C1q; such bispecific antigen-binding molecules are also referred to herein as “anti-protein Ax anti-C1q bispecific molecules.” The anti-protein A antigen binding domain of the anti-protein A×anti-C1q bispecific molecule is useful for targeting microbes that express protein A (e.g., Staphylococcus aureus), and the anti-C1q antigen binding domain of the bispecific molecule is useful for promoting deposition of C1q on the Staphylococcus species. The simultaneous binding of antigen binding molecules to a target antigen, such as protein A, on a microbe and to human C1q facilitates directed killing (cell lysis) of the targeted microbe. The anti-protein A×anti-C1q bispecific antigen-binding molecules of the disclosure are therefore useful, inter alia, for treating diseases and disorders related to or caused by microbes (e.g., infection, sepsis, and the like).

According to certain exemplary embodiments, the bispecific antigen-binding molecules bind to the Staphylococcus species target antigen heme binding protein IsdA and to the complement component C1q, e.g., human C1q; such bispecific antigen-binding molecules are also referred to herein as “anti-IsdA×anti-C1q bispecific molecules.” The anti-IsdA antigen binding domain of the anti-IsdA×anti-C1q bispecific molecule is useful for targeting microbes that express IsdA (e.g., Staphylococcus aureus), and the anti-C1q antigen binding domain of the bispecific molecule is useful for promoting deposition of C1q on the Staphylococcus species. The simultaneous binding of antigen binding molecules to a target antigen, such as IsdA, on a microbe and to human C1q facilitates directed killing (cell lysis) of the targeted microbe. The anti-protein A×anti-C1q bispecific antigen-binding molecules of the disclosure are therefore useful, inter alia, for treating diseases and disorders related to or caused by microbes (e.g., infection, sepsis, and the like).

According to certain exemplary embodiments, the bispecific antigen-binding molecules bind to the Staphylococcus species target antigen exopolysaccharide Poly-N-Acetylglucosamine (PNAG) and to the complement component C1q, e.g., human C1q; such bispecific antigen-binding molecules are also referred to herein as “anti-PNAG×anti-C1q bispecific molecules.” The anti-PNAG antigen binding domain of the anti-PNAG×anti-C1q bispecific molecule is useful for targeting microbes that express PNAG (e.g., Staphylococcus aureus), and the anti-C1q antigen binding domain of the bispecific molecule is useful for promoting deposition of C1q on the Staphylococcus species. The simultaneous binding of antigen binding molecules to a target antigen, such as PNAG, on a microbe and to human C1q facilitates directed killing (cell lysis) of the targeted microbe. The anti-PNAG×anti-C1q bispecific antigen-binding molecules of the disclosure are therefore useful, inter alia, for treating diseases and disorders related to or caused by microbes (e.g., infection, sepsis, and the like). Exemplary anti-PNAG antibodies are described in U.S. Pat. No. 7,786,255.

According to certain exemplary embodiments, the bispecific antigen-binding molecules bind to the Staphylococcus species target antigen Clumping factor A (ClfA) and to the complement component C1q, e.g., human C1q; such bispecific antigen-binding molecules are also referred to herein as “anti-ClfA×anti-C1q bispecific molecules.” The anti-ClfA antigen binding domain of the anti-ClfA×anti-C1q bispecific molecule is useful for targeting microbes that express ClfA (e.g., Staphylococcus aureus), and the anti-C1q antigen binding domain of the bispecific molecule is useful for promoting deposition of C1q on the Staphylococcus species. The simultaneous binding of antigen binding molecules to a target antigen, such as ClfA, on a microbe and to human C1q facilitates directed killing (cell lysis) of the targeted microbe. The anti-ClfA×anti-C1q bispecific antigen-binding molecules of the disclosure are therefore useful, inter alia, for treating diseases and disorders related to or caused by microbes (e.g., infection, sepsis, and the like). Exemplary antibodies are described in U.S. Publication No. US2003/0099656.

In some embodiments, the bispecific antigen-binding molecule binds to a Gram-negative bacterial target antigen. In embodiments, the bacterial target antigen is from a species of Pseudomonas. In embodiments, a Pseudomonas species is Pseudomonas aeruginosa PAO-1. In embodiments, the Pseudomonas species target antigen comprises an exopolysaccharide Psl antigen. According to certain exemplary embodiments, the bispecific antigen-binding molecules bind to a Pseudomonas species target antigen Psl and to the complement component C1q, e.g., human C1q; such bispecific antigen-binding molecules are also referred to herein as “anti-Psl×anti-C1q bispecific molecules.” The anti-Psl antigen binding domain of the anti-Psl×anti-C1q bispecific molecule is useful for targeting microbes that express Psl (e.g., Pseudomonas aeruginosa), and the anti-C1q antigen binding domain of the bispecific molecule is useful for promoting deposition of C1q on the Pseudomonas species. The anti-Psl×anti-C1q bispecific antigen-binding molecules of the disclosure are therefore useful, inter alia, for treating diseases and disorders related to or caused by microbes (e.g., infection, sepsis, and the like). Exemplary anti-Psl antibodies are described in WO2012/170807.

In some embodiments, the bispecific antigen-binding molecule binds to a viral target antigen. In embodiments, the viral target antigen is from an enveloped virus such as Influenza. In embodiments, a target antigen on Influenza or an Influenza infected cell is hemagglutinin (HA). In embodiments, the viral target antigen is from a virus strain that is capable of infecting a eukaryotic host, e.g., a human host, such Influenza A or Influenza B. Influenza strains in the A group can be further subtyped by the subtype of HA and the subtype of Neuraminidase (NA). Pathogenic strains include strains having H1, H3, H5, H7, or H9. In embodiments, the viral target antigen is from a pathogenic influenza strain (e.g., a strain having H1, H3, H5, H7, or H9). Exemplary anti-HA antibodies are described in U.S. Publication No. 2016/0176953.

According to certain exemplary embodiments, the bispecific antigen-binding molecules bind to an Influenza target antigen HA and to the complement component C1q, e.g., human C1q; such bispecific antigen-binding molecules are also referred to herein as “anti-HA×anti-C1q bispecific molecules.” The anti-HA antigen binding domain of the anti-HA×anti-C1q bispecific molecule is useful for targeting viruses or viral infected cells that express HA (e.g., Influenza virus A/Puerto Rico/08/1934 (H1N1)), and the anti-C1q antigen binding domain of the bispecific molecule is useful for promoting deposition of C1q on the Influenza strains or Influenza infected cells. The anti-HA×anti-C1q bispecific antigen-binding molecules of the disclosure are therefore useful, inter alia, for treating diseases and disorders related to or caused by Influenza.

In some embodiments, the bispecific antigen-binding molecule binds to a target antigen from a eukaryotic host, e.g., a target antigen that is expressed on the surface of a eukaryotic cell such as an immune cell or a cancer cell, or a soluble target antigen within the eukaryotic host. In embodiments, the target antigen is a cancer cell surface antigen. In embodiments, the target antigen is an immune cell surface antigen, e.g., a T cell surface antigen, a B cell surface antigen, a dendritic cell surface antigen, a lymphocyte surface antigen, a monocyte surface antigen, a macrophage surface antigen, or an antigen on another type of immune cell. In some embodiments, the target cell is an antigen receptor-expressing cell (e.g., a B cell with a B-cell receptor or a T cell with a T-cell receptor) and the target antigen is the receptor expressed on the surface of the target immune cell in the context of its receptor.

According to certain exemplary embodiments, the bispecific antigen-binding molecules bind to a cancer cell surface antigen and to the complement component C1q, e.g., human C1q. Cancer cell surface antigens include antigens found on cancer cells that are unique to the cancer cell or more highly expressed on a cancer. In embodiments, a cancer antigen is CD20 antigen, which is expressed in B cell-derived cancers such as lymphoma and leukemia. Such bispecific antigen-binding molecules are also referred to herein as “anti-CD20×anti-C1q bispecific molecules.” The anti-CD20 antigen binding domain of the anti-CD20×anti-C1q bispecific molecule is useful for targeting cancer cells that express CD20, and the anti-C1q antigen binding domain of the bispecific molecule is useful for promoting deposition of C1q on the cancer cells. The anti-CD20×anti-C1q bispecific antigen-binding molecules of the disclosure are therefore useful, inter alia, for treating cancer. Exemplary anti-CD20 antibodies are described in U.S. Pat. No. 9,657,102, US Publication No. 2015/0266966, and US Publication No. 2018/0194841.

According to certain exemplary embodiments, the bispecific antigen-binding molecules bind to a T cell surface antigen and to the complement component C1q, e.g., human C1q. T cell surface antigens include antigens found on regulatory T cells. In embodiments, a T cell antigen is a glucocorticoid induced tumor necrosis factor receptor (GITR) antigen found on regulatory T cells. Such bispecific antigen-binding molecules are also referred to herein as “anti-GITR×anti-C1q bispecific molecules.” The anti-GITR antigen binding domain of the anti-GITR×anti-C1q bispecific molecule is useful for targeting T cells that express GITR, and the anti-C1q antigen binding domain of the bispecific molecule is useful for promoting deposition of C1q on the T cells. The anti-GITR×anti-C1q bispecific antigen-binding molecules of the disclosure are therefore useful, inter alia, for reducing or depleting immune cells such as regulatory T cells. Exemplary anti-GITR antibodies are described in U.S. Publication No. US2017/0355774.

The bispecific antigen-binding molecules according to this aspect of the present disclosure comprise a first antigen-binding domain (D1) that specifically binds a target antigen, and a second antigen-binding domain (D2) that specifically binds human C1q. The present disclosure includes anti-C1q bispecific molecules (e.g., bispecific antibodies) wherein each antigen-binding domain comprises a heavy chain variable region (HCVR) paired with a light chain variable region (LCVR). In certain exemplary embodiments of the disclosure, the anti-C1q antigen-binding domain (D2) and the anti-target (D1) antigen-binding domain each comprise different, distinct HCVRs paired with the same or a different LCVR. In the exemplary molecules disclosed herein, the pairing of an HCVR from an anti-target antibody with an LCVR having the sequences described herein in Table 18 creates an antigen-binding domain that specifically binds the target antigen (but does not bind C1q). In the exemplary molecules disclosed herein, the pairing of an HCVR from an anti-C1q antibody with an LCVR having the sequences described herein in Table 18 creates an antigen-binding domain that specifically binds C1q (but does not bind IsdB).

An antigen-binding molecule, as used in the context of the present disclosure, includes polypeptides that bind a particular antigen (e.g., a target molecule [T] or a portion thereof) with a K_{D} of less than about 25 nM, less than about 20 nM, less than about 15 nM, less than about 10 nM, less than about 5 nM, less than about 1 nM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured in a surface plasmon resonance assay.

An antigen-binding domain includes polypeptides that bind a complement component, such as C1q, with a K_{D} of less than about 25 nM, less than about 20 nM, less than about 15 nM, less than about 10 nM, less than about 5 nM, less than about 1 nM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured in a surface plasmon resonance assay.

In embodiments, the second antigen-binding domain D2 that specifically binds C1q comprises heavy chain variable region (HCVR) CDRs contained within a HCVR comprising an amino acid sequence of SEQ ID NO: 2 or an amino acid sequence of SEQ ID NO: 18; and light chain variable region (LCVR) CDRs contained within a LCVR region comprising an amino acid sequence of SEQ ID NO: 10. In embodiments, the second antigen-binding domain D2 comprises any of the HCVR/HCDR amino acid sequences as set forth in Table 1. The second antigen-binding domain D2 may also comprise any of the LCVR/LCDR amino acid sequences as set forth in Table 1. According to certain embodiments, the second antigen-binding domain D2 comprises a HCVR/LCVR pair comprising amino acid sequences of SEQ ID NOs:2/10, or SEQ ID NOs: 18/10. The present disclosure also provides anti-C1q bispecific molecules, wherein the second antigen-binding domain D2 comprises any of the heavy chain CDR1-CDR2-CDR3 amino acid sequences as set forth in Table 1, and/or any of the light chain CDR1-CDR2-CDR3 amino acid sequences as set forth in Table 1.

According to certain embodiments, the present disclosure provides anti-C1q bispecific molecules, wherein the second antigen-binding domain D2 comprises a heavy chain variable region (HCVR) comprising an amino acid sequence of SEQ ID NO: 2, or SEQ ID NO: 18, or a substantially similar sequence thereof having at least 90%, at least 95%, at least 98% or at least 99% sequence identity.

The present disclosure also provides anti-C1q bispecific molecules, wherein the second antigen-binding domain D2 comprises a light chain variable region (LCVR) comprising an amino acid sequence of SEQ ID NO:10, or a substantially similar sequence thereof having at least 90%, at least 95%, at least 98% or at least 99% sequence identity.

The present disclosure also provides anti-C1q bispecific antigen binding molecules, wherein the second antigen-binding domain D2 specifically binds a human C1q. In embodiments, the bispecific antigen-binding molecule blocks the binding of the human C1q to human IgG1-k with an IC₅₀ of about 10 nM or less. In other embodiments, the bispecific antigen-binding molecule blocks the binding of the human C1q to human IgM with an IC₅₀ of about 50 nM or less. In embodiments, the bispecific antigen binding molecule provides for deposition of C1q with an EC₅₀ of about 10 nM or less.

In embodiments, the first antigen-binding domain D1 specifically binds IsdB. Exemplary first antigen binding domains comprise HCVR CDRs contained within a HCVR comprising an amino acid sequence of SEQ ID NO: 26 or substantially similar sequence thereof having at least 90%, at least 95%, at least 98% or at least 99% sequence identity.

The present disclosure also provides anti-IsdB×anti-C1q bispecific molecules, wherein the first antigen-binding domain D1 comprises LCVR CDRs contained within a LCVR comprising an amino acid sequence of SEQ ID NO: 10, or a substantially similar sequence thereof having at least 90%, at least 95%, at least 98% or at least 99% sequence identity.

The present disclosure also provides anti-IsdB×anti-C1q bispecific molecules, wherein the first antigen-binding domain D1 comprises a HCVR and LCVR (HCVR/LCVR) pair comprising amino acid sequences of SEQ ID NOs: 26/10.

The present disclosure also provides anti-IsdB×anti-C1q bispecific antigen-binding molecules, wherein the first antigen-binding domain D1 comprises a heavy chain CDR1 (HCDR1) comprising an amino acid sequence of SEQ ID NO: 28, or a substantially similar sequence thereof having at least 90%, at least 95%, at least 98% or at least 99% sequence identity; a heavy chain CDR2 (HCDR2) comprising an amino acid sequence of SEQ ID NO: 30, or a substantially similar sequence thereof having at least 90%, at least 95%, at least 98% or at least 99% sequence identity; and a heavy chain CDR3 (HCDR3) comprising an amino acid sequence of SEQ ID NO:32, or a substantially similar sequence thereof having at least 90%, at least 95%, at least 98% or at least 99% sequence identity. In embodiments, the first antigen-binding domain D1 comprises a light chain CDR1 (LCDR1) comprising an amino acid sequence of SEQ ID NO:12 or a substantially similar sequence thereof having at least 90%, at least 95%, at least 98% or at least 99% sequence identity; a light chain CDR2 (LCDR2) comprising an amino acid sequence of SEQ ID NO:14 or a substantially similar sequence thereof having at least 90%, at least 95%, at least 98% or at least 99% sequence identity; and a light chain CDR3 (LCDR3) comprising an amino acid sequence of SEQ ID NO:16 or a substantially similar sequence thereof having at least 90%, at least 95%, at least 98% or at least 99% sequence identity.

In embodiments, the first antigen-binding domain D1 specifically binds Protein A. Exemplary first antigen binding domains comprises HCVR CDRs contained within a HCVR comprising an amino acid sequence of SEQ ID NO: 58 or substantially similar sequence thereof having at least 90%, at least 95%, at least 98% or at least 99% sequence identity.

The present disclosure also provides anti-protein A×anti-C1q bispecific molecules, wherein the first antigen-binding domain D1 comprises LCVR CDRs contained within a LCVR comprising an amino acid sequence of SEQ ID NO: 10, or a substantially similar sequence thereof having at least 90%, at least 95%, at least 98% or at least 99% sequence identity.

The present disclosure also provides anti-protein A×anti-C1q bispecific molecules, wherein the first antigen-binding domain D1 comprises a HCVR and LCVR (HCVR/LCVR) pair comprising amino acid sequences of SEQ ID NOs: 58/10.

The present disclosure also provides anti-protein A×anti-C1q bispecific antigen-binding molecules, wherein the first antigen-binding domain D1 comprises a heavy chain CDR1 (HCDR1) comprising an amino acid sequence of SEQ ID NO: 60, or a substantially similar sequence thereof having at least 90%, at least 95%, at least 98% or at least 99% sequence identity; a heavy chain CDR2 (HCDR2) comprising an amino acid sequence of SEQ ID NO: 62, or a substantially similar sequence thereof having at least 90%, at least 95%, at least 98% or at least 99% sequence identity; and a heavy chain CDR3 (HCDR3) comprising an amino acid sequence of SEQ ID NO:64 or a substantially similar sequence thereof having at least 90%, at least 95%, at least 98% or at least 99% sequence identity. In embodiments, the first antigen-binding domain D1 comprises a light chain CDR1 (LCDR1) comprising an amino acid sequence of SEQ ID NO:12 or a substantially similar sequence thereof having at least 90%, at least 95%, at least 98% or at least 99% sequence identity; a light chain CDR2 (LCDR2) comprising an amino acid sequence of SEQ ID NO:14 or a substantially similar sequence thereof having at least 90%, at least 95%, at least 98% or at least 99% sequence identity; and a light chain CDR3 (LCDR3) comprising an amino acid sequence of SEQ ID NO:16 or a substantially similar sequence thereof having at least 90%, at least 95%, at least 98% or at least 99% sequence identity.

In embodiments, the present disclosure provides anti-IsdA×anti-C1q bispecific antibodies. In embodiments, the first antigen-binding domain D1 comprises HCVR CDRs contained within a HCVR comprising an amino acid sequence of SEQ ID NO: 66 or substantially similar sequence thereof having at least 90%, at least 95%, at least 98% or at least 99% sequence identity.

The present disclosure also provides anti-IsdA×anti-C1q bispecific molecules, wherein the first antigen-binding domain D1 comprises LCVR CDRs contained within a LCVR comprising an amino acid sequence of SEQ ID NO: 10, or a substantially similar sequence thereof having at least 90%, at least 95%, at least 98% or at least 99% sequence identity.

The present disclosure also provides anti-IsdA×anti-C1q bispecific molecules, wherein the first antigen-binding domain D1 comprises a HCVR and LCVR (HCVR/LCVR) pair comprises amino acid sequences of SEQ ID NOs: 66/10.

The present disclosure also provides anti-IsdA×anti-C1q bispecific antigen-binding molecules, wherein the first antigen-binding domain D1 comprises a heavy chain CDR1 (HCDR1) comprising an amino acid sequence of SEQ ID NO: 68, or a substantially similar sequence thereof having at least 90%, at least 95%, at least 98% or at least 99% sequence identity; a heavy chain CDR2 (HCDR2) comprising an amino acid sequence of SEQ ID NO: 70, or a substantially similar sequence thereof having at least 90%, at least 95%, at least 98% or at least 99% sequence identity; and a heavy chain CDR3 (HCDR3) comprising an amino acid sequence of SEQ ID NO:72 or a substantially similar sequence thereof having at least 90%, at least 95%, at least 98% or at least 99% sequence identity. In embodiments, the first antigen-binding domain D1 comprises a light chain CDR1 (LCDR1) comprising an amino acid sequence of SEQ ID NO:12 or a substantially similar sequence thereof having at least 90%, at least 95%, at least 98% or at least 99% sequence identity; a light chain CDR2 (LCDR2) comprising an amino acid sequence of SEQ ID NO:14 or a substantially similar sequence thereof having at least 90%, at least 95%, at least 98% or at least 99% sequence identity; and a light chain CDR3 (LCDR3) comprising an amino acid sequence of SEQ ID NO:16 or a substantially similar sequence thereof having at least 90%, at least 95%, at least 98% or at least 99% sequence identity.

In embodiments, the present disclosure provides anti-ClfA×anti-C1q bispecific antibodies. In embodiments, the first antigen-binding domain D1 comprises HCVR CDRs contained within a heavy chain (HC) comprising an amino acid sequence of SEQ ID NO: 73 or a substantially similar sequence thereof having at least 90%, at least 95%, at least 98% or at least 99% sequence identity.

The present disclosure also provides anti-ClfA×anti-C1q bispecific molecules, wherein the first antigen-binding domain D1 comprises LCVR CDRs contained within a light chain (LC) comprising an amino acid sequence of SEQ ID NO: 74, or a substantially similar sequence thereof having at least 90%, at least 95%, at least 98% or at least 99% sequence identity.

The present disclosure also provides anti-ClfA×anti-C1q bispecific molecules, wherein the first antigen-binding domain D1 comprises a HCVR and LCVR (HCVR/LCVR) pair comprising amino acid sequences contained within the HC/LC amino acid sequence pair of SEQ ID NOs: 73/74.

In embodiments, the present disclosure provides anti-PNAG×anti-C1q bispecific antibodies. In embodiments, the first antigen-binding domain D1 comprises HCVR CDRs contained within a HC comprising an amino acid sequence of SEQ ID NO: 76 or a substantially similar sequence thereof having at least 90%, at least 95%, at least 98% or at least 99% sequence identity.

The present disclosure also provides anti-PNAG×anti-C1q bispecific molecules, wherein the first antigen-binding domain D1 comprises LCVR CDRs contained within a LC comprising an amino acid sequence of SEQ ID NO: 77, or a substantially similar sequence thereof having at least 90%, at least 95%, at least 98% or at least 99% sequence identity.

The present disclosure also provides anti-PNAG×anti-C1q bispecific molecules, wherein the first antigen-binding domain D1 comprises a HCVR and LCVR (HCVR/LCVR) pair comprising amino acid sequences contained within the HC/LC amino acid sequence pair of SEQ ID NOs: 76/77.

In embodiments, the present disclosure provides anti-Psl×anti-C1q bispecific antibodies. In embodiments, the first antigen-binding domain D1 comprises HCVR CDRs contained within a HC comprising an amino acid sequence of SEQ ID NO: 78 or a substantially similar sequence thereof having at least 90%, at least 95%, at least 98% or at least 99% sequence identity.

The present disclosure also provides anti-Psl×anti-C1q bispecific molecules, wherein the first antigen-binding domain D1 comprises LCVR CDRs contained within a LC comprising an amino acid sequence of SEQ ID NO: 79, or a substantially similar sequence thereof having at least 90%, at least 95%, at least 98% or at least 99% sequence identity.

The present disclosure also provides anti-Psl×anti-C1q bispecific molecules, wherein the first antigen-binding domain D1 comprises a HCVR and LCVR (HCVR/LCVR) pair comprising amino acid sequences contained within the HC/LC amino acid sequence pair of SEQ ID NOs: 78/79.

In embodiments, the present disclosure provides anti-CD20×anti-C1q bispecific antibodies. In embodiments, the first antigen-binding domain D1 comprises HCVR CDRs contained within a HCVR comprising an amino acid sequence of SEQ ID NO: 34 or a substantially similar sequence thereof having at least 90%, at least 95%, at least 98% or at least 99% sequence identity.

The present disclosure also provides anti-CD20×anti-C1q bispecific molecules, wherein the first antigen-binding domain D1 comprises LCVR CDRs contained within a LCVR comprising an amino acid sequence of SEQ ID NO: 10, or a substantially similar sequence thereof having at least 90%, at least 95%, at least 98% or at least 99% sequence identity.

The present disclosure also provides anti-CD20×anti-C1q bispecific molecules, wherein the first antigen-binding domain D1 comprises a HCVR and LCVR (HCVR/LCVR) pair comprising amino acid sequences of SEQ ID NOs: 34/10.

The present disclosure also provides anti-CD20×anti-C1q bispecific antigen-binding molecules, wherein the first antigen-binding domain D1 comprises a heavy chain CDR1 (HCDR1) comprising an amino acid sequence of SEQ ID NO: 36, or a substantially similar sequence thereof having at least 90%, at least 95%, at least 98% or at least 99% sequence identity; a heavy chain CDR2 (HCDR2) comprising an amino acid sequence of SEQ ID NO: 38, or a substantially similar sequence thereof having at least 90%, at least 95%, at least 98% or at least 99% sequence identity; and a heavy chain CDR3 (HCDR3) comprising an amino acid sequence of SEQ ID NO:40 or a substantially similar sequence thereof having at least 90%, at least 95%, at least 98% or at least 99% sequence identity. In embodiments, the first antigen-binding domain D1 comprises a light chain CDR1 (LCDR1) comprising an amino acid sequence of SEQ ID NO:12 or a substantially similar sequence thereof having at least 90%, at least 95%, at least 98% or at least 99% sequence identity; a light chain CDR2 (LCDR2) comprising an amino acid sequence of SEQ ID NO:14 or a substantially similar sequence thereof having at least 90%, at least 95%, at least 98% or at least 99% sequence identity; and a light chain CDR3 (LCDR3) comprising an amino acid sequence of SEQ ID NO:16 or a substantially similar sequence thereof having at least 90%, at least 95%, at least 98% or at least 99% sequence identity.

In embodiments, the present disclosure provides anti-HA×anti-C1q bispecific antibodies. In embodiments, the first antigen-binding domain D1 comprises HCVR CDRs contained within a HCVR comprising an amino acid sequence of SEQ ID NO: 42 or substantially similar sequence thereof having at least 90%, at least 95%, at least 98% or at least 99% sequence identity.

The present disclosure also provides anti-HA×anti-C1q bispecific molecules, wherein the first antigen-binding domain D1 comprises LCVR CDRs contained within a LCVR comprising an amino acid sequence of SEQ ID NO: 10, or a substantially similar sequence thereof having at least 90%, at least 95%, at least 98% or at least 99% sequence identity.

The present disclosure also provides anti-HA×anti-C1q bispecific molecules, wherein the first antigen-binding domain D1 comprises a HCVR and LCVR (HCVR/LCVR) pair comprising amino acid sequences of SEQ ID NOs: 42/10.

The present disclosure also provides anti-HA×anti-C1q bispecific antigen-binding molecules, wherein the first antigen-binding domain D1 comprises a heavy chain CDR1 (HCDR1) comprising an amino acid sequence of SEQ ID NO: 44, or a substantially similar sequence thereof having at least 90%, at least 95%, at least 98% or at least 99% sequence identity; a heavy chain CDR2 (HCDR2) comprising an amino acid sequence of SEQ ID NO: 46, or a substantially similar sequence thereof having at least 90%, at least 95%, at least 98% or at least 99% sequence identity; and a heavy chain CDR3 (HCDR3) comprising an amino acid sequence of SEQ ID NO:48 or a substantially similar sequence thereof having at least 90%, at least 95%, at least 98% or at least 99% sequence identity. In embodiments, the first antigen-binding domain D1 comprises a light chain CDR1 (LCDR1) comprising an amino acid sequence of SEQ ID NO:12 or a substantially similar sequence thereof having at least 90%, at least 95%, at least 98% or at least 99% sequence identity; a light chain CDR2 (LCDR2) comprising an amino acid sequence of SEQ ID NO:14 or a substantially similar sequence thereof having at least 90%, at least 95%, at least 98% or at least 99% sequence identity; and a light chain CDR3 (LCDR3) comprising an amino acid sequence of SEQ ID NO:16 or a substantially similar sequence thereof having at least 90%, at least 95%, at least 98% or at least 99% sequence identity.

In embodiments, the present disclosure provides anti-GITR×anti-C1q bispecific antibodies. In embodiments, the first antigen-binding domain D1 comprises HCVR CDRs contained within a HCVR comprising an amino acid sequence of SEQ ID NO: 50 or substantially similar sequence thereof having at least 90%, at least 95%, at least 98% or at least 99% sequence identity.

The present disclosure also provides anti-GITR×anti-C1q bispecific molecules, wherein the first antigen-binding domain D1 comprises LCVR CDRs contained within a LCVR comprising an amino acid sequence of SEQ ID NO: 10, or a substantially similar sequence thereof having at least 90%, at least 95%, at least 98% or at least 99% sequence identity.

The present disclosure also provides anti-GITR×anti-C1q bispecific molecules, wherein the first antigen-binding domain D1 comprises a HCVR and LCVR (HCVR/LCVR) pair comprising amino acid sequences of SEQ ID NOs: 50/10.

The present disclosure also provides anti-GITR×anti-C1q bispecific antigen-binding molecules, wherein the first antigen-binding domain D1 comprises a heavy chain CDR1 (HCDR1) comprising an amino acid sequence of SEQ ID NO: 52, or a substantially similar sequence thereof having at least 90%, at least 95%, at least 98% or at least 99% sequence identity; a heavy chain CDR2 (HCDR2) comprising an amino acid sequence of SEQ ID NO:54, or a substantially similar sequence thereof having at least 90%, at least 95%, at least 98% or at least 99% sequence identity; and a heavy chain CDR3 (HCDR3) comprising an amino acid sequence of SEQ ID NO: 56 or a substantially similar sequence thereof having at least 90%, at least 95%, at least 98% or at least 99% sequence identity. In embodiments, the first antigen-binding domain D1 comprises a light chain CDR1 (LCDR1) comprising an amino acid sequence of SEQ ID NO:12 or a substantially similar sequence thereof having at least 90%, at least 95%, at least 98% or at least 99% sequence identity; a light chain CDR2 (LCDR2) comprising an amino acid sequence of SEQ ID NO:14 or a substantially similar sequence thereof having at least 90%, at least 95%, at least 98% or at least 99% sequence identity; and a light chain CDR3 (LCDR3) comprising an amino acid sequence of SEQ ID NO:16 or a substantially similar sequence thereof having at least 90%, at least 95%, at least 98% or at least 99% sequence identity.

In another aspect, the present disclosure provides isolated bispecific antigen binding molecules comprising: (a) a first antigen-binding domain that specifically binds a target antigen; and (b) a second antigen-binding domain that specifically binds human C1q. In some embodiments, the target antigen is (a) expressed on the surface of a cell or organism; or (b) a soluble antigen. In some embodiments, the second antigen-binding domain comprises a HCDR1 comprising an amino acid sequence selected from the group consisting of SEQ ID NO:4 and SEQ ID NO:20, a HCDR2 comprising an amino acid sequence selected from the group consisting of SEQ ID NO:6 and SEQ ID NO:22, and a HCDR3 comprising an amino acid sequence selected from the group consisting of SEQ ID NO:8 and SEQ ID NO:24. In some embodiments, the second antigen-binding domain comprises a HCDR1 comprising the amino acid sequence of SEQ ID NO:4, a HCDR2 comprising the amino acid sequence of SEQ ID NO:6, and a HCDR3 comprising the amino acid sequence of SEQ ID NO:8; or a HCDR1 comprising the amino acid sequence of SEQ ID NO:20, a HCDR2 comprising the amino acid sequence of SEQ ID NO:22, and a HCDR3 comprising the amino acid sequence of SEQ ID NO:24. In some embodiments, the second antigen-binding domain comprises a heavy chain variable region comprising the amino acid sequence of SEQ ID NO:2 or SEQ ID NO:18.

In some embodiments, the first antigen-binding domain comprises a heavy chain comprising:
a HCDR1 comprising the amino acid sequence of SEQ ID NO:28, a HCDR2 comprising the amino acid sequence of SEQ ID NO:30, and a HCDR3 comprising the amino acid sequence of SEQ ID NO:32; or
a HCDR1 comprising the amino acid sequence of SEQ ID NO:36, a HCDR2 comprising the amino acid sequence of SEQ ID NO:38, and a HCDR3 comprising the amino acid sequence of SEQ ID NO:40; or
a HCDR1 comprising the amino acid sequence of SEQ ID NO:44, a HCDR2 comprising the amino acid sequence of SEQ ID NO:46, and a HCDR3 comprising the amino acid sequence of SEQ ID NO:48; or
a HCDR1 comprising the amino acid sequence of SEQ ID NO:52, a HCDR2 comprising the amino acid sequence of SEQ ID NO:54, and a HCDR3 comprising the amino acid sequence of SEQ ID NO:56; or
a HCDR1 comprising the amino acid sequence of SEQ ID NO:60, a HCDR2 comprising the amino acid sequence of SEQ ID NO:62, and a HCDR3 comprising the amino acid sequence of SEQ ID NO:64; or
a HCDR1 comprising the amino acid sequence of SEQ ID NO:68, a HCDR2 comprising the amino acid sequence of SEQ ID NO:70, and a HCDR3 comprising the amino acid sequence of SEQ ID NO:72; or
a HCDR1 comprising amino acids 26-33 of SEQ ID NO:73, a HCDR2 comprising amino acids 51-58 of SEQ ID NO:73, and a HCDR3 comprising amino acids 96-106 of SEQ ID NO:73; or
a HCDR1 comprising amino acids 27-32 of SEQ ID NO:76, a HCDR2 comprising amino acids 51-58 of SEQ ID NO:76, and a HCDR3 comprising amino acids 96-106 of SEQ ID NO:76; or
a HCDR1 comprising amino acids 31-35 of SEQ ID NO:78, a HCDR2 comprising amino acids 50-65 of SEQ ID NO:78, and a HCDR3 comprising amino acids 98-109 of SEQ ID NO:78.

In some embodiments, the bispecific antigen binding molecule further comprises a light chain comprising:
a LCDR1 comprising the amino acid sequence of SEQ ID NO:12, a LCDR2 comprising the amino acid sequence of SEQ ID NO:14, and a LCDR3 comprising the amino acid sequence of SEQ ID NO:16; or
a LCDR1 comprising amino acids 27-32 of SEQ ID NO:74, a LCDR2 comprising amino acids 51-55 of SEQ ID NO:74, and a LCDR3 comprising amino acids 89-98 of SEQ ID NO:74; or
a LCDR1 comprising amino acids 27-32 of SEQ ID NO:77, a LCDR2 comprising amino acids 50-52 of SEQ ID NO:77, and a LCDR3 comprising amino acids 98-101 of SEQ ID NO:77; or
a LCDR1 comprising amino acids 23-33 of SEQ ID NO:79, a LCDR2 comprising amino acids 49-55 of SEQ ID NO:79, and a LCDR3 comprising amino acids 88-99 of SEQ ID NO:79.

In some embodiments, the light chain comprises a LCDR1 comprising the amino acid sequence of SEQ ID NO:12, a LCDR2 comprising the amino acid sequence of SEQ ID NO:14, and a LCDR3 comprising the amino acid sequence of SEQ ID NO:16. In some embodiments, the light chain variable region comprises the amino acid sequence of SEQ ID NO:10.

In another aspect, the present disclosure provides nucleic acid molecules encoding any of the HCVR, LCVR or CDR sequences of the anti-C1q bispecific antigen-binding molecules disclosed herein, including nucleic acid molecules coding for the amino acid sequences set forth in Table 18. In other embodiments, molecules comprise one or more of the polynucleotide sequences as set forth in Tables 2, 4, 6, 8, 10, 12, 13 and 15 in any functional combination or arrangement thereof. Recombinant expression vectors carrying the nucleic acids of the disclosure, and host cells into which such vectors have been introduced, are also encompassed by the disclosure, as are methods of producing the antibodies by culturing the host cells under conditions permitting production of the antibodies, and recovering the antibodies produced.

The present disclosure includes anti-C1q bispecific antigen-binding molecules wherein any of the aforementioned antigen-binding domains that specifically bind C1q are combined, connected or otherwise associated with any of the aforementioned target antigen-binding domains to form a bispecific antigen-binding molecule that binds C1q and a target antigen. The antigen-binding domains can be combined in a number of different ways including single chain Fv, VH or VL domain antibodies, VHH, VNAR based nanobodies and other scaffold proteins, dual affinity retargeting proteins, tandem diabodies, knob and hole pairings, charge pairings, cross Mab, double variable domain Ig, s-Fab, Fv-Fc, Tandem Fabs (2+2 and 3+3 Tandem Fabs), 2+1 N-Fab, and 2+1 C-Fabs as described herein.

The present disclosure includes anti-C1q bispecific antigen-binding molecules having a modified glycosylation pattern. In some applications, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC) (e.g. adding a terminal galactose enhances ADCC).

In another aspect, the disclosure provides a pharmaceutical composition comprising an anti-C1q bispecific antigen-binding molecule as disclosed herein and a pharmaceutically acceptable carrier. In a related aspect, the disclosure features a composition, which is a combination of an anti-C1q bispecific antigen-binding molecule and a second therapeutic agent. In one embodiment, the second therapeutic agent is any agent that is advantageously combined with an anti-C1q bispecific antigen-binding molecule. Exemplary agents that may be advantageously combined with an anti-C1q bispecific antigen-binding molecule include an antibiotic, an antibody that binds a Staphylococcus antigen, an antibody that binds a Staphylococcus toxin, an antibody that binds a Pseudomonas antigen, an antibody that binds an Influenza antigen, an anti-viral agent, a vaccine for Influenza, anti-tumor cell antibody, anti-T cell antibody, an antibody that binds human or cynomolgus C1q, a vaccine specific for Staphylococcus species, a cancer vaccine, and an antibody drug conjugate (e.g. a bispecific antibody combined with an antibiotic).

In another aspect, the disclosure provides methods for activating or enhancing cell killing comprising contacting the cell with an isolated bispecific antigen binding molecule as disclosed herein or a pharmaceutical composition comprising the bispecific antigen binding molecule. In embodiments, the cell is a bacterial cell. In embodiments, the cell is a eukaryotic cell, e.g., a human cell. In embodiments, the cell is a bacterially infected eukaryotic cell (e.g., a cell infected with Staphylococcus or Pseudomonas). In embodiments, the cell is a virally infected eukaryotic cell (e.g., a cell infected with Influenza virus). In embodiments, the cell is a cancer cell. In embodiments, the cell is a non-diseased cell (e.g., an immune cell, a progenitor cell, a stem cell, or another type of cell disclosed herein). In embodiments, the cell expresses one or more target antigens as disclosed herein. In embodiments, the contacting step is carried out in vitro. In embodiments, the contacting step is carried out in vivo. In embodiments, the contacting step is carried out ex vivo.

In yet another aspect, the disclosure provides therapeutic methods for inhibiting the growth of a bacteria (e.g., Staphylococcus species) in a subject comprising administering the isolated bispecific antigen-binding molecule that specifically binds a Staphylococcus target antigen as described herein or a pharmaceutical composition comprising the bispecific antigen binding molecule to the subject. In embodiments, the method is a method of treating an infection with a Staphylococcus species in a subject. In some embodiments, the method is for treating a subject that has a nosocomial or hospital acquired infection caused by a Staphylococcus species. In some embodiments, the method is for treating a subject that has a disease or disorder caused by a Staphylococcus species, wherein the disease or disorder is a skin infection, cellulitis, pneumonia, meningitis, urinary tract infection, toxic shock syndrome, endocarditis, pericarditis, osteomyelitis, bacteremia, or sepsis. In some embodiments, the method is for treating a subject who has undergone, or who is planning on undergoing a surgical procedure, wherein the subject is at risk of acquiring a Staphylococcus infection. In some embodiments, the surgical procedure involves implantation of a prosthetic, including a prosthetic limb, such as an arm or a leg, or a hip replacement. In some embodiments, the prosthetic is a cosmetic prosthetic, including an ocular prosthetic, silicone hands, fingers, toes, breasts, or a facial implant.

In yet another aspect, the disclosure provides therapeutic methods for inhibiting the growth of a bacteria (e.g., Pseudomonas species) in a subject comprising administering the isolated bispecific antigen-binding molecule that specifically binds to a Pseudomonas target antigen as described herein or a pharmaceutical composition comprising the bispecific antigen binding molecule to the subject. In embodiments, the method is a method of treating an infection with a Pseudomonas species in a subject. In some embodiments, the method is for treating a subject that has a disease or disorder caused by a Pseudomonas species. In some embodiments, the method is for treating a subject that has a nosocomial or hospital acquired infection caused by a Pseudomonas species. In some embodiments, the method is for treating a subject who has undergone, or who is planning on undergoing a surgical procedure, wherein the subject is at risk of acquiring a Pseudomonas infection. In some embodiments, the surgical procedure involves implantation of a prosthetic, including a prosthetic limb, such as an arm or a leg, or a hip replacement. In some embodiments, the prosthetic is a cosmetic prosthetic, including an ocular prosthetic, silicone hands, fingers, toes, breasts, or a facial implant.

In yet another aspect, the disclosure provides therapeutic methods for inhibiting the growth of an Influenza strain in a subject comprising administering the isolated bispecific antigen-binding molecule that specifically binds to an Influenza target antigen as described herein or a pharmaceutical composition comprising the bispecific antigen binding molecule to the subject. In embodiments, the method is a method of treating an infection with Influenza strain in a subject.

In yet another aspect, the disclosure provides therapeutic methods for treating cancer in a subject comprising administering the isolated bispecific antigen-binding molecule that specifically binds to cancer cell specific target antigen as described herein or a pharmaceutical composition comprising the bispecific antigen binding molecule to the subject. In embodiments, the cancer cell specific antigen is a B cell antigen found on leukemia or lymphoma cells. In embodiments, the cancer specific target antigen is CD20.

In yet another aspect, the disclosure provides therapeutic methods for treating cancer in a subject comprising administering the isolated bispecific antigen-binding molecule that specifically binds to a T regulatory cell antigen as described herein or a pharmaceutical composition comprising the bispecific antigen binding molecule to the subject. In embodiments, the T regulatory cell specific antigen is GITR.

The present disclosure also includes the use of an anti-C1q bispecific antigen-binding molecule of the disclosure in the manufacture of a medicament for the treatment of a disease or disorder.

Other embodiments will become apparent from a review of the ensuing detailed description.

### BRIEF DESCRIPTION OF THE FIGURES

FIG. 1. S. aureus incubation with normal human serum (NHS) results in a change to the cell surface. S. aureus was incubated with 50% NHS (A, top) or media (B, bottom) for 8 h and then visualized by scanning electron microscopy (SEM).

FIG. 2. A complete terminal complement pathway is necessary for C5b9 formation and NHS mediated reduction in the growth of S. aureus. The effect of human serum on growth of S. aureus was measured using serum killing assays. S. aureus was incubated with 50% of the indicated sera or medium for 24 h. After incubation bacteria were enumerated by serial dilution and plating. Results are plotted as mean with standard deviation. * P<0.05, *** P<0.001, **** P<0.0001, one-way ANOVA with Dunnett's test showing significance compared to the NHS sample.

FIG. 3. S. aureus treatment with NHS leads to reduced cell viability. S. aureus was incubated with 50% NHS, 50% C1q-depleted serum, or 50% C5-depleted serum for 0, 10, and 24 h at 37° C. Uptake of the viability dye propidium iodide (PI) was assessed using by measuring fluorescence (excitation 533 nm, emission 617 nm) on a 96-well plate reader. Results are plotted as mean with standard deviation. * P<0.05, **** P<0.001, two-way ANOVA showing values significantly different than NHS.

FIG. 4. A complete terminal complement pathway is necessary for NHS mediated reduction in the growth of S. aureus as measured using BacTiter-Glo™ assay. The effect of human serum on growth of S. aureus was measured using serum killing assays. S. aureus was incubated with 50% of the indicated sera or medium for 24 h. After incubation bacterial concentration was measured using the BacTiter-Glo™ assay, a luminescent assay which measures the number of viable cells in a culture via ATP release. Increased luminescence corresponds to higher concentrations of bacteria. Results are plotted as mean with standard deviation. *** P<0.001, **** P<0.0001, one-way ANOVA with Dunnett's test showing significance compared to the NHS sample.

FIG. 5. C1q deposition on S. aureus was measured using a plate-based format. S. aureus Newman was coated onto plates and then incubated with normal human serum in the presence of anti-IsdB×anti-C1q bsAb, an anti-IsdB bivalent antibody, or an isotype control antibody. C1q deposition was measured with a primary antibody specific for C1q and a horse-radish peroxidase (HRP)-conjugated secondary antibody. Results are plotted as mean±standard deviation.

FIG. 6. Whole blood assays were performed by incubating S. aureus Newman with blood alone (far left red column), blood with an anti-IsdB bivalent antibody (columns 2-7; purple) or blood with anti-IsdB×anti-C1q bsAb (columns 8-13; orange). The antibodies were combined with either a C5 blocking Fab (columns 3 and 9), an isotype control Fab (columns 4 and 10), or cytochalasin D (columns 5 and 11), both the C5-blocking antibody and cytochalasin D (columns 6 and 12) or both the isotype control Fab and cytochalasin D (columns 7 and 13). Bacterial growth was enumerated by plating and is represented as a percent increase in colony forming units (CFUs) present after 24 h compared to the initial number of CFUs. Results are plotted as mean with standard deviation. **** P<0.0001, one-way ANOVA with Dunnett's test showing samples significantly different from no treatment sample.

FIG. 7. Bispecific antibodies targeting IsdB and C1q can increase complement deposition on the surface of S. aureus leading to cell death. bsAbs were produced where each recognized C1q and a different protein on the surface of S. aureus (left to right): protein A, ClfA, PNAG, IsdA, and IsdB. Blood assays were performed as described above with two concentrations of antibody. Full length-antibodies were tested at 10 and 100 pg/ml, and antibodies with a scFv arm were tested at 8.5 and 85 ug/ml (the molar equivalent of the full-length antibodies). Results are plotted as mean with standard deviation. * P<0.05, ** P<0.01, *** P<0.001, **** P<0.0001, one-way ANOVA showing bsAb samples significantly different from the corresponding bivalent antibody.

FIG. 8. The IsdB x C1q bispecific antibody increases clearance of S. aureus in mice with humanized C1q heads. Quantitative measurement of the expression of chimeric C1q peptides was assessed via LC-MS/MS. Serum was isolated from individual C1g humanized mice and the concentration of humanized mouse C1q specific peptides was determined. Results are plotted as median and interquartile range.

FIG. 9. The IsdB x C1q bispecific antibody increases clearance of S. aureus in mice with humanized C1q heads. C1q humanized mice were infected with 1.5×10⁸ CFU S. aureus Newman (an MSSA strain). One day after infection they were treated with the indicated antibody. Weights were recorded each day for four days and percent weight change compared to day 0 is shown (left). Mean weight change for each group of mice is plotted. On day 4, kidneys were harvested, dissociated, and serially diluted. Organ burden was determined by plating (right). The organ burden of each mouse is indicated along with the median value for each group. ** P<0.01, *** P<0.001, nonparametric Kruskal-Wallis ANOVA with Dunn's test showing values significantly different from the no treatment samples.

FIG. 10. The IsdB x C1q bispecific antibody increases clearance of S. aureus in mice with humanized C1q heads. C1q humanized mice were infected with 1.3×10⁸ CFU S. aureus MW2 (a MRSA strain) on day 1. One day after infection they were treated with either PBS (no treatment), a single dose of anti-IsdB×anti-C1q antibody, 110 mg/kg vancomycin twice daily, 50 mg/kg daptomycin once daily, or 80 mg/kg linezolid twice daily. On day 4 kidneys were harvested, dissociated, and serially diluted. Organ burden was determined by plating. ** P<0.01, *** P<0.001, nonparametric Kruskal-Wallis ANOVA with Dunn's test showing values significantly different from the no treatment samples.

FIG. 11. Anti-IsdB×anti-C1q bsAb reduces the bacterial burden of S. aureus in kidneys of C1q humanized mice. C1q humanized mice were infected with 1.4×10⁸ CFU S. aureus CA-127 (an MRSA strain). One day after infection they were treated with the indicated antibody. Weights were recorded each day for four days and percent weight change compared to day 0 is shown (left). Mean weight change for each group of mice is plotted. On day 4, kidneys were harvested, dissociated, and serially diluted. Organ burden was determined by plating (right).

FIG. 12. Anti-IsdB×anti-C1q bsAb reduces the bacterial burden of several strains of S. aureus in hearts of C1q humanized mice. C1q humanized mice were infected with either 1.5×10⁸ CFU S. aureus Newman (an MSSA strain) or 1.4×10⁸ CFU S. aureus CA-127 (an MRSA strain) on day 0. One day after infection they were treated with the indicated antibody intraperitoneally. On day 4, hearts were harvested, dissociated, and serially diluted to enumerate bacterial load. The organ burden of each mouse is indicated along with the median value for each group. * P<0.05, ** P<0.01, *** P<0.001, **** P<0.0001, nonparametric Kruskal-Wallis ANOVA with Dunn's test.

FIG. 13. Anti-IsdB×anti-C1q bsAb reduces the bacterial burden of several strains of S. aureus in livers of C1q humanized mice. C1q humanized mice were infected with either 1.5×10⁸ CFU S. aureus Newman (an MSSA strain) or 1.4×10⁸ CFU S. aureus CA-127 (an MRSA strain) on day 0. One day after infection they were treated with the indicated antibody intraperitoneally. On day 4, livers were harvested, dissociated, and serially diluted to enumerate bacterial load. The organ burden of each mouse is indicated along with the median value for each group.* P<0.05, ** P<0.01, *** P<0.001, **** P<0.0001, nonparametric Kruskal-Wallis ANOVA with Dunn's test.

FIG. 14. Anti-IsdB×anti-C1q bsAb reduces the bacterial burden of several strains of S. aureus in lungs of C1q humanized mice. C1q humanized mice were infected with either 1.5×10⁸ CFU S. aureus Newman (an MSSA strain) or 1.4×10⁸ CFU S. aureus CA-127 (an MRSA strain) on day 0. One day after infection they were treated with the indicated antibody intraperitoneally. On day 4, lungs were harvested, dissociated, and serially diluted to enumerate bacterial load. The organ burden of each mouse is indicated along with the median value for each group.* P<0.05, ** P<0.01, *** P<0.001, **** P<0.0001, nonparametric Kruskal-Wallis ANOVA with Dunn's test.

FIG. 15. Anti-IsdB×anti-C1q bsAb reduces the bacterial burden of several strains of S. aureus in spleen of C1q humanized mice. C1q humanized mice were infected with either 1.5×10⁸ CFU S. aureus Newman (an MSSA strain) or 1.4×10⁸ CFU S. aureus CA-127 (an MRSA strain) on day 0. One day after infection they were treated with the indicated antibody intraperitoneally. On day 4, spleens were harvested, dissociated, and serially diluted to enumerate bacterial load. The organ burden of each mouse is indicated along with the median value for each group.* P<0.05, ** P<0.01, *** P<0.001, **** P<0.0001, nonparametric Kruskal-Wallis ANOVA with Dunn's test.

FIG. 16. Dose response curves of REGN6093 (FluA group 1×C1q bispecific), parental or control antibodies in an ELISA assay with A/Puerto Rico/08/1934 (H1N1)-infected MDCK cells. MDCK cells were infected with the virus for 24 h before their fixation with 4% paraformaldehyde. The antibodies were added onto the fixed cells and bound antibodies were detected with an a-human IgG (H+L) specific secondary antibody. All values are reported in Relative Fluorescent Units (RLUs). Shown are REGN6093 (anti-HA×anti-C1q bispecific, black squares), mAb11829 (parental HA bivalent antibody, clear circles), mAb27250 (one-arm parental HA antibody; gray diamonds), mAb20067 (FluA group 1×FcγRIII bispecific; gray triangles) along with controls REGN5066 (IsdB×C1q bispecific; clear squares), negative control #2 (clear inverted triangles), REGN5408 (IsdBxC1q scFv; clear triangles), negative control #3 (gray crosses), and REGN1932 (IgG1 isotype control; clear hexagon).

FIG. 17. Dose response curves of REGN6093 (FluA group 1×C1q bispecific), parental or control antibodies in a CDC assay with A/Puerto Rico/08/1934 (H1N1)-infected MDCK cells and normal human serum complement. MDCK cells were infected with the virus for 24 h. Infected cells were collected and mixed with the antibodies and 5% normal human serum complement for 2 hours at room temperature. Complement dependent cell lysis was measured with the CytoTox-Glo kit (Promega). Percent specific lysis is expressed relative to minimum and maximum lysis controls. Shown are REGN6093 (anti-HA×anti-C1q bispecific, black squares), mAb11829 (parental HA bivalent antibody, clear circles), mAb27250 (one-arm parental HA antibody; gray diamonds), mAb20067 (FluA group 1×FcγRIII bispecific; gray triangles) along with controls REGN5066 (IsdBxC1q bispecific; clear squares), negative control #2 (clear inverted triangles), REGN5408 (IsdBxC1q scFv; clear triangles), negative control #3 (gray crosses), and REGN1932 (IgG1 isotype control; clear hexagon).

FIG. 18. Raji cells were seeded onto a 96-well while assay plates with clear bottom at 10,000 cells/well in 1% BSA containing RPMI 1640. The test antibodies (anti-CD20×anti-C1q bispecific antibody REGN5177 [triangles], anti-CD20 parental bivalent hIgG1 antibody mAb14303 [inverted triangles], Rituximab [asterisks], and the anti-C1q parental bivalent hIgG1 antibody REGN5201 [diamonds]) were serially diluted 1:8 from 100 nM to 0.004 nM and added to the cells, along with human serum at a final concentration of 5%. Cytotoxicity was measured after 1 hour of incubation at 37° C. and in 5% CO₂, followed by a 30-minute incubation at 25° C., and addition of CytoTox-Glo™ reagent (Promega, #G9291). CytoTox-Glo™ is a luminescence-based reagent that measures cell killing such that increased luminescence is observed with increased cytotoxicity (measured in relative light units, RLUs). Untreated cells in control wells were lysed by treatment with digitonin immediately after addition of CytoTox-Glo™ reagent to determine maximal killing of cells. Plates were read for luminescence using a Spectramax 13.

FIG. 19. Jurkat/hGITR/hCD20 cells were incubated with a range of concentrations (1 pM to 1 μM) of anti-GITR×anti-C1q bispecific antibody REGN6545 (blue circles), anti-GITR×anti-C1q bispecific antibody REGN6546 (red squares), anti-GITR bivalent antibody REGN5372 (orange diamonds), and an IgG1 isotype-matched control antibody REGN1932 (black asterisks). Cell-surface binding of each antibody was detected by an Alexa647-anti-hIgG secondary antibody and the resultant fluorescence intensity of Alexa647 staining was plotted as MFI. Representative data from an assay performed in triplicate wells are presented as mean±SD. The x-axis indicates the molar concentration of antibodies. Abbreviations: SD, standard deviation; MFI, mean fluorescence intensity.

FIG. 20. Jurkat/hGITR/hCD20 cells were incubated with 5% NHS and a range of concentrations (0.5 pM to 500 nM) of anti-GITR×anti-C1q REGN6545 (blue circles), anti-GITR×anti-C1q bispecific antibody REGN6546 (red squares), anti-GITR bivalent antibody REGN5372 (orange diamonds), and an IgG1 isotype-matched control antibody REGN1932 (black asterisks). Cytotoxicity was determined using the commercially available CytoTox-Glo assay which measures the number of dead cells within a general cell population using a luminescence readout. Data from an assay performed in triplicate wells are plotted as mean±SD.

FIG. 21. Healthy (uninfected) humanized C1q (C1q^{Hu/Hu}) mice in a mixed background (MAID1615), males and females, were subcutaneously administered an anti-IsdB×anti-C1q IgG1 bispecific antibody (REGN5066), an anti-IsdB×anti-C1q IgG1 uber stealth bispecific antibody (REGN7017), an anti-IsdB×anti-C1q IgG4 bispecific antibody (REGN5331) or a control antibody (REGN1932). Blood was collected 1 hr, 24 h, 48 h, and 72 h post antibody treatment and serum was prepared using gold-capped serum separator tubes (BD). Serum was diluted 1:2000 and analyzed by ELISA to determine C1q levels.

FIG. 22. Humanized C1q (C1q^{Hu/Hu}) mice in a mixed background (MAID1615) were infected intraperitoneally with S. aureus Newman (1.5×10⁸ colony forming units (CFUs) per mouse). All three S. aureus strains was grown to log phase, OD₆₀₀≤1, in TSB at 37° C. and washed 3 times in PBS and adjusted to the desired density for infection. Infected mice were subcutaneously administered an anti-IsdB×anti-C1q IgG1 bispecific antibody (REGN5066), an anti-IsdB×anti-C1q IgG1 uber stealth bispecific antibody (REGN7017), an anti-IsdB×anti-C1q IgG4 bispecific antibody (REGN5331) or a control antibody (REGN1932). Blood was collected prior to infection and at 1 hr, 24 h, 48 h, and 72 h post antibody treatment and serum was prepared using gold-capped serum separator tubes (BD). Serum was diluted 1:2000 and analyzed by ELISA to determine C1q levels.

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present disclosure, the preferred methods and materials are now described. All patents, applications and non-patent publications mentioned in this specification are incorporated herein by reference in their entireties.

### I. Definitions

To provide background for the following definitions, the bispecific antigen-binding molecules of the present disclosure comprise two separate antigen-binding domains (D1 and D2). One antigen-binding domain (D1) specifically binds to a target antigen. In some embodiments, the target antigen is on the surface of a cell or an organism, e.g., an antigen on the surface of a bacterium or an antigen on the surface of a eukaryotic cell. The cell may be any type of cell, e.g., from any tissue or organ, stem cell, progenitor cell, differentiated cell, etc. In some embodiments, the target antigen is a soluble target antigen, i.e., not bound to a cell. The other antigen-binding domain (D2) specifically binds to a component of the complement pathway (“complement component”), e.g., C1q, C1r, C1s, C2, C3, C4, C5, C6, C7, C8, C9, or a subunit or fragment thereof.

In some embodiments, one antigen-binding domain (D1) specifically binds to a target antigen on the surface of a cell or organism, where the target antigen is a marker of a disease state of a subject and the second antigen-binding domain (D2) binds to a complement component, e.g. C1q. For example, one antigen-binding domain (D1) specifically binds to a target antigen on the surface of a bacteria, on the surface of a virus, on the surface of a bacterial or virus infected cell, on the surface of a cancer cell, or on the surface of an immune cell, and the second antigen-binding domain (D2) binds to a complement component, e.g. C1q. For example, one antigen-binding domain (D1) specifically binds to a Staphylococcus species target antigen (e.g., IsdB, Protein A, IsdA, PNAG, ClfA), an Influenza virus target antigen (e.g., Influenza HA), a Pseudomonas species target antigen (e.g., Psl), an immune cell surface antigen such as a tumor necrosis factor receptor antigen (e.g., GITR), or a cancer-cell surface antigen (e.g., CD20), and the second antigen-binding domain (D2) binds to a complement component, e.g. C1q.

As used herein, “an antibody that binds a complement component” or an “anti-complement component antibody” includes antibodies and antigen-binding fragments thereof that specifically recognize a complement component. Such antibodies include antibodies and antigen-binding fragments thereof that specifically recognize a subunit of the complement component, such as a single C1q subunit (e.g., component A, B, or C), as well as antibodies and antigen-binding fragments thereof that specifically recognize more than one subunit, for example a multimer such as a trimer or a hexamer. In one embodiment, the antigen binding molecules of the disclosure bind the complement component C1q, or a subunit of C1q, or a multimeric form of C1q. As used herein, “C1q” includes natural C1q proteins as well as recombinant C1q protein, or variants thereof. In some embodiments, the C1q is human C1q. Human complement C1q is a hexamer that comprises three unique subunits: A (29 kDa); B (26 kDa); and C (22 kDa). An exemplary sequence of subunit A is found at amino acids 1-245 of accession number NP_057075. An exemplary sequence of subunit B is found at amino acids 1-253 of accession number NP_000482. An exemplary sequence of subunit C is found at amino acids 1-245 of accession number NP 758957.

As used herein, the expression “an antibody that binds a complement component”, an “anti-complement component antibody”, or an “anti-C1q antibody” includes both monovalent antibodies with a single specificity, as well as bispecific antibodies comprising an arm that binds a C1q, wherein the anti-C1q arm comprises any of the HCVR/LCVR or CDR sequences as set forth in Table 1. Examples of anti-C1q bispecific antibodies are described elsewhere herein.

As used herein, the expression “antigen-binding molecule” means a protein, polypeptide or molecular complex having binding affinity for a particular antigen. In embodiments, the antigen-binding molecule comprises or consists of at least one complementarity determining region (CDR) that alone, or in combination with one or more additional CDRs and/or framework regions (FRs), specifically binds to a particular antigen. In embodiments, an antigen binding molecule comprises one or more antigen binding domains. In embodiments, an antigen binding molecule is a bispecific antigen binding molecule having a first antigen binding domain with binding affinity for a first antigen and a second antigen binding domain with binding affinity for a second antigen. The term “antigen-binding molecule” includes antibodies and antigen-binding fragments of antibodies, including, e.g., bispecific antibodies.

As used herein, “target antigen” refers to an antigen found on the surface of an organism or cell. In some embodiments, the target antigen is a marker for one or more cell types. In some embodiments, the target antigen is a marker of a disease state in a subject. In embodiments, a target antigen is found on a bacterial pathogen, a viral pathogen, a bacteria- or virus-infected cell, a cancer cell, or an immune cell. For example, bacterial and viral pathogens include Staphylococcus species, Influenza virus, or Pseudomonas species. In embodiments, the target antigen is unique to a specific cell type, or is abundantly expressed by a specific cell type, or is overexpressed by a specific cell type as compared to other types of cells. In other embodiments, the target antigen is unique to the cancer cell or is more highly expressed on the cancer cell as compared to non-cancer cells. In yet other embodiments, the target antigen is found on an immune cell, such as T regulatory cell.

The expression “Staphylococcus species” as used herein, refers to a species of the microbe Staphylococcus. In embodiments, the Staphylococcus species is a species that is associated with infections in humans including but not limited to Staphylococcus epidermidis, Staphylococcus aureus, Staphylococcus lugdunensis, and/or Staphylococcus saprophyticus.

The expression “Influenza virus” as used herein, refers to an Influenza A, Influenza B, Influenza C, and/or Influenza D virus. Influenza viruses can be further subtyped based on hemagglutinin (HA) and neuraminidase (NA). In embodiments, the Influenza virus is a strain that is associated with infections in humans and includes, but is not limited to, Influenza A H1, H3, H5, H7 or H9 strains.

The expression “Pseudomonas species” as used herein, refers to a species of the gram-negative bacteria Pseudomonas. In embodiments, the Pseudomonas species is a species that is associated with infections in humans, including but not limited to Pseudomonas aeruginosa PAO-1.

The expression “tumor necrosis factor receptor antigen” as used herein, refers to the tumor necrosis factor receptor family and includes antigens like glucocorticoid-induced TNFR-related protein (GITR).

The expression “immune cell surface antigen” refers to an antigen that is expressed on the surface of one or more types of immune cells such as, but not limited to, B cells, dendritic cells, granulocytes, lymphoid cells, monocytes, macrophages, T cells, and thymocytes.

The expression “B-cell surface antigen” refers to an antigen that is expressed on the surface of a B cell (e.g., B lymphocytes). In some embodiments, a B-cell surface antigen includes antigens like CD20, which is expressed on the surface of B-cells.

The expression “T-cell surface antigen” refers to an antigen that is expressed on the surface of a T cell (e.g., a regulatory T cell). In some embodiments, a T-cell surface antigen includes antigens like GITR.

The expression “cancer-cell surface antigen” refers to an antigen found uniquely or more highly expressed on the surface of a cancer cell. Exemplary antigens include antigens like CD20, which is expressed on the surface of leukemia and lymphoma cells.

As used herein, “an antibody that binds a Staphylococcus species target antigen” or an “anti-Staphylococcus species target antibody” includes antibodies and antigen-binding fragments thereof that specifically recognize a target antigen on the cell surface of a Staphylococcus species. In one embodiment, the antigen binding molecules of the disclosure bind the target antigen referred to as “iron regulated surface protein B”, or “IsdB”. An exemplary IsdB protein has the amino acid sequence of amino acids 3 to 575 of SEQ ID NO: 80. In another embodiment, the antigen binding molecules of the disclosure bind the target antigen referred to as “Protein A.” In another embodiment, the antigen binding molecules of the disclosure bind the target antigen referred to as “iron regulated surface protein A”, or “IsdA.” In another embodiment, the antigen binding molecules of the disclosure bind the target antigen referred to as “anti-Poly-N-acetylglucosamine”, or “PNAG.” In another embodiment, the antigen binding molecules of the disclosure bind the target antigen referred to as “anti-Clumping factor A”, or “ClfA.”

As used herein, the expression “an antibody that binds a Staphylococcus species target antigen” or an “anti-Staphylococcus species target antibody” includes, but is not limited to, both monovalent antibodies with a single specificity, as well as bispecific antibodies comprising a first arm that binds IsdB, Protein A, IsdA, PNAG, or ClfA and a second arm that binds a C1q, wherein the anti-IsdB arm, anti-Protein A arm, anti-IsdA arm, anti-PNAG arm, or anti-ClfA arm comprises any of the HCVR/LCVR or CDR sequences as set forth in Tables 1, 3, 12, 14, 16, and 17, respectively.

As used herein, “an antibody that binds an Influenza virus target antigen” or an “anti-Influenza virus target antigen” includes antibodies and antigen-binding fragments thereof that specifically recognize a target antigen on the cell surface of an Influenza infected cell, or in an Influenza strain. In one embodiment, the antigen binding molecules of the disclosure bind the target antigen referred to as “Influenza HA.” An exemplary Influenza HA protein has the amino acid sequence of amino acids 1 to 519 of SEQ ID NO: 81.

As used herein, the expression “an antibody that binds an Influenza virus target antigen”, an “anti-Influenza virus target antibody”, or an “anti-Influenza HA antibody” includes, but is not limited to, both monovalent antibodies with a single specificity, as well as bispecific antibodies comprising a first arm that binds Influenza HA and a second arm that binds a C1q, wherein the anti-Influenza HA arm comprises any of the HCVR/LCVR or CDR sequences as set forth in Table 7 herein. Examples of anti-Influenza HA bispecific antibodies are described elsewhere herein.

As used herein, “an antibody that binds a Pseudomonas species target antigen” or an “anti-Pseudomonas species target antigen” includes antibodies and antigen-binding fragments thereof that specifically recognize a target antigen on the cell surface of, or in a Pseudomonas species. In one embodiment, the antigen binding molecules of the disclosure bind the target antigen referred to as “Psl.” As used herein, the expression “an antibody that binds a Pseudomonas species target antigen”, an “anti-Pseudomonas species target antibody”, or an “anti-Psl antibody” includes, but is not limited to, both monovalent antibodies with a single specificity, as well as bispecific antibodies comprising a first arm that binds Psl and a second arm that binds a C1q, wherein the anti-Psl arm comprises any of the HCVR/LCVR or CDR sequences as set forth in Table 11 herein. Examples of anti-Psl bispecific antibodies are described elsewhere herein.

As used herein, “an antibody that binds a glucocorticoid induced tumor necrosis factor receptor (GITR) antigen” or an “anti-GITR” includes antibodies and antigen-binding fragments thereof that specifically recognize a glucocorticoid induced tumor necrosis factor receptor. In one embodiment, the antigen binding molecules of the disclosure bind to a glucocorticoid-induced tumor necrosis factor receptor or “GITR.”, found on T cells. An exemplary GITR protein has the amino acid sequence of amino acids 1 to 135 of SEQ ID NO: 82.

As used herein, the expression “an antibody that binds a glucocorticoid induced tumor necrosis factor receptor antigen”, or an “anti-GITR antibody” includes, but is not limited to, both monovalent antibodies with a single specificity, as well as bispecific antibodies comprising a first arm that binds GITR and a second arm that binds a C1q, wherein the anti-GITR arm comprises any of the HCVR/LCVR or CDR sequences as set forth in Table 9 herein. Examples of anti-GITR bispecific antibodies are described elsewhere herein.

As used herein, “an antibody that binds a cancer-cell surface antigen” or an “anti-cancer-cell surface antigen” includes antibodies and antigen-binding fragments thereof that specifically recognize a target antigen on the cell surface of a cancer cell. In one embodiment, the antigen binding molecules of the disclosure bind the target antigen referred to as “CD20.”

As used herein, the expression “an antibody that binds a cancer cell surface antigen”, an “anti-cancer cell surface antigen antibody”, or an “anti-CD20 antibody” includes, but is not limited to, both monovalent antibodies with a single specificity, as well as bispecific antibodies comprising a first arm that binds CD20 and a second arm that binds a C1q, wherein the anti-CD20 arm comprises any of the HCVR/LCVR or CDR sequences as set forth in Table 5 herein. Examples of anti-CD20 bispecific antibodies are described elsewhere herein.

The term “antibody”, as used herein, means any antigen-binding molecule or molecular complex comprising at least one complementarity determining region (CDR) that specifically binds to or interacts with a particular antigen (e.g., C1q). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-C1q antibody (or antigen-binding portion thereof) may be identical to the human germline sequences, or may be naturally or artificially modified. In different embodiments of the disclosure, the FRs of the anti-IsdB antibody (or antigen-binding portion thereof) may be identical to the human germline sequences, or may be naturally or artificially modified. An amino acid consensus sequence may be defined based on a side-by-side analysis of two or more CDRs.

The term “antibody”, as used herein, also includes antigen-binding fragments of full antibody molecules. The terms “antigen-binding portion” of an antibody, “antigen binding domain” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, the expression “antigen-binding domain” means any peptide, polypeptide, nucleic acid molecule, scaffold-type molecule, peptide display molecule, or polypeptide-containing construct that is capable of specifically binding a particular antigen of interest (e.g., a complement component, Staphylococcus species target antigen, Influenza virus target antigen, Pseudomonas species target antigen tumor necrosis factor receptor antigen, cancer-cell surface antigen).

The term “specifically binds” or the like, as used herein, means that the antigen-binding domain forms a complex with a particular antigen characterized by a dissociation constant (K_{D}) of 25 nM or less. Exemplary categories of antigen-binding domains that can be used in the context of the present disclosure include antibodies, antigen-binding portions of antibodies, peptides that specifically interact with a particular antigen (e.g., peptibodies), receptor molecules that specifically interact with a particular antigen, proteins comprising a ligand-binding portion of a receptor that specifically binds a particular antigen, antigen-binding scaffolds (e.g., DARPins, HEAT repeat proteins, ARM repeat proteins, tetratricopeptide repeat proteins, and other scaffolds based on naturally occurring repeat proteins, etc., [see, e.g., Boersma and Pluckthun, 2011, Curr. Opin. Biotechnol. 22:849-857, and references cited therein]), and aptamers or portions thereof.

Methods for determining whether two molecules specifically bind one another are well known in the art and include, for example, equilibrium dialysis, surface plasmon resonance, and the like. For example, an antigen-binding molecule, as used in the context of the present disclosure, includes polypeptides that bind a particular antigen (e.g., a target molecule [T] or a portion thereof) with a K_{D} of less than about 25 nM, less than about 20 nM, less than about 15 nM, less than about 10 nM, less than about 5 nM, less than about 1 nM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured in a surface plasmon resonance assay.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; (vii) an antigen binding domain, and (viii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may comprise or consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present disclosure using routine techniques available in the art.

The antibodies of the present disclosure may function through complement-dependent cytotoxicity (CDC) or antibody-dependent cell-mediated cytotoxicity (ADCC). “Complement-dependent cytotoxicity” (CDC) refers to lysis of antigen-expressing cells by an antibody of the disclosure in the presence of complement. “Antibody-dependent cell-mediated cytotoxicity” (ADCC) refers to a cell-mediated reaction in which nonspecific cytotoxic cells that express Fc receptors (FcRs) (e.g., Natural Killer (NK) cells, neutrophils, and macrophages) recognize bound antibody on a target cell and thereby lead to lysis of the target cell. CDC and ADCC can be measured using assays that are well known and available in the art. (See, e.g., U.S. Pat. Nos. 5,500,362 and 5,821,337, and Clynes et al. (1998) Proc. Natl. Acad. Sci. (USA) 95:652-656).

In certain embodiments of the disclosure, the anti-C1q, anti-IsdB, anti-Psl, anti-Influenza HA, anti-CD20, anti-Protein A, anti-IsdA, anti-PNAG, anti-ClfA, and/or anti-GITR antibodies of the disclosure (monospecific or bispecific) are human antibodies. The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences. The term includes antibodies recombinantly produced in a non-human mammal, or in cells of a non-human mammal. The term is not intended to include antibodies isolated from or generated in a human subject.

The antibodies of the disclosure may, in some embodiments, be recombinant human antibodies. The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant disclosure encompasses antibodies having one or more mutations in the hinge, C_{H}2 or C_{H}3 region which may be desirable, for example, in production, to improve the yield of the desired antibody form.

The antibodies of the disclosure may be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present disclosure. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The anti-C1q, anti-target antigen, or bispecific antigen binding molecules disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes antigen binding molecules, antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antigen binding molecules, antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antigen binding molecules, antibodies and antigen-binding fragments of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antigen binding molecules, antibodies and antigen-binding fragments that contain one or more germline mutations can be easily tested for one or more desired properties such as, improved binding specificity, increased binding affinity, improved or enhanced antagonistic or agonistic biological properties (as the case may be), reduced immunogenicity, etc. Antigen binding molecules, antibodies and antigen-binding fragments obtained in this general manner are encompassed within the present disclosure.

The present disclosure also includes one-arm antibodies that bind C1q, IsdB, Psl, Influenza HA, CD20, Protein A, IsdA, PNAG, ClfA, or GITR. As used herein, a “one-arm antibody” means an antigen-binding molecule comprising a single antibody heavy chain and a single antibody light chain. The one-arm antibodies of the present disclosure may comprise any of the HCVR/LCVR or CDR amino acid sequences as set forth in Tables 1, 3, 5, 7, 9, 11, 12, 14, 16, 17, and 18 herein.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, e.g., at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, e.g., at least 95% sequence identity, even more preferably at least 96%, 97%, 98%, or 99% sequence identity. Preferably, residue positions which are not identical differ by conservative amino acid substitutions. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutant thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the disclosure to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

### II. Complement Component Binding Molecules

According to one aspect of the present disclosure, anti-complement component binding molecules, e.g., antibodies and antigen-binding fragment thereof, are provided. Complement components comprise C1q, C1r, C1s, C2, C3, C4, C5, C6, C7, C8, C9, or any fragments thereof (including proteolytic fragments generated by the complement cascade). In embodiments, antibodies that bind C1q (e.g. monospecific anti-C1q antibodies) are provided. In embodiments, C1q is a human C1q. Human complement C1q is a hexamer that comprises three unique subunits: A (29 kDa); B (26 kDa); and C (22 kDa). An exemplary sequence of subunit A is found at amino acids 1-245 of accession number NP_057075. An exemplary sequence of subunit B is found at amino acids 1-253 of accession number NP_000482. An exemplary sequence of subunit C is found at amino acids 1-245 of accession number NP_758957. The antibodies according to this aspect of the disclosure are useful, inter alia, for targeting C1q and complement activation to specific targets.

Exemplary anti-C1q antibodies are described in U.S. Ser. No. 16/143,901 and PCT/US2018/053064, incorporated by reference herein. The anti-C1q antibodies of the disclosure, or antigen-binding portions thereof, may be included in a bispecific antigen-binding molecule that directs C1q-mediated complement activation to specific target antigens.

In embodiments, the anti-C1q antibodies are selected that inhibit C1q binding to immunoglobulin molecules, inhibit hemolysis, and/or complement dependent cytotoxicity. In embodiments, the bispecific antigen-binding molecule blocks the binding of the human C1q to human IgG1-k with an IC₅₀ of about 10 nM or less. In other embodiments, the bispecific antigen binding molecule blocks the binding of the human C1q to human IgM with an IC₅₀ of about 50 nM or less. In embodiments, the bispecific antigen binding molecule inhibits complement dependent cytotoxicity with an IC₅₀ of about 100 nM or less. In yet other embodiments, the bispecific antigen binding molecule provides for deposition of human C1q with an EC₅₀ of about 10 nM or less. In certain embodiments, an antigen binding molecule of the invention may not exhibit one or more of the following: blocking or inhibition of IgG binding, or hemolysis, or complement dependent cytotoxicity, as described herein, but may still be effective for preventing a Staphylococcus infection, or for treating an existing infection in a subject.

In specific embodiments, sequences of exemplary anti-C1q are listed in Tables 1 and 2 herein. Table 1 sets forth the amino acid sequence identifiers of the heavy chain variable regions (HCVRs) and light chain variable regions (LCVRs), as well as heavy chain complementarity determining regions (HCDR1, HCDR2 and HCDR3), and light chain complementarity determining regions (LCDR1, LCDR2 and LCDR3) of the exemplary anti-C1q antibodies. Table 2 sets forth the sequence identifiers of the nucleic acid molecules encoding the HCVRs, LCVRs, HCDR1, HCDR2, HCDR3, LCDR1, LCDR2 and LCDR3 of the exemplary anti-C1q antibodies.

The present disclosure provides antibodies, or antigen-binding fragments thereof that bind human C1q, comprising heavy chain variable region CDRs contained within a HCVR comprising an amino acid sequence selected from any of the HCVR amino acid sequences listed in Table 1, or a substantially similar sequence thereof having at least 90%, at least 91%, at least 92%, at least 93%, at least 94%, at least 95%, at least 96%, at least 97%, at least 98% or at least 99% sequence identity thereto.

The present disclosure also provides anti-C1q antibodies, or antigen-binding fragments thereof, comprising light chain variable region CDRs contained within a LCVR comprising an amino acid sequence selected from any of the LCVR amino acid sequences listed in Table 1, or a substantially similar sequence thereof having at least 90%, at least 91%, at least 92%, at least 93%, at least 94%, at least 95%, at least 96%, at least 97%, at least 98% or at least 99% sequence identity thereto.

The present disclosure also provides anti-C1q antibodies, or antigen-binding fragments thereof, comprising an HCVR and an LCVR pair (HCVR/LCVR) comprising any of the HCVR amino acid sequences listed in Table 1 paired with any of the LCVR amino acid sequences listed in Table 1. In certain embodiments, an anti-C1q antibody comprises a HCVR/LCVR pair, the HCVR/LCVR pair comprising amino acid sequence of SEQ ID NOs: 2/10 (e.g., mAb17736); SEQ ID NOs: 18/10 (e.g., mAb18395).

The present disclosure also provides antibodies, or antigen-binding fragments thereof, that bind to human C1q comprising a heavy chain CDR1 (HCDR1) comprising an amino acid sequence selected from any of the HCDR1 amino acid sequences listed in Table 1 or a substantially similar sequence thereof having at least 90%, at least 95%, at least 98% or at least 99% sequence identity.

The present disclosure also provides antibodies, or antigen-binding fragments thereof, that bind to human C1q comprising a heavy chain CDR2 (HCDR2) comprising an amino acid sequence selected from any of the HCDR2 amino acid sequences listed in Table 1 or a substantially similar sequence thereof having at least 90%, at least 95%, at least 98% or at least 99% sequence identity.

The present disclosure also provides antibodies, or antigen-binding fragments thereof, that bind to human C1q comprising a heavy chain CDR3 (HCDR3) comprising an amino acid sequence selected from any of the HCDR3 amino acid sequences listed in Table 1 or a substantially similar sequence thereof having at least 90%, at least 95%, at least 98% or at least 99% sequence identity.

The present disclosure also provides antibodies, or antigen-binding fragments thereof, that bind to human C1q comprising a light chain CDR1 (LCDR1) comprising an amino acid sequence selected from any of the LCDR1 amino acid sequences listed in Table 1 or a substantially similar sequence thereof having at least 90%, at least 95%, at least 98% or at least 99% sequence identity.

The present disclosure also provides antibodies, or antigen-binding fragments thereof, that bind to human C1q comprising a light chain CDR2 (LCDR2) comprising an amino acid sequence selected from any of the LCDR2 amino acid sequences listed in Table 1 or a substantially similar sequence thereof having at least 90%, at least 95%, at least 98% or at least 99% sequence identity.

The present disclosure also provides antibodies, or antigen-binding fragments thereof, that bind to human C1q comprising a light chain CDR3 (LCDR3) comprising an amino acid sequence selected from any of the LCDR3 amino acid sequences listed in Table 1 or a substantially similar sequence thereof having at least 90%, at least 95%, at least 98% or at least 99% sequence identity.

The present disclosure also provides antibodies, or antigen-binding fragments thereof, that bind to human C1q comprising an HCDR3 and an LCDR3 pair (HCDR3/LCDR3) comprising any of the HCDR3 amino acid sequences listed in Table 1 paired with any of the LCDR3 amino acid sequences listed in Table 1. According to certain embodiments, the present disclosure provides antibodies, or antigen-binding fragments thereof, comprising an HCDR3/LCDR3 pair contained within any of the exemplary anti-C1q antibodies listed in Table 1. In certain embodiments, the HCDR3/LCDR3 pair comprises amino acid sequences of SEQ ID NOs: 8/16 (e.g., mAb17736), or of SEQ ID NOs: 24/16 (e.g., mAb18395).

The present disclosure also provides antibodies, or antigen-binding fragments thereof, that bind to human C1q comprising a set of six CDRs (i.e., HCDR1-HCDR2-HCDR3-LCDR1-LCDR2-LCDR3) contained within any of the exemplary anti-C1q antibodies listed in Table 1. In certain embodiments, the HCDR1-HCDR2-HCDR3-LCDR1-LCDR2-LCDR3 set comprises the amino acid sequences of SEQ ID NOs: 4, 6, 8, 12, 14, and 16 (e.g., mAb17736); of the amino acid sequences of 20, 22, 24, 12, 14, and 16 (e.g., mAb18395).

In a related embodiment, the present disclosure provides antibodies, or antigen-binding fragments thereof that bind to human C1q, comprising a set of six CDRs (i.e., HCDR1-HCDR2-HCDR3-LCDR1-LCDR2-LCDR3) contained within an HCVR/LCVR pair as defined by any of the exemplary anti-C1q antibodies listed in Table 1. For example, the present disclosure includes antibodies, or antigen-binding fragments thereof, comprising the HCDR1-HCDR2-HCDR3-LCDR1-LCDR2-LCDR3 set contained within an HCVR/LCVR pair comprising amino acid sequences of SEQ ID NOs: 2/10 (e.g., mAb17736); or of SEQ ID NOs: 18/10 (e.g., mAb18395).

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

The present disclosure also provides nucleic acid molecules encoding anti-C1q antibodies or portions thereof. For example, the present disclosure provides nucleic acid molecules encoding any of the HCVR amino acid sequences listed in Table 1; in certain embodiments the nucleic acid molecule comprises a polynucleotide sequence selected from any of the HCVR nucleic acid sequences listed in Table 2, or a substantially similar sequence thereof having at least 90%, at least 91%, at least 92%, at least 93%, at least 94%, at least 95%, at least 96%, at least 97%, at least 98% or at least 99% sequence identity thereto.

The present disclosure also provides nucleic acid molecules encoding any of the LCVR amino acid sequences listed in Table 1; in certain embodiments the nucleic acid molecule comprises a polynucleotide sequence selected from any of the LCVR nucleic acid sequences listed in Table 2, or a substantially similar sequence thereof having at least 90%, at least 91%, at least 92%, at least 93%, at least 94%, at least 95%, at least 96%, at least 97%, at least 98% or at least 99% sequence identity thereto.

The present disclosure also provides nucleic acid molecules encoding any of the HCDR1 amino acid sequences listed in Table 1; in certain embodiments the nucleic acid molecule comprises a polynucleotide sequence selected from any of the HCDR1 nucleic acid sequences listed in Table 2, or a substantially similar sequence thereof having at least 90%, at least 95%, at least 98% or at least 99% sequence identity thereto.

The present disclosure also provides nucleic acid molecules encoding any of the HCDR2 amino acid sequences listed in Table 1; in certain embodiments the nucleic acid molecule comprises a polynucleotide sequence selected from any of the HCDR2 nucleic acid sequences listed in Table 2, or a substantially similar sequence thereof having at least 90%, at least 95%, at least 98% or at least 99% sequence identity thereto.

The present disclosure also provides nucleic acid molecules encoding any of the HCDR3 amino acid sequences listed in Table 1; in certain embodiments the nucleic acid molecule comprises a polynucleotide sequence selected from any of the HCDR3 nucleic acid sequences listed in Table 2, or a substantially similar sequence thereof having at least 90%, at least 95%, at least 98% or at least 99% sequence identity thereto.

The present disclosure also provides nucleic acid molecules encoding any of the LCDR1 amino acid sequences listed in Table 1; in certain embodiments the nucleic acid molecule comprises a polynucleotide sequence selected from any of the LCDR1 nucleic acid sequences listed in Table 2, or a substantially similar sequence thereof having at least 90%, at least 95%, at least 98% or at least 99% sequence identity thereto.

The present disclosure also provides nucleic acid molecules encoding any of the LCDR2 amino acid sequences listed in Table 1; in certain embodiments the nucleic acid molecule comprises a polynucleotide sequence selected from any of the LCDR2 nucleic acid sequences listed in Table 2, or a substantially similar sequence thereof having at least 90%, at least 95%, at least 98% or at least 99% sequence identity thereto.

The present disclosure also provides nucleic acid molecules encoding any of the LCDR3 amino acid sequences listed in Table 1; in certain embodiments the nucleic acid molecule comprises a polynucleotide sequence selected from any of the LCDR3 nucleic acid sequences listed in Table 2, or a substantially similar sequence thereof having at least 90%, at least 95%, at least 98% or at least 99% sequence identity thereto.

The present disclosure also provides nucleic acid molecules encoding an HCVR, wherein the HCVR comprises a set of three CDRs (i.e., HCDR1-HCDR2-HCDR3), wherein the HCDR1-HCDR2-HCDR3 set is as defined by any of the exemplary anti-C1q antibodies listed in Table 1.

The present disclosure also provides nucleic acid molecules encoding an LCVR, wherein the LCVR comprises a set of three CDRs (i.e., LCDR1-LCDR2-LCDR3), wherein the LCDR1-LCDR2-LCDR3 set is as defined by any of the exemplary anti-C1q antibodies listed in Table 1.

The present disclosure also provides nucleic acid molecules encoding both an HCVR and an LCVR, wherein the HCVR comprises an amino acid sequence of any of the HCVR amino acid sequences listed in Table 1, and wherein the LCVR comprises an amino acid sequence of any of the LCVR amino acid sequences listed in Table 1. In certain embodiments, the nucleic acid molecule comprises a polynucleotide sequence selected from any of the HCVR nucleic acid sequences listed in Table 2, or a substantially similar sequence thereof having at least 90%, at least 95%, at least 98% or at least 99% sequence identity thereto, and a polynucleotide sequence selected from any of the LCVR nucleic acid sequences listed in Table 2, or a substantially similar sequence thereof having at least 90%, at least 95%, at least 98% or at least 99% sequence identity thereto. In certain embodiments according to this aspect of the disclosure, the nucleic acid molecule encodes an HCVR and LCVR, wherein the HCVR and LCVR are both derived from the same anti-C1q antibody listed in Table 1.

The present disclosure also provides recombinant expression vectors capable of expressing a polypeptide comprising a heavy or light chain variable region of an anti-C1q antibody. For example, the present disclosure includes recombinant expression vectors comprising any of the nucleic acid molecules mentioned above, i.e., nucleic acid molecules encoding any of the HCVR, LCVR, and/or CDR sequences as set forth in Table 1. Also included within the scope of the present disclosure are host cells into which such vectors have been introduced, as well as methods of producing the antibodies or portions thereof by culturing the host cells under conditions permitting production of the antibodies or antibody fragments, and recovering the antibodies and antibody fragments so produced.

The present disclosure includes anti-C1q antibodies having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

### III. Target Antigen Binding Molecules

In another aspect, the present disclosure provides binding molecules, e.g., antibodies and antigen-binding fragments thereof, that bind a target antigen as disclosed herein. In some embodiments, the target antigen is expressed on or bound to the surface of a cell or organism, e.g., a target antigen that is found on the surface of a bacterium, or a target antigen that is expressed on the surface of a cell (e.g., a eukaryotic cell) that is to be targeted for elimination, such as but not limited to a bacterially- or virally-infected cell, an immune cell, or a cancer cell. In some embodiments, the target antigen is soluble, i.e., not bound to the cell or organism surface. For example, in some embodiments, the target antigen is a soluble multimer that the complement component (e.g., C1q) opsonizes for clearance via macrophages or immunogenicity via binding to the complement receptor CR2/CD21 on B cells.

### A. Staphylococcus Target Antigens

In another aspect, the present disclosure provides binding molecules, e.g., antibodies and antigen-binding fragments thereof, that bind a Staphylococcus species target antigen (e.g. a monospecific antibody to a Staphylococcus species target antigen). In embodiments, the Staphylococcus species is a species that causes an infection in humans. In embodiments, the Staphylococcus species comprises Staphylococcus epidermidis, Staphylococcus aureus, Staphylococcus lugdunensis, and/or Staphylococcus saprophyticus. In embodiments, the Staphylococcus species target antigen comprises capsular polysaccharide type 5, capsular polysaccharide type 8, protein A, IsdB, IsdA, IsdH, lipoteichoic acid, wall teichoic acid, clumping factor A (ClfA), poly-N-acetyl glucosamine (PNAG), lipase, V8 lipase, fatty acid modifying enzyme, microbial surface components recognizing adhesive matrix molecules (e.g., adhesins, fibrinogen binding molecules, fibronectin binding protein A, fibronectin binding protein B), or combinations thereof.

In one embodiment, the antibody or antigen binding fragment thereof specifically binds IsdB. Exemplary anti-IsdB antibodies are disclosed in U.S. Ser. No. 16/143,901 and PCT/US2018/053064, incorporated by reference herein. In one embodiment, the antibody or antigen binding fragment thereof that specifically binds IsdB binds to an IsdB comprising an amino acid sequence of amino acids 3 to 575 of SEQ ID NO: 80. The anti-IsdB antibodies of the disclosure, or antigen-binding portions thereof, may be included in a bispecific antigen-binding molecule that binds specific target cell types such as infectious agents. In embodiments, the bispecific antigen-binding molecule binds to the Staphylococcus species with an IC₅₀ of about 10 nM or less.

Exemplary anti-IsdB antibodies of the present disclosure are listed in Tables 3 and 4. Table 3 sets forth the amino acid sequence identifiers of the heavy chain variable regions (HCVRs) and light chain variable regions (LCVRs), as well as heavy chain complementarity determining regions (HCDR1, HCDR2 and HCDR3), and light chain complementarity determining regions (LCDR1, LCDR2 and LCDR3) of the exemplary anti-IsdB antibodies. Table 4 sets forth the sequence identifiers of the nucleic acid molecules encoding the HCVRs, LCVRs, HCDR1, HCDR2, HCDR3, LCDR1, LCDR2 and LCDR3 of the exemplary anti-IsdB antibodies

The present disclosure provides antibodies, or antigen-binding fragments thereof that bind IsdB comprising heavy chain variable region (HCVR) CDRs contained within a HCVR comprising an amino acid sequence of SEQ ID NO: 26, or a substantially similar sequence thereof having at least 90%, at least 91%, at least 92%, at least 93%, at least 94%, at least 95%, at least 96%, at least 97%, at least 98%, or at least 99% sequence identity thereto; and light chain variable region (LCVR) CDRs contained within a LCVR region comprising an amino acid sequence of SEQ ID NO: 10, or a substantially similar sequence thereof having at least 90%, at least 91%, at least 92%, at least 93%, at least 94%, at least 95%, at least 96%, at least 97%, at least 98%, or at least 99% sequence identity thereto.

The present disclosure also provides antibodies, or antigen-binding fragments thereof, comprising an HCVR and an LCVR pair (HCVR/LCVR) comprising any of the HCVR amino acid sequences listed in Table 3 paired with any of the LCVR amino acid sequences listed in Table 3. According to certain embodiments, the present disclosure provides antibodies, or antigen-binding fragments thereof, comprising an HCVR/LCVR pair contained within any of the exemplary anti-IsdB antibodies listed in Table 3. In certain embodiments, the HCVR/LCVR pair comprises amino acid sequences of SEQ ID NOs: 26/10.

The present disclosure also provides antibodies, or antigen-binding fragments thereof, that bind to IsdB comprising a heavy chain CDR1 (HCDR1) comprising an amino acid sequence selected from any of the HCDR1 amino acid sequences listed in Table 3, or a substantially similar sequence thereof having at least 90%, at least 95%, at least 98% or at least 99% sequence identity.

The present disclosure also provides antibodies, or antigen-binding fragments thereof, that bind to human IsdB comprising a heavy chain CDR2 (HCDR2) comprising an amino acid sequence selected from any of the HCDR2 amino acid sequences listed in Table 3, or a substantially similar sequence thereof having at least 90%, at least 95%, at least 98% or at least 99% sequence identity.

The present disclosure also provides antibodies, or antigen-binding fragments thereof, that bind to IsdB comprising a heavy chain CDR3 (HCDR3) comprising an amino acid sequence selected from any of the HCDR3 amino acid sequences listed in Table 3, or a substantially similar sequence thereof having at least 90%, at least 95%, at least 98% or at least 99% sequence identity.

The present disclosure also provides antibodies, or antigen-binding fragments thereof, that bind to IsdB comprising a light chain CDR1 (LCDR1) comprising an amino acid sequence selected from any of the LCDR1 amino acid sequences listed in Table 3, or a substantially similar sequence thereof having at least 90%, at least 95%, at least 98% or at least 99% sequence identity.

The present disclosure also provides antibodies, or antigen-binding fragments thereof, that bind to IsdB comprising a light chain CDR2 (LCDR2) comprising an amino acid sequence selected from any of the LCDR2 amino acid sequences listed in Table 3, or a substantially similar sequence thereof having at least 90%, at least 95%, at least 98% or at least 99% sequence identity.

The present disclosure also provides antibodies, or antigen-binding fragments thereof, that bind to IsdB comprising a light chain CDR3 (LCDR3) comprising an amino acid sequence selected from any of the LCDR3 amino acid sequences listed in Table 3, or a substantially similar sequence thereof having at least 90%, at least 95%, at least 98% or at least 99% sequence identity.

The present disclosure also provides antibodies, or antigen-binding fragments thereof, that bind to IsdB comprising an HCDR3 and an LCDR3 pair (HCDR3/LCDR3) comprising any of the HCDR3 amino acid sequences listed in Table 3 paired with any of the LCDR3 amino acid sequences listed in Table 3. According to certain embodiments, the present disclosure provides antibodies, or antigen-binding fragments thereof, comprising an HCDR3/LCDR3 pair contained within any of the exemplary IsdB antibodies listed in Table 3. In certain embodiments, the first antigen-binding domain that specifically binds IsdB comprises a HCDR3/LCDR3 pair comprising amino acid sequences of SEQ ID NOs: 32/16.

The present disclosure also provides antibodies, or antigen-binding fragments thereof, that bind to IsdB comprising a heavy chain CDR1 (HCDR1) comprising an amino acid sequence of SEQ ID NO:28, or a substantially similar sequence thereof having at least 90%, at least 95%, at least 98% or at least 99% sequence identity; a heavy chain CDR2 (HCDR2) comprising an amino acid sequence of SEQ ID NO:30, or a substantially similar sequence thereof having at least 90%, at least 95%, at least 98% or at least 99% sequence identity; and a heavy chain CDR3 (HCDR3) comprising an amino acid sequence of SEQ ID NO:32, or a substantially similar sequence thereof having at least 90%, at least 95%, at least 98% or at least 99% sequence identity. In embodiments, the first antigen-binding domain that specifically binds IsdB comprises a light chain CDR1 (LCDR1) comprising an amino acid sequence of SEQ ID NO:12, or a substantially similar sequence thereof having at least 90%, at least 95%, at least 98% or at least 99% sequence identity; a light chain CDR2 (LCDR2) comprising an amino acid sequence of SEQ ID NO:14, or a substantially similar sequence thereof having at least 90%, at least 95%, at least 98% or at least 99% sequence identity; and a light chain CDR3 (LCDR3) comprising an amino acid sequence of SEQ ID NO:16, or a substantially similar sequence thereof having at least 90%, at least 95%, at least 98% or at least 99% sequence identity.

Certain non-limiting, exemplary antibodies, or antigen-binding fragments thereof, that bind to IsdB comprise a set of six CDRs HCDR1-HCDR2-HCDR3-LCDR1-LCDR2-LCDR3, respectively, the set comprising amino acid sequences of SEQ ID NOs: 28, 30, 32, 12, 14, 16.

In another aspect, the present disclosure provides nucleic acid molecules encoding any of the HCVR, LCVR or CDR sequences of the antibodies, or antigen-binding fragments thereof, that bind to IsdB including nucleic acid molecules comprising the polynucleotide sequences as set forth in Table 4 herein, as well as nucleic acid molecules comprising two or more of the polynucleotide sequences as set forth in Tables 2 and 4 in any functional combination or arrangement thereof. Recombinant expression vectors carrying the nucleic acids of the disclosure, and host cells into which such vectors have been introduced, are also encompassed by the disclosure, as are methods of producing the antibodies by culturing the host cells under conditions permitting production of the antibodies, and recovering the antibodies produced.

The present disclosure also provides nucleic acid molecules encoding anti-IsdB antibodies or portions thereof. For example, the present disclosure provides nucleic acid molecules encoding any of the HCVR amino acid sequences listed in Table 3; in certain embodiments the nucleic acid molecule comprises a polynucleotide sequence selected from any of the HCVR nucleic acid sequences listed in Table 4, or a substantially similar sequence thereof having at least 90%, at least 95%, at least 98% or at least 99% sequence identity thereto.

The present disclosure also provides nucleic acid molecules encoding any of the LCVR amino acid sequences listed in Table 3; in certain embodiments the nucleic acid molecule comprises a polynucleotide sequence selected from any of the LCVR nucleic acid sequences listed in Table 4, or a substantially similar sequence thereof having at least 90%, at least 95%, at least 98% or at least 99% sequence identity thereto.

The present disclosure also provides nucleic acid molecules encoding any of the HCDR1 amino acid sequences listed in Table 3; in certain embodiments the nucleic acid molecule comprises a polynucleotide sequence selected from any of the HCDR1 nucleic acid sequences listed in Table 4, or a substantially similar sequence thereof having at least 90%, at least 95%, at least 98% or at least 99% sequence identity thereto.

The present disclosure also provides nucleic acid molecules encoding any of the HCDR2 amino acid sequences listed in Table 3; in certain embodiments the nucleic acid molecule comprises a polynucleotide sequence selected from any of the HCDR2 nucleic acid sequences listed in Table 4, or a substantially similar sequence thereof having at least 90%, at least 95%, at least 98% or at least 99% sequence identity thereto.

The present disclosure also provides nucleic acid molecules encoding any of the HCDR3 amino acid sequences listed in Table 3; in certain embodiments the nucleic acid molecule comprises a polynucleotide sequence selected from any of the HCDR3 nucleic acid sequences listed in Table 4, or a substantially similar sequence thereof having at least 90%, at least 95%, at least 98% or at least 99% sequence identity thereto.

The present disclosure also provides nucleic acid molecules encoding any of the LCDR1 amino acid sequences listed in Table 3; in certain embodiments the nucleic acid molecule comprises a polynucleotide sequence selected from any of the LCDR1 nucleic acid sequences listed in Table 4, or a substantially similar sequence thereof having at least 90%, at least 95%, at least 98% or at least 99% sequence identity thereto.

The present disclosure also provides nucleic acid molecules encoding any of the LCDR2 amino acid sequences listed in Table 3; in certain embodiments the nucleic acid molecule comprises a polynucleotide sequence selected from any of the LCDR2 nucleic acid sequences listed in Table 4, or a substantially similar sequence thereof having at least 90%, at least 95%, at least 98% or at least 99% sequence identity thereto.

The present disclosure also provides nucleic acid molecules encoding any of the LCDR3 amino acid sequences listed in Table 3; in certain embodiments the nucleic acid molecule comprises a polynucleotide sequence selected from any of the LCDR3 nucleic acid sequences listed in Table 4, or a substantially similar sequence thereof having at least 90%, at least 95%, at least 98% or at least 99% sequence identity thereto.

The present disclosure also provides nucleic acid molecules encoding an HCVR, wherein the HCVR comprises a set of three CDRs (i.e., HCDR1-HCDR2-HCDR3), wherein the HCDR1-HCDR2-HCDR3 set is as defined by any of the exemplary anti-IsdB antibodies listed in Table 3.

The present disclosure also provides nucleic acid molecules encoding an LCVR, wherein the LCVR comprises a set of three CDRs (i.e., LCDR1-LCDR2-LCDR3), wherein the LCDR1-LCDR2-LCDR3 set is as defined by any of the exemplary anti-IsdB antibodies listed in Table 3.

The present disclosure also provides nucleic acid molecules encoding both an HCVR and an LCVR, wherein the HCVR comprises an amino acid sequence of any of the HCVR amino acid sequences listed in Table 3, and wherein the LCVR comprises an amino acid sequence of any of the LCVR amino acid sequences listed in Table 3. In certain embodiments, the nucleic acid molecule comprises a polynucleotide sequence selected from any of the HCVR nucleic acid sequences listed in Table 4, or a substantially similar sequence thereof having at least 90%, at least 95%, at least 98% or at least 99% sequence identity thereto, and a polynucleotide sequence selected from any of the LCVR nucleic acid sequences listed in Table 4, or a substantially similar sequence thereof having at least 90%, at least 95%, at least 98% or at least 99% sequence identity thereto. In certain embodiments according to this aspect of the disclosure, the nucleic acid molecule encodes an HCVR and LCVR, wherein the HCVR and LCVR are both derived from the same anti-IsdB antibody listed in Table 3.

In one embodiment, the antibody or antigen binding fragment thereof specifically binds IsdA. An embodiment of the antigen comprises amino acids as shown in Genbank Accession No. AFH 69349. The anti-IsdA antibodies of the disclosure, or antigen-binding portions thereof, may be included in a bispecific antigen-binding molecule that binds specific target cell types such as infectious agents. In embodiments, the bispecific antigen-binding molecule binds to the Staphylococcus species with an IC₅₀ of about 10 nM or less.

Exemplary anti-IsdA antibodies of the present disclosure are listed in Tables 14 and 15. Table 14 sets forth the amino acid sequence identifiers of the heavy chain variable regions (HCVRs) and light chain variable regions (LCVRs), as well as heavy chain complementarity determining regions (HCDR1, HCDR2 and HCDR3), and light chain complementarity determining regions (LCDR1, LCDR2 and LCDR3) of the exemplary anti-IsdA antibodies. Table 15 sets forth the sequence identifiers of the nucleic acid molecules encoding the HCVRs, LCVRs, HCDR1, HCDR2, HCDR3, LCDR1, LCDR2 and LCDR3 of the exemplary anti-IsdA antibodies.

The present disclosure provides antibodies, or antigen-binding fragments thereof that bind IsdA comprising heavy chain variable region (HCVR) CDRs contained within a HCVR comprising an amino acid sequence of SEQ ID NO: 66, or a substantially similar sequence thereof having at least 90%, at least 91%, at least 92%, at least 93%, at least 94%, at least 95%, at least 96%, at least 97%, at least 98%, or at least 99% sequence identity thereto; and light chain variable region (LCVR) CDRs contained within a LCVR region comprising an amino acid sequence of SEQ ID NO: 10, or a substantially similar sequence thereof having at least 90%, at least 91%, at least 92%, at least 93%, at least 94%, at least 95%, at least 96%, at least 97%, at least 98%, or at least 99% sequence identity thereto.

The present disclosure also provides antibodies, or antigen-binding fragments thereof, comprising an HCVR and an LCVR pair (HCVR/LCVR) comprising any of the HCVR amino acid sequences listed in Table 14 paired with any of the LCVR amino acid sequences listed in Table 14. According to certain embodiments, the present disclosure provides antibodies, or antigen-binding fragments thereof, comprising an HCVR/LCVR pair contained within any of the exemplary anti-IsdA antibodies listed in Table 14. In certain embodiments, the HCVR/LCVR pair comprises amino acid sequences of SEQ ID NOs: 66/10.

The present disclosure also provides antibodies, or antigen-binding fragments thereof, that bind to IsdA comprising a heavy chain CDR1 (HCDR1) comprising an amino acid sequence selected from any of the HCDR1 amino acid sequences listed in Table 14, or a substantially similar sequence thereof having at least 90%, at least 95%, at least 98% or at least 99% sequence identity.

The present disclosure also provides antibodies, or antigen-binding fragments thereof, that bind to human IsdA comprising a heavy chain CDR2 (HCDR2) comprising an amino acid sequence selected from any of the HCDR2 amino acid sequences listed in Table 14, or a substantially similar sequence thereof having at least 90%, at least 95%, at least 98% or at least 99% sequence identity.

The present disclosure also provides antibodies, or antigen-binding fragments thereof, that bind to IsdA comprising a heavy chain CDR3 (HCDR3) comprising an amino acid sequence selected from any of the HCDR3 amino acid sequences listed in Table 14, or a substantially similar sequence thereof having at least 90%, at least 95%, at least 98% or at least 99% sequence identity.

The present disclosure also provides antibodies, or antigen-binding fragments thereof, that bind to IsdA comprising a light chain CDR1 (LCDR1) comprising an amino acid sequence selected from any of the LCDR1 amino acid sequences listed in Table 14, or a substantially similar sequence thereof having at least 90%, at least 95%, at least 98% or at least 99% sequence identity.

The present disclosure also provides antibodies, or antigen-binding fragments thereof, that bind to IsdA comprising a light chain CDR2 (LCDR2) comprising an amino acid sequence selected from any of the LCDR2 amino acid sequences listed in Table 14, or a substantially similar sequence thereof having at least 90%, at least 95%, at least 98% or at least 99% sequence identity.

The present disclosure also provides antibodies, or antigen-binding fragments thereof, that bind to IsdA comprising a light chain CDR3 (LCDR3) comprising an amino acid sequence selected from any of the LCDR3 amino acid sequences listed in Table 14, or a substantially similar sequence thereof having at least 90%, at least 95%, at least 98% or at least 99% sequence identity.

The present disclosure also provides antibodies, or antigen-binding fragments thereof, that bind to IsdA comprising an HCDR3 and an LCDR3 pair (HCDR3/LCDR3) comprising any of the HCDR3 amino acid sequences listed in Table 14 paired with any of the LCDR3 amino acid sequences listed in Table 14. According to certain embodiments, the present disclosure provides antibodies, or antigen-binding fragments thereof, comprising an HCDR3/LCDR3 pair contained within any of the exemplary IsdA antibodies listed in Table 14. In certain embodiments, the first antigen-binding domain that specifically binds IsdA comprises a HCDR3/LCDR3 pair comprising amino acid sequences of SEQ ID NOs: 72/16.

The present disclosure also provides antibodies, or antigen-binding fragments thereof, that bind to IsdA comprising a heavy chain CDR1 (HCDR1) comprising an amino acid sequence of SEQ ID NO:68, or a substantially similar sequence thereof having at least 90%, at least 95%, at least 98% or at least 99% sequence identity; a heavy chain CDR2 (HCDR2) comprising an amino acid sequence of SEQ ID NO:70, or a substantially similar sequence thereof having at least 90%, at least 95%, at least 98% or at least 99% sequence identity; and a heavy chain CDR3 (HCDR3) comprising an amino acid sequence of SEQ ID NO:72, or a substantially similar sequence thereof having at least 90%, at least 95%, at least 98% or at least 99% sequence identity. In embodiments, the first antigen-binding domain that specifically binds IsdA comprises a light chain CDR1 (LCDR1) comprising an amino acid sequence of SEQ ID NO:12, or a substantially similar sequence thereof having at least 90%, at least 95%, at least 98% or at least 99% sequence identity; a light chain CDR2 (LCDR2) comprising an amino acid sequence of SEQ ID NO:14, or a substantially similar sequence thereof having at least 90%, at least 95%, at least 98% or at least 99% sequence identity; and a light chain CDR3 (LCDR3) comprising an amino acid sequence of SEQ ID NO:16, or a substantially similar sequence thereof having at least 90%, at least 95%, at least 98% or at least 99% sequence identity.

Certain non-limiting, exemplary antibodies, or antigen-binding fragments thereof, that bind to IsdA comprise a set of six CDRs HCDR1-HCDR2-HCDR3-LCDR1-LCDR2-LCDR3, respectively, the set comprising amino acid sequences of SEQ ID NOs: 68, 70, 72, 12, 14, 16.

In another aspect, the present disclosure provides nucleic acid molecules encoding any of the HCVR, LCVR or CDR sequences of the antibodies, or antigen-binding fragments thereof, that bind to IsdA including nucleic acid molecules comprising the polynucleotide sequences as set forth in Table 15 herein, as well as nucleic acid molecules comprising two or more of the polynucleotide sequences as set forth in Tables 2 and 15 in any functional combination or arrangement thereof. Recombinant expression vectors carrying the nucleic acids of the disclosure, and host cells into which such vectors have been introduced, are also encompassed by the disclosure, as are methods of producing the antibodies by culturing the host cells under conditions permitting production of the antibodies, and recovering the antibodies produced.

The present disclosure also provides nucleic acid molecules encoding anti-IsdA antibodies or portions thereof. For example, the present disclosure provides nucleic acid molecules encoding any of the HCVR amino acid sequences listed in Table 14; in certain embodiments the nucleic acid molecule comprises a polynucleotide sequence selected from any of the HCVR nucleic acid sequences listed in Table 15, or a substantially similar sequence thereof having at least 90%, at least 95%, at least 98% or at least 99% sequence identity thereto.

The present disclosure also provides nucleic acid molecules encoding any of the LCVR amino acid sequences listed in Table 14; in certain embodiments the nucleic acid molecule comprises a polynucleotide sequence selected from any of the LCVR nucleic acid sequences listed in Table 15, or a substantially similar sequence thereof having at least 90%, at least 95%, at least 98% or at least 99% sequence identity thereto.

The present disclosure also provides nucleic acid molecules encoding any of the HCDR1 amino acid sequences listed in Table 14; in certain embodiments the nucleic acid molecule comprises a polynucleotide sequence selected from any of the HCDR1 nucleic acid sequences listed in Table 15, or a substantially similar sequence thereof having at least 90%, at least 95%, at least 98% or at least 99% sequence identity thereto.

The present disclosure also provides nucleic acid molecules encoding any of the HCDR2 amino acid sequences listed in Table 14; in certain embodiments the nucleic acid molecule comprises a polynucleotide sequence selected from any of the HCDR2 nucleic acid sequences listed in Table 15, or a substantially similar sequence thereof having at least 90%, at least 95%, at least 98% or at least 99% sequence identity thereto.

The present disclosure also provides nucleic acid molecules encoding any of the HCDR3 amino acid sequences listed in Table 14; in certain embodiments the nucleic acid molecule comprises a polynucleotide sequence selected from any of the HCDR3 nucleic acid sequences listed in Table 15, or a substantially similar sequence thereof having at least 90%, at least 95%, at least 98% or at least 99% sequence identity thereto.

The present disclosure also provides nucleic acid molecules encoding any of the LCDR1 amino acid sequences listed in Table 14; in certain embodiments the nucleic acid molecule comprises a polynucleotide sequence selected from any of the LCDR1 nucleic acid sequences listed in Table 15, or a substantially similar sequence thereof having at least 90%, at least 95%, at least 98% or at least 99% sequence identity thereto.

The present disclosure also provides nucleic acid molecules encoding any of the LCDR2 amino acid sequences listed in Table 14; in certain embodiments the nucleic acid molecule comprises a polynucleotide sequence selected from any of the LCDR2 nucleic acid sequences listed in Table 15, or a substantially similar sequence thereof having at least 90%, at least 95%, at least 98% or at least 99% sequence identity thereto.

The present disclosure also provides nucleic acid molecules encoding any of the LCDR3 amino acid sequences listed in Table 14; in certain embodiments the nucleic acid molecule comprises a polynucleotide sequence selected from any of the LCDR3 nucleic acid sequences listed in Table 15, or a substantially similar sequence thereof having at least 90%, at least 95%, at least 98% or at least 99% sequence identity thereto.

The present disclosure also provides nucleic acid molecules encoding an HCVR, wherein the HCVR comprises a set of three CDRs (i.e., HCDR1-HCDR2-HCDR3), wherein the HCDR1-HCDR2-HCDR3 set is as defined by any of the exemplary anti-IsdA antibodies listed in Table 14.

The present disclosure also provides nucleic acid molecules encoding an LCVR, wherein the LCVR comprises a set of three CDRs (i.e., LCDR1-LCDR2-LCDR3), wherein the LCDR1-LCDR2-LCDR3 set is as defined by any of the exemplary anti-IsdA antibodies listed in Table 14.

The present disclosure also provides nucleic acid molecules encoding both an HCVR and an LCVR, wherein the HCVR comprises an amino acid sequence of any of the HCVR amino acid sequences listed in Table 14, and wherein the LCVR comprises an amino acid sequence of any of the LCVR amino acid sequences listed in Table 14. In certain embodiments, the nucleic acid molecule comprises a polynucleotide sequence selected from any of the HCVR nucleic acid sequences listed in Table 15, or a substantially similar sequence thereof having at least 90%, at least 95%, at least 98% or at least 99% sequence identity thereto, and a polynucleotide sequence selected from any of the LCVR nucleic acid sequences listed in Table 15, or a substantially similar sequence thereof having at least 90%, at least 95%, at least 98% or at least 99% sequence identity thereto. In certain embodiments according to this aspect of the disclosure, the nucleic acid molecule encodes an HCVR and LCVR, wherein the HCVR and LCVR are both derived from the same anti-IsdA antibody listed in Table 14.

In one embodiment, the antibody or antigen binding fragment thereof specifically binds Protein A. In one embodiment, Protein A comprises amino acids as shown in Genbank Accession No. AGE10362. The anti-Protein A antibodies of the disclosure, or antigen-binding portions thereof, may be included in a bispecific antigen-binding molecule that binds specific target cell types such as infectious agents. In embodiments, the bispecific antigen-binding molecule binds to the Staphylococcus species with an IC₅₀ of about 10 nM or less.

Exemplary anti-Protein A antibodies of the present disclosure are listed in Tables 12 and 13. Table 12 sets forth the amino acid sequence identifiers of the heavy chain variable regions (HCVRs) and light chain variable regions (LCVRs), as well as heavy chain complementarity determining regions (HCDR1, HCDR2 and HCDR3), and light chain complementarity determining regions (LCDR1, LCDR2 and LCDR3) of the exemplary anti-Protein A antibodies. Table 13 sets forth the sequence identifiers of the nucleic acid molecules encoding the HCVRs, LCVRs, HCDR1, HCDR2, HCDR3, LCDR1, LCDR2 and LCDR3 of the exemplary anti-Protein A antibodies.

The present disclosure provides antibodies, or antigen-binding fragments thereof that bind Protein A comprising heavy chain variable region (HCVR) CDRs contained within a HCVR comprising an amino acid sequence of SEQ ID NO: 58, or a substantially similar sequence thereof having at least 90%, at least 91%, at least 92%, at least 93%, at least 94%, at least 95%, at least 96%, at least 97%, at least 98%, or at least 99% sequence identity thereto; and light chain variable region (LCVR) CDRs contained within a LCVR region comprising an amino acid sequence of SEQ ID NO: 10, or a substantially similar sequence thereof having at least 90%, at least 91%, at least 92%, at least 93%, at least 94%, at least 95%, at least 96%, at least 97%, at least 98%, or at least 99% sequence identity thereto.

The present disclosure also provides antibodies, or antigen-binding fragments thereof, comprising an HCVR and an LCVR pair (HCVR/LCVR) comprising any of the HCVR amino acid sequences listed in Table 12 paired with any of the LCVR amino acid sequences listed in Table 12. According to certain embodiments, the present disclosure provides antibodies, or antigen-binding fragments thereof, comprising an HCVR/LCVR pair contained within any of the exemplary anti-Protein A antibodies listed in Table 12. In certain embodiments, the HCVR/LCVR pair comprises amino acid sequences of SEQ ID NOs: 58/10.

The present disclosure also provides antibodies, or antigen-binding fragments thereof, that bind to Protein A comprising a heavy chain CDR1 (HCDR1) comprising an amino acid sequence selected from any of the HCDR1 amino acid sequences listed in Table 12, or a substantially similar sequence thereof having at least 90%, at least 95%, at least 98% or at least 99% sequence identity.

The present disclosure also provides antibodies, or antigen-binding fragments thereof, that bind to human Protein A comprising a heavy chain CDR2 (HCDR2) comprising an amino acid sequence selected from any of the HCDR2 amino acid sequences listed in Table 12, or a substantially similar sequence thereof having at least 90%, at least 95%, at least 98% or at least 99% sequence identity.

The present disclosure also provides antibodies, or antigen-binding fragments thereof, that bind to Protein A comprising a heavy chain CDR3 (HCDR3) comprising an amino acid sequence selected from any of the HCDR3 amino acid sequences listed in Table 12, or a substantially similar sequence thereof having at least 90%, at least 95%, at least 98% or at least 99% sequence identity.

The present disclosure also provides antibodies, or antigen-binding fragments thereof, that bind to Protein A comprising a light chain CDR1 (LCDR1) comprising an amino acid sequence selected from any of the LCDR1 amino acid sequences listed in Table 12, or a substantially similar sequence thereof having at least 90%, at least 95%, at least 98% or at least 99% sequence identity.

The present disclosure also provides antibodies, or antigen-binding fragments thereof, that bind to Protein A comprising a light chain CDR2 (LCDR2) comprising an amino acid sequence selected from any of the LCDR2 amino acid sequences listed in Table 12, or a substantially similar sequence thereof having at least 90%, at least 95%, at least 98% or at least 99% sequence identity.

The present disclosure also provides antibodies, or antigen-binding fragments thereof, that bind to Protein A comprising a light chain CDR3 (LCDR3) comprising an amino acid sequence selected from any of the LCDR3 amino acid sequences listed in Table 12, or a substantially similar sequence thereof having at least 90%, at least 95%, at least 98% or at least 99% sequence identity.

The present disclosure also provides antibodies, or antigen-binding fragments thereof, that bind to Protein A comprising an HCDR3 and an LCDR3 pair (HCDR3/LCDR3) comprising any of the HCDR3 amino acid sequences listed in Table 12 paired with any of the LCDR3 amino acid sequences listed in Table 12. According to certain embodiments, the present disclosure provides antibodies, or antigen-binding fragments thereof, comprising an HCDR3/LCDR3 pair contained within any of the exemplary Protein A antibodies listed in Table 12. In certain embodiments, the first antigen-binding domain that specifically binds Protein A comprises a HCDR3/LCDR3 pair comprising amino acid sequences of SEQ ID NOs: 64/16.

The present disclosure also provides antibodies, or antigen-binding fragments thereof, that bind to Protein A comprising a heavy chain CDR1 (HCDR1) comprising an amino acid sequence of SEQ ID NO:60, or a substantially similar sequence thereof having at least 90%, at least 95%, at least 98% or at least 99% sequence identity; a heavy chain CDR2 (HCDR2) comprising an amino acid sequence of SEQ ID NO:62, or a substantially similar sequence thereof having at least 90%, at least 95%, at least 98% or at least 99% sequence identity; and a heavy chain CDR3 (HCDR3) comprising an amino acid sequence of SEQ ID NO:64, or a substantially similar sequence thereof having at least 90%, at least 95%, at least 98% or at least 99% sequence identity. In embodiments, the first antigen-binding domain that specifically binds Protein A comprises a light chain CDR1 (LCDR1) comprising an amino acid sequence of SEQ ID NO:12, or a substantially similar sequence thereof having at least 90%, at least 95%, at least 98% or at least 99% sequence identity; a light chain CDR2 (LCDR2) comprising an amino acid sequence of SEQ ID NO:14, or a substantially similar sequence thereof having at least 90%, at least 95%, at least 98% or at least 99% sequence identity; and a light chain CDR3 (LCDR3) comprising an amino acid sequence of SEQ ID NO:16, or a substantially similar sequence thereof having at least 90%, at least 95%, at least 98% or at least 99% sequence identity.

Certain non-limiting, exemplary antibodies, or antigen-binding fragments thereof, that bind to Protein A comprise a set of six CDRs HCDR1-HCDR2-HCDR3-LCDR1-LCDR2-LCDR3, respectively, the set comprising amino acid sequences of SEQ ID NOs: 60, 62, 64, 12, 14, 16.

In another aspect, the present disclosure provides nucleic acid molecules encoding any of the HCVR, LCVR or CDR sequences of the antibodies, or antigen-binding fragments thereof, that bind to Protein A including nucleic acid molecules comprising the polynucleotide sequences as set forth in Table 13 herein, as well as nucleic acid molecules comprising two or more of the polynucleotide sequences as set forth in Tables 2 and 13 in any functional combination or arrangement thereof. Recombinant expression vectors carrying the nucleic acids of the disclosure, and host cells into which such vectors have been introduced, are also encompassed by the disclosure, as are methods of producing the antibodies by culturing the host cells under conditions permitting production of the antibodies, and recovering the antibodies produced.

The present disclosure also provides nucleic acid molecules encoding anti-Protein A antibodies or portions thereof. For example, the present disclosure provides nucleic acid molecules encoding any of the HCVR amino acid sequences listed in Table 12; in certain embodiments the nucleic acid molecule comprises a polynucleotide sequence selected from any of the HCVR nucleic acid sequences listed in Table 13, or a substantially similar sequence thereof having at least 90%, at least 95%, at least 98% or at least 99% sequence identity thereto.

The present disclosure also provides nucleic acid molecules encoding any of the LCVR amino acid sequences listed in Table 12; in certain embodiments the nucleic acid molecule comprises a polynucleotide sequence selected from any of the LCVR nucleic acid sequences listed in Table 13, or a substantially similar sequence thereof having at least 90%, at least 95%, at least 98% or at least 99% sequence identity thereto.

The present disclosure also provides nucleic acid molecules encoding any of the HCDR1 amino acid sequences listed in Table 12; in certain embodiments the nucleic acid molecule comprises a polynucleotide sequence selected from any of the HCDR1 nucleic acid sequences listed in Table 13, or a substantially similar sequence thereof having at least 90%, at least 95%, at least 98% or at least 99% sequence identity thereto.

The present disclosure also provides nucleic acid molecules encoding any of the HCDR2 amino acid sequences listed in Table 12; in certain embodiments the nucleic acid molecule comprises a polynucleotide sequence selected from any of the HCDR2 nucleic acid sequences listed in Table 13, or a substantially similar sequence thereof having at least 90%, at least 95%, at least 98% or at least 99% sequence identity thereto.

The present disclosure also provides nucleic acid molecules encoding any of the HCDR3 amino acid sequences listed in Table 12; in certain embodiments the nucleic acid molecule comprises a polynucleotide sequence selected from any of the HCDR3 nucleic acid sequences listed in Table 13, or a substantially similar sequence thereof having at least 90%, at least 95%, at least 98% or at least 99% sequence identity thereto.

The present disclosure also provides nucleic acid molecules encoding any of the LCDR1 amino acid sequences listed in Table 12; in certain embodiments the nucleic acid molecule comprises a polynucleotide sequence selected from any of the LCDR1 nucleic acid sequences listed in Table 13, or a substantially similar sequence thereof having at least 90%, at least 95%, at least 98% or at least 99% sequence identity thereto.

The present disclosure also provides nucleic acid molecules encoding any of the LCDR2 amino acid sequences listed in Table 12; in certain embodiments the nucleic acid molecule comprises a polynucleotide sequence selected from any of the LCDR2 nucleic acid sequences listed in Table 13, or a substantially similar sequence thereof having at least 90%, at least 95%, at least 98% or at least 99% sequence identity thereto.

The present disclosure also provides nucleic acid molecules encoding any of the LCDR3 amino acid sequences listed in Table 12; in certain embodiments the nucleic acid molecule comprises a polynucleotide sequence selected from any of the LCDR3 nucleic acid sequences listed in Table 13, or a substantially similar sequence thereof having at least 90%, at least 95%, at least 98% or at least 99% sequence identity thereto.

The present disclosure also provides nucleic acid molecules encoding an HCVR, wherein the HCVR comprises a set of three CDRs (i.e., HCDR1-HCDR2-HCDR3), wherein the HCDR1-HCDR2-HCDR3 set is as defined by any of the exemplary anti-Protein A antibodies listed in Table 12.

The present disclosure also provides nucleic acid molecules encoding an LCVR, wherein the LCVR comprises a set of three CDRs (i.e., LCDR1-LCDR2-LCDR3), wherein the LCDR1-LCDR2-LCDR3 set is as defined by any of the exemplary anti-Protein A antibodies listed in Table 12.

The present disclosure also provides nucleic acid molecules encoding both an HCVR and an LCVR, wherein the HCVR comprises an amino acid sequence of any of the HCVR amino acid sequences listed in Table 12, and wherein the LCVR comprises an amino acid sequence of any of the LCVR amino acid sequences listed in Table 12. In certain embodiments, the nucleic acid molecule comprises a polynucleotide sequence selected from any of the HCVR nucleic acid sequences listed in Table 13, or a substantially similar sequence thereof having at least 90%, at least 95%, at least 98% or at least 99% sequence identity thereto, and a polynucleotide sequence selected from any of the LCVR nucleic acid sequences listed in Table 13, or a substantially similar sequence thereof having at least 90%, at least 95%, at least 98% or at least 99% sequence identity thereto. In certain embodiments according to this aspect of the disclosure, the nucleic acid molecule encodes an HCVR and LCVR, wherein the HCVR and LCVR are both derived from the same anti-Protein A antibody listed in Table 13.

In one embodiment, the antibody or antigen binding fragment thereof specifically binds PNAG. In one embodiment, the PNAG antigen comprises a capsular extract obtained as described by Cramton et. al., Infect. Immun. 2001.69:4079. The anti-PNAG antibodies of the disclosure, or antigen-binding portions thereof, may be included in a bispecific antigen-binding molecule that binds specific target cell types such as infectious agents. In embodiments, the bispecific antigen-binding molecule binds to the Staphylococcus species with an IC₅₀ of about 10 nM or less.

Exemplary anti-PNAG antibodies of the present disclosure are described in U.S. Pat. No. 7,786,255. Exemplary antibodies are also listed in Table 16. Table 16 sets forth the amino acid sequence identifiers of the heavy chain (HC) and light chain (LC) of the exemplary anti-PNAG antibodies.

The present disclosure provides antibodies, or antigen-binding fragments thereof that bind PNAG comprising heavy chain variable region (HCVR) CDRs contained within a HC comprising an amino acid sequence of SEQ ID NO: 76, or a substantially similar sequence thereof having at least 90%, at least 95%, at least 98%, or at least 99% sequence identity thereto; and light chain variable region (LCVR) CDRs contained within a LC comprising an amino acid sequence of SEQ ID NO: 77, or a substantially similar sequence thereof having at least 90%, at least 95%, at least 98%, or at least 99% sequence identity thereto. In embodiments, the antibody or antigen-binding fragment thereof comprises HCVR CDRs contained within a HCVR comprising an amino acid sequence of amino acids 1-118 of SEQ ID NO:76, or a substantially similar sequence thereof having at least 90%, at least 91%, at least 92%, at least 93%, at least 94%, at least 95%, at least 96%, at least 97%, at least 98%, or at least 99% sequence identity thereto; and LCVR CDRs contained within a LCVR region comprising an amino acid sequence of amino acids 1-108 of SEQ ID NO: 77, or a substantially similar sequence thereof having at least 90%, at least 91%, at least 92%, at least 93%, at least 94%, at least 95%, at least 96%, at least 97%, at least 98%, or at least 99% sequence identity thereto.

The present disclosure also provides antibodies, or antigen-binding fragments thereof, comprising an HCVR and an LCVR pair (HCVR/LCVR) comprising any of the HCVR amino acid sequences contained within the HC sequence listed in Table 16 paired with any of the LCVR amino acid sequences contained within the LC sequence listed in Table 16. According to certain embodiments, the present disclosure provides antibodies, or antigen-binding fragments thereof, comprising an HCVR/LCVR pair contained within any of the exemplary anti-PNAG antibodies listed in Table 16. In certain embodiments, the HCVR/LCVR pair comprises amino acid sequences contained within the HC/LC of SEQ ID NOs: 76/77.

In another aspect, the present disclosure provides nucleic acid molecules encoding any of the HCVR or LCVR sequences of the antibodies, or antigen-binding fragments thereof, that bind to PNAG. Recombinant expression vectors carrying the nucleic acids of the disclosure, and host cells into which such vectors have been introduced, are also encompassed by the disclosure, as are methods of producing the antibodies by culturing the host cells under conditions permitting production of the antibodies, and recovering the antibodies produced.

The present disclosure also provides nucleic acid molecules encoding anti-PNAG antibodies or portions thereof. For example, the present disclosure also provides nucleic acid molecules encoding both an HCVR and an LCVR, wherein the HCVR comprises an amino acid sequence contained within the HC amino acid sequence listed in Table 16, and wherein the LCVR comprises an amino acid sequence contained within the LC amino acid sequence listed in Table 16. In certain embodiments according to this aspect of the disclosure, the nucleic acid molecule encodes an HCVR and LCVR, wherein the HCVR and LCVR are both derived from the same anti-PNAG antibody listed in Table 16.

In one embodiment, the antibody or antigen binding fragment thereof specifically binds ClfA. An embodiment of the antigen comprises amino acids as shown in Genbank Accession No. RUQ 57133. The anti-ClfA antibodies of the disclosure, or antigen-binding portions thereof, may be included in a bispecific antigen-binding molecule that binds specific target cell types such as infectious agents. In embodiments, the bispecific antigen-binding molecule binds to the Staphylococcus species with an IC₅₀ of about 10 nM or less.

Exemplary antibodies are described in U.S. Publication No. US2003/0099656. Exemplary anti-ClfA antibodies of the present disclosure are listed in Table 17. Table 17 sets forth the amino acid sequence identifiers of the heavy chain (HC) and light chain (LC) of the exemplary anti-ClfA antibodies.

The present disclosure provides antibodies, or antigen-binding fragments thereof that bind ClfA comprising heavy chain variable region (HCVR) CDRs contained within a HC amino acid sequence of SEQ ID NO: 73, or a substantially similar sequence thereof having at least 90%, at least 95%, at least 98%, or at least 99% sequence identity thereto; and light chain variable region (LCVR) CDRs contained within a LC amino acid sequence of SEQ ID NO: 74, or a substantially similar sequence thereof having at least 90%, at least 95%, at least 98%, or at least 99% sequence identity thereto. In embodiments, the antibody or antigen-binding fragment thereof comprises HCVR CDRs contained within a HCVR comprising an amino acid sequence of amino acids 1-118 of SEQ ID NO:73, or a substantially similar sequence thereof having at least 90%, at least 91%, at least 92%, at least 93%, at least 94%, at least 95%, at least 96%, at least 97%, at least 98%, or at least 99% sequence identity thereto; and LCVR CDRs contained within a LCVR region comprising an amino acid sequence of amino acids 1-108 of SEQ ID NO: 74, or a substantially similar sequence thereof having at least 90%, at least 91%, at least 92%, at least 93%, at least 94%, at least 95%, at least 96%, at least 97%, at least 98%, or at least 99% sequence identity thereto.

The present disclosure also provides antibodies, or antigen-binding fragments thereof, comprising an HCVR and an LCVR pair (HCVR/LCVR) comprising any of the HCVR amino acid sequences contained within the HC listed in Table 17 paired with any of the LCVR amino acid sequences contained within the LC listed in Table 17. According to certain embodiments, the present disclosure provides antibodies, or antigen-binding fragments thereof, comprising an HCVR/LCVR pair contained within any of the exemplary anti-ClfA antibodies listed in Table 17. In certain embodiments, the HCVR/LCVR pair comprises amino acid sequences contained within the HC/LC of SEQ ID NOs: 73/74.

In another aspect, the present disclosure provides nucleic acid molecules encoding any of the HCVR or LCVR sequences of the antibodies, or antigen-binding fragments thereof that bind to ClfA. Recombinant expression vectors carrying the nucleic acids of the disclosure, and host cells into which such vectors have been introduced, are also encompassed by the disclosure, as are methods of producing the antibodies by culturing the host cells under conditions permitting production of the antibodies, and recovering the antibodies produced.

The present disclosure also provides nucleic acid molecules encoding anti-ClfA antibodies or portions thereof. For example, the present disclosure also provides nucleic acid molecules encoding both an HCVR and an LCVR, wherein the HCVR comprises an amino acid sequence contained within the HC of the amino acid sequence listed in Table 17, and wherein the LCVR comprises an amino acid sequence contained within the LC amino acid sequence listed in Table 17. In certain embodiments according to this aspect of the disclosure, the nucleic acid molecule encodes an HCVR and LCVR, wherein the HCVR and LCVR are both derived from the same anti-ClfA antibody listed in Table 17.

The present disclosure also provides recombinant expression vectors capable of expressing a polypeptide comprising a heavy or light chain variable region of any of the antibodies described herein. For example, the present disclosure includes recombinant expression vectors comprising any of the nucleic acid molecules mentioned above, i.e., nucleic acid molecules encoding any of the HCVR or LCVR sequences as set forth in Tables 3, 12, 14, 16, or 17. Also included within the scope of the present disclosure are host cells into which such vectors have been introduced, as well as methods of producing the antibodies or portions thereof by culturing the host cells under conditions permitting production of the antibodies or antibody fragments, and recovering the antibodies and antibody fragments so produced.

The present disclosure includes antibodies as described herein having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

### B. Pseudomonas Target Antigens

In another aspect, the present disclosure provides binding molecules, e.g., antibodies and antigen-binding fragments thereof, that bind a Pseudomonas species target antigen (e.g. a monospecific antibody to a Pseudomonas species target antigen). In embodiments, the Pseudomonas species is a species that causes an infection in humans, such as but not limited to Pseudomonas aeruginosa PAO-1. In embodiments, the Pseudomonas species target antigen comprises an extracellular polysaccharide such as but not limited to Psl. In embodiments, the Pseudomonas species target antigen comprises Psl, other Pseudomonas target antigens, or combinations thereof. An embodiment of the Psl antigen comprises a polysaccharide as described by Byrd et. al. Mol. Microb. 2009. 73:622. In one embodiment, the antibody or antigen binding fragment thereof specifically binds Psl.

Exemplary anti-Psl antibodies are described in WO2012/170807, incorporated by reference herein. The anti-Psl antibodies of the disclosure, or antigen-binding portions thereof, may be included in a bispecific antigen-binding molecule that binds specific target cell types such as infectious agents. In embodiments, the bispecific antigen-binding molecule binds to the Pseudomonas species with an IC₅₀ of about 10 nM or less.

Exemplary anti-Psl antibodies of the present disclosure are listed in Table 11. Table 11 sets forth the amino acid sequence identifiers of the heavy chain (HC) and light chain (LC) of an exemplary anti-Psl antibody. The present disclosure provides antibodies, or antigen-binding fragments thereof that bind Psl comprising heavy chain variable region (HCVR) CDRs contained within a HCVR comprising an amino acid sequence of amino acids 1-118 of SEQ ID NO:78, or a substantially similar sequence thereof having at least 90%, at least 91%, at least 92%, at least 93%, at least 94%, at least 95%, at least 96%, at least 97%, at least 98%, or at least 99% sequence identity thereto; and light chain variable region (LCVR) CDRs contained within a LCVR region comprising an amino acid sequence of amino acids 1-108 of SEQ ID NO: 79, or a substantially similar sequence thereof having at least 90%, at least 91%, at least 92%, at least 93%, at least 94%, at least 95%, at least 96%, at least 97%, at least 98%, or at least 99% sequence identity thereto.

The present disclosure also provides antibodies, or antigen-binding fragments thereof, comprising an HCVR and an LCVR pair (HCVR/LCVR) comprising any of the HCVR amino acid sequences contained within the HC listed in Table 11 paired with any of the LCVR amino acid sequences contained within the LC listed in Table 11. According to certain embodiments, the present disclosure provides antibodies, or antigen-binding fragments thereof, comprising an HCVR/LCVR pair contained within the HC/LC of the exemplary anti-Psl antibodies listed in Table 11. In certain embodiments, the HCVR/LCVR pair comprises amino acid sequences contained within the HC/LC pair of SEQ ID NOs: 78 (amino acids 1-118)/79 (amino acids 1-108).

The present disclosure also provides antibodies, or antigen-binding fragments thereof, that bind to Psl comprising a heavy chain CDR1 (HCDR1) comprising an amino acid sequence selected from any of the HCDR1 amino acid sequences listed in Table 11, or a substantially similar sequence thereof having at least 90%, at least 95%, at least 98% or at least 99% sequence identity.

The present disclosure also provides antibodies, or antigen-binding fragments thereof, that bind to human Psl comprising a heavy chain CDR2 (HCDR2) comprising an amino acid sequence selected from any of the HCDR2 amino acid sequences listed in Table 11, or a substantially similar sequence thereof having at least 90%, at least 95%, at least 98% or at least 99% sequence identity.

The present disclosure also provides antibodies, or antigen-binding fragments thereof, that bind to Psl comprising a heavy chain CDR3 (HCDR3) comprising an amino acid sequence selected from any of the HCDR3 amino acid sequences listed in Table 11, or a substantially similar sequence thereof having at least 90%, at least 95%, at least 98% or at least 99% sequence identity.

The present disclosure also provides antibodies, or antigen-binding fragments thereof, that bind to Psl comprising a light chain CDR1 (LCDR1) comprising an amino acid sequence selected from any of the LCDR1 amino acid sequences listed in Table 11, or a substantially similar sequence thereof having at least 90%, at least 95%, at least 98% or at least 99% sequence identity.

The present disclosure also provides antibodies, or antigen-binding fragments thereof, that bind to Psl comprising a light chain CDR2 (LCDR2) comprising an amino acid sequence selected from any of the LCDR2 amino acid sequences listed in Table 11, or a substantially similar sequence thereof having at least 90%, at least 95%, at least 98% or at least 99% sequence identity.

The present disclosure also provides antibodies, or antigen-binding fragments thereof, that bind to Psl comprising a light chain CDR3 (LCDR3) comprising an amino acid sequence selected from any of the LCDR3 amino acid sequences listed in Table 11, or a substantially similar sequence thereof having at least 90%, at least 95%, at least 98% or at least 99% sequence identity.

The present disclosure also provides antibodies, or antigen-binding fragments thereof, that bind to Psl comprising an HCDR3 and an LCDR3 pair (HCDR3/LCDR3) comprising any of the HCDR3 amino acid sequences listed in Table 11 paired with any of the LCDR3 amino acid sequences listed in Table 11. According to certain embodiments, the present disclosure provides antibodies, or antigen-binding fragments thereof, comprising an HCDR3/LCDR3 pair contained within any of the exemplary Psl antibodies listed in Table 11. In certain embodiments, the first antigen-binding domain that specifically binds Psl comprises a HCDR3/LCDR3 pair comprising amino acid sequences of SEQ ID NOs:78 (amino acids 98-109)/79 (amino acids 88-99).

The present disclosure also provides antibodies, or antigen-binding fragments thereof, that bind to Psl comprising a heavy chain CDR1 (HCDR1) comprising an amino acid sequence of amino acids 31-35 of SEQ ID NO:78, or a substantially similar sequence thereof having at least 90%, at least 95%, at least 98% or at least 99% sequence identity; a heavy chain CDR2 (HCDR2) comprising an amino acid sequence of amino acids 50-65 of SEQ ID NO:78 or a substantially similar sequence thereof having at least 90%, at least 95%, at least 98% or at least 99% sequence identity; and a heavy chain CDR3 (HCDR3) comprising an amino acid sequence of amino acids 98-109 of SEQ ID NO:78, or a substantially similar sequence thereof having at least 90%, at least 95%, at least 98% or at least 99% sequence identity. In embodiments, the first antigen-binding domain that specifically binds Psl comprises a light chain CDR1 (LCDR1) comprising an amino acid sequence of amino acids 23-33 of SEQ ID NO:79, or a substantially similar sequence thereof having at least 90%, at least 95%, at least 98% or at least 99% sequence identity; a light chain CDR2 (LCDR2) comprising an amino acid sequence of amino acids 49-55 of SEQ ID NO:79 or a substantially similar sequence thereof having at least 90%, at least 95%, at least 98% or at least 99% sequence identity; and a light chain CDR3 (LCDR3) comprising an amino acid sequence of amino acids 88-99 of SEQ ID NO:79 or a substantially similar sequence thereof having at least 90%, at least 95%, at least 98% or at least 99% sequence identity.

Certain non-limiting, exemplary antibodies, or antigen-binding fragments thereof, that bind to Psl comprise a set of six CDRs HCDR1-HCDR2-HCDR3-LCDR1-LCDR2-LCDR3, respectively, the set comprising amino acid sequences of SEQ ID NOs:78 (amino acids 31-35)/78 (amino acids 50-65)/78 (amino acids 98-109)/79 (amino acids 23-33)/79 (amino acids 49-55)/79 (amino acids 88-99).

In another aspect, the present disclosure provides nucleic acid molecules encoding any of the HCVR, LCVR or CDR sequences of the antibodies, or antigen-binding fragments thereof, that bind to Psl. Recombinant expression vectors carrying the nucleic acids of the disclosure, and host cells into which such vectors have been introduced, are also encompassed by the disclosure, as are methods of producing the antibodies by culturing the host cells under conditions permitting production of the antibodies, and recovering the antibodies produced.

The present disclosure also provides nucleic acid molecules encoding anti-Psl antibodies or portions thereof. For example, the present disclosure provides nucleic acid molecules encoding any of the HCVR amino acid sequences listed in Table 11.

The present disclosure also provides nucleic acid molecules encoding both an HCVR and an LCVR, wherein the HCVR comprises an amino acid sequence contained within the HC amino acid sequences listed in Table 11, and wherein the LCVR comprises an amino acid sequence contained within the LC amino acid sequences listed in Table 11. In certain embodiments according to this aspect of the disclosure, the nucleic acid molecule encodes an HCVR and LCVR, wherein the HCVR and LCVR are both derived from the same anti-Psl antibody listed in Table 11.

The present disclosure also provides recombinant expression vectors capable of expressing a polypeptide comprising a heavy or light chain variable region of an anti-Psl antibody. For example, the present disclosure includes recombinant expression vectors comprising any of the nucleic acid molecules mentioned above, i.e., nucleic acid molecules encoding any of the HCVR, LCVR, and/or CDR sequences contained within the HC and LC as set forth in Table 11. Also included within the scope of the present disclosure are host cells into which such vectors have been introduced, as well as methods of producing the antibodies or portions thereof by culturing the host cells under conditions permitting production of the antibodies or antibody fragments, and recovering the antibodies and antibody fragments so produced.

The present disclosure includes anti-Psl antibodies having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

### C. Virus Target Antigens

In another aspect, the present disclosure provides binding molecules, e.g., antibodies and antigen-binding fragments thereof, that bind a virus target antigen. Exemplary viruses that can be targeted include, but are not limited to, influenza virus, smallpox virus, hepatitis virus (e.g., hepatitis A, hepatitis B, or hepatitis C virus), human papillomavirus, human immunodeficiency virus (HIV), respiratory syncytial virus (RSV), herpesvirus, coronavirus, cowpox virus, coxsackievirus, Ebola virus, Epstein-Barr virus, measles virus, mumps virus, rubella virus, rotavirus, rabies virus, yellow fever virus, meningitis, poliovirus, rotavirus, vaccinia virus, varicella-zoster virus, variola virus, and Zika virus. In some embodiments, the target antigen is a viral glycoprotein.

In some embodiments the present disclosure provides antibodies and antigen-binding fragments thereof that bind an Influenza virus target antigen (e.g. a monospecific antibody to an Influenza virus target antigen). In embodiments, the Influenza virus antigen is a species that causes an infection in humans, including but not limited to Influenza A, Influenza B, Influenza C, and Influenza D. In embodiments, the Influenza virus target antigen comprises Influenza HA, other Influenza virus target antigens, or combinations thereof. An embodiment of the antigen comprises amino acids as shown in Genbank AAP34324. An example of the amino acid sequence of an Influenza HA antigen, identified as H1N1 Ecto foldon-foldon-Bir-His6, is amino acids 1 to 519 of Genbank AAP34324 with an amino acid substitution at position 108 of a Phe (F) for a Tyr (Y) having the amino acid sequence of SEQ ID NO: 81. Exemplary anti-HA antibodies are described in U.S. Ser. No. 14/974,361 having Publication No. 2016/0176953.

The anti-Influenza HA antibodies of the disclosure, or antigen-binding portions thereof, may be included in a bispecific antigen-binding molecule that binds specific target cell types such as infectious agents. In embodiments, the bispecific antigen-binding molecule binds to the Influenza HA with an IC₅₀ of about 10 nM or less.

Exemplary anti-Influenza HA antibodies of the present disclosure are listed in Tables 7 and 8. Table 7 sets forth the amino acid sequence identifiers of the heavy chain variable regions (HCVRs) and light chain variable regions (LCVRs), as well as heavy chain complementarity determining regions (HCDR1, HCDR2 and HCDR3), and light chain complementarity determining regions (LCDR1, LCDR2 and LCDR3) of the exemplary anti-Influenza HA antibodies. Table 8 sets forth the sequence identifiers of the nucleic acid molecules encoding the HCVRs, LCVRs, HCDR1, HCDR2, HCDR3, LCDR1, LCDR2 and LCDR3 of the exemplary anti-Influenza HA antibodies.

The present disclosure provides antibodies, or antigen-binding fragments thereof that bind Influenza HA comprising heavy chain variable region (HCVR) CDRs contained within a HCVR comprising an amino acid sequence of SEQ ID NO: 42, or a substantially similar sequence thereof having at least 90%, at least 91%, at least 92%, at least 93%, at least 94%, at least 95%, at least 96%, at least 97%, at least 98%, or at least 99% sequence identity thereto; and light chain variable region (LCVR) CDRs contained within a LCVR region comprising an amino acid sequence of SEQ ID NO: 10, or a substantially similar sequence thereof having at least 90%, at least 91%, at least 92%, at least 93%, at least 94%, at least 95%, at least 96%, at least 97%, at least 98%, or at least 99% sequence identity thereto.

The present disclosure also provides antibodies, or antigen-binding fragments thereof, comprising an HCVR and an LCVR pair (HCVR/LCVR) comprising any of the HCVR amino acid sequences listed in Table 7 paired with any of the LCVR amino acid sequences listed in Table 7. According to certain embodiments, the present disclosure provides antibodies, or antigen-binding fragments thereof, comprising an HCVR/LCVR pair contained within any of the exemplary anti-Influenza HA antibodies listed in Table 7. In certain embodiments, the HCVR/LCVR pair comprises amino acid sequences of SEQ ID NOs: 42/10.

The present disclosure also provides antibodies, or antigen-binding fragments thereof, that bind to Influenza HA comprising a heavy chain CDR1 (HCDR1) comprising an amino acid sequence selected from any of the HCDR1 amino acid sequences listed in Table 7, or a substantially similar sequence thereof having at least 90%, at least 95%, at least 98% or at least 99% sequence identity.

The present disclosure also provides antibodies, or antigen-binding fragments thereof, that bind to human Influenza HA comprising a heavy chain CDR2 (HCDR2) comprising an amino acid sequence selected from any of the HCDR2 amino acid sequences listed in Table 7, or a substantially similar sequence thereof having at least 90%, at least 95%, at least 98% or at least 99% sequence identity.

The present disclosure also provides antibodies, or antigen-binding fragments thereof, that bind to Influenza HA comprising a heavy chain CDR3 (HCDR3) comprising an amino acid sequence selected from any of the HCDR3 amino acid sequences listed in Table 7, or a substantially similar sequence thereof having at least 90%, at least 95%, at least 98% or at least 99% sequence identity.

The present disclosure also provides antibodies, or antigen-binding fragments thereof, that bind to Influenza HA comprising a light chain CDR1 (LCDR1) comprising an amino acid sequence selected from any of the LCDR1 amino acid sequences listed in Table 7, or a substantially similar sequence thereof having at least 90%, at least 95%, at least 98% or at least 99% sequence identity.

The present disclosure also provides antibodies, or antigen-binding fragments thereof, that bind to Influenza HA comprising a light chain CDR3 (LCDR3) comprising an amino acid sequence selected from any of the LCDR3 amino acid sequences listed in Table 7, or a substantially similar sequence thereof having at least 90%, at least 95%, at least 98% or at least 99% sequence identity.

The present disclosure also provides antibodies, or antigen-binding fragments thereof, that bind to Influenza HA comprising an HCDR3 and an LCDR3 pair (HCDR3/LCDR3) comprising any of the HCDR3 amino acid sequences listed in Table 7 paired with any of the LCDR3 amino acid sequences listed in Table 7. According to certain embodiments, the present disclosure provides antibodies, or antigen-binding fragments thereof, comprising an HCDR3/LCDR3 pair contained within any of the exemplary Influenza HA antibodies listed in Table 7. In certain embodiments, the first antigen-binding domain that specifically binds Influenza HA comprises a HCDR3/LCDR3 pair comprising amino acid sequences of SEQ ID NOs: 48/16.

The present disclosure also provides antibodies, or antigen-binding fragments thereof, that bind to Influenza HA comprising a heavy chain CDR1 (HCDR1) comprising an amino acid sequence of SEQ ID NO:44, or a substantially similar sequence thereof having at least 90%, at least 95%, at least 98% or at least 99% sequence identity; a heavy chain CDR2 (HCDR2) comprising an amino acid sequence of SEQ ID NO:46 or a substantially similar sequence thereof having at least 90%, at least 95%, at least 98% or at least 99% sequence identity; and a heavy chain CDR3 (HCDR3) comprising an amino acid sequence of SEQ ID NO:48, or a substantially similar sequence thereof having at least 90%, at least 95%, at least 98% or at least 99% sequence identity. In embodiments, the first antigen-binding domain that specifically binds Influenza HA comprises a light chain CDR1 (LCDR1) comprising an amino acid sequence of SEQ ID NO:12, or a substantially similar sequence thereof having at least 90%, at least 95%, at least 98% or at least 99% sequence identity; a light chain CDR2 (LCDR2) comprising an amino acid sequence of SEQ ID NO:14, or a substantially similar sequence thereof having at least 90%, at least 95%, at least 98% or at least 99% sequence identity; and a light chain CDR3 (LCDR3) comprising an amino acid sequence of SEQ ID NO:16, or a substantially similar sequence thereof having at least 90%, at least 95%, at least 98% or at least 99% sequence identity.

Certain non-limiting, exemplary antibodies, or antigen-binding fragments thereof, that bind to Influenza HA comprise a set of six CDRs HCDR1-HCDR2-HCDR3-LCDR1-LCDR2-LCDR3, respectively, the set comprising amino acid sequences of SEQ ID NOs:44/46/48/12/14/16.

In another aspect, the present disclosure provides nucleic acid molecules encoding any of the HCVR, LCVR or CDR sequences of the antibodies, or antigen-binding fragments thereof, that bind to Influenza HA including nucleic acid molecules comprising the polynucleotide sequences as set forth in Table 8 herein, as well as nucleic acid molecules comprising two or more of the polynucleotide sequences as set forth in Tables 2 and 8 in any functional combination or arrangement thereof. Recombinant expression vectors carrying the nucleic acids of the disclosure, and host cells into which such vectors have been introduced, are also encompassed by the disclosure, as are methods of producing the antibodies by culturing the host cells under conditions permitting production of the antibodies, and recovering the antibodies produced.

The present disclosure also provides nucleic acid molecules encoding anti-Influenza HA antibodies or portions thereof. For example, the present disclosure provides nucleic acid molecules encoding any of the HCVR amino acid sequences listed in Table 7; in certain embodiments the nucleic acid molecule comprises a polynucleotide sequence selected from any of the HCVR nucleic acid sequences listed in Table 8, or a substantially similar sequence thereof having at least 90%, at least 95%, at least 98% or at least 99% sequence identity thereto.

The present disclosure also provides nucleic acid molecules encoding any of the LCVR amino acid sequences listed in Table 7; in certain embodiments the nucleic acid molecule comprises a polynucleotide sequence selected from any of the LCVR nucleic acid sequences listed in Table 8, or a substantially similar sequence thereof having at least 90%, at least 95%, at least 98% or at least 99% sequence identity thereto.

The present disclosure also provides nucleic acid molecules encoding any of the HCDR1 amino acid sequences listed in Table 7; in certain embodiments the nucleic acid molecule comprises a polynucleotide sequence selected from any of the HCDR1 nucleic acid sequences listed in Table 8, or a substantially similar sequence thereof having at least 90%, at least 95%, at least 98% or at least 99% sequence identity thereto.

The present disclosure also provides nucleic acid molecules encoding any of the HCDR2 amino acid sequences listed in Table 7; in certain embodiments the nucleic acid molecule comprises a polynucleotide sequence selected from any of the HCDR2 nucleic acid sequences listed in Table 8, or a substantially similar sequence thereof having at least 90%, at least 95%, at least 98% or at least 99% sequence identity thereto.

The present disclosure also provides nucleic acid molecules encoding any of the HCDR3 amino acid sequences listed in Table 7; in certain embodiments the nucleic acid molecule comprises a polynucleotide sequence selected from any of the HCDR3 nucleic acid sequences listed in Table 8, or a substantially similar sequence thereof having at least 90%, at least 95%, at least 98% or at least 99% sequence identity thereto.

The present disclosure also provides nucleic acid molecules encoding any of the LCDR1 amino acid sequences listed in Table 7; in certain embodiments the nucleic acid molecule comprises a polynucleotide sequence selected from any of the LCDR1 nucleic acid sequences listed in Table 8, or a substantially similar sequence thereof having at least 90%, at least 95%, at least 98% or at least 99% sequence identity thereto.

The present disclosure also provides nucleic acid molecules encoding any of the LCDR2 amino acid sequences listed in Table 7; in certain embodiments the nucleic acid molecule comprises a polynucleotide sequence selected from any of the LCDR2 nucleic acid sequences listed in Table 8, or a substantially similar sequence thereof having at least 90%, at least 95%, at least 98% or at least 99% sequence identity thereto.

The present disclosure also provides nucleic acid molecules encoding any of the LCDR3 amino acid sequences listed in Table 7; in certain embodiments the nucleic acid molecule comprises a polynucleotide sequence selected from any of the LCDR3 nucleic acid sequences listed in Table 8, or a substantially similar sequence thereof having at least 90%, at least 95%, at least 98% or at least 99% sequence identity thereto.

The present disclosure also provides nucleic acid molecules encoding an HCVR, wherein the HCVR comprises a set of three CDRs (i.e., HCDR1-HCDR2-HCDR3), wherein the HCDR1-HCDR2-HCDR3 set is as defined by any of the exemplary anti-Influenza HA antibodies listed in Table 7.

The present disclosure also provides nucleic acid molecules encoding an LCVR, wherein the LCVR comprises a set of three CDRs (i.e., LCDR1-LCDR2-LCDR3), wherein the LCDR1-LCDR2-LCDR3 set is as defined by any of the exemplary anti-Influenza HA antibodies listed in Table 7.

The present disclosure also provides nucleic acid molecules encoding both an HCVR and an LCVR, wherein the HCVR comprises an amino acid sequence of any of the HCVR amino acid sequences listed in Table 7, and wherein the LCVR comprises an amino acid sequence of any of the LCVR amino acid sequences listed in Table 7. In certain embodiments, the nucleic acid molecule comprises a polynucleotide sequence selected from any of the HCVR nucleic acid sequences listed in Table 8, or a substantially similar sequence thereof having at least 90%, at least 95%, at least 98% or at least 99% sequence identity thereto, and a polynucleotide sequence selected from any of the LCVR nucleic acid sequences listed in Table 8, or a substantially similar sequence thereof having at least 90%, at least 95%, at least 98% or at least 99% sequence identity thereto. In certain embodiments according to this aspect of the disclosure, the nucleic acid molecule encodes an HCVR and LCVR, wherein the HCVR and LCVR are both derived from the same anti-Influenza HA antibody listed in Table 7.

The present disclosure also provides recombinant expression vectors capable of expressing a polypeptide comprising a heavy or light chain variable region of an anti-Influenza HA antibody. For example, the present disclosure includes recombinant expression vectors comprising any of the nucleic acid molecules mentioned above, i.e., nucleic acid molecules encoding any of the HCVR, LCVR, and/or CDR sequences as set forth in Table 7. Also included within the scope of the present disclosure are host cells into which such vectors have been introduced, as well as methods of producing the antibodies or portions thereof by culturing the host cells under conditions permitting production of the antibodies or antibody fragments, and recovering the antibodies and antibody fragments so produced.

The present disclosure includes anti-Influenza HA antibodies having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

### D. Cancer Cell Target Antigens

In another aspect, the present disclosure provides binding molecules, e.g., antibodies and antigen-binding fragments thereof, that bind a cancer cell surface antigen (e.g. a monospecific antibody to a cancer-cell surface target antigen). In embodiments, the cancer cell surface target antigen comprises CD20, other cancer cell surface target antigens such as tumor-specific antigens, or combinations thereof. An embodiment of the antigen comprises amino acids as shown in Genbank NP_690605.

Exemplary anti-CD20 antibodies are described in U.S. Pat. No. 9,657,102, US Publication No. 2015/0266966, and US Publication No. 2018/0194841, incorporated by reference herein. In one embodiment, the antibody or antigen binding fragment thereof specifically binds CD20. The anti-CD20 antibodies of the disclosure, or antigen-binding portions thereof, may be included in a bispecific antigen-binding molecule that binds specific target cell types such as infectious agents. In embodiments, the bispecific antigen-binding molecule binds to the CD20 with an IC₅₀ of about 10 nM or less.

Exemplary anti-CD20 antibodies of the present disclosure are listed in Tables 5 and 6. Table 5 sets forth the amino acid sequence identifiers of the heavy chain variable regions (HCVRs) and light chain variable regions (LCVRs), as well as heavy chain complementarity determining regions (HCDR1, HCDR2 and HCDR3), and light chain complementarity determining regions (LCDR1, LCDR2 and LCDR3) of the exemplary anti-CD20 antibodies. Table 6 sets forth the sequence identifiers of the nucleic acid molecules encoding the HCVRs, LCVRs, HCDR1, HCDR2, HCDR3, LCDR1, LCDR2 and LCDR3 of the exemplary anti-CD20 antibodies.

The present disclosure provides antibodies, or antigen-binding fragments thereof that bind CD20 comprising heavy chain variable region (HCVR) CDRs contained within a HCVR comprising an amino acid sequence of SEQ ID NO: 34, or a substantially similar sequence thereof having at least 90%, at least 91%, at least 92%, at least 93%, at least 94%, at least 95%, at least 96%, at least 97%, at least 98%, or at least 99% sequence identity thereto; and light chain variable region (LCVR) CDRs contained within a LCVR region comprising an amino acid sequence of SEQ ID NO: 10, or a substantially similar sequence thereof having at least 90%, at least 91%, at least 92%, at least 93%, at least 94%, at least 95%, at least 96%, at least 97%, at least 98%, or at least 99% sequence identity thereto.

The present disclosure also provides antibodies, or antigen-binding fragments thereof, comprising an HCVR and an LCVR pair (HCVR/LCVR) comprising any of the HCVR amino acid sequences listed in Table 5 paired with any of the LCVR amino acid sequences listed in Table 5. According to certain embodiments, the present disclosure provides antibodies, or antigen-binding fragments thereof, comprising an HCVR/LCVR pair contained within any of the exemplary anti-CD20 antibodies listed in Table 5. In certain embodiments, the HCVR/LCVR pair comprises amino acid sequences of SEQ ID Nos: 34/10.

The present disclosure also provides antibodies, or antigen-binding fragments thereof, that bind to CD20 comprising a heavy chain CDR1 (HCDR1) comprising an amino acid sequence selected from any of the HCDR1 amino acid sequences listed in Table 5, or a substantially similar sequence thereof having at least 90%, at least 95%, at least 98% or at least 99% sequence identity.

The present disclosure also provides antibodies, or antigen-binding fragments thereof, that bind to human CD20 comprising a heavy chain CDR2 (HCDR2) comprising an amino acid sequence selected from any of the HCDR2 amino acid sequences listed in Table 5, or a substantially similar sequence thereof having at least 90%, at least 95%, at least 98% or at least 99% sequence identity.

The present disclosure also provides antibodies, or antigen-binding fragments thereof, that bind to CD20 comprising a heavy chain CDR3 (HCDR3) comprising an amino acid sequence selected from any of the HCDR3 amino acid sequences listed in Table 5, or a substantially similar sequence thereof having at least 90%, at least 95%, at least 98% or at least 99% sequence identity.

The present disclosure also provides antibodies, or antigen-binding fragments thereof, that bind to CD20 comprising a light chain CDR1 (LCDR1) comprising an amino acid sequence selected from any of the LCDR1 amino acid sequences listed in Table 5, or a substantially similar sequence thereof having at least 90%, at least 95%, at least 98% or at least 99% sequence identity.

The present disclosure also provides antibodies, or antigen-binding fragments thereof, that bind to CD20 comprising a light chain CDR2 (LCDR2) comprising an amino acid sequence selected from any of the LCDR2 amino acid sequences listed in Table 5, or a substantially similar sequence thereof having at least 90%, at least 95%, at least 98% or at least 99% sequence identity.

The present disclosure also provides antibodies, or antigen-binding fragments thereof, that bind to CD20 comprising a light chain CDR3 (LCDR3) comprising an amino acid sequence selected from any of the LCDR3 amino acid sequences listed in Table 5, or a substantially similar sequence thereof having at least 90%, at least 95%, at least 98% or at least 99% sequence identity.

The present disclosure also provides antibodies, or antigen-binding fragments thereof, that bind to CD20 comprising an HCDR3 and an LCDR3 pair (HCDR3/LCDR3) comprising any of the HCDR3 amino acid sequences listed in Table 5 paired with any of the LCDR3 amino acid sequences listed in Table 5. According to certain embodiments, the present disclosure provides antibodies, or antigen-binding fragments thereof, comprising an HCDR3/LCDR3 pair contained within any of the exemplary CD20 antibodies listed in Table 5. In certain embodiments, the first antigen-binding domain that specifically binds CD20 comprises a HCDR3/LCDR3 pair comprising amino acid sequences of SEQ ID NOs: 40/16.

The present disclosure also provides antibodies, or antigen-binding fragments thereof, that bind to CD20 comprising a heavy chain CDR1 (HCDR1) comprising an amino acid sequence of SEQ ID NO:36, or a substantially similar sequence thereof having at least 90%, at least 95%, at least 98% or at least 99% sequence identity; a heavy chain CDR2 (HCDR2) comprising an amino acid sequence of SEQ ID NO:38, or a substantially similar sequence thereof having at least 90%, at least 95%, at least 98% or at least 99% sequence identity; and a heavy chain CDR3 (HCDR3) comprising an amino acid sequence of SEQ ID NO:40, or a substantially similar sequence thereof having at least 90%, at least 95%, at least 98% or at least 99% sequence identity. In embodiments, the first antigen-binding domain that specifically binds CD20 comprises a light chain CDR1 (LCDR1) comprising an amino acid sequence of SEQ ID NO:12, or a substantially similar sequence thereof having at least 90%, at least 95%, at least 98% or at least 99% sequence identity; a light chain CDR2 (LCDR2) comprising an amino acid sequence of SEQ ID NO:14, or a substantially similar sequence thereof having at least 90%, at least 95%, at least 98% or at least 99% sequence identity; and a light chain CDR3 (LCDR3) comprising an amino acid sequence of SEQ ID NO:16, or a substantially similar sequence thereof having at least 90%, at least 95%, at least 98% or at least 99% sequence identity.

Certain non-limiting, exemplary antibodies, or antigen-binding fragments thereof, that bind to CD20 comprise a set of six CDRs HCDR1-HCDR2-HCDR3-LCDR1-LCDR2-LCDR3, respectively, the set comprising amino acid sequences of SEQ ID NOs:36/38/40/12/14/16.

In another aspect, the present disclosure provides nucleic acid molecules encoding any of the HCVR, LCVR or CDR sequences of the antibodies, or antigen-binding fragments thereof, that bind to CD20 including nucleic acid molecules comprising the polynucleotide sequences as set forth in Table 6 herein, as well as nucleic acid molecules comprising two or more of the polynucleotide sequences as set forth in Tables 2 and 6 in any functional combination or arrangement thereof. Recombinant expression vectors carrying the nucleic acids of the disclosure, and host cells into which such vectors have been introduced, are also encompassed by the disclosure, as are methods of producing the antibodies by culturing the host cells under conditions permitting production of the antibodies, and recovering the antibodies produced.

The present disclosure also provides nucleic acid molecules encoding anti-CD20 antibodies or portions thereof. For example, the present disclosure provides nucleic acid molecules encoding any of the HCVR amino acid sequences listed in Table 5; in certain embodiments the nucleic acid molecule comprises a polynucleotide sequence selected from any of the HCVR nucleic acid sequences listed in Table 6, or a substantially similar sequence thereof having at least 90%, at least 95%, at least 98% or at least 99% sequence identity thereto.

The present disclosure also provides nucleic acid molecules encoding any of the LCVR amino acid sequences listed in Table 5; in certain embodiments the nucleic acid molecule comprises a polynucleotide sequence selected from any of the LCVR nucleic acid sequences listed in Table 6, or a substantially similar sequence thereof having at least 90%, at least 95%, at least 98% or at least 99% sequence identity thereto.

The present disclosure also provides nucleic acid molecules encoding any of the HCDR1 amino acid sequences listed in Table 5; in certain embodiments the nucleic acid molecule comprises a polynucleotide sequence selected from any of the HCDR1 nucleic acid sequences listed in Table 6, or a substantially similar sequence thereof having at least 90%, at least 95%, at least 98% or at least 99% sequence identity thereto.

The present disclosure also provides nucleic acid molecules encoding any of the HCDR2 amino acid sequences listed in Table 5; in certain embodiments the nucleic acid molecule comprises a polynucleotide sequence selected from any of the HCDR2 nucleic acid sequences listed in Table 6, or a substantially similar sequence thereof having at least 90%, at least 95%, at least 98% or at least 99% sequence identity thereto.

The present disclosure also provides nucleic acid molecules encoding any of the HCDR3 amino acid sequences listed in Table 5; in certain embodiments the nucleic acid molecule comprises a polynucleotide sequence selected from any of the HCDR3 nucleic acid sequences listed in Table 6, or a substantially similar sequence thereof having at least 90%, at least 95%, at least 98% or at least 99% sequence identity thereto.

The present disclosure also provides nucleic acid molecules encoding any of the LCDR1 amino acid sequences listed in Table 5; in certain embodiments the nucleic acid molecule comprises a polynucleotide sequence selected from any of the LCDR1 nucleic acid sequences listed in Table 6, or a substantially similar sequence thereof having at least 90%, at least 95%, at least 98% or at least 99% sequence identity thereto.

The present disclosure also provides nucleic acid molecules encoding any of the LCDR2 amino acid sequences listed in Table 5; in certain embodiments the nucleic acid molecule comprises a polynucleotide sequence selected from any of the LCDR2 nucleic acid sequences listed in Table 6, or a substantially similar sequence thereof having at least 90%, at least 95%, at least 98% or at least 99% sequence identity thereto.

The present disclosure also provides nucleic acid molecules encoding any of the LCDR3 amino acid sequences listed in Table 5; in certain embodiments the nucleic acid molecule comprises a polynucleotide sequence selected from any of the LCDR3 nucleic acid sequences listed in Table 6, or a substantially similar sequence thereof having at least 90%, at least 95%, at least 98% or at least 99% sequence identity thereto.

The present disclosure also provides nucleic acid molecules encoding an HCVR, wherein the HCVR comprises a set of three CDRs (i.e., HCDR1-HCDR2-HCDR3), wherein the HCDR1-HCDR2-HCDR3 set is as defined by any of the exemplary anti-CD20 antibodies listed in Table 5.

The present disclosure also provides nucleic acid molecules encoding an LCVR, wherein the LCVR comprises a set of three CDRs (i.e., LCDR1-LCDR2-LCDR3), wherein the LCDR1-LCDR2-LCDR3 set is as defined by any of the exemplary anti-CD20 antibodies listed in Table 5.

The present disclosure also provides nucleic acid molecules encoding both an HCVR and an LCVR, wherein the HCVR comprises an amino acid sequence of any of the HCVR amino acid sequences listed in Table 5, and wherein the LCVR comprises an amino acid sequence of any of the LCVR amino acid sequences listed in Table 5. In certain embodiments, the nucleic acid molecule comprises a polynucleotide sequence selected from any of the HCVR nucleic acid sequences listed in Table 6, or a substantially similar sequence thereof having at least 90%, at least 95%, at least 98% or at least 99% sequence identity thereto, and a polynucleotide sequence selected from any of the LCVR nucleic acid sequences listed in Table 6, or a substantially similar sequence thereof having at least 90%, at least 95%, at least 98% or at least 99% sequence identity thereto. In certain embodiments according to this aspect of the disclosure, the nucleic acid molecule encodes an HCVR and LCVR, wherein the HCVR and LCVR are both derived from the same anti-CD20 antibody listed in Table 5.

The present disclosure also provides recombinant expression vectors capable of expressing a polypeptide comprising a heavy or light chain variable region of an anti-CD20 antibody. For example, the present disclosure includes recombinant expression vectors comprising any of the nucleic acid molecules mentioned above, i.e., nucleic acid molecules encoding any of the HCVR, LCVR, and/or CDR sequences as set forth in Table 5. Also included within the scope of the present disclosure are host cells into which such vectors have been introduced, as well as methods of producing the antibodies or portions thereof by culturing the host cells under conditions permitting production of the antibodies or antibody fragments, and recovering the antibodies and antibody fragments so produced.

The present disclosure includes anti-CD20 antibodies having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

### E. Immune Cell Target Antigens

In another aspect, the present disclosure provides binding molecules, e.g., antibodies and antigen-binding fragments thereof, that target an antigen that is expressed on an immune cell (e.g., a target antigen expressed on B cells, dendritic cells, granulocytes, lymphoid cells, monocytes, macrophages, T cells, or thymocytes). For example, in some embodiments, the binding molecule, e.g., antibody or antigen-binding fragment thereof, binds to a target antigen that is expressed on the surface of T cells or a subset of T cells, such as glucocorticoid induced tumor necrosis factor receptor (GITR).

In embodiments, the present disclosure provides antibodies and antigen-binding fragments thereof that bind a glucocorticoid induced tumor necrosis factor receptor (e.g. a monospecific antibody to a tumor necrosis factor receptor antigen). An embodiment of the antigen comprises amino acids as shown in Genbank NP_683700. Another embodiment of GITR, identified as hGITR.mmm, has amino acids Gln (Q) at position 26 to Pro (P) at position 162 of the sequence of Accession No. NP_683700 shown in SEQ ID NO: 82 and mmh domain of amino acids 136-165 of SEQ ID NO: 82. In embodiments, the tumor necrosis factor receptor antigen comprises glucocorticoid-induced TNFR-related gene (“GITR”), other tumor necrosis factor receptor target antigens or antigens on the surface of T cells, or combinations thereof.

Exemplary anti-GITR antibodies are described in U.S. Ser. No. 15/619,068 having publication number US2017/0355774, incorporated by reference herein. The anti-GITR antibodies of the disclosure, or antigen-binding portions thereof, may be included in a bispecific antigen-binding molecule that binds specific target cell types such as infectious agents. In embodiments, the bispecific antigen-binding molecule binds to the GITR with an IC₅₀ of about 10 nM or less.

Exemplary anti-GITR antibodies of the present disclosure are listed in Tables 9 and 10. Table 9 sets forth the amino acid sequence identifiers of the heavy chain variable regions (HCVRs) and light chain variable regions (LCVRs), as well as heavy chain complementarity determining regions (HCDR1, HCDR2 and HCDR3), and light chain complementarity determining regions (LCDR1, LCDR2 and LCDR3) of the exemplary anti-GITR antibodies. Table 10 sets forth the sequence identifiers of the nucleic acid molecules encoding the HCVRs, LCVRs, HCDR1, HCDR2, HCDR3, LCDR1, LCDR2 and LCDR3 of the exemplary anti-GITR antibodies.

The present disclosure provides antibodies, or antigen-binding fragments thereof that bind GITR comprising heavy chain variable region (HCVR) CDRs contained within a HCVR comprising an amino acid sequence of SEQ ID NO: 50, or a substantially similar sequence thereof having at least 90%, at least 91%, at least 92%, at least 93%, at least 94%, at least 95%, at least 96%, at least 97%, at least 98%, or at least 99% sequence identity thereto; and light chain variable region (LCVR) CDRs contained within a LCVR region comprising an amino acid sequence of SEQ ID NO: 10, or a substantially similar sequence thereof having at least 90%, at least 91%, at least 92%, at least 93%, at least 94%, at least 95%, at least 96%, at least 97%, at least 98%, or at least 99% sequence identity thereto.

The present disclosure also provides antibodies, or antigen-binding fragments thereof, comprising an HCVR and an LCVR pair (HCVR/LCVR) comprising any of the HCVR amino acid sequences listed in Table 9 paired with any of the LCVR amino acid sequences listed in Table 9. According to certain embodiments, the present disclosure provides antibodies, or antigen-binding fragments thereof, comprising an HCVR/LCVR pair contained within any of the exemplary anti-GITR antibodies listed in Table 9. In certain embodiments, the HCVR/LCVR pair comprises amino acid sequences of SEQ ID Nos: 50/10.

The present disclosure also provides antibodies, or antigen-binding fragments thereof, that bind to GITR comprising a heavy chain CDR1 (HCDR1) comprising an amino acid sequence selected from any of the HCDR1 amino acid sequences listed in Table 9, or a substantially similar sequence thereof having at least 90%, at least 95%, at least 98% or at least 99% sequence identity.

The present disclosure also provides antibodies, or antigen-binding fragments thereof, that bind to human GITR comprising a heavy chain CDR2 (HCDR2) comprising an amino acid sequence selected from any of the HCDR2 amino acid sequences listed in Table 9, or a substantially similar sequence thereof having at least 90%, at least 95%, at least 98% or at least 99% sequence identity.

The present disclosure also provides antibodies, or antigen-binding fragments thereof, that bind to GITR comprising a heavy chain CDR3 (HCDR3) comprising an amino acid sequence selected from any of the HCDR3 amino acid sequences listed in Table 9, or a substantially similar sequence thereof having at least 90%, at least 95%, at least 98% or at least 99% sequence identity.

The present disclosure also provides antibodies, or antigen-binding fragments thereof, that bind to GITR comprising a light chain CDR1 (LCDR1) comprising an amino acid sequence selected from any of the LCDR1 amino acid sequences listed in Table 9, or a substantially similar sequence thereof having at least 90%, at least 95%, at least 98% or at least 99% sequence identity.

The present disclosure also provides antibodies, or antigen-binding fragments thereof, that bind to GITR comprising a light chain CDR2 (LCDR2) comprising an amino acid sequence selected from any of the LCDR2 amino acid sequences listed in Table 9, or a substantially similar sequence thereof having at least 90%, at least 95%, at least 98% or at least 99% sequence identity.

The present disclosure also provides antibodies, or antigen-binding fragments thereof, that bind to GITR comprising a light chain CDR3 (LCDR3) comprising an amino acid sequence selected from any of the LCDR3 amino acid sequences listed in Table 9, or a substantially similar sequence thereof having at least 90%, at least 95%, at least 98% or at least 99% sequence identity.

The present disclosure also provides antibodies, or antigen-binding fragments thereof, that bind to GITR comprising an HCDR3 and an LCDR3 pair (HCDR3/LCDR3) comprising any of the HCDR3 amino acid sequences listed in Table 9 paired with any of the LCDR3 amino acid sequences listed in Table 9. According to certain embodiments, the present disclosure provides antibodies, or antigen-binding fragments thereof, comprising an HCDR3/LCDR3 pair contained within any of the exemplary GITR antibodies listed in Table 9. In certain embodiments, the first antigen-binding domain that specifically binds GITR comprises a HCDR3/LCDR3 pair comprising amino acid sequences of SEQ ID NOs: 56/16.

The present disclosure also provides antibodies, or antigen-binding fragments thereof, that bind to GITR comprising a heavy chain CDR1 (HCDR1) comprising an amino acid sequence of SEQ ID NO:52, or a substantially similar sequence thereof having at least 90%, at least 95%, at least 98% or at least 99% sequence identity; a heavy chain CDR2 (HCDR2) comprising an amino acid sequence of SEQ ID NO:54, or a substantially similar sequence thereof having at least 90%, at least 95%, at least 98% or at least 99% sequence identity; and a heavy chain CDR3 (HCDR3) comprising an amino acid sequence of SEQ ID NO:56, or a substantially similar sequence thereof having at least 90%, at least 95%, at least 98% or at least 99% sequence identity. In embodiments, the first antigen-binding domain that specifically binds GITR comprises a light chain CDR1 (LCDR1) comprising an amino acid sequence of SEQ ID NO:12, or a substantially similar sequence thereof having at least 90%, at least 95%, at least 98% or at least 99% sequence identity; a light chain CDR2 (LCDR2) comprising an amino acid sequence of SEQ ID NO:14, or a substantially similar sequence thereof having at least 90%, at least 95%, at least 98% or at least 99% sequence identity; and a light chain CDR3 (LCDR3) comprising an amino acid sequence of SEQ ID NO:16, or a substantially similar sequence thereof having at least 90%, at least 95%, at least 98% or at least 99% sequence identity.

Certain non-limiting, exemplary antibodies, or antigen-binding fragments thereof, that bind to GITR comprise a set of six CDRs HCDR1-HCDR2-HCDR3-LCDR1-LCDR2-LCDR3, respectively, the set comprising amino acid sequences of SEQ ID NOs: 52/54/56/12/14/16.

In another aspect, the present disclosure provides nucleic acid molecules encoding any of the HCVR, LCVR or CDR sequences of the antibodies, or antigen-binding fragments thereof, that bind to GITR including nucleic acid molecules comprising the polynucleotide sequences as set forth in Table 10 herein, as well as nucleic acid molecules comprising two or more of the polynucleotide sequences as set forth in Tables 2 and 10 in any functional combination or arrangement thereof. Recombinant expression vectors carrying the nucleic acids of the disclosure, and host cells into which such vectors have been introduced, are also encompassed by the disclosure, as are methods of producing the antibodies by culturing the host cells under conditions permitting production of the antibodies, and recovering the antibodies produced.

The present disclosure also provides nucleic acid molecules encoding anti-GITR antibodies or portions thereof. For example, the present disclosure provides nucleic acid molecules encoding any of the HCVR amino acid sequences listed in Table 9; in certain embodiments the nucleic acid molecule comprises a polynucleotide sequence selected from any of the HCVR nucleic acid sequences listed in Table 10, or a substantially similar sequence thereof having at least 90%, at least 95%, at least 98% or at least 99% sequence identity thereto.

The present disclosure also provides nucleic acid molecules encoding any of the LCVR amino acid sequences listed in Table 9; in certain embodiments the nucleic acid molecule comprises a polynucleotide sequence selected from any of the LCVR nucleic acid sequences listed in Table 10, or a substantially similar sequence thereof having at least 90%, at least 95%, at least 98% or at least 99% sequence identity thereto.

The present disclosure also provides nucleic acid molecules encoding any of the HCDR1 amino acid sequences listed in Table 9; in certain embodiments the nucleic acid molecule comprises a polynucleotide sequence selected from any of the HCDR1 nucleic acid sequences listed in Table 10, or a substantially similar sequence thereof having at least 90%, at least 95%, at least 98% or at least 99% sequence identity thereto.

The present disclosure also provides nucleic acid molecules encoding any of the HCDR2 amino acid sequences listed in Table 9; in certain embodiments the nucleic acid molecule comprises a polynucleotide sequence selected from any of the HCDR2 nucleic acid sequences listed in Table 10, or a substantially similar sequence thereof having at least 90%, at least 95%, at least 98% or at least 99% sequence identity thereto.

The present disclosure also provides nucleic acid molecules encoding any of the HCDR3 amino acid sequences listed in Table 9; in certain embodiments the nucleic acid molecule comprises a polynucleotide sequence selected from any of the HCDR3 nucleic acid sequences listed in Table 10, or a substantially similar sequence thereof having at least 90%, at least 95%, at least 98% or at least 99% sequence identity thereto.

The present disclosure also provides nucleic acid molecules encoding any of the LCDR1 amino acid sequences listed in Table 9; in certain embodiments the nucleic acid molecule comprises a polynucleotide sequence selected from any of the LCDR1 nucleic acid sequences listed in Table 10, or a substantially similar sequence thereof having at least 90%, at least 95%, at least 98% or at least 99% sequence identity thereto.

The present disclosure also provides nucleic acid molecules encoding any of the LCDR2 amino acid sequences listed in Table 9; in certain embodiments the nucleic acid molecule comprises a polynucleotide sequence selected from any of the LCDR2 nucleic acid sequences listed in Table 10, or a substantially similar sequence thereof having at least 90%, at least 95%, at least 98% or at least 99% sequence identity thereto.

The present disclosure also provides nucleic acid molecules encoding any of the LCDR3 amino acid sequences listed in Table 9; in certain embodiments the nucleic acid molecule comprises a polynucleotide sequence selected from any of the LCDR3 nucleic acid sequences listed in Table 10, or a substantially similar sequence thereof having at least 90%, at least 95%, at least 98% or at least 99% sequence identity thereto.

The present disclosure also provides nucleic acid molecules encoding an HCVR, wherein the HCVR comprises a set of three CDRs (i.e., HCDR1-HCDR2-HCDR3), wherein the HCDR1-HCDR2-HCDR3 set is as defined by any of the exemplary anti-GITR antibodies listed in Table 9.

The present disclosure also provides nucleic acid molecules encoding an LCVR, wherein the LCVR comprises a set of three CDRs (i.e., LCDR1-LCDR2-LCDR3), wherein the LCDR1-LCDR2-LCDR3 set is as defined by any of the exemplary anti-GITR antibodies listed in Table 9.

The present disclosure also provides nucleic acid molecules encoding both an HCVR and an LCVR, wherein the HCVR comprises an amino acid sequence of any of the HCVR amino acid sequences listed in Table 9, and wherein the LCVR comprises an amino acid sequence of any of the LCVR amino acid sequences listed in Table 9. In certain embodiments, the nucleic acid molecule comprises a polynucleotide sequence selected from any of the HCVR nucleic acid sequences listed in Table 10, or a substantially similar sequence thereof having at least 90%, at least 95%, at least 98% or at least 99% sequence identity thereto, and a polynucleotide sequence selected from any of the LCVR nucleic acid sequences listed in Table 10, or a substantially similar sequence thereof having at least 90%, at least 95%, at least 98% or at least 99% sequence identity thereto. In certain embodiments according to this aspect of the disclosure, the nucleic acid molecule encodes an HCVR and LCVR, wherein the HCVR and LCVR are both derived from the same anti-GITR antibody listed in Table 9.

The present disclosure also provides recombinant expression vectors capable of expressing a polypeptide comprising a heavy or light chain variable region of an anti-GITR antibody. For example, the present disclosure includes recombinant expression vectors comprising any of the nucleic acid molecules mentioned above, i.e., nucleic acid molecules encoding any of the HCVR, LCVR, and/or CDR sequences as set forth in Table 9. Also included within the scope of the present disclosure are host cells into which such vectors have been introduced, as well as methods of producing the antibodies or portions thereof by culturing the host cells under conditions permitting production of the antibodies or antibody fragments, and recovering the antibodies and antibody fragments so produced.

The present disclosure includes anti-GITR antibodies having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

In another aspect, the present disclosure provides binding molecules that bind to antigen receptor-expressing cells, e.g., B-cell or T-cells. In some embodiments, the target antigen is a B-cell receptor (BCR) on a B-cell, and the antigen binding domain comprises a binding molecule that is recognized by or has affinity for the BCR (e.g., a ligand that binds to the B-cell receptor or an antibody or antigen binding fragment thereof that binds to the B-cell receptor). In embodiments, the target antigen is a T-cell receptor on a T-cell, and the antigen binding domain comprises a binding molecule having a specific peptide-MHC that is recognized by or has affinity for the TCR (such as, but not limited to, single-chain peptide-β2M-MHC-I trimers; single-chain peptide-MHC-IIα/MHC-IIβ multimers heterodimerized by domains including but not limited to fos:jun leucine zippers; or single-chain peptide-MHC-IIO/MHC-IIa multimers heterodimerized by domains including but not limited to fos:jun leucine zippers). Single-chain peptide-MHC molecules and methods of generating them are described, for example, in Kotsiou et al., Antiox Redox Signal., 2011, 15(3):645-66, and Wen et al., Methods Mol Biol. 2013; 1061:245-264.

### IV. Bispecific Antigen-Binding Molecules

The bispecific antigen-binding molecules of the present disclosure comprise two separate antigen-binding domains (D1 and D2). In some embodiments, the first antigen-binding domain (D1) specifically binds to a target antigen, e.g., a target antigen that is expressed on or bound to the surface of a cell or organism or a target antigen or a soluble target antigen, and the second antigen-binding domain (D2) binds to a complement component, e.g., C1q. In some embodiments, the target antigen is expressed on the surface of a cell that is to be targeted for elimination. In some embodiments, the target antigen is a soluble antigen that is to be targeted for elimination.

In some embodiments, one antigen-binding domain (D1) specifically binds to a target antigen on the surface of a cell or organism, where the target antigen is a marker of a disease state of a subject and the second antigen-binding domain (D2) binds to a complement component, e.g. C1q. For example, one antigen-binding domain (D1) specifically binds to a target antigen on the surface of a bacteria, on the surface of a virus, on the surface of a virus infected cell, on the surface of a cancer cell, or on the surface of an immune cell, and the second antigen-binding domain (D2) binds to a complement component, e.g. C1q. For example, one antigen-binding domain (D1) specifically binds to a Staphylococcus species target antigen (e.g. IsdB, IsdA, Protein A, PNAG, ClfA), an Influenza virus target antigen (e.g., Influenza HA), a Pseudomonas species target antigen (e.g., Psl), a tumor necrosis factor receptor antigen (e.g., GITR), or a cancer-cell surface antigen (e.g., CD20), and the second antigen-binding domain (D2) binds to a complement component, e.g. C1q. In some embodiments, the bispecific antigen-binding molecule comprises a target antigen-binding antibody as disclosed in Section III above and an anti-complement antibody as disclosed in Section II above.

In some embodiments, the first antigen binding domain specifically binds to a bacterial target antigen. In embodiments, the bacterial target antigen is from a Staphylococcus species. In embodiments, the bacterial target antigen is from a species of bacteria other than a Staphylococcus species. In embodiments, the bacterial target antigen is a Gram-positive bacterial target antigen. In embodiments, the bacterial target antigen is a Gram-positive bacterial target antigen from a species of bacteria other than a Staphylococcus species. In embodiments, the bacterial target antigen is a Gram-negative bacterial target antigen.

In embodiments, the first antigen binding domain binds a Staphylococcus species target antigen (e.g. the first antigen binding domain comprises a monospecific antibody that binds to a Staphylococcus species target antigen, or an antigen-binding fragment thereof). In embodiments, the Staphylococcus species is a species that causes an infection in humans. In embodiments, the Staphylococcus species comprises Staphylococcus epidermidis, Staphylococcus aureus, Staphylococcus lugdunensis, and/or Staphylococcus saprophyticus. In embodiments, the Staphylococcus species target antigen comprises capsular polysaccharide type 5, capsular polysaccharide type 8, IsdB, IsdA, IsdH, lipoteichoic acid, wall teichoic acid, clumping factor A (clfA), poly-N-acetyl glucosamine (PNAG), lipase, V8 lipase, fatty acid modifying enzyme, microbial surface components recognizing adhesive matrix molecules (e.g., adhesins, fibrinogen binding molecules, fibronectin binding protein A, fibronectin binding protein B), protein A, or combinations thereof. In certain embodiments, the Staphylococcus species target antigen is IsdB, IsdA, Protein A, PNAG, or ClfA.

In some embodiments, the first antigen binding domain binds to a target antigen of a bacteria other than Staphylococcus. In embodiments, the target antigen comprises capsular polysaccharide type 5, capsular polysaccharide type 8, IsdB, IsdA, IsdH, lipoteichoic acid, wall teichoic acid, clumping factor A (clfA), poly-N-acetyl glucosamine (PNAG), lipase, V8 lipase, fatty acid modifying enzyme, microbial surface components recognizing adhesive matrix molecules (e.g. adhesins, fibrinogen binding molecules, fibronectin binding protein A, fibronectin binding protein B), protein A, or combinations thereof, wherein the target antigen is not from a species of Staphylococcus.

In embodiments, the first antigen binding domain binds a target antigen from a Gram-negative species of bacteria. In embodiments, the first antigen binding domain binds a Pseudomonas species target antigen (e.g. the first antigen binding domain comprises a monospecific antibody that binds to a Pseudomonas species target antigen, or an antigen-binding fragment thereof). In embodiments, the Staphylococcus species is a species that causes an infection in humans. In certain embodiments, the Pseudomonas species target antigen is Psl.

In embodiments, the first antigen binding domain binds a viral target antigen, e.g., a viral glycoprotein. In embodiments, the first antigen binding domain binds a viral target antigen from a virus selected from the group consisting of influenza virus, smallpox virus, hepatitis virus (e.g., hepatitis A, hepatitis B, or hepatitis C virus), human papillomavirus, human immunodeficiency virus (HIV), respiratory syncytial virus (RSV), herpesvirus, coronavirus, cowpox virus, coxsackievirus, Ebola virus, Epstein-Barr virus, measles virus, mumps virus, rubella virus, rotavirus, rabies virus, yellow fever virus, meningitis, poliovirus, rotavirus, vaccinia virus, varicella-zoster virus, variola virus, and Zika virus.

In embodiments, the first antigen binding domain binds an Influenza virus target antigen (e.g. the first antigen binding domain comprises a monospecific antibody that binds to an Influenza virus target antigen, or an antigen-binding fragment thereof). In embodiments, the Influenza virus is a virus that causes an infection in humans. In certain embodiments, the Influenza virus target antigen is Influenza HA.

In embodiments, the first antigen binding domain binds a target antigen that is present in or on a eukaryotic cell or organism, e.g., a target antigen on the surface of a eukaryotic cell or a soluble target antigen. In embodiments, the first antigen binding domain binds a target antigen on an immune cell that is targeted for elimination, e.g., a B cell, dendritic cell, granulocyte, lymphoid cell, monocyte, macrophage, T cell, or thymocyte. In embodiments, the target antigen is a B-cell receptor on a B-cell, and the first antigen binding domain binds to the B-cell receptor (e.g., a ligand that binds to the B-cell receptor or an antibody that binds to the B-cell receptor). In embodiments, the target antigen is a T-cell receptor on a T-cell, and the first antigen binding domain binds to the T-cell receptor (e.g., a peptide-MHC fusion protein or complex that binds to the T-cell receptor, such as a single-chain peptide-β2M-MHC-I trimer; a peptide-MHC-IIα/MHC-IIβ heterodimerized by domains including but not limited to fos:jun leucine zippers; or a peptide-MHC-IIβ/MHC-IIα heterodimerized by domains including but not limited to fos:jun leucine zippers).

In embodiments, the first antigen binding domain binds a glucocorticoid induced tumor necrosis factor receptor target antigen (e.g. the first antigen binding domain comprises a monospecific antibody that binds to a tumor necrosis factor receptor target antigen, or an antigen-binding fragment thereof). In certain embodiments, the tumor necrosis factor receptor target antigen is GITR.

In embodiments, the first antigen binding domain binds a cancer-cell surface target antigen (e.g. the first antigen binding domain comprises a monospecific antibody that binds to a B-cell surface target antigen, or an antigen-binding fragment thereof). In certain embodiments, the B-cell surface target antigen is CD20.

In embodiments, the second antigen-binding domain (D2) binds to any one of the complement components. Complement components include C1q, C1r, Cis, C2, C3, C4, C5, C6, C7, C8, C9, or any fragments thereof (including proteolytic fragments generated by the complement cascade). In certain embodiments, the complement component is C1q, e.g., human C1q. Complement is activated through a series of proteolytic steps which are activated by three major pathways: the Classical Pathway (CP), which is typically activated by immune-complexes, the Alternative Pathway (AP) that can be induced by unprotected cell surfaces, and the Mannose Binding Lectin (MBL) pathway. The Classical Pathway is initiated by C1q/r₂/s₂ binding to the Fc region of IgM and IgG or directly to a pathogen surface, leading to autoactivation of serine protease C1r which activates serine protease C1s, which in turns cleaves/activates C2 and C4, which then leads to cleavage of C3 and C5, C5b combines with C6, C7, C8, and C9 to form the membrane attack complex (MAC) resulting in cell lysis.

An antigen-binding domain includes polypeptides that bind a Staphylococcus species target antigen (e.g., IsdB, IsdA, Protein A, PNAG, ClfA), an Influenza virus target antigen (e.g., Influenza HA), a Pseudomonas species target antigen (e.g., Psl), a tumor necrosis factor receptor antigen (e.g., GITR), or a B-cell surface antigen (e.g., CD20) (e.g., a target molecule [T] or a portion thereof) with a K_{D} of less than about 25 nM, less than about 20 nM, less than about 15 nM, less than about 10 nM, less than about 5 nM, less than about 1 nM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured in a surface plasmon resonance assay. In embodiments, an antigen-binding domain includes polypeptides that bind IsdB with a K_{D} of less than about less than about 5 nM, less than about 1 nM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured in a surface plasmon resonance assay.

An antigen-binding domain includes polypeptides that bind a complement component, such as C1q, with a K_{D} of less than about 25 nM, less than about 20 nM, less than about 15 nM, less than about 10 nM, less than about 5 nM, less than about 1 nM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured in a surface plasmon resonance assay. In embodiments, an antigen-binding domain includes polypeptides that bind a C1q with a K_{D} of less than about less than about 1 nM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured in a surface plasmon resonance assay.

While the present disclosure includes bispecific antigen binding molecules wherein one arm of an immunoglobulin binds a Staphylococcus species target antigen, an Influenza virus target antigen, a Pseudomonas species target antigen, a tumor necrosis factor receptor antigen, or a B-cell surface antigen (e.g. a first antigen-binding domain, D1) and the other arm of the immunoglobulin is specific for a complement component, such as C1q (e.g. the second antigen-binding domain, D2), the target antigen can be any antigen expressed on the surface of a cell or organism, e.g., an antigen on the surface of a bacteria, an antigen on the surface of a virus, an antigen on the surface of a virus infected cell, an antigen on the surface of a eukaryotic cell, an antigen on the surface of a human cell, an antigen on the surface of a cancer cell, or an antigen on the surface of an immune cell against which a targeted immune response is desired. In embodiments, the complement component can comprise C1q, C1r, C1s, C2, C3, C4, C5, C6, C7, C8, C9, or any fragments thereof (including proteolytic fragments generated by the complement cascade). In certain embodiments, the complement component is C1q. In some embodiments, the D1 and/or D2 components of the bispecific antigen-binding molecules of the present disclosure may comprise or consist of antigen-binding fragments of full antibody molecules.

In the context of bispecific antigen binding molecules of the present disclosure wherein one arm of the antibody binds C1q and the other arm binds a Staphylococcus species target antigen, the target antigen can be a cell surface antigen. Non-limiting examples of specific target antigens include capsular polysaccharide type 5, capsular polysaccharide type 8, IsdB, IsdA, IsdH, lipoteichoic acid, wall teichoic acid, clumping factor A (clfA), poly-N-acetyl glucosamine (PNAG), lipase, V8 lipase, fatty acid modifying enzyme, microbial surface components recognizing adhesive matrix molecules (e.g. adhesins, fibrinogen binding molecules, fibronectin binding protein A, fibronectin binding protein B), protein A, or combinations thereof.

In the context of the bispecific antigen binding molecules of the present disclosure, wherein one arm of the antibody binds C1q and the other arm binds a Staphylococcus species target antigen, the bispecific antigen binding molecules bind to the Staphylococcus species target antigen with an EC₅₀ of about 10 nM or less. In embodiments, the Staphylococcus species comprises Staphylococcus epidermidis, Staphylococcus aureus, Staphylococcus lugdunensis, or Staphylococcus saprophyticus. In the context of bispecific antigen binding molecule of the present disclosure wherein one arm of the antibody binds C1q and the other arm binds a Staphylococcus species target antigen, the bispecific antigen binding molecule promotes human C1q deposition on the Staphylococcus species with an EC₅₀ of about 10 nM or less.

According to certain exemplary embodiments, the present disclosure includes bispecific antigen-binding molecules that specifically bind C1q and IsdB. According to certain exemplary embodiments, the present disclosure includes bispecific antigen-binding molecules that specifically bind C1q and IsdA. According to certain exemplary embodiments, the present disclosure includes bispecific antigen-binding molecules that specifically bind C1q and Protein A. According to certain exemplary embodiments, the present disclosure includes bispecific antigen-binding molecules that specifically bind C1q and PNAG. According to certain exemplary embodiments, the present disclosure includes bispecific antigen-binding molecules that specifically bind C1q and ClfA.

In the context of bispecific antigen binding molecules of the present disclosure wherein one arm of the antibody binds C1q and the other arm binds a Pseudomonas species target antigen, the target antigen can be a cell surface antigen. Non-limiting examples of specific target antigens include any cell surface antigen associated with the Pseudomonas species, including Psl, or combinations thereof. According to certain exemplary embodiments, the present disclosure includes bispecific antigen-binding molecules that specifically bind C1q and Psl.

In the context of the bispecific antigen binding molecules of the present disclosure, wherein one arm of the antibody binds C1q and the other arm binds a Pseudomonas species target antigen, the bispecific antigen binding molecules bind to the Pseudomonas species target antigen with an EC₅₀ of about 10 nM or less. In the context of bispecific antigen binding molecule of the present disclosure wherein one arm of the antibody binds C1q and the other arm binds a Pseudomonas species target antigen, the bispecific antigen binding molecule promotes human C1q deposition on the Pseudomonas species with an EC₅₀ of about 10 nM or less.

In the context of bispecific antigen binding molecules of the present disclosure wherein one arm of the antibody binds C1q and the other arm binds an Influenza virus target antigen, the target antigen can be a cell surface antigen. Non-limiting examples of specific target antigens include any cell surface antigen associated with the Influenza virus, including Influenza HA, or combinations thereof. According to certain exemplary embodiments, the present disclosure includes bispecific antigen-binding molecules that specifically bind C1q and Influenza HA.

In the context of the bispecific antigen binding molecules of the present disclosure, wherein one arm of the antibody binds C1q and the other arm binds an Influenza virus target antigen, the bispecific antigen binding molecules bind to an Influenza virus target antigen with an EC₅₀ of about 10 nM or less. In the context of bispecific antigen binding molecule of the present disclosure wherein one arm of the antibody binds C1q and the other arm binds an Influenza virus target antigen, the bispecific antigen binding molecule promotes human C1q deposition on the Influenza strains or influenza infected cells with an EC₅₀ of about 10 nM or less.

In the context of bispecific antigen binding molecules of the present disclosure wherein one arm of the antibody binds C1q and the other arm binds a glucocorticoid induced tumor necrosis factor receptor target antigen, the target antigen can be a cell surface antigen. Non-limiting examples of specific target antigens include any cell surface antigen associated with the tumor necrosis factor receptor, including GITR, or combinations thereof. According to certain exemplary embodiments, the present disclosure includes bispecific antigen-binding molecules that specifically bind C1q and GITR.

In the context of the bispecific antigen binding molecules of the present disclosure, wherein one arm of the antibody binds C1q and the other arm binds a glucocorticoid induced tumor necrosis factor receptor target antigen, the bispecific antigen binding molecules bind to the glucocorticoid induced tumor necrosis factor receptor target antigen with an EC₅₀ of about 10 nM or less.

In the context of bispecific antigen binding molecules of the present disclosure wherein one arm of the antibody binds C1q and the other arm binds a glucocorticoid induced tumor necrosis factor receptor target antigen, the bispecific antigen binding molecule promotes human C1q deposition on the T cell with an EC₅₀ of about 10 nM or less.

In the context of bispecific antigen binding molecules of the present disclosure wherein one arm of the antibody binds C1q and the other arm binds a cancer-cell surface target antigen, the target antigen can be a cell surface antigen. Non-limiting examples of specific target antigens include any cell surface antigen associated with a cancer cell, including CD20, or combinations thereof. According to certain exemplary embodiments, the present disclosure includes bispecific antigen-binding molecules that specifically bind C1q and CD20.

In the context of the bispecific antigen binding molecules of the present disclosure, wherein one arm of the antibody binds C1q and the other arm binds a cancer-cell surface target antigen, the bispecific antigen binding molecules bind to the B-cell surface target antigen with an EC₅₀ of about 10 nM or less. In the context of bispecific antigen binding molecule of the present disclosure wherein one arm of the antibody binds C1q and the other arm binds a cancer-cell surface target antigen, the bispecific antigen binding molecule promotes human C1q deposition on CD20 bearing cancer cell with an EC₅₀ of about 10 nM or less.

In some embodiments, a bispecific antigen binding molecule comprises a first antigen-binding domain that specifically binds a target antigen as disclosed herein and comprises a second antigen-binding domain that specifically binds to human C1q. In some embodiments, the bispecific antigen binding molecule comprises a first antigen-binding domain that specifically binds a target antigen as disclosed herein and comprises a second antigen-binding domain that comprises a HCDR1 comprising an amino acid sequence selected from the group consisting of SEQ ID NO:4 and SEQ ID NO:20, a HCDR2 comprising an amino acid sequence selected from the group consisting of SEQ ID NO:6 and SEQ ID NO:22, a HCDR3 comprising an amino acid sequence selected from the group consisting of SEQ ID NO:8 and SEQ ID NO:24, a LCDR1 comprising an amino acid sequence of SEQ ID NO:12, a LCDR2 comprising an amino acid sequence of SEQ ID NO:14, and a LCDR3 comprising an amino acid sequence of SEQ ID NO:16. In some embodiments, the bispecific antigen binding molecule comprises a first antigen-binding domain that specifically binds a target antigen as disclosed herein and comprises a second antigen-binding domain that comprises a HCVR comprising the amino acid sequence of SEQ ID NO:2 or SEQ ID NO:18 and a LCVR comprising the amino acid sequence of SEQ ID NO:10.

In some embodiments, a bispecific antigen binding molecule comprises:
(a) a first antigen-binding domain that specifically binds a target antigen, wherein the first antigen-binding domain comprises a heavy chain comprising:
   (1) a HCDR1 comprising SEQ ID NO:28, a HCDR2 comprising SEQ ID NO:30, and a HCDR3 comprising SEQ ID NO:32; or
   (2) a HCDR1 comprising SEQ ID NO:36, a HCDR2 comprising SEQ ID NO:38, and a HCDR3 comprising SEQ ID NO:40; or
   (3) a HCDR1 comprising SEQ ID NO:44, a HCDR2 comprising SEQ ID NO:46, and a HCDR3 comprising SEQ ID NO:48; or
   (4) a HCDR1 comprising SEQ ID NO:52, a HCDR2 comprising SEQ ID NO:54, and a HCDR3 comprising SEQ ID NO:56; or
   (5) a HCDR1 comprising SEQ ID NO:60, a HCDR2 comprising SEQ ID NO:62, and a HCDR3 comprising SEQ ID NO:64; or
   (6) a HCDR1 comprising SEQ ID NO:68, a HCDR2 comprising SEQ ID NO:70, and a HCDR3 comprising SEQ ID NO:72; or
   (7) a HCDR1 comprising amino acids 26-33 of SEQ ID NO:73, a HCDR2 comprising amino acids 51-58 of SEQ ID NO:73, and a HCDR3 comprising amino acids 96-106 of SEQ ID NO:73; or
   (8) a HCDR1 comprising amino acids 27-32 of SEQ ID NO:76, a HCDR2 comprising amino acids 51-58 of SEQ ID NO:76, and a HCDR3 comprising amino acids 96-106 of SEQ ID NO:76; or
   (9) a HCDR1 comprising amino acids 31-35 of SEQ ID NO:78, a HCDR2 comprising amino acids 50-65 of SEQ ID NO:78, and a HCDR3 comprising amino acids 98-109 of SEQ ID NO:78;
(b) a second antigen-binding domain that specifically binds to human C1q, wherein the second antigen-binding domain comprises a heavy chain comprising:
   (1) a HCDR1 comprising SEQ ID NO:4, a HCDR2 comprising SEQ ID NO:6, and a HCDR3 comprising SEQ ID NO:8; or
   (2) a HCDR1 comprising SEQ ID NO:20, a HCDR2 comprising SEQ ID NO:22, and a HCDR3 comprising SEQ ID NO:24; and
(c) a light chain comprising:
   (1) a LCDR1 comprising SEQ ID NO:12, a LCDR2 comprising SEQ ID NO:14, and a LCDR3 comprising SEQ ID NO:16; or
   (2) a LCDR1 comprising amino acids 27-32 of SEQ ID NO:74, a LCDR2 comprising amino acids 51-55 of SEQ ID NO:74, and a LCDR3 comprising amino acids 89-98 of SEQ ID NO:74; or
   (3) a LCDR1 comprising amino acids 27-32 of SEQ ID NO:77, a LCDR2 comprising amino acids 50-52 of SEQ ID NO:77, and a LCDR3 comprising amino acids 98-101 of SEQ ID NO:77; or
   (4) a LCDR1 comprising amino acids 23-33 of SEQ ID NO:79, a LCDR2 comprising amino acids 49-55 of SEQ ID NO:79, and a LCDR3 comprising amino acids 88-99 of SEQ ID NO:79.

In some embodiments, a bispecific antigen binding molecule comprises:
(a) a first antigen-binding domain that specifically binds a target antigen, wherein the first antigen-binding domain comprises a heavy chain comprising a HCVR selected from the group consisting of SEQ ID NO:26, SEQ ID NO:34, SEQ ID NO:42, SEQ ID NO:50, SEQ ID NO:58, SEQ ID NO:66, amino acids 1-118 of SEQ ID NO:73, amino acids 1-118 of SEQ ID NO:76, and amino acids 1-118 of SEQ ID NO:78;
(b) a second antigen-binding domain that specifically binds to human C1q, wherein the second antigen-binding domain comprises a heavy chain comprising a HCVR selected from the group consisting of SEQ ID NO:2, SEQ ID NO:18, and SEQ ID NO:75; and
(c) a light chain comprising a LCVR selected from the group consisting of SEQ ID NO:10, amino acids 1-108 of SEQ ID NO:74, amino acids 1-108 of SEQ ID NO:77, and amino acids 1-108 of SEQ ID NO:79.

In some embodiments, a bispecific antigen-binding domain comprises a second antigen-binding domain that comprises a heavy chain comprising a HCDR1 comprising SEQ ID NO:4, a HCDR2 comprising SEQ ID NO:6, and a HCDR3 comprising SEQ ID NO:8, and a light chain comprising a LCDR1 comprising SEQ ID NO:12, a LCDR2 comprising SEQ ID NO:14, and a LCDR3 comprising SEQ ID NO:16. In some embodiments, a bispecific antigen-binding domain comprises a second antigen-binding domain that comprises a heavy chain comprising a HCVR comprising SEQ ID NO:2 and a LCVR comprising SEQ ID NO:10.

In some embodiments, a bispecific antigen-binding domain comprises a second antigen-binding domain that comprises a heavy chain comprising a HCDR1 comprising SEQ ID NO:20, a HCDR2 comprising SEQ ID NO:22, and a HCDR3 comprising SEQ ID NO:24, and a light chain comprising a LCDR1 comprising SEQ ID NO:12, a LCDR2 comprising SEQ ID NO:14, and a LCDR3 comprising SEQ ID NO:16. In some embodiments, a bispecific antigen-binding domain comprises a second antigen-binding domain that comprises a heavy chain comprising a HCVR comprising SEQ ID NO:18 and a LCVR comprising SEQ ID NO:10.

In certain exemplary embodiments of the present disclosure, the bispecific antigen-binding molecule comprises a first antigen-binding domain (D1) and a second antigen-binding domain (D2). The first antigen-binding domain and the second antigen-binding domain may be directly or indirectly connected to one another to form a bispecific antigen-binding molecule of the present disclosure. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin C_{H}3 domain. A non-limiting example of a multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group.

In some embodiments, the bispecific antigen-binding molecules of the present disclosure comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerizing domain is an Fc fragment or an amino acid sequence of 1 to about 200 amino acids in length containing at least one cysteine residues. In other embodiments, the multimerizing domain is a cysteine residue, or a short cysteine-containing peptide. Other multimerizing domains include peptides or polypeptides comprising or consisting of a leucine zipper, a helix-loop motif, or a coiled-coil motif. In embodiments, a multimerizing domain can include an amino acid alteration in the C_{H}3 region that cause a knob to be formed on one multimerization domain and an amino acid alteration in the C_{H}3 region that cause a corresponding hole on another multimerization domain that provides for pairing of the multimerization domains. In embodiments, amino acid alterations can provide for charge pairing of the two multimerization domains.

In some embodiments, a bispecific antigen-binding molecule of the present disclosure comprises a heavy chain constant region comprising one or more amino acid alterations in a hinge region. In some embodiments, the amino acid alteration(s) in the hinge region reduces binding to an Fcγ receptor. Exemplary modifications are disclosed in US 2018/0282411, incorporated by reference herein. In some embodiments, a bispecific antigen-binding molecule comprises a modification within positions 233-236 by EU numbering by replacing naturally occurring residues with glycine(s) and/or deletion(s). In some embodiments, each of positions 233-236 by EU number is occupied by G or is unoccupied, for example, GGG- (233-236), GG-- (233-236), G--- (233-236), or ---- (233-236), with “-” representing an unoccupied position. In some embodiments, the heavy chain constant region comprising the modification(s) is of a human IgG1 isotype. In some embodiments, the heavy chain constant region comprising the modification(s) is of a human IgG4 isotype. In some embodiments, the heavy chain constant region comprising the modification(s) is a hybrid in which domains are of different isotypes, e.g., a hybrid of IgG1 and IgG4 isotypes in which one or more domains (e.g., C_{H}1, C_{H}2, or C_{H}3 domain) and/or a hinge region is of one isotype while the remaining domains are of a different isotype.

Any bispecific antigen binding molecule format or technology may be used to make the bispecific antigen-binding molecules of the present disclosure. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present disclosure include, without limitation, e.g., scFv-based or diabody bispecific formats, V_{H} or V_{L} domain antibodies, VHH, IgG-scFv fusions, Tandem Fabs (e.g., 2+2 and 3+3 Tandem Fabs), trivalent or tetravalent formats (e.g., 2+1 N-Fabs or 2+1 C-Fabs), dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, VNAR based nanobody, and Mab² bispecific formats (see, e.g., Klein et al. 2012, MAbs 4(6):1-11; Klein et al., 2016, MAbs 8(96):1010-20; Brinkmann et al., 2017, Mabs 9(2):182-212; and references cited therein, for a review of the foregoing formats).

For purposes of clarity, exemplary bispecific antibodies of the disclosure are summarized in Table 18, which shows the particular amino acid sequence identifiers for the HCVR and HCDRs assigned to the first antigen-binding domain (D1, anti-IsdB), the amino acid sequence identifiers for the HCVR and HCDRs assigned to the second antigen-binding domain (D2, anti-C1q) and the amino acid sequence identifiers assigned to the LCVR and LCDRs. Each anti-IsdB×anti-C1q bispecific antibody was assigned a new antibody identification number (PID) shown in the first column on the left.

As a non-limiting illustrative example, the present disclosure includes anti-IsdB×anti-C1q bispecific antigen binding molecules comprising a D1 antigen-binding domain and a D2 antigen-binding domain, wherein the D1 antigen binding domain comprises an HCVR/LCVR pair comprising amino acid sequences of SEQ ID NOs:26/10, or a set of heavy and light chain CDRs (HCDR1-HCDR2-HCDR3-LCDR1-LCDR2-LCDR3), the set comprising amino acid sequences of SEQ ID NOs: 28-30-32-12-14-16, and wherein the D2 antigen-binding domain comprises an HCVR/LCVR pair comprising amino acid sequences of SEQ ID NOs: 2/10 or a set of heavy and light chain CDRs (HCDR1-HCDR2-HCDR3-LCDR1-LCDR2-LCDR3), the set comprising amino acid sequences of SEQ ID NOs: 4-6-8-12-14-16. An exemplary anti-IsdB×anti-C1q bispecific antibody having these sequence characteristics is the bispecific antibody designated REGN5066, which comprises a D1 derived from mAb20295 and a D2 derived from mAb17736. Another exemplary anti-IsdB×anti-C1q bispecific antibody having these sequence characteristics is the bispecific antibody designated REGN5067, which comprises a D1 derived from mAb20295 and a D2 derived from mAb18395 (see Table 18 herein).

As a non-limiting illustrative example, the present disclosure includes anti-IsdA×anti-C1q bispecific antigen binding molecules comprising a D1 antigen-binding domain and a D2 antigen-binding domain, wherein the D1 antigen binding domain comprises an HCVR/LCVR pair comprising amino acid sequences of SEQ ID NOs: 66/10, or a set of heavy and light chain CDRs (HCDR1-HCDR2-HCDR3-LCDR1-LCDR2-LCDR3), the set comprising amino acid sequences of SEQ ID NOs: 68-70-72-12-14-16, and wherein the D2 antigen-binding domain comprises an HCVR/LCVR pair comprising amino acid sequences of SEQ ID NOs: 2/10, or a set of heavy and light chain CDRs (HCDR1-HCDR2-HCDR3-LCDR1-LCDR2-LCDR3), the set comprising amino acid sequences of SEQ ID NOs: 4-6-8-12-14-16. An exemplary anti-IsdA×anti-C1q bispecific antibody having these sequence characteristics is the bispecific antibody designated REGN5553, which comprises a D1 derived from mAb20334 and a D2 derived from mAb17736 (see Table 18 herein).

As a non-limiting illustrative example, the present disclosure includes anti-Protein A×anti-C1q bispecific antigen binding molecules comprising a D1 antigen-binding domain and a D2 antigen-binding domain, wherein the D1 antigen binding domain comprises an HCVR/LCVR pair comprising amino acid sequences of SEQ ID NOs: 58/10, or a set of heavy and light chain CDRs (HCDR1-HCDR2-HCDR3-LCDR1-LCDR2-LCDR3), the set comprising amino acid sequences of SEQ ID NOs: 60-62-64-12-14-16, and wherein the D2 antigen-binding domain comprises an HCVR/LCVR pair comprising amino acid sequences of SEQ ID NOs: 2/10, or a set of heavy and light chain CDRs (HCDR1-HCDR2-HCDR3-LCDR1-LCDR2-LCDR3), the set comprising amino acid sequences of SEQ ID NOs: 4-6-8-12-14-16. An exemplary anti-Protein A×anti-C1q bispecific antibody having these sequence characteristics is the bispecific antibody designated REGN5551, which comprises a D1 derived from mAb15144 and a D2 derived from mAb17736 (see Table 18 herein).

As a non-limiting illustrative example, the present disclosure includes anti-PNAG×anti-C1q bispecific antigen binding molecules comprising a D1 antigen-binding domain and a D2 antigen-binding domain, wherein the D1 antigen binding domain comprises an HCVR/LCVR pair comprising amino acid sequences contained within the HC/LC pair of SEQ ID NOs: 76/77, and wherein the D2 antigen-binding domain comprises an scFv comprising the amino acid sequence of SEQ ID NO: 75. An exemplary anti-PNAG×anti-C1q bispecific antibody having these sequence characteristics is the bispecific antibody designated REGN5536 which comprises a D1 derived from REGN1335 and a D2 derived from mAb17736 (see Table 18 herein).

As a non-limiting illustrative example, the present disclosure includes anti-ClfA×anti-C1q bispecific antigen binding molecules comprising a D1 antigen-binding domain and a D2 antigen-binding domain, wherein the D1 antigen binding domain comprises an HCVR/LCVR pair comprising amino acid sequences contained within the HC/LC pair of SEQ ID NOs:73/74, and wherein the D2 antigen-binding domain comprises an scFv comprising the amino acid sequence of SEQ ID NO:75. An exemplary anti-ClfA×anti-C1q bispecific antibody having these sequence characteristics is the bispecific antibody designated REGN5738 having HCVR/LCVR pairs contained within the HC/LC pair of SEQ ID NO: 73/74 and a D2 derived from mAb17736 (see Table 18 herein).

As a non-limiting illustrative example, the present disclosure includes anti-Psl×anti-C1q bispecific antigen binding molecules comprising a D1 antigen-binding domain and a D2 antigen-binding domain, wherein the D1 antigen binding domain comprises an HCVR/LCVR pair comprising amino acid sequences contained within the HC/LC pair of SEQ ID NOs:78/79, and wherein the D2 antigen-binding domain comprises an scFv comprising the amino acid sequence of SEQ ID NO:75. An exemplary anti-Psl×anti-C1q bispecific antibody having these sequence characteristics is the bispecific antibody designated REGN5409 which comprises a D1 having an HCVR/LCVR contained within the HC/LC pair of SEQ ID NOs: 78/79 and a D2 derived from mAb17736 (see Table 18 herein).

As a non-limiting illustrative example, the present disclosure includes anti-Influenza HA×anti-C1q bispecific antigen binding molecules comprising a D1 antigen-binding domain and a D2 antigen-binding domain, wherein the D1 antigen binding domain comprises an HCVR/LCVR pair comprising amino acid sequences of SEQ ID NOs: 42/10, or a set of heavy and light chain CDRs (HCDR1-HCDR2-HCDR3-LCDR1-LCDR2-LCDR3) comprising a set of amino acid sequences SEQ ID NOs: 44-46-48-12-14-16. The D2 antigen-binding domain comprises an HCVR/LCVR pair comprising amino acid sequences of SEQ ID Nos:2/10, or a set of heavy and light chain CDRs (HCDR1-HCDR2-HCDR3-LCDR1-LCDR2-LCDR3), the set comprising amino acid sequences of SEQ ID NOs:4-6-8-12-14-16. An exemplary anti-Influenza HA×anti-C1q bispecific antibody having these sequence characteristics is the bispecific antibody designated REGN 6093, which comprises a D1 derived from mAb 11829 and a D2 derived from mAb17736 (see Table 18 herein).

As a non-limiting illustrative example, the present disclosure includes anti-CD20×anti-C1q bispecific antigen binding molecules comprising a D1 antigen-binding domain and a D2 antigen-binding domain, wherein the D1 antigen binding domain comprises an HCVR/LCVR pair comprising amino acid sequences of SEQ ID NOs: 34/10, or a set of heavy and light chain CDRs (HCDR1-HCDR2-HCDR3-LCDR1-LCDR2-LCDR3), the set comprising amino acid sequences of SEQ ID NOs: 36-38-40-12-14-16 and wherein the D2 antigen-binding domain comprises an HCVR/LCVR pair comprising amino acid sequences of SEQ ID NOs:2/10, or a set of heavy and light chain CDRs (HCDR1-HCDR2-HCDR3-LCDR1-LCDR2-LCDR3), the set comprising amino acid sequences of SEQ ID NOs:4-6-8-12-14-16. An exemplary anti-CD20×anti-C1q bispecific antibody having these sequence characteristics is the bispecific antibody designated REGN 5177, which comprises a D1 derived from mAb14303 and a D2 derived from mAb17736 (see Table 18 herein).

As a non-limiting illustrative example, the present disclosure includes anti-GITR×anti-C1q bispecific antigen binding molecules comprising a D1 antigen-binding domain and a D2 antigen-binding domain, wherein the D1 antigen binding domain comprises an HCVR/LCVR pair comprising amino acid sequences of SEQ ID NOs: 50/10, or a set of heavy and light chain CDRs (HCDR1-HCDR2-HCDR3-LCDR1-LCDR2-LCDR3), the set comprising amino acid sequences of SEQ ID NOs: 52-54-56-12-14-16 and wherein the D2 antigen-binding domain comprises an HCVR/LCVR pair comprising amino acid sequences of SEQ ID NOs:2/10 or a set of heavy and light chain CDRs (HCDR1-HCDR2-HCDR3-LCDR1-LCDR2-LCDR3), the set comprising amino acid sequences of SEQ ID NOs:4-6-8-12-14-16. An exemplary anti-GITR×anti-C1q bispecific antibody having these sequence characteristics is the bispecific antibody designated REGN 6545, which comprises a D1 derived from mAb14536 and a D2 derived from mAb17736 (see Table 18 herein). Another exemplary anti-GITR×anti-C1q bispecific antibody having these sequence characteristics is the bispecific antibody designated REGN 6546, which comprises a D1 derived from mAb14536 and a D2 derived from mAb18395 (see Table 18 herein).

### Sequence Variants

The antibodies and bispecific antigen-binding molecules of the present disclosure may comprise one or more amino acid substitutions, insertions and/or deletions in the framework and/or CDR regions of the heavy and light chain variable domains as compared to the corresponding germline sequences from which the individual antigen-binding domains were derived as described herein. The present disclosure also includes antigen-binding molecules wherein one or both antigen-binding domains comprise variants of any of the HCVR, LCVR, and/or CDR amino acid sequences disclosed herein having one or more conservative substitutions. For example, the present disclosure includes antigen-binding molecules comprising an antigen-binding domain having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein. Conservative amino acid substitutions are described elsewhere herein.

The present disclosure also includes antigen-binding molecules comprising an antigen-binding domain with an HCVR, LCVR, and/or CDR amino acid sequence that is substantially identical to any of the HCVR, LCVR, and/or CDR amino acid sequences disclosed herein.

### Bioequivalents

The present disclosure encompasses antigen-binding molecules having amino acid sequences that vary from those of the exemplary molecules disclosed herein but that retain the ability to bind a complement component (e.g., C1q) and/or a target antigen (e.g., IsdB, IsdA, Protein A, PNAG, CflA, Psl, Influenza HA, CD20, or GITR). Such variant molecules may comprise one or more additions, deletions, or substitutions of amino acids when compared to parent sequence, but exhibit biological activity that is essentially equivalent to that of the described bispecific antigen-binding molecules.

The present disclosure includes antigen-binding molecules that are bioequivalent to any of the exemplary antigen-binding molecules set forth herein. Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antigen-binding proteins will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antigen-binding protein.

Bioequivalent variants of the exemplary bispecific antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antigen-binding proteins may include variants of the exemplary bispecific antigen-binding molecules set forth herein comprising amino acid changes which modify the glycosylation characteristics of the molecules, e.g., mutations which eliminate or remove glycosylation.

### V. Therapeutic Formulation and Administration

The present disclosure provides pharmaceutical compositions comprising the antigen-binding molecules of the present disclosure. The pharmaceutical compositions of the disclosure are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antigen-binding molecule administered to a patient may vary depending upon the age and the size of the patient, target disease, conditions, route of administration, and the like. The preferred dose is typically calculated according to body weight or body surface area. When a bispecific antigen-binding molecule of the present disclosure is used for therapeutic purposes in an adult patient, it may be advantageous to administer (e.g., intravenously administer or subcutaneously administer) the bispecific antigen-binding molecule of the present disclosure normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. Effective dosages and schedules for administering a bispecific antigen-binding molecule may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In some embodiments, a pharmaceutical composition comprising an antigen-binding molecule of the present disclosure is contained in a container, including but not limited to a syringe, pen delivery device, autoinjector, glass vial, or microinfuser. In some embodiments, a pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

### VI. Therapeutic Uses of the Antigen-Binding Molecules

In another aspect, the present disclosure provides for methods of using the bispecific antigen-binding molecules disclosed herein. In some embodiments, the present disclosure provides methods of activating or enhancing cell killing by contacting the cell with a bispecific antigen-binding molecule that specifically binds a complement component (e.g., C1q) and a target antigen as disclosed herein. In some embodiments, the cell is a bacterial cell. In some embodiments, the cell is a eukaryotic cell. In some embodiments, the cell is a virally infected cell, an immune cell, or a cancer cell. In some embodiments, the cell is a non-diseased cell. In some embodiments, the method is performed in vitro. In some embodiments, the method is performed in vivo. In some embodiments, the method is performed ex vivo.

In some embodiments, the bispecific antigen-binding molecules disclosed herein can be used for reducing or depleting a population of cells that express a target antigen of interest. The population of cells that is targeted for reduction or depletion can comprise diseased cells, or the population of cells can comprise non-diseased cells that are nonetheless desirable to target for reduction or depletion.

The present disclosure includes methods of inhibiting the growth of a bacteria (e.g., Staphylococcus species) in a subject comprising administering a therapeutic composition comprising a bispecific antigen-binding molecule that specifically binds a complement component (e.g., C1q) and a Staphylococcus species target antigen (e.g., IsdB) to a subject in need thereof. The therapeutic composition can comprise any of the antibodies or fragments thereof or bispecific antigen-binding molecules as disclosed herein and a pharmaceutically acceptable carrier or diluent.

The present disclosure includes methods of inhibiting the growth of a bacteria (e.g., Pseudomonas species) in a subject comprising administering a therapeutic composition comprising a bispecific antigen-binding molecule that specifically binds a complement component (e.g., C1q) and Pseudomonas species target antigen (e.g., Psl) to a subject in need thereof. The therapeutic composition can comprise any of the antibodies or fragments thereof or bispecific antigen-binding molecules as disclosed herein and a pharmaceutically acceptable carrier or diluent.

The antigen-binding molecules of the present disclosure may be used to treat diseases or disorders associated with infection with bacterial species, especially antibiotic resistant species, including a skin infection, cellulitis, pneumonia, meningitis, urinary tract infection, toxic shock syndrome, endocarditis, pericarditis, osteomyelitis, bacteremia, or sepsis. The antigen-binding molecules of the present disclosure may also be used to prevent infections with a bacterial species that may arise as a result of a long hospital stay (i.e. a nosocomial or hospital acquired infection). The antigen-binding molecules of the present disclosure may also be used to prevent infections with a bacterial species that may arise during or following a surgical procedure, such as surgery that involves implantation of a prosthetic. In certain embodiments, the prosthetic may be a prosthetic limb, such as an arm or a leg. In certain embodiments, the prosthetic may be a hip replacement. In certain embodiments, the prosthetic may be a cosmetic prosthetic, such as, but not limited to an ocular prosthetic, silicone hands, fingers, breasts, feet, toes, or a facial implant.

The antigen-binding molecules of the present disclosure may be used to treat diseases or disorders associated with infection with a virus, e.g., influenza virus, smallpox virus, hepatitis virus (e.g., hepatitis A, hepatitis B, or hepatitis C virus), human papillomavirus, human immunodeficiency virus (HIV), respiratory syncytial virus (RSV), herpesvirus, coronavirus, cowpox virus, coxsackievirus, Ebola virus, Epstein-Barr virus, measles virus, mumps virus, rubella virus, rotavirus, rabies virus, yellow fever virus, meningitis, poliovirus, rotavirus, vaccinia virus, varicella-zoster virus, variola virus, or Zika virus. The present disclosure includes methods of inhibiting the growth of a virus (e.g., Influenza virus) in a subject comprising administering a therapeutic composition comprising a bispecific antigen-binding molecule that specifically binds a complement component (e.g., C1q) and a virus target antigen (e.g., Influenza HA) to a subject in need thereof. The therapeutic composition can comprise any of the antibodies or fragments thereof or bispecific antigen-binding molecules as disclosed herein and a pharmaceutically acceptable carrier or diluent.

The present disclosure includes methods of killing or inhibiting the growth of a cell, e.g., a diseased cell, in a subject, by administering a bispecific antigen-binding molecule that specifically binds a complement component (e.g., C1q) and a target antigen expressed on the surface of the cell (e.g., a target antigen expressed by the diseased cell).

In embodiments, the antigen-binding molecules of the present disclosure may be used to treat diseases or disorders associated with cancer, e.g., a CD20-expressing or CD20-overexpressing cancer. The present disclosure includes methods of inhibiting the growth of cancer in a subject comprising administering a therapeutic composition comprising a bispecific antigen-binding molecule that specifically binds a complement component (e.g., C1q) and a cancer-cell specific antigen (e.g., CD20) to a subject in need thereof. The therapeutic composition can comprise any of the antibodies or fragments thereof or bispecific antigen-binding molecules as disclosed herein and a pharmaceutically acceptable carrier or diluent.

The antigen-binding molecules of the present disclosure may be used to treat diseases or disorders associated with an immune cell, e.g., a glucocorticoid induced tumor necrosis factor receptor (GITR) antigen-expressing cell. The antigen-binding molecules of the present disclosure may also be used to treat disease states associated with regulatory T cells. In embodiments, inhibition of regulatory T cells can be useful in the treatment of cancer.

### Combination Therapy

The present disclosure provides methods, which comprise administering a pharmaceutical composition comprising any of the exemplary antibodies and bispecific antigen-binding molecules described herein in combination with one or more additional therapeutic agents. Exemplary additional therapeutic agents that may be combined with or administered in combination with a bispecific antigen-binding molecule of the present disclosure comprise, but are not limited to, an antibiotic, an antibody that binds a Staphylococcus antigen, an antibody that binds an Influenza virus antigen, anti-viral agent, an antibody that binds a Pseudomonas antigen, an antibody that binds to a cancer target cell antigen, an antibody that binds a glucocorticoid induced tumor necrosis factor receptor antigen, an antibody that binds human or cynomolgus C1q, a vaccine specific for a bacterial infection, a vaccine for a viral infection, a vaccine for cancer, Staphylococcus species, an Influenza virus, Pseudomonas species, a B-cell, or a tumor necrosis factor receptor, an antibody drug conjugate (e.g. a bispecific antibody conjugated to an antibiotic), or combinations thereof.

In various embodiments, bispecific antigen-binding molecule of the present disclosure may be used in combination with an antibody to PD-L1, an antibody to PD-1 (e.g., nivolumab), a LAG-3 inhibitor, a CTLA-4 inhibitor (e.g., ipilimumab), a TIM3 inhibitor, a BTLA inhibitor, a MET inhibitor, a TIGIT inhibitor, a CD47 inhibitor, an antagonist of a T-cell co-inhibitor or ligand (e.g., an antibody to CD-28, 2B4, LY108, LAIR1, ICOS, CD160 or VISTA), an indoleamine-2,3-dioxygenase (IDO) inhibitor, a VEGF antagonist [e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab) or a small molecule kinase inhibitor of VEGF receptor (e.g., sunitinib, sorafenib, or pazopanib)], an Ang2 inhibitor (e.g., nesvacumab), a TGFβ inhibitor, an EGFR inhibitor (e.g., erlotinib or cetuximab), an agonist to a co-stimulatory receptor (e.g., an agonist to glucocorticoid-induced TNFR-related protein), an antibody to a tumor-specific antigen (e.g., CA9, CA125, MAGEA3, carcinoembryonic antigen (CEA), vimentin, tumor-M2-PK, prostate-specific antigen (PSA), mucin-1, MART-1, or CA19-9), a vaccine (e.g., Bacillus Calmette-Guerin, influenza virus, smallpox, hepatitis virus (e.g., hepatitis A, hepatitis B, or hepatitis C virus), human papillomavirus, human immunodeficiency virus (HIV), respiratory syncytial virus (RSV), zoster, chickenpox, herpesvirus, coronavirus, Ebola virus, measles, mumps, rubella, rabies, yellow fever, meningitis, poliovirus, rotavirus, vaccinia virus, pertussis, diphtheria, tetanus, tuberculosis, typhoid, cholera, malaria, anthrax, Lyme disease, and/or a cancer vaccine), an anti-viral agent (e.g., anti-viral antibodies, neuraminidase inhibitors, nucleoside analogs, interferons, an antiretroviral agent, rimantadine, oseltamivir (e.g., TAMIFLU®), zanamivir (RELENZA®), peramivir (RAPIVAB®), or baloxavir marboxil (XOFLUZA®), an adjuvant to increase antigen presentation (e.g., granulocyte-macrophage colony-stimulating factor), a bispecific antibody (e.g., CD3×CD20, PSMA×CD3, MUC16×CD3, BCMA×CD3, CD3×CD28, bispecific antibody), a cytotoxin, a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, or vincristine), cyclophosphamide, radiotherapy, an IL-6R inhibitor (e.g., sarilumab), an IL-4R inhibitor (e.g., dupilumab), an IL-10 inhibitor, an IL-33 inhibitor, a cytokine such as IL-2, IL-7, IL-21, and IL-15, an antibody-drug conjugate (ADC), an antibiotic (e.g., amoxicillin, cefazolin, cefuroxime, cephalexin, ciprofloaxicin, clindamycin, daptomycin, dicloxacillin, doxycycline, fosfomycin, flucloxacillin, lincomycin, linezolid, methicillin, nafcillin, oxacillin, penicillin, rifampin, telavancin, vancomycin, an aminoglyside, a carbapenem, a cephalosporin, a fluoroquinolone, a monobactam, or a polymyxin), an anti-inflammatory drug (e.g., corticosteroids or non-steroidal anti-inflammatory drugs), a dietary supplement such as anti-oxidants, or palliative care.

In some embodiments, the additional therapeutic agent is an anti-cancer agent. The anti-cancer agent may target the same or a different antigen as the antigen-binding molecule of the disclosure. As a non-limiting example, an antigen-binding molecule that specifically binds CD20 can be administered in combination with a second therapeutic agent (e.g., anti-cancer agent) that also targets CD20, or can be administered in combination with a therapeutic agent (e.g., anti-cancer agent) that targets a different antigen, such as CD3, PD-1, PD-L1, or other antigens. In some embodiments, the additional therapeutic agent is an antibody (e.g., a monospecific, bispecific, or multispecific antibody). In some embodiments, the additional therapeutic agent is a bispecific or multispecific antibody in which one arm targets the same antigen as the antigen-binding molecule of the disclosure. For example, an antigen-binding molecule that specifically binds CD20 can be administered in combination with a bispecific antibody having an anti-CD20 arm (e.g., an anti-CD3×anti-CD20 arm). In some embodiments, the additional therapeutic agent is a bispecific or multispecific antibody that does not target the same antigen as the antigen-binding molecule of the disclosure.

The additional therapeutically active component(s) may be administered prior to, concurrent with, or shortly after the administration of an antigen-binding molecule of the present disclosure. For the purposes of the present disclosure, such administration regimens are considered the administration of an antigen-binding molecule “in combination with” an additional therapeutically active component.

The present disclosure includes pharmaceutical compositions in which an antigen-binding molecule of the present disclosure is co-formulated with one or more of the additional therapeutically active component(s) as described elsewhere herein.

### EXAMPLES

The following examples are put forth so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Preparation of Anti-C1q Antibodies

Anti-C1q antibodies were obtained by immunizing a VELOCIMMUNE® mouse (i.e., an engineered mouse comprising DNA encoding human Immunoglobulin heavy and kappa light chain variable regions; see U.S. Pat. No. 6,596,541) with a human complement component 1q (C1q). Exemplary anti-C1q antibodies are described in U.S. Ser. No. 16/143,901 and PCT/US2018/053064.

Human complement C1q is a hexamer that comprises three unique subunits: A (29 kDa); B (26 kDa); and C (22 kDa). An exemplary sequence of subunit A is found at amino acids 1-245 of accession number NP_057075. An exemplary sequence of subunit B is found at amino acids 1-253 of accession number NP_000482. An exemplary sequence of subunit C is found at amino acids 1-245 of accession number NP_758957. Human C1q is also commercially available from a source such as Quidel. In embodiments, the commercially available human C1q from Quidel was used as the immunogen. The antibody immune response was monitored by a C1q-specific immunoassay.

Several fully human anti-C1q antibodies were isolated directly from antigen-positive B cells from the VELOCIMMUNE® mouse without fusion to myeloma cells, as described in U.S. Pat. No. 7,582,298, which is incorporated by reference herein. As will be appreciated by a person of ordinary skill in the art, an antibody having a particular Fc isotype can be converted to an antibody with a different Fc isotype (e.g., an antibody with a mouse IgG1 Fc can be converted to an antibody with a human IgG4, etc.), but in any event, the variable domains (including the CDRs)—which are indicated by the numerical identifiers shown in Table 1—will remain the same, and the binding properties are expected to be identical or substantially similar regardless of the nature of the Fc domain.

Once the antibodies were isolated, the nucleic acid and amino acid sequences were obtained. The amino acid sequence identifiers of the exemplary anti-C1q antibodies mAb17736 and mAb18395, including heavy chain variable region (HCVR), heavy chain complementarity determining region 1 (HCDR1), heavy chain complementarity determining region 2 (HCDR2), heavy chain complementarity determining region 3 (HCDR3), light chain variable region (LCVR), light chain complementarity determining region 1 (LCDR1), light chain complementarity determining region 2 (HCDR2), light chain complementarity determining region 3 (HCDR3), heavy chain (HC), and light chain (LC) are shown in Table 1 below. The nucleic acid sequence identifiers are shown in Table 2.

TABLE 1
Amino Acid Sequence Identifiers for anti-hC1q antibodies
SEQ ID NOs:

| Antibody | | | | | | | | | | |
|---|---|---|---|---|---|---|---|---|---|---|
| designation | HCVR | HCDR1 | HCDR2 | HCDR3 | LCVR | LCDR1 | LCDR2 | LCDR3 | HC | LC |
| mAb17736 | 2 | 4 | 6 | 8 | 10 | 12 | 14 | 16 | 83 | 85 |
| mAb18395 | 18 | 20 | 22 | 24 | 10 | 12 | 14 | 16 | 89 | 85 |

TABLE 2
Nucleic Acid Sequence Identifiers for anti-hC1q antibodies
SEQ ID NOs:

| Antibody | | | | | | | | |
|---|---|---|---|---|---|---|---|---|
| designation | HCVR | HCDR1 | HCDR2 | HCDR3 | LCVR | LCDR1 | LCDR2 | LCDR3 |
| mAb17736 | 1 | 3 | 5 | 7 | 9 | 11 | 13 | 15 |
| mAb18395 | 17 | 19 | 21 | 23 | 9 | 11 | 13 | 15 |

### Example 2. Preparation of Anti-IsdB Antibodies

Anti-IsdB antibodies were obtained by immunizing a VELOCIMMUNE® mouse (i.e., an engineered mouse comprising DNA encoding human Immunoglobulin heavy and kappa light chain variable regions, see U.S. Pat. No. 6,596,541) with an antigen derived from a heme uptake protein IsdB from Staphylococcus aureus (IsdB). Exemplary anti-IsdB antibodies are described in U.S. Ser. No. 16/143,901 and PCT/US2018/053064.

An embodiment of the antigen comprises amino acids Alanine 41 to threonine 613 of Accession No. WP_099560907. Another embodiment of the antigen, identified as IsdB.6× (His), is used in assays. IsdB.6× (His) includes linkers and 6 histidines and has the sequence of SEQ ID NO: 80. The antibody immune response was monitored by an IsdB-specific immunoassay.

Several fully human anti-IsdB antibodies were isolated directly from antigen-positive B cells VELOCIMMUNE® mouse without fusion to myeloma cells, as described in U.S. Pat. No. 7,582,298. Once the antibodies were isolated, the nucleic acid and amino acid sequences were obtained. The amino acid sequence identifiers of the anti-C1q antibodies exemplary anti-IsdB antibody mAb20295, including heavy chain variable region (HCVR), heavy chain complementarity determining region 1 (HCDR1), heavy chain complementarity determining region 2 (HCDR2), heavy chain complementarity determining region 3 (HCDR3), light chain variable region (LCVR), light chain complementarity determining region 1 (LCDR1), light chain complementarity determining region 2 (HCDR2), light chain complementarity determining region 3 (HCDR3), heavy chain (HC), and light chain (LC), are shown in Table 3 below. The nucleic acid sequence identifiers are shown in Table 4.

TABLE 3
Amino Acid Sequence Identifiers for anti-IsdB antibodies
SEQ ID NOs:

| Antibody | | | | | | | | | | |
|---|---|---|---|---|---|---|---|---|---|---|
| designation | HCVR | HCDR1 | HCDR2 | HCDR3 | LCVR | LCDR1 | LCDR2 | LCDR3 | HC | LC |
| mAb20295 | 26 | 28 | 30 | 32 | 10 | 12 | 14 | 16 | 84 | 85 |

TABLE 4
Nucleic Acid Sequence identifiers for anti-IsdB antibodies
SEQ ID NOs:

| Antibody | | | | | | | | |
|---|---|---|---|---|---|---|---|---|
| designation | HCVR | HCDR1 | HCDR2 | HCDR3 | LCVR | LCDR1 | LCDR2 | LCDR3 |
| mAb20295 | 25 | 27 | 29 | 31 | 9 | 11 | 13 | 15 |

### Example 3. Preparation of Anti-CD20 Antibodies

Anti-CD20 antibodies were obtained by immunizing a VELOCIMMUNE® mouse (i.e., an engineered mouse comprising DNA encoding human Immunoglobulin heavy and kappa light chain variable regions, see U.S. Pat. No. 6,596,541) with an antigen derived from human B lymphocyte restricted antigen found on mature B cells. Exemplary anti-CD20 antibodies are described in U.S. Pat. No. 9,657,102, US Publication No. 2015/0266966, and US Publication No. 2018/0194841.

An embodiment of the antigen comprises amino acids as shown in Genbank NP_690605. Mice can also be immunized with CD20 expressing Raji cells. The antibody immune response was monitored by a CD20-specific immunoassay.

Several fully human anti-CD20 antibodies were isolated directly from antigen-positive B cells VELOCIMMUNE® mouse without fusion to myeloma cells, as described in U.S. Pat. No. 7,582,298. Once the antibodies were isolated, the nucleic acid and amino acid sequences were obtained. The amino acid sequence identifiers of an exemplary anti-CD20 antibody mAb14303, including heavy chain variable region (HCVR), heavy chain complementarity determining region 1 (HCDR1), heavy chain complementarity determining region 2 (HCDR2), heavy chain complementarity determining region 3 (HCDR3), light chain variable region (LCVR), light chain complementarity determining region 1 (LCDR1), light chain complementarity determining region 2 (HCDR2), light chain complementarity determining region 3 (HCDR3), heavy chain (HC), and light chain (LC), are shown in Table 5 below. The nucleic acid sequence identifiers are shown in Table 6.

TABLE 5
Amino Acid Sequence Identifiers for Anti-CD20 Antibodies
SEQ ID NOs:

| Antibody | | | | | | | | | | |
|---|---|---|---|---|---|---|---|---|---|---|
| designation | HCVR | HCDR1 | HCDR2 | HCDR3 | LCVR | LCDR1 | LCDR2 | LCDR3 | HC | LC |
| mAb14303 | 34 | 36 | 38 | 40 | 10 | 12 | 14 | 16 | 86 | 85 |

TABLE 6
Nucleic Acid Sequence Identifiers for Anti-CD20 Antibodies
SEQ ID NOs:

| Antibody | | | | | | | | |
|---|---|---|---|---|---|---|---|---|
| designation | HCVR | HCDR1 | HCDR2 | HCDR3 | LCVR | LCDR1 | LCDR2 | LCDR3 |
| mAb14303 | 33 | 35 | 37 | 39 | 9 | 11 | 13 | 15 |

### Example 4. Preparation of Anti-Influenza HA Antibodies

Anti-Influenza HA antibodies were obtained by immunizing a VELOCIMMUNE® mouse (i.e., an engineered mouse comprising DNA encoding human Immunoglobulin heavy and kappa light chain variable regions, see U.S. Pat. No. 6,596,541) with an antigen derived from an H1N1 strain of Influenza. Exemplary anti-HA antibodies are described in U.S. Ser. No. 14/974,361 having Publication No. 2016/0176953.

An embodiment of the antigen comprises amino acids as shown in Genbank AAP34324. An example of the amino acid sequence of an Influenza HA antigen, identified as H1N1 Ecto foldon-foldon-Bir-His6, is amino acids 1 to 519 of Genbank AAP34324 with an amino acid substitution at position 108 of a Phe (F) for a Tyr (Y) having the amino acid sequence of SEQ ID NO: 81. The foldon domain provides for trimerization of the H1N1 ectodomain of HA. The antibody immune response was monitored by an Influenza HA-specific immunoassay.

Several fully human anti-Influenza HA antibodies were isolated directly from antigen-positive B cells VELOCIMMUNE® mouse without fusion to myeloma cells, as described in U.S. Pat. No. 7,582,298. Once the antibodies were isolated, the nucleic acid and amino acid sequences were obtained. The amino acid sequence identifiers of an exemplary anti-Influenza HA antibody mAb11829, including heavy chain variable region (HCVR), heavy chain complementarity determining region 1 (HCDR1), heavy chain complementarity determining region 2 (HCDR2), heavy chain complementarity determining region 3 (HCDR3), light chain variable region (LCVR), light chain complementarity determining region 1 (LCDR1), light chain complementarity determining region 2 (HCDR2), light chain complementarity determining region 3 (HCDR3), heavy chain (HC), and light chain (LC), are shown in Table 7 below. The nucleic acid sequence identifiers are shown in Table 8.

TABLE 7
Amino Acid Sequence Identifiers for Anti-Influenza HA Antibodies
SEQ ID NOs:

| Antibody | | | | | | | | | | |
|---|---|---|---|---|---|---|---|---|---|---|
| designation | HCVR | HCDR1 | HCDR2 | HCDR3 | LCVR | LCDR1 | LCDR2 | LCDR3 | HC | LC |
| mAb11829 | 42 | 44 | 46 | 48 | 10 | 12 | 14 | 16 | 87 | 85 |

TABLE 8
Nucleic Acid Sequence Identifiers for Anti- Influenza HA Antibodies
SEQ ID NOs:

| Antibody | | | | | | | | |
|---|---|---|---|---|---|---|---|---|
| designation | HCVR | HCDR1 | HCDR2 | HCDR3 | LCVR | LCDR1 | LCDR2 | LCDR3 |
| mAb11829 | 41 | 43 | 45 | 47 | 9 | 11 | 13 | 15 |

### Example 5. Preparation of Anti-GITR Antibodies

Anti-GITR antibodies were obtained by immunizing a VELOCIMMUNE® mouse (i.e., an engineered mouse comprising DNA encoding human Immunoglobulin heavy and kappa light chain variable regions, see U.S. Pat. No. 6,596,541) with an antigen derived from human tumor necrosis factor receptor superfamily member 18. Exemplary anti-GITR antibodies are described in U.S. Ser. No. 15/619,068 having publication number US2017/0355774.

An embodiment of the antigen comprises amino acids as shown in Genbank NP 683700. Another embodiments of GITR, identified as hGITR.mmm, has amino acids Gln (Q) at position 26 to Pro (P) at position 162 of the sequence of Accession No. NP_683700 and mmh domain of amino acids 136-165 of SEQ ID NO: 82. Mice can be immunized with HEK293 cells expressing GITR or CHO-K1 cells expressing GITR. The antibody immune response was monitored by a GITR-specific immunoassay.

Several fully human anti-GITR antibodies were isolated directly from antigen-positive B cells VELOCIMMUNE® mouse without fusion to myeloma cells, as described in U.S. Pat. No. 7,582,298. Once the antibodies were isolated, the nucleic acid and amino acid sequences were obtained. The amino acid sequence identifiers of an exemplary anti-GITR antibody mAb14536, including heavy chain variable region (HCVR), heavy chain complementarity determining region 1 (HCDR1), heavy chain complementarity determining region 2 (HCDR2), heavy chain complementarity determining region 3 (HCDR3), light chain variable region (LCVR), light chain complementarity determining region 1 (LCDR1), light chain complementarity determining region 2 (HCDR2), light chain complementarity determining region 3 (HCDR3), heavy chain (HC), and light chain (LC), are shown in Table 9 below. The nucleic acid sequence identifiers are shown in Table 10.

TABLE 9
Amino Acid Sequence Identifiers for Anti-GITR Antibodies
SEQ ID NOs:

| Antibody | | | | | | | | | | |
|---|---|---|---|---|---|---|---|---|---|---|
| designation | HCVR | HCDR1 | HCDR2 | HCDR3 | LCVR | LCDR1 | LCDR2 | LCDR3 | HC | LC |
| mAb14536 | 50 | 52 | 54 | 56 | 10 | 12 | 14 | 16 | 88 | 85 |

TABLE 10
Nucleic Acid Sequence Identifiers for Anti-GITR Antibodies
SEQ ID NOs:

| Antibody | | | | | | | | |
|---|---|---|---|---|---|---|---|---|
| designation | HCVR | HCDR1 | HCDR2 | HCDR3 | LCVR | LCDR1 | LCDR2 | LCDR3 |
| mAb14536 | 49 | 51 | 53 | 55 | 9 | 11 | 13 | 15 |

### Example 6. Preparation of Anti-Psl Antibodies

Recombinant C1q bispecific REGN 5409 was produced in CHO cells after transfection with two expression plasmids. One of the expression plasmids encodes the heavy chain (hIgG1) and light chain of an antibody that binds Psl and the second expression plasmid encodes a C1q binding scFv-Fc (hIgG1), whose Fc contains the “Fc*” mutation. Specifically, the C1q binding scFv-Fc* was constructed as follows: (1-39 ULC VK_(G4S) 3 Linker_17736PVH.hIgG1(amino acids D104-K330; H318R,Y319F (UniProtKB/Swiss-Prot: P01857.1). The scFv containing bispecific molecules were purified as described previously (Sci Rep. 2015 Dec. 11; 5:17943).

The amino acid sequence identifiers of an exemplary anti-Psl antibody, including heavy chain variable region (HCVR), heavy chain complementarity determining region 1 (HCDR1), heavy chain complementarity determining region 2 (HCDR2), heavy chain complementarity determining region 3 (HCDR3), light chain variable region (LCVR), light chain complementarity determining region 1 (LCDR1), light chain complementarity determining region 2 (HCDR2), light chain complementarity determining region 3 (HCDR3), heavy chain (HC), and light chain (LC), are shown in Table 11 below.

TABLE 11
Amino Acid Sequence Identifiers for exemplary anti-Psl antibody

| Antibody | | | | | | | | | | |
|---|---|---|---|---|---|---|---|---|---|---|
| Designation | HC | HCVR | HCDR1 | HCDR2 | HCDR3 | LC | LCVR | LCDR1 | LCDR2 | LCDR3 |
| REGN | 78 | residues | 31-35 of | 50-65 of | 98-109 of | 79 | residues | 23-33 of | 49-55 of | 88-99 of |
| 5409 | | 1-118 of | SEQ ID | SEQ ID | SEQ ID | | 1-108 of | SEQ ID | SEQ ID | SEQ ID |
| | | SEQ ID | NO: 78 | NO: 78 | NO: 78 | | SEQ ID | NO: 79 | NO: 79 | NO: 79 |
| | | NO: 78 | | | | | NO: 79 | | | |

### Example 7: Preparation of Anti-Protein A Antibodies

Anti-protein A antibodies were obtained by immunizing a VELOCIMMUNE® mouse (i.e., an engineered mouse comprising DNA encoding human Immunoglobulin heavy and kappa light chain variable regions, see U.S. Pat. No. 6,596,541) with an antigen derived from protein A of Staphylococcus aureus. An embodiment of the protein A antigen comprises amino acids as shown in Genbank Accession No. AGE10362. The antibody immune response was monitored by a protein A-specific immunoassay.

Several fully human anti-Protein A antibodies were isolated directly from antigen-positive B cells VELOCIMMUNE® mouse without fusion to myeloma cells, as described in U.S. Pat. No. 7,582,298. Once the antibodies were isolated, the nucleic acid and amino acid sequences were obtained. The amino acid sequence identifiers of an exemplary anti-protein A antibody mAb15144, including heavy chain variable region (HCVR), heavy chain complementarity determining region 1 (HCDR1), heavy chain complementarity determining region 2 (HCDR2), heavy chain complementarity determining region 3 (HCDR3), light chain variable region (LCVR), light chain complementarity determining region 1 (LCDR1), light chain complementarity determining region 2 (HCDR2), light chain complementarity determining region 3 (HCDR3), heavy chain (HC), and light chain (LC), are shown in Table 12 below. The nucleic acid sequence identifiers are shown in Table 13.

TABLE 12
Amino Acid Sequence Identifiers for Anti-Protein A Antibodies
SEQ ID NOs:

| Antibody | | | | | | | | | | |
|---|---|---|---|---|---|---|---|---|---|---|
| designation | HCVR | HCDR1 | HCDR2 | HCDR3 | LCVR | LCDR1 | LCDR2 | LCDR3 | HC | LC |
| mAb15144 | 58 | 60 | 62 | 64 | 10 | 12 | 14 | 16 | 90 | 85 |

TABLE 13
Nucleic Acid Sequence Identifiers for Anti-Protein A Antibodies
SEQ ID NOs:

| Antibody | | | | | | | | |
|---|---|---|---|---|---|---|---|---|
| designation | HCVR | HCDR1 | HCDR2 | HCDR3 | LCVR | LCDR1 | LCDR2 | LCDR3 |
| mAb15144 | 57 | 59 | 61 | 63 | 9 | 11 | 13 | 15 |

### Example 8: Preparation of Anti-IsdA Antibodies

Anti-IsdA antibodies were obtained by immunizing a VELOCIMMUNE® mouse (i.e., an engineered mouse comprising DNA encoding human Immunoglobulin heavy and kappa light chain variable regions, see U.S. Pat. No. 6,596,541) with an antigen derived from heme uptake protein IsdA from Staphylococcus aureus (IsdA). An embodiment of the antigen comprises amino acids as shown in Genbank Accession No. AFH 69349. The antibody immune response was monitored by an IsdA-specific immunoassay.

Several fully human anti-IsdA antibodies were isolated directly from antigen-positive B cells VELOCIMMUNE® mouse without fusion to myeloma cells, as described in U.S. Pat. No. 7,582,298. Once the antibodies were isolated, the nucleic acid and amino acid sequences were obtained. The amino acid sequence identifiers of an exemplary anti-IsdA antibody mAb20334, including heavy chain variable region (HCVR), heavy chain complementarity determining region 1 (HCDR1), heavy chain complementarity determining region 2 (HCDR2), heavy chain complementarity determining region 3 (HCDR3), light chain variable region (LCVR), light chain complementarity determining region 1 (LCDR1), light chain complementarity determining region 2 (HCDR2), light chain complementarity determining region 3 (HCDR3), heavy chain (HC), and light chain (LC), are shown in Table 14 below. The nucleic acid sequence identifiers are shown in Table 15.

TABLE 14
Amino Acid Sequence Identifiers for Anti-IsdA Antibodies
SEQ ID NOs:

| Antibody | | | | | | | | | | |
|---|---|---|---|---|---|---|---|---|---|---|
| designation | HCVR | HCDR1 | HCDR2 | HCDR3 | LCVR | LCDR1 | LCDR2 | LCDR3 | HC | LC |
| mAb20334 | 66 | 68 | 70 | 72 | 10 | 12 | 14 | 16 | 91 | 85 |

TABLE 15
Nucleic Acid Sequence Identifiers for Anti-IsdA Antibodies
SEQ ID NOs:

| Antibody | | | | | | | | |
|---|---|---|---|---|---|---|---|---|
| designation | HCVR | HCDR1 | HCDR2 | HCDR3 | LCVR | LCDR1 | LCDR2 | LCDR3 |
| mAb20334 | 65 | 67 | 69 | 71 | 9 | 11 | 13 | 15 |

### Example 9. Preparation of Anti-Poly-N-Acetylglucosamine (PNAG)

Recombinant C1q bispecific antibody REGN5536 was produced in CHO cells after transfection with two expression plasmids. One of the expression plasmids encodes the heavy chain (hIgG1) and light chain of an antibody that binds PNAG and the second expression plasmid encodes a C1q binding scFv-Fc (hIgG1), whose Fc contains the “Fc*” mutation. Specifically, the C1q binding scFv-Fc* was constructed as follows: (1-39 ULC VK (G4S) 3 Linker 17736PVH.hIgG1 (amino acids D104-K330; H318R, Y319F (UniProtKB/Swiss-Prot: P01857.1). The scFv containing bispecific molecules were purified as described previously (Sci Rep. 2015 Dec. 11; 5:17943).

The amino acid sequence identifiers of an exemplary anti-PNAG antibody, including heavy chain variable region (HCVR), heavy chain complementarity determining region 1 (HCDR1), heavy chain complementarity determining region 2 (HCDR2), heavy chain complementarity determining region 3 (HCDR3), light chain variable region (LCVR), light chain complementarity determining region 1 (LCDR1), light chain complementarity determining region 2 (HCDR2), light chain complementarity determining region 3 (HCDR3), heavy chain (HC), and light chain (LC), are shown in Table 16 below.

TABLE 16
Amino Acid Sequence Identifiers for exemplary anti-PNAG antibody

| Designation | | | | | | | | | | |
|---|---|---|---|---|---|---|---|---|---|---|
| Antibody | HC | HCVR | HCDR1 | HCDR2 | HCDR3 | LC | LCVR | LCDR1 | LCDR2 | LCDR3 |
| REGN5536 | 76 | residues | 26-33 of | 51-58 of | 96-106 of | 77 | residues | 27-32 of | 50-52 of | 93-101 of |
| | | 1-118 of | SEQ ID | SEQ ID | SEQ ID | | 1-108 of | SEQ ID | SEQ ID | SEQ ID |
| | | SEQ ID | NO: 76 | NO: 76 | NO: 76 | | SEQ ID | NO: 77 | NO: 77 | NO: 77 |
| | | NO: 76 | | | | | NO: 77 | | | |

### Example 10. Preparation of Anti-Clumping Factor a (ClfA)

Recombinant C1q bispecific antibody REGN5738 was produced in CHO cells after transfection with two expression plasmids. One of the expression plasmids encodes the heavy chain (hIgG1) and light chain of an antibody that binds ClfA and the second expression plasmid encodes a C1q binding scFv-Fc (hIgG1), whose Fc contains the “Fc*” mutation. Specifically, the C1q binding scFv-Fc* was constructed as follows: (1-39 ULC VK_(G4S) 3 Linker_17736PVH.hIgG1 (amino acids D104-K330; H318R, Y319F (UniProtKB/Swiss-Prot: P01857.1). The scFv containing bispecific molecules were purified as described previously (Sci Rep. 2015 Dec. 11; 5:17943).

Once the antibodies were isolated, the nucleic acid and amino acid sequences were obtained. The amino acid sequence identifiers of an exemplary anti-ClfA antibody, including heavy chain variable region (HCVR), heavy chain complementarity determining region 1 (HCDR1), heavy chain complementarity determining region 2 (HCDR2), heavy chain complementarity determining region 3 (HCDR3), light chain variable region (LCVR), light chain complementarity determining region 1 (LCDR1), light chain complementarity determining region 2 (HCDR2), light chain complementarity determining region 3 (HCDR3), heavy chain (HC), and light chain (LC), are shown in Table 17 below.

TABLE 17
Amino Acid Sequence Identifiers for exemplary anti-ClfA antibody

| Designation | | | | | | | | | | |
|---|---|---|---|---|---|---|---|---|---|---|
| Antibody | HC | HCVR | HCDR1 | HCDR2 | HCDR3 | LC | LCVR | LCDR1 | LCDR2 | LCDR3 |
| REGN5738 | 73 | residues | 26-33 of | 51-58 of | 96-106 of | 74 | residues | 27-32 of | 51-55 of | 89-98 of |
| | | 1-118 of | SEQ ID | SEQ ID | SEQ ID | | 1-108 of | SEQ ID | SEQ ID | SEQ ID |
| | | SEQ ID | NO: 73 | NO: 73 | NO: 73 | | SEQ ID | NO: 74 | NO: 74 | NO: 74 |
| | | NO: 73 | | | | | NO: 74 | | | |

### Example 11. Preparation of Bispecific Anti-C1q Bispecific Antibodies

Bispecific antibodies comprising an anti-C1q-specific binding domain and an target antigen-specific binding domain were constructed using standard methodologies wherein a heavy chain and light chain variable domain from an anti-C1q antibody were combined a target antigen-specific antigen binding domain comprising a heavy chain and light chain variable domain from an anti-IsdB antibody; a heavy chain and light chain variable domain from an anti-CD20 antibody; a heavy chain and light chain variable domain from an anti-GITR antibody; a heavy chain and light chain variable domain from an anti-Psl antibody; a heavy chain and light chain variable domain from an anti-protein A antibody; a heavy chain and light chain variable domain from an anti-IsdA antibody; a heavy chain and light chain variable domain from an anti-PNAG antibody; or a heavy chain and light chain variable domain from an anti-ClfA antibody. The antibodies used to construct the bispecific antibodies (bsAb) of this example were obtained as described in Examples 1-10. The sequence identification numbers for the bispecific antibodies described herein are presented in Table 18.

TABLE 18
Amino Acid Sequences of Bispecific Antibodies

| | SEQ ID NOs: | | | | | | | | | | | |
|---|---|---|---|---|---|---|---|---|---|---|---|---|
| | Anti-IsdB | | | | Anti-C1q | | | | ULC1-39 | | | |
| bsAb | HCVR | HCDR1 | HCDR2 | HCDR3 | HCVR | HCDR1 | HCDR2 | HCDR3 | LCVR | LCDR1 | LCDR2 | LCDR3 |
| REGN5066 | 26 | 28 | 30 | 32 | N | 4 | 6 | 8 | 10 | 12 | 14 | 16 |
| REGN5067 | 26 | 28 | 30 | 32 | 18 | 20 | 22 | 24 | 10 | 12 | 14 | 16 |
| | Anti-CD20 | | | | Anti-C1q | | | | ULC1-39 | | | |
| REGN5177 | 34 | 36 | 38 | 40 | 2 | 4 | 6 | 8 | 10 | 12 | 14 | 16 |

### Example 12. Human Serum Complement Dependent Killing of S. aureus

| | Anti-Influenza HA | | | | Anti-C1q | | | | ULC1-39 | | | |
|---|---|---|---|---|---|---|---|---|---|---|---|---|
| REGN6093 | 42 | 44 | 46 | 48 | 2 | 4 | 6 | 8 | 10 | 12 | 14 | 16 |
| | Anti-GITR | | | | Anti-C1q | | | | ULC1-39 | | | |
| REGN6545 | 50 | 52 | 54 | 56 | 2 | 4 | 6 | 8 | 10 | 12 | 14 | 16 |
| REGN6546 | 50 | 52 | 54 | 56 | 18 | 20 | 22 | 24 | 10 | 12 | 14 | 16 |
| | Anti-Protein A | | | | Anti-C1q | | | | ULC1-39 | | | |
| REGN5551 | 58 | 60 | 62 | 64 | 2 | 4 | 6 | 8 | 10 | 12 | 14 | 16 |
| | Anti-IsdA | | | | Anti-C1q | | | | UCL-39 | | | |
| REGN5553 | 66 | 68 | 70 | 72 | 2 | 4 | 6 | 8 | 10 | 12 | 14 | 16 |
| | Anti-Psl | | | | Anti-C1q | | | | Anti-Psl LC | | | |
| REGN5409 | Aa 1- | Aa 31- | Aa 50- | Aa 98- | 75 | 4 | 6 | 8 | Aa 1- | Aa 23- | Aa 49- | Aa 88- |
| | 118 of | 35 of | 65 of | 109 of | | | | | 108 of | 33 of | 55 of | 99 of |
| | SEQ ID | SEQ ID | SEQ ID | SEQ ID | | | | | SEQ ID | SEQ ID | SEQ ID | SEQ ID |
| | NO: 78 | NO: 78 | NO: 78 | NO: 78 | | | | | NO: 79 | NO: 79 | NO: 79 | NO: 79 |
| | Anti-PNAG | | | | Anti-C1q | | | | Anti-PNAG LC | | | |
| REGN5536 | Aa 1- | Aa 27- | Aa 51- | Aa 96- | 75 | 4 | 6 | 8 | Aa 1- | Aa 27- | Aa 50- | Aa 93- |
| | 118 of | 32 of | 58 of | 106 of | | | | | 108 of | 32 of | 52 of | 101 of |
| | SEQID | SEQ ID | SEQ ID | SEQ D | | | | | SEQ ID | SEQ ID | SEQ ID | SEQ ID |
| | NO: 76 | NO: 76 | NO: 76 | NO: 76 | | | | | NO: 77 | NO: 77 | NO: 77 | NO: 77 |
| | Anti-CflA | | | | Anti-C1q | | | | Anti-ClfA LC | | | |
| REGN5738 | Aa 1- | Aa 26- | Aa 51- | Aa 96- | 75 | 4 | 6 | 8 | Aa 1- | Aa 27- | Aa 51- | Aa 89- |
| | 118 of | 33 of | 58 of | 106 of | | | | | 108 of | 32 of | 55 of | 98 of |
| | SEQ ID | SEQ ID | SEQ ID | SEQ ID | | | | | SEQ ID | SEQ ID | SEQ ID | SEQ ID |
| | NO: 73 | NO: 73 | NO: 73 | NO: 73 | | | | | NO: 74 | NO: 74 | NO: 74 | NO: 74 |

### Materials and Methods

### Bacterial Strains and Growth Conditions.

S. aureus Newman wild-type (wt) and S. aureus Newman Δspa were grown in phenol-free RPMI (Gibco) at 37° C. unless otherwise noted.

Scanning Electron Microscopy (SEM) Visualization of S. aureus.

S. aureus Newman Δspa was grown in phenol-free RPMI medium (Gibco) at 37° C. overnight. The bacteria were washed two times with PBS and resuspended in phenol-free RPMI, 0.05% BSA to an OD₆₀₀˜1. 500 μl of bacteria was mixed with 500 μl of NHS (Bioreclamation) or 500 ul phenol-free RPMI, 0.05% BSA. The samples were incubated at 37° C. for 8 h with shaking. The bacteria were then washed three times with PBS. For EM analyses, samples were fixed 1:1 with a 2×fixative of 5% glutaraldehyde, 4% paraformaldehyde in 0.2M Sodium Cacodylate buffer, and dehydrated through a graded series of ethanol. Samples were critical point dried using liquid carbon dioxide in a Tousimis Samdri 795 Critical Point Drier (Rockville MD), coated with carbon in a Quorum EMS 150T ES (Quorum Technologies Ltd, United Kingdom) and examined in a Zeiss Supra Field Emission Scanning Electron Microscope (Carl Zeiss Microscopy, LLC North America), using an accelerating voltage of 8 KV.

### Determination of Complement Deposition by Fluorescent Microscopy.

S. aureus Newman Δspa was grown in phenol-free RPMI medium (Gibco) at 37° C. overnight. The bacteria were washed one time with PBS and resuspended in 3% bovine serum albumin (BSA) in PBS. They were blocked for 30 min at room temperature and then washed two times with PBS and resuspended in phenol-free RPMI, 0.05% BSA to an OD₆₀₀˜1. 500 μl of bacteria was mixed with 500 μl of 20% normal human serum (NHS, Bioreclamation), 500 μl 20% C6-depleted serum (Quidel), or 500 μl phenol-free RPMI, 0.05% BSA. The samples were incubated for 1 h at 37° C. with shaking. After 1 h, the bacteria were washed three times with PBS and resuspended in 1 ml of 1% BSA in PBS. Samples were aliquoted and then probed with goat anti-human C1q (Genway), mouse anti-human C3 (Biolegend), and mouse anti-human C5b9 (Abcam) as primary antibodies at a final concentration of 1 pg/ml. As controls non-specific mouse IgG1 and mouse IgG2a antibodies were used. Samples were incubated with primary antibodies for 1 h at room temperature and then washed three times with PBS. They were resuspended in 100 μl of 1% BSA in PBS and then probed with donkey anti-goat IgG conjugated to Alexa Fluor 488 (1:4000, ThermoFisher) or goat anti-mouse IgG conjugated to Alexa Fluor 488 (1:4000, ThermoFisher) secondary antibody. DAPI (4′,6-diamidino-2-phenylindole, dihydrochloride, Biolegend) was added to samples at 5 pg/ml and they were incubated for 45 min at room temperature. The samples were then visualized on a Zeiss LSM780 confocal microscope.

### Serum Killing Assay.

S. aureus Newman was grown in phenol-free RPMI medium (Gibco) at 37° C. overnight. The bacteria were washed one time with PBS and resuspended in RPMI, 0.05% bovine serum albumin to an OD₆₀₀˜0.5. They were then diluted to a final concentration of ˜1.0×10⁵ cfu/ml (a 1,000-fold dilution). Bacteria were subsequently mixed with an equal volume of normal human serum (Quidel) or human serum depleted of a terminal complement protein (Quidel) such that the final concentration of serum was 50%. The samples were incubated at 37° C. for 24 h with shaking. Bacterial survival was measured using BacTiter-Glo™ (Promega) according the manufacturer's instructions. Briefly BacTiter-Glo™ buffer was added to BacTiter-Glo™ substrate and then 100 μl of this reagent was added to 100 μl of bacterial sample in a 96-well plate. The plate was mixed for 30 s on an orbital plate shaker (Eppendorf) and then incubated for 5 min at room temperature. Luminescence was then measured on a SpectraMax i3×plate reader.

### Visualizing MAC Mediated Killing by Viability Dye Uptake.

S. aureus Newman Δspa was grown in phenol-free RPMI medium (Gibco) until it reached an OD₆₀₀˜1.0. The bacteria were washed one time with PBS and resuspended in 3% bovine serum albumin (BSA) in PBS. They were blocked for 30 min at room temperature and then washed two times with PBS and resuspended in phenol-free RPMI, 0.05% BSA to an OD₆₀₀˜1. 200 μl of bacteria was mixed with 200 μl of NHS (Quidel) or 200 μl C1-depleted serum (Quidel). Separate samples were prepared in triplicate for 0, 10 h, and 24 h analysis. Samples were incubated for the indicated time at 37° C. with shaking. After incubation propidium iodide (PI) was added to each sample to a final concentration of 1 μg/ml and incubated at room temperature for 5 min. Fluorescence was measured on a SpectraMax i3×plate reader (Excitation 533 nm, emission 617 nm). A separate aliquot of sample was analyzed using the BacTiter-Glo™ kit as described above.

### Results and Conclusion

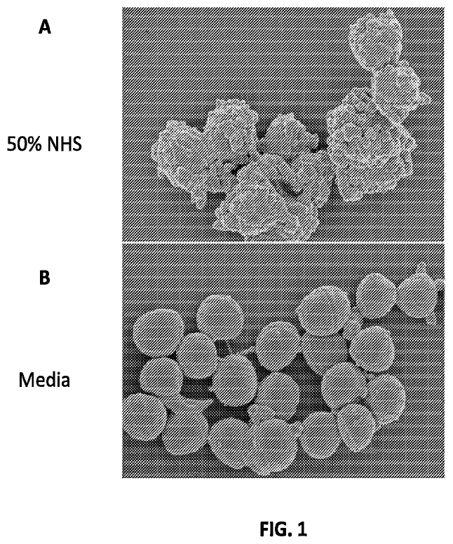

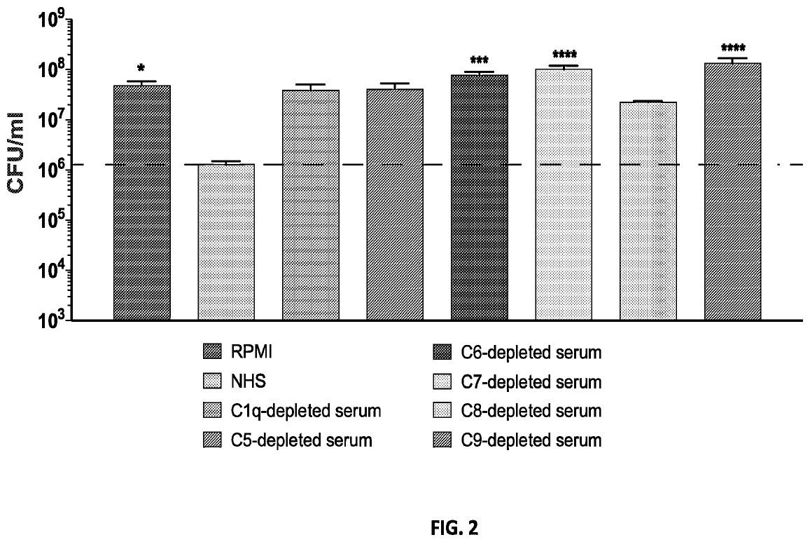

Previous work has shown complement proteins, including C5b9 complexes, deposit on the surface of Gram-positive organisms after short incubations (1-2 h) in 10% normal human serum (NHS), however no functional consequence of deposition was observed. To better understand how complement affects Gram-positive organisms, S. aureus incubated with either 50% NHS (FIG. 1a), which is closer to physiological levels than previously tested, or media (FIG. 1b) was visualized. Scanning electron microscopy (SEM) showed striking differences to the surface of S. aureus after 8 h of incubation with NHS (FIG. 1a). Complement deposition on S. aureus in the presence of NHS but not media was confirmed (data not shown).

Bacteria were incubated with 50% NHS, 50% C5-depleted serum, or media for 1 h at 37° C. Complement deposition was visualized using a primary antibody specific for C1q, C3, or a neo-epitope that forms upon C5b9 complex formation. In the presence of 50% NHS, surface-bound C1q, C3, and C5b9 proteins were observed (data not shown) with similar distribution. Depletion of C5 resulted in C1q and C3 deposition, but not C5b9, indicating the need for a complete terminal pathway for C5b9 formation.

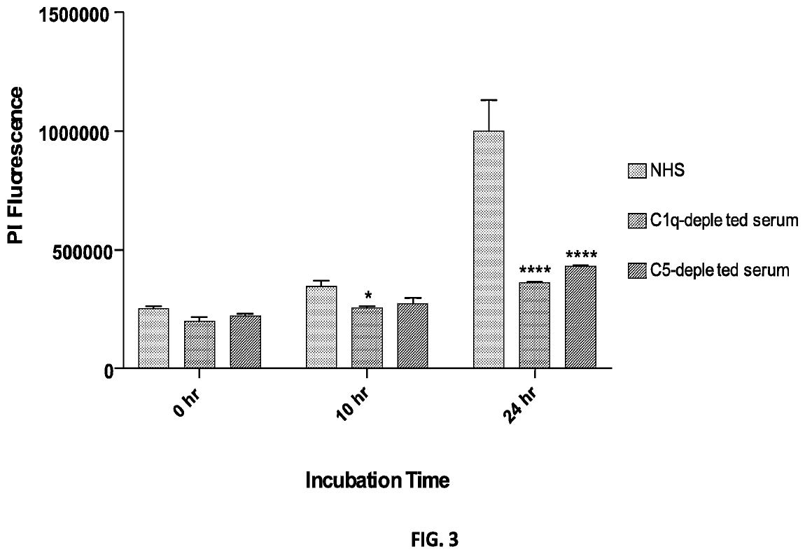

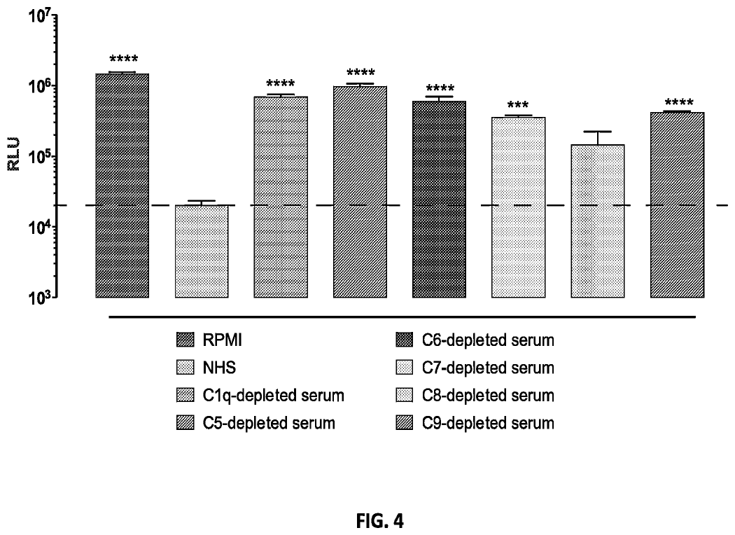

Incubation with NHS resulted in killing of S. aureus and is dependent on an intact terminal complement pathway (FIG. 2). For this experiment, bacteria were incubated for 24 h at 37° C. with a more physiological concentration of serum, 50% NHS, or 50% NHS depleted of individual terminal complement components (C1q, C5, C6, C7, C8, or C9). In 50% NHS viable bacteria were reduced 100-fold compared to media alone, as measured by serial dilution and plating (FIG. 2). Similar results were observed when ATP release was used to quantify viable bacteria (FIG. 4). When serum was depleted of C1q or any terminal complement component (C5, C6, C7, C8, C9; FIG. 2), growth was restored comparable to levels seen in media, confirming that S. aureus killing is dependent on formation of the MAC. Furthermore, incubation of S. aureus with NHS, but not serum depleted of C1q or C5, resulted in uptake of a viability dye, propidium iodide (PI) (FIG. 3). S. aureus was incubated with 50% NHS, 50% C1q-depleted serum, or 50% C5-depleted serum for 0, 10, or 24 h followed by addition of PI. While the level of PI staining was similar at 0 h, at 24 h there was a significant increase in the amount of PI taken up in the NHS treated samples indicating loss of viability which was not observed in complement depleted sera (FIG. 3). This indicates that complement activation can directly reduce the viability of S. aureus.

### Example 13. Bispecific Antibodies Anti-IsdB×Anti-C1q, Anti-Protein A×Anti-C1q, Anti-IsdA×Anti C1q, Anti-PNAG×Anti-C1q, and Anti-Clf A×Anti-C1q Bind to S. aureus and Facilitate Complement Deposition and Killing of S. aureus

This example shows that bispecific antibodies directing C1q to S. aureus enhanced complement deposition and resulted in cytotoxicity.

### Materials and Methods

### Bispecific Antibodies

Bispecific antibodies (bsAb) binding to C1q and S. aureus target antigens IsdB, IsdA, protein A, PNAG, or ClfA were prepared as described in Example 11.

### Surface Plasmon Resonance—Biacore

Equilibrium dissociation constants (K_{D} values) for binding of bispecific antibodies to target antigens were determined in various configurations. All Biacore binding studies were performed in a buffer composed of 0.01M HEPES pH 7.4, 0.15M NaCl, 3 mM EDTA, 0.05% v/v Surfactant P20 (running buffer). Binding parameters are shown in Table 19.

In one configuration, binding of human C1q (Quidel), and IsdB.6×(His) to respective anti-hC1q, and anti-IsdB-6×His arms of anti-C1q bispecific antibodies (bsC1q×IdsB) or bivalent anti-IsdB antibodies were determined using a real-time surface plasmon resonance biosensor (Biacore T200). The CM5 Biacore sensor surface was derivatized by amine coupling of anti-human IgG F (ab′)2 fragment specific antibody (Jackson, Cat nr. 109-005-006, Lot nr. 98960) or anti-Fc receptor antibody to capture purified anti-IsdB×anti-C1q bispecific antibody, and bivalent anti-IsdB. IsdB.6×(His) was injected across anti-C1q×anti-IsdB surface.

In another configuration, binding of anti-C1q×anti-IsdB to surface captured IsdB.6×(His) or biotinylated human C1q (Bt hC1q) was measured.

In another configuration, binding of anti-IsdA×anti-C1q (bsC1q×IsdA) to surface captured IsdA-6×His was measured.

In yet another configuration, binding of human C1q (Quidel) and anti-C1q×anti-IsdB to surface captured IsdB.6×(His) was determined using a real-time surface plasmon resonance biosensor (Biacore T200).

Antibody Dependent C1q Complement Deposition on S. aureus.

Antibodies were tested for complement deposition on S. aureus Newman in an ELISA based assay format. Briefly, S. aureus was grown in RPMI overnight, washed in PBS and adjusted to an OD₆₀₀ of 0.25. MaxiSorp 96 well microtiter plates (ThermoFisher) were coated overnight with 100 μl of S. aureus culture per well. Plates were fixed with 2% paraformaldehyde and blocked with 3% BSA prior to addition of a 1:3 titration of antibody ranging from 0.14 nM-33.3 nM with 1:3 dilutions of antibodies for 1 h at 25° C. After washing, 5% protein A/G adsorbed NHS (Bioreclamation) was added for 1.5 h at 37° C. Goat anti-C1q antibody (Genway Biotech, Inc.) was added to detect C1q deposition, followed by donkey anti-goat HRP secondary antibody (Jackson Immunoresearch). Plates were washed in a plate washer in between each step. SuperSignal Pico luminescence reagent (ThermoFisher) was then added to the wells and signal was detected in a plate reader (SpectraMax i3). Luminescence values were analyzed by a four-parameter logistic equation over a 7-point response curve (GraphPad Prism) to calculate the binding EC50 of the antibodies.

S. aureus Whole Blood Survival Assays.

Antibodies were assessed for bactericidal activity against S. aureus Newman in a whole blood survival assay. Briefly, S. aureus Newman was grown in phenol-free RPMI overnight, washed in PBS, and resuspended to a concentration of 1.25×10⁸ colony forming units (CFU)/ml in PBS. In triplicate, 10 μL of the S. aureus suspension was mixed with 68 nM or 680 nM test and control antibodies along with 100 μl of whole human blood (in sodium citrate as anti-coagulant with additional 500 nM dabigatran to prevent clot formation). Where indicated, the blood was preincubated with C5-blocking Fab and Cytochaslasin D for 10 min at room temperature. The samples were incubated in 96 well plates at 37° C. with shaking (100 rpm) for 24 h. After incubation, 100 μl of agglutination lysis buffer (PBS supplemented with 200 U Streptokinase, 2 μg/ml RNase, 10 μg/ml DNase, 0.5% saponin, 100 μg trypsin per ml of PBS) was added to the samples and they were vigorously vortexed until the pellet dissolved. 50 μl from each sample was serially diluted in PBS and plated onto LB agar plates for enumeration of CFUs.

Copy Number Determination of S. aureus Surface Antigens.

Standard curves for quantum MESF (molecules of equivalent soluble fluorochrome) beads (Bangs Laboratories, Inc.) were generated according to the manufacturer's procedure. Briefly one drop of the reference microspheres was added to 400 μl FACS buffer, 2% FBS (Gibco) in PBS. The microspheres were analyzed on a FACSCantII flow cytometer and values were used to generate a standard curve using the Bangs Laboratories, Inc. quantitative analysis template, QuickCal. The flow cytometer settings used for this analysis were used for the entire experiment. Next an antibody binding-bead standard curve was generated using Quantum™ Simply Cellular anti-IgG kit (Bangs Laboratories, Inc.). 1 drop of microspheres was added to 100 μl of buffer. Fluorochrome-conjugated antibodies specific for each surface antigen were serially diluted in buffer starting at a concentration of 450 nM. 50 μl of beads was mixed with 50 μl of each antibody dilution and incubated on ice for 30 min. 1 μl of ice cold buffer was then added to the samples, and they were washed twice with buffer. They were resuspended in 400 μl of fresh buffer and analyzed on a FACSCantoII using the same settings as utilized for the previous standard curve. To determine copy number of each surface antigen, S. aureus Newman Δspa was grown in phenol-free RPMI medium (Gibco) overnight.

The bacteria were washed twice with PBS and resuspended in 2% FBS (Gibco) in PBS. PI was added to each sample to a final concentration of 2.5 ug/ml and allowed to incubate with the bacteria for 2 min at room temperature. Bacterial viability was then determined by flow cytometry. 50 μl of bacteria was mixed with 50 μl of the serially diluted antibodies previously prepared. Samples were incubated on ice for 30 min and then 1 ml of ice cold buffer was added. The samples were washed twice and resuspended in 200 μl of buffer. They were analyzed on a FACSCantoII using the same settings as utilized previously. The MESF values for the saturating doses of the antibody were calculated from the MFI values, using the Bead Standard Curve generated above. The antibody-bead standard curve was used to determine the F/P ratio (fluorochrome/protein) by dividing MESF by the known antibody binding capacity of the beads. Copy number was then calculated using the following formula: MESF/FP Ratio=Copy number.

### Results and Conclusions

After demonstrating that complement can kill S. aureus, a strategy was developed to increase both complement deposition and bacterial killing. A C1q-targeting bsAb was tested to determine if it could recruit C1q, enhance complement deposition, and activate complement independent of antigen density and the geometry of antibody binding. Engagement of multiple C1q globular heads to target antigens is required for complement activation and MAC deposition. Furthermore, multiple MAC pores are required to efficiently kill target cells. To circumvent this, a fully human IgG1 bsAb was designed to recruit C1q directly to the S. aureus surface. The well-characterized, highly conserved, and abundant bacterial surface protein iron-regulated surface determinant protein B (IsdB) was selected as the target. VelocImmune™ mice were immunized with purified human C1q and recombinant S. aureus IsdB as described in Example 11 to generate fully human antibodies against these two antigens. IsdB binding arms were paired with multiple C1q effector arms and the bsAbs with maximal S. aureus killing activity in serum killing assays were selected.

The unique binding properties of the anti-IsdB×anti-C1q bsAb were characterized by surface plasmon resonance (SPR)-Biacore, as shown in Table 19.

TABLE 19
Binding parameters for bispecific antibodies
Biacore kinetic parameters for anti-Clq bispecific antibody
binding to IsdB, IsdA, and Clq at 37° C.

| Capture | | | | | |
|---|---|---|---|---|---|
| Surface | Test ligand | Ka (M⁻¹s⁻¹) | k_{d} (⁻¹) | K_{D} (M) | T_{1/2} (min) |
| bsC1q × IsdB | IsdB 6xHis | 1.63 × 10⁵ | 5.57 × 10⁻⁴ | 3.43 × 10⁻⁹ | 21 |
| anti-IsdB bival | IsdB 6xHis | 3.65 × 10⁵ | 6.4 × 10⁻⁴ | 1.75 × 10⁻⁹ | 18 |
| Bt hC1q | bsC1q × IsdB | 2.62 × 10⁴ | 9.64 × 10⁻² | 3.67 × 10⁻⁴ | .12 |
| IsdB.6x(His) | hC1q + bsC1q × IsdB | 1.03 × 10⁶ | 3.34 × 10⁻³ | 3.23 × 10⁻⁹ | 3.5 |
| IsdB.6x (His) | bsC1q × IsdB | 8.74 × 10⁵ | 6.57 × 10⁻³ | 7.52 × 10⁻⁹ | 1.8 |
| IsdA.6x (His) | bsC1q × IsdA | 2.7 × 10⁵ | 9.77 × 10⁻⁴ | 3.62 × 10⁻⁹ | 11.8 |

The bsC1q×IsdB and the parental bivalent anti-IsdB antibody were captured on the sensor chip surface and IsdB.6×(His) was injected over the surfaces at 37° C. Both bsC1q×IsdB and bivalent antibodies had nanomolar affinity for IsdB. When IsdB.6×(His) or IsdA.6×(His) were captured on the chip surface and bsC1q×IsdB and bsC1q×IsdA were injected over the surface of the chip, both bispecific antibodies had nanomolar affinity for their respective antigens. Human C1q was biotinylated and captured on the chip at low density, and the bsC1q×IsdB was injected, resulting in a Kd of 3.67 pM, reflecting the interaction in solution. To mimic the surface of S. aureus, IsdB.6×(His) was first captured on the chip surface, followed by injection of the bsC1q×IsdB, and finally C1q, and the dissociation of C1q was measured. In this format, the affinity to C1q increased to 3.23 nM. The 1000-fold tighter binding results from avidity-driven interactions between the anti-C1q arm and multiple C1q heads.

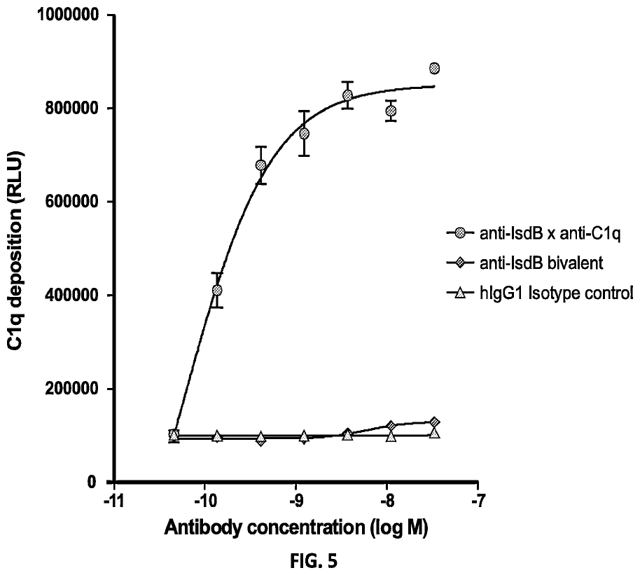

Anti-IsdB×anti-C1q also dramatically increased recruitment of C1q to S. aureus. C1q deposition was measured on the surface of S. aureus Newman Δspa (a methicillin-sensitive strain, MSSA) resulting from binding of anti-IsdB×anti-C1q bsAb, the bivalent IsdB antibody, or an isotype control antibody. While minimal C1q deposition was observed with the bivalent or isotype control antibody, incubation with anti-IsdB×anti-C1q bsAb resulted in robust, dose-dependent increase in C1q deposited on S. aureus (EC₅₀=127 pM; FIG. 5).

As shown in Table 20, multiple anti-C1q bispecific antibodies bound to S. aureus using ELISA. S. aureus Newman Δspa was coated onto ELISA plates; incubated with a titration of the anti-IsdB×anti-C1q bsAb, the bivalent IsdB antibody, anti-IsdA×anti-C1q bsAb, anti-PNAG×anti-C1q bsAb, anti-ClfA×anti-C1q bsAb, anti-protein A×anti-C1q bsAb, or an isotype control antibody; washed; and then binding measurements were made by luminescence (Table 20).

TABLE 20
ELISA binding to S. aureus Δspa

| Test Ligand | Max RLU | EC₅₀ (M) |
|---|---|---|
| bsC1q × IsdB | 7.93 × 10⁵ | 1.9 × 10⁻¹⁰ |
| bsC1q × IsdA | 1.33 × 10⁶ | 8.36 × 10⁻¹⁰ |
| bsC1q × PNAG | 1.62 × 10⁵ | 2.01 × 10⁻⁹ |
| bsC1q × ClfA | 3.04 × 10⁵ | 5.69 × 10⁻¹⁰ |
| bsC1q × Protein A | 1.91 × 10⁵ | 1.48 × 10⁻¹⁰ |

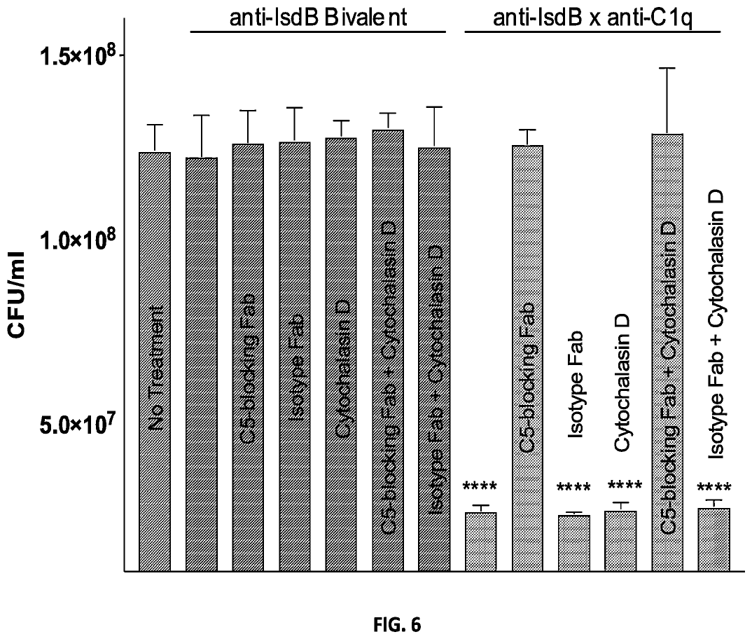

The functional consequence of bsAb-mediated enhancement of MAC deposition was shown using a whole blood assay, in which anti-IsdB×anti-C1q significantly inhibited S. aureus growth (FIG. 6). While the bivalent IsdB antibody incubated with S. aureus Newman in human blood for 24 h had no effect on bacterial growth, incubation with anti-IsdB×anti-C1q bsAb reduced bacterial growth nearly 10-fold (FIG. 6). This activity was dependent on complement activity: blocking phagocytosis with cytochalasin D had no effect on anti-IsdB×anti-C1q activity, whereas C5 blockade completely abrogated bsAb cytotoxicity (FIG. 6).

To assess the role of antigen density on the C1q-recruiting bsAb activity, the same C1q-targeting arm was paired with antibody arms targeting Protein A, clumping factor A (ClfA), poly-N-acetylglucosamine (PNAG), and IsdA. These S. aureus target antigens are present at different densities on the surface of S. aureus Newman Δspa (Table 21), as determined using flow cytometry

TABLE 21
Relative levels of S. aureus surface antigens

| Surface Antigen | Copy number related to Isotype Control |
|---|---|
| IsdB | 7.26 |
| ClfA | 0.90 |
| PNAG | 0.71 |
| IsdA | 13.10 |

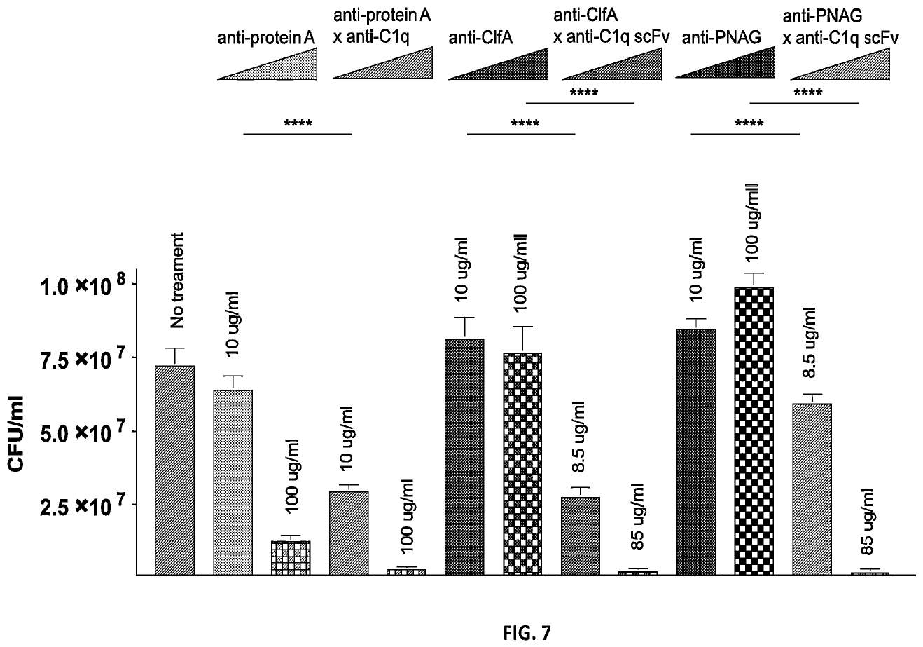

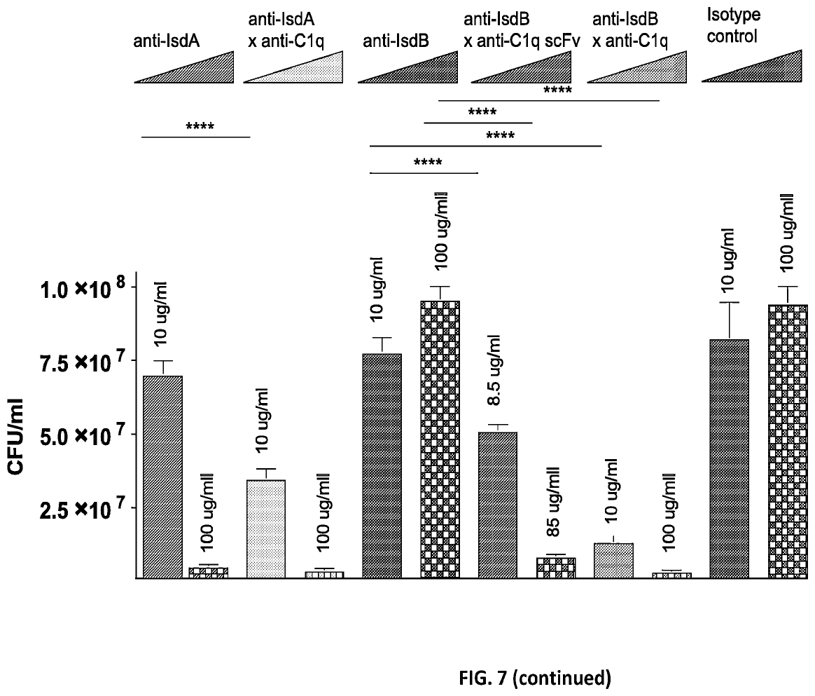

Each of the tested C1q-targeting bispecific antibodies bound C1q and S. aureus with high affinity (Table 22), and increased S. aureus killing compared to the corresponding bivalent antibody (FIG. 7 and Table 23).

TABLE 22
Binding parameters for 5 nM C1q binding as the test ligand to antibody
captured chip surfaces
Biacore kinetic parameters for binding at 37° C.

| | | | | T_{1/2} |
|---|---|---|---|---|
| Capture surface | Ka (M⁻¹s⁻¹) | k_{d} (⁻¹) | K_{D} (M) | (min) |
| bs hC1q × IsdB | 3.22 × 10⁶ | 5.02 × 10⁻³ | 1.56 × 10⁻⁹ | 2.3 |
| bs hC1q × IsdB scFv | 1.23 × 10⁷ | 9.2 × 10⁻³ | 7.48 × 10⁻¹⁰ | 1.3 |
| bs hC1q × IsdA | 3.62 × 10⁶ | 4.69 × 10⁻³ | 1.3 × 10⁻⁹ | 2.5 |
| bs hC1q × PNAG | 1.61 × 10⁷ | 1.02 × 10⁻² | 6.32 × 10⁻¹⁰ | 1.1 |
| bs hC1q × ClfA | 4.35 × 10⁶ | 9.2 × 10⁻³ | 7.4810⁻¹⁰ | 1.3 |
| bs hC1q × protein A | 3.06 × 10⁶ | 4.88 × 10⁻³ | 1.3610⁻⁹ | 2.3 |

As shown in Table 23, the overall growth in whole human blood in the absence of test antibody is normalized to 100%. Survival of S. aureus in both 68 nM and 680 nM negative isotype control antibodies ranged from 114-146%. S. aureus survival was reduced at least by 50% when treated with 68 nM anti-S. aureus×anti-C1q bispecific antibody compared to its equivalent bivalent anti-S. aureus antibody. When treated with 680 nM anti-S. aureus×anti-C1q bispecific antibody, survival ranged from 4-14%, which was also at least 50% lower than its equivalent bivalent anti-S. aureus antibody, except in the case of IsdA. For IsdA, in one experiment of two, the anti-IsdA×anti-C1q antibody activity was ˜40% more than the anti-IsdA bivalent antibody. Thus, with each anti-S. aureus×anti-C1q antibody, there was increased anti-Staphylococcal activity with increased antibody concentration.

The similar potency of these bsAbs, despite the variability in surface densities (Table 21) of the S. aureus target antigens, indicates that enhancing local concentrations of C1q on the cell surface overcomes the low efficiency of complement-mediated cytotoxicity. These results demonstrate that anti-Staphylococcus×anti-C1q antibodies enhance killing of S. aureus and supports a C1q-recruiting bsAb approach to augment complement activity.

TABLE 23
S. aureus Newman survival in whole human blood after treatment
with anti-S. aureus × anti-C1q bispecific antibodies

| | % survival of S. aureus compared to whole | | | |
|---|---|---|---|---|
| | blood ± standard deviation | | | |
| | (no treatment, normalized to 100%) | | | |
| | Expt 1 | Expt 2 | Expt 1 | Expt 2 |
| Antibody | 68 nM | 68 nM | 680 nM | 680 nM |
| Blood + S. aureus (no | 100 | 100 | 100 | 100 |
| treatment) | | | | |
| Anti-Protein A × | 25 ± 1 | 42 ± 2 | 7 ± 0.4 | 5 ± 0.2 |
| anti-C1q | | | | |
| Anti-ClfA × | 30 ± 1 | 39 ± 3 | 14 ± 1 | 4 ± 0.4 |
| anti-C1q | | | | |
| Anti-PNAG × | 26 ± 1 | 82 ± 3 | 13 ± 1 | 4 ± 1 |
| anti-C1q | | | | |
| Anti-IsdA × | 10 ± 1 | 48 ± 4 | 7 ± 1 | 5 ± 0.5 |
| anti-C1q | | | | |
| Anti-IsdB × | 26 ± 1 | 19 ± 2 | 11 ± 1 | 5 ± 0.2 |
| anti-C1q | | | | |
| Anti-IsdB × | 68 ± 6 | 71 ± 2 | 23 ± 1 | 12 ± 0.4 |
| anti-C1q_scFv | | | | |
| Anti-Protein A | 106 ± 9 | 89 ± 5 | 15 ± 2 | 18 ± 2 |
| Anti-ClfA | 105 ± 6 | 113 ± 9 | 119 ± 7 | 106 ± 11 |
| Anti-PNAG | 127 ± 14 | 117 ± 4 | 198 ± 11 | 137 ± 6 |
| Anti-IsdA | 47 ± 8 | 97 ± 6 | 29 ± 1 | 8 ± 0.2 |
| Anti-IsdB | 121 ± 14 | 107 ± 6 | 135 ± 12 | 132 ± 6 |
| Isotype control | 138 ± 11 | 114 ± 16 | 146 ± 26 | 130 ± 8 |
| (REGN1932) | | | | |

### Example 14. Anti-IsdB×Anti-C1q Improves Clearance of S. aureus in C1q-Humanized Mice

### Materials and Methods

### Quantification of C1q Concentration in Mouse Serum Using LC-MS/MS.

Protein from humanized mouse serum samples were denatured, reduced, and subsequently alkylated and digested with trypsin. The digested peptide mixture was then separated by reversed-phase liquid chromatography using an Agilent 1290 Infinity II LC Systems with an ACQUITY UPLC BEH130 C18 column (2.1×50 mm, 1.7 μm; Waters). 0.1% formic acid in water and 0.1% formic acid in acetonitrile were used as mobile phase A and mobile phase B, respectively. Signature peptides unique to human C1q protein sequences were selected from each of the individual C1q subunits (A, B, and C) as surrogate peptides for C1q quantification. Calibration curves for the surrogate peptide from each C1q subunit were generated using a series of concentrations of C1q protein standard (Quidel), which allows the quantitation of total C1q by any of the three subunits based on the assay response of the respective surrogate peptides. Data acquisition by LC-MS/MS under selected reaction monitoring (SRM) mode was performed using an Agilent 6495 TripleQuad mass spectrometer with Agilent Jet Stream electrospray ionization source. Pre-selected mass to charge ratio (m/z) of precursor and product ion pair of each surrogate peptide were fragmented with optimized collision energy and detected in the mass spectrometer. Agilent LC/MS Data Acquisition for 6400 Series Triple Quadrupole, version B.08.00, was used to run the LC/MS system. Agilent MassHunter Quantitative Analysis, version B.06.00, was used for data analysis. In this assay all standards, controls, and test samples were prepared in parallel. Disseminated S. aureus infection model

Humanized C1q (C1q^{Hu/Hu}) mice in a mixed background (MAID1615) were randomized by weight on day 0 and then infected intraperitoneally with 200 μl volume of S. aureus Newman (1.5×10⁸ CFUs/mouse), S. aureus CA-127 (1.5×10⁸ CFU/ml), or S. aureus MW2 (1.3×10⁸ CFUs/mouse). The S. aureus strains were grown overnight in TSB. The culture was then diluted 1:100 in fresh TSB, grown to log phase (OD₆₀₀˜1) in TSB at 37° C., washed 3 times in PBS and adjusted to the desired density for infection. Where indicated antibiotics were administered starting 18 h following infection. Mice were treated with 110 mg/kg of vancomycin twice daily administered subcutaneously, 50 mg/kg daptomycin once daily administered subcutaneously, or 80 mg/kg linezolid twice daily administered orally (100 μl volume) on Days 1, 2 and 3. 24 h post infection, mice were administered with a single dose of the indicated antibody or PBS in a 100 μl volume, intraperitoneally. Mice were weighed daily until day 4 post infection and change in weight was recorded. Mice were euthanized on day 4 and kidneys, heart, liver, lungs and spleen were collected to determine organ burden. Briefly, the organs were homogenized in 5 mL PBS using program “Multi-C” on a gentleMACS Octo Dissociator (Miltenyi Biotec). Homogenate was then diluted in PBS and 10-fold serial dilutions were plated on LB agar plates. Plates were incubated at 37° C. overnight. Resultant colonies were counted, and results were reported as CFUs/gram tissue.

### Results and Conclusions

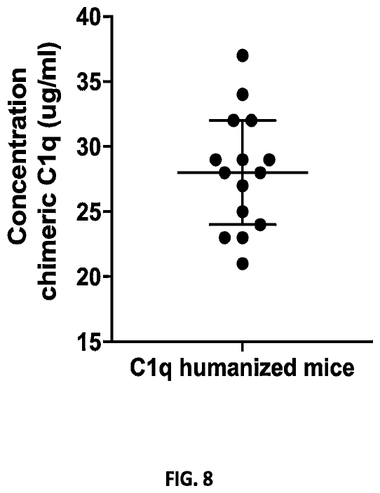

After showing in vitro efficacy of anti-IsdB×anti-C1q bsAb, it was evaluated whether enhanced cytotoxicity could be observed in vivo. Because the anti-C1q arm of the bsAb is specific for human C1q heads, and does not bind mouse C1q, mice were generated that encode the human sequences of the globular head of C1q to enable in vivo efficacy models. Mouse C1qA, C1qB, and C1qC genes were replaced with chimeric versions, in which the N-terminus of the gene encoding the collagen-like tail was mouse sequence, and the C-terminus of the gene encoding the globular head was human. This resulted in a C1q protein that could bind the bsAb while still interacting with the rest of the mouse complement pathway. The genes remained under the control of the native mouse C1q promoter and expression of the chimeric proteins in serum was confirmed by mass spectrometry. On average the mice expressed 28 μg/mL of chimeric C1q with values ranging from 21 to 37 μg/mL (FIG. 8).

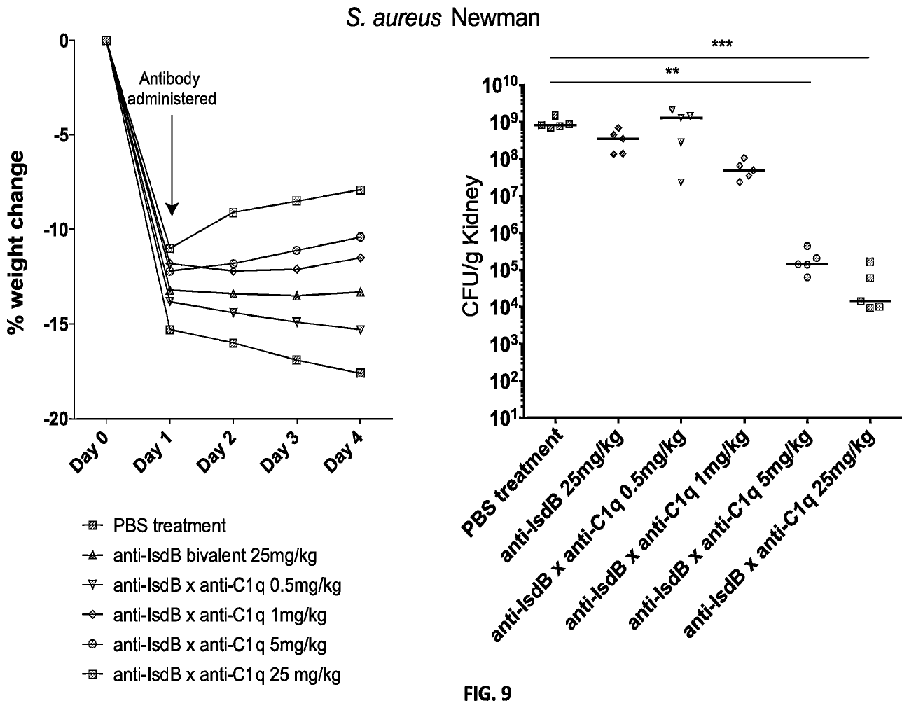

Using the C1q humanized mice, it was demonstrated that a single dose of anti-IsdB×anti-C1q bsAb significantly improved clearance of S. aureus in vivo. Prior to infection mice were randomized to have similar average group body weight, then infected intraperitoneally with S. aureus Newman one day later. The day after infection, mice were intraperitoneally dosed with PBS, the bivalent anti-IsdB antibody (25 mg/kg), or varying concentrations of anti-IsdB×anti-C1q (0.5-25 mg/kg). Mice were weighed daily and on Day 4 organs were collected and bacterial burdens determined. One day after infection, the mice in each group lost 10-15% of their body weight. In PBS treated animals, weight loss continued after day 1 (FIG. 9, left panel, red squares), whereas weights of 25 mg/kg anti-IsdB treated mice stabilized after day 1 (FIG. 9, left panel, plum triangles). Remarkably, treatment with as little as 1 mg/kg anti-IsdB×anti-C1q stabilized weight after Day 1, and doses of 5 and 25 mg/kg resulted in weight gains suggesting rapid reduction in bacterial load (FIG. 9, left panel, orange diamonds, circles, and squares).

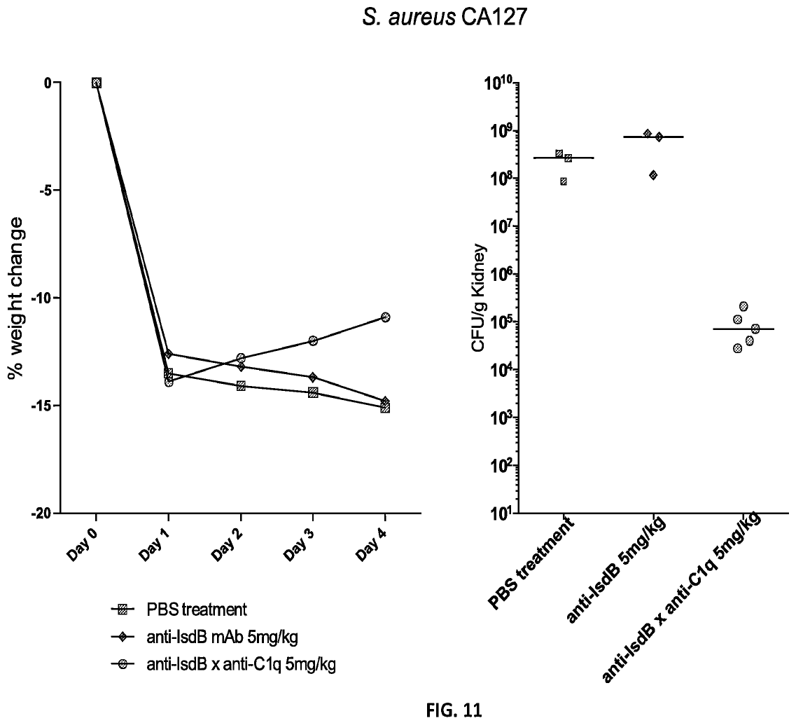

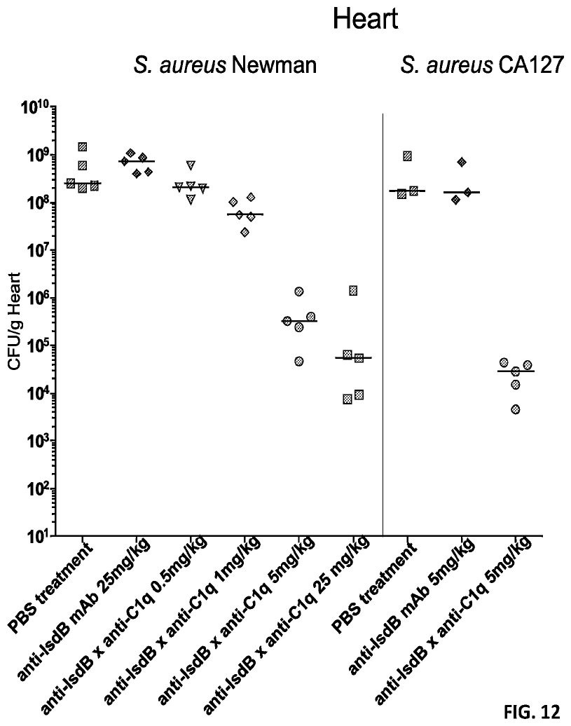

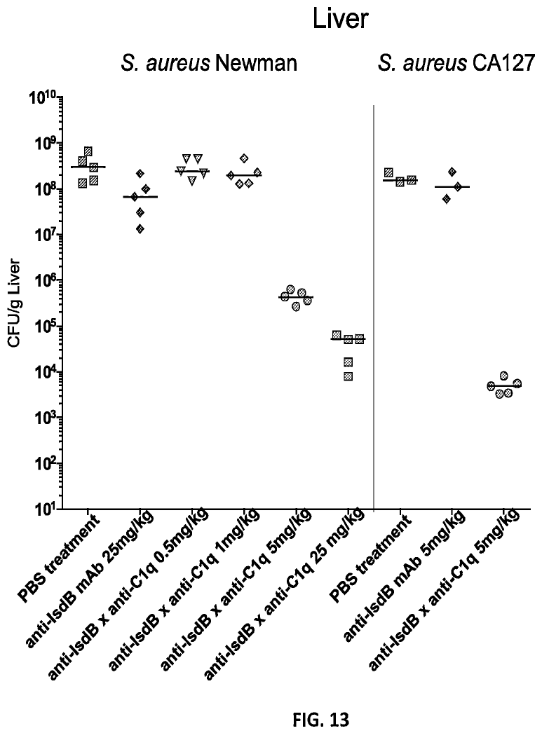

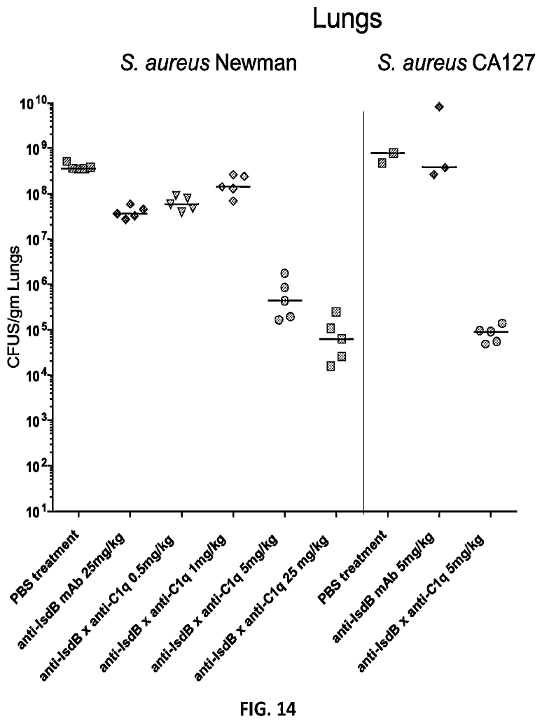

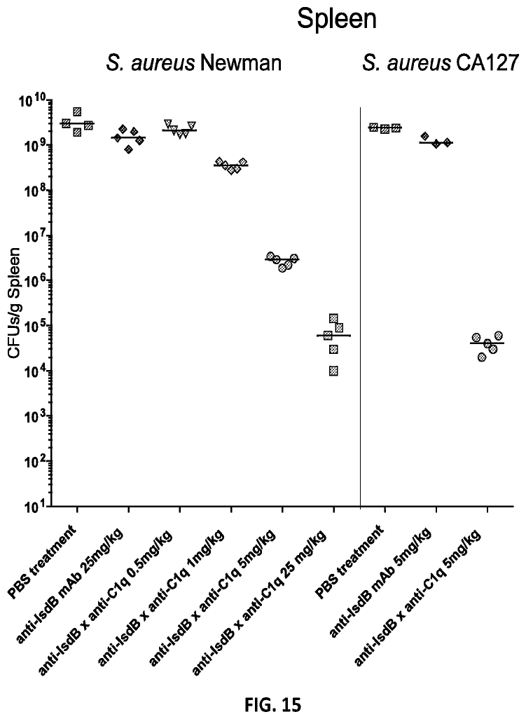

At day 4, bacterial burden in the organs was quantified: mice treated with PBS and 25 mg/kg of anti-IsdB bivalent antibody had 108 bacteria per gram in their kidneys (FIG. 9, right panel). Conversely, treatment with as little as 1 mg/kg anti-IsdB×anti-C1q reduced kidney bacterial burden by at least 1-log, with a 4-log reduction observed at the 5 mg/kg dose (FIG. 9, right panel). Furthermore, similar trends in body weight and bacterial organ burdens were observed with a methicillin resistant (MRSA) strain, S. aureus CA127 (FIG. 11). Comparable reductions in bacterial burdens were observed in the hearts, liver, lungs and spleen (FIG. 12-15, respectively). These results demonstrate that a bivalent anti-IsdB IgG1 antibody is not sufficient to clear S. aureus in vivo, while the anti-IsdB×anti-C1q bsAb enhances C1q-dependent complement recruitment and killing.

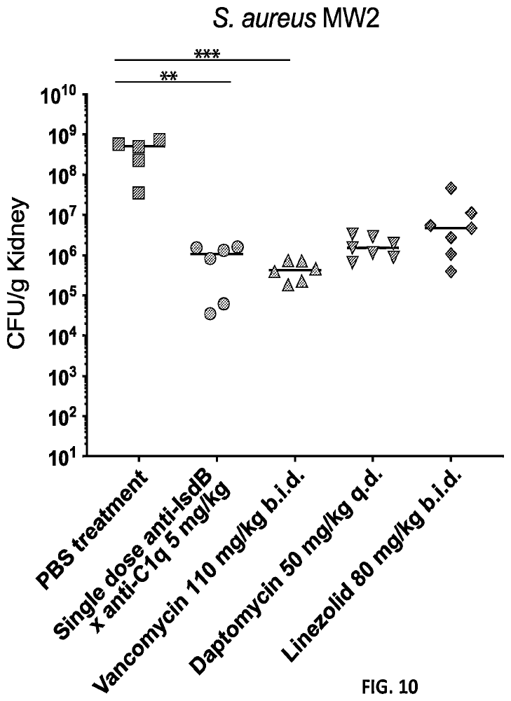

A single dose of anti-IsdB×anti-C1q is as effective at reducing S. aureus burden as daily dosing of the standard of care antibiotics vancomycin, linezolid, and daptomycin. Mice were infected intraperitoneally with S. aureus MW2, a MRSA strain. One day after infection mice were intraperitoneally administered either PBS, 5 mg/kg anti-IsdB×anti-C1q bsAb, or standard of care antibiotics: either 110 mg/kg vancomycin twice daily (b.i.d.) administered subcutaneously, 50 mg/kg daptomycin once daily (q.d.) administered subcutaneously, or 80 mg/kg linezolid twice daily (b.i.d.) administered orally. Mice were weighed daily for 4 days, kidneys were collected, and bacterial burdens determined. While all antibiotics resulted in lower bacterial burdens compared to untreated mice (daptomycin ˜330-fold reduction; linezolid ˜100-fold reduction), mice treated with either anti-IsdB×anti-C1q bsAb or vancomycin had significantly lower burden of S. aureus in their kidneys, with greater than 500-fold reduction (FIG. 10). A single dose of the bsAb was as effective in clearing bacteria as daily doses of the antibiotics tested.

To harness the potent killing activity of the complement system for therapeutic potential, we have developed a bispecific antibody platform that recruits C1q to lyse pathogenic cells. To demonstrate the utility of this platform we initially chose S. aureus, an organism thought to be resistant to the cytotoxic effect of MAC. Using the C1q-directed bispecific approach, we enhanced complement deposition and killing in vitro and in vivo. Depletion of C5, a terminal complement component, abrogated the effect, demonstrating a dependence on MAC-mediated lysis. We further showed that this approach works using other S. aureus targets with different densities.

Gram-positive bacteria are susceptible to direct complement-mediated lysis. MAC was not thought to play a role in control of Gram-positive infections in humans since direct killing had not been observed and both terminal complement deficiencies and blockade of C5 with eculizumab result only in increased susceptibility to the Gram-negative bacteria Neisseria. However, S. aureus do possess terminal complement inhibitors such as SSL7 indicating a role for blocking MAC activity and humans with deficiencies in early complement components (e.g. C1q, C2, C3, C4) are susceptible to multiple bacterial infections including S. aureus. Therefore, the efficacy of our bsAb suggests that increasing localized complement can be harnessed to kill Gram-positive bacteria through the activity of MAC.

### Example 15. Kinetics of Human C1q, IsdB-6×his, Influenza HA Foldon-6×his and hGITR.Mmh Binding to Anti-IsdB×Anti-C1q, Anti-Influenza HA×Anti-C1q, Anti-GITR×Anti-C1q, Anti-Psl×Anti-C1q scFv and Anti-CD20×Anti-C1q Bispecific mAbs at 25° C. and 37° C.

Having demonstrated efficacy of a C1q-recruiting bsAb against S. aureus, we investigated whether C1q-mediated activity could be used to effectively lyse Gram-negative bacteria and malignant mammalian cells with vastly different cell surfaces. The C1q-recruiting effector arm was paired with surface antigen targeting arms on diverse cell types: Psl on Gram-negative Pseudomonas aeruginosa, CD20 on B-cells, glucocorticoid-induced TNFR-related protein (GITR) on T-cells, and hemagglutinin (HA) on influenza-infected cells. The bsAbs were prepared as described in Example 11 and were tested for binding kinetics.

### Materials and Methods

Equilibrium dissociation constants (K_{D} values) for human C1q (Quidel), IsdB-6×His (SEQ ID NO: 80), HA Foldon-6×His (SEQ ID NO: 81) and hGITR.mmh (SEQ ID NO: 82) binding to respective anti-hC1q, anti-IsdB-6×His, anti-HA Foldon-6×.His and anti-GITR.mmh arms of anti-C1q bispecific antibodies were determined using a real-time surface plasmon resonance biosensor (Biacore T200). The CM5 Biacore sensor surface was derivatized by amine coupling of anti-human IgG F(ab′)2 fragment specific antibody (Jackson, Cat nr. 109-005-006, Lot nr. 98960) to capture purified anti-IsdB×anti-C1q, anti-Influenza HA×anti-C1q, anti-GITR×anti-C1q, anti-Psl×anti-C1q scFv and anti-CD20×anti-C1q bispecific antibodies. All Biacore binding studies were performed in a buffer composed of 0.01M HEPES pH 7.4, 0.15M NaCl, 3 mM EDTA, 0.05% v/v Surfactant P20 (running buffer).

Human C1q in concentrations ranging from 0.625 nM to 5 nM, recombinant bacterial IsdB expressed with a hexahistidine tag (IsdB-6×His; SEQ ID NO: 80) and hGITR expressed with a myc-myc-His tag(hGITR-mmh; SEQ ID NO:82) in concentrations ranging from 1.11 nM-90 nM, and Influenza A group I HA foldon expressed with a hexahistidine tag (HA-6×His; SEQ ID NO: 81) in concentrations ranging from 50 nM to 0.78 nM were injected over the relevant anti-antigen (Ag)×C1q bispecific antibody-captured surface at a flow rate of 50 μL/minute. Antibody-reagent association was monitored for 2 minutes, while dissociation was monitored for 5 minutes. All kinetic binding experiments were performed at 25° C. or 37° C. Kinetic association (kₐ) and dissociation (k_{d}) rate constants were determined by fitting the real-time sensorgrams to a 1:1 binding model using Scrubber 2.0c curve fitting software. Binding dissociation equilibrium constants (K_{D}) and dissociative half-lives (t½) were calculated from the kinetic rate constants as:

$${K}_{D} ( M ) = \frac{k d}{k a} , and t 1 / 2 ( min ) = \frac{ln ( 2 )}{60 * k d}$$

### Results and Conclusions

As shown in Table 24, at 25° C., all of the 7 anti-C1q antibodies of the disclosure bound to human C1q (Quidel) with K_{D} values ranging from 451 pM to 842 pM. As shown in Table 25, at 37° C., all of the 7 anti-C1q bispecific antibodies of the disclosure tested bound to hC1q with K_{D} values ranging from 481 pM-4.63 nM.

As shown in Table 26, at 25° C., the 2 anti-IsdB×anti-C1q antibodies of the disclosure tested (REGN5066 and REGN5067) bound to IsdB.6×His with K_{D} values of 2.36 nM and 1.7 nM, respectively. At 37° C., the 2 anti-IsdBxC1q antibodies of the disclosure bound to IsdB-6×His with K_{D} values of 3.43 nM and 3.33 nM.

As shown in Table 27, at 25° C., the 2 anti-GITR×anti-C1q antibodies of the disclosure tested (REGN6545 and REGN6546) bound to hGITR.6×His with K_{D} values of 9.86 nM and 7.52 nM, respectively. At 37° C., the 2 anti-GITR×anti-C1q antibodies of the disclosure tested bound to hGITR.6×His with K_{D} values of 49.2 nM and 20.9 nM.

As shown in Table 28, at 25° C., binding of the anti-HA×anti-C1q antibody of the disclosure (REGN6093) to Influenza HA Foldon.6×His was inconclusive. At 37° C., binding of the anti-HA×anti-C1q antibody of the disclosure to Influenza A group 1 HA Foldon.6×His was not observed under the conditions tested.

Binding of human C1q, IsdB-6×His, HA.6×His and hGITR.mmh to isotype control antibody (REGN1932) was not observed under any of the experimental conditions described above.

TABLE 24
Biacore kinetics of hC1q binding to anti-HA × anti-C1q (REGN6093), anti-GITR ×
anti-C1q (REGN6545, REGN6546), anti-IsdB × anti-C1q (REGN5066, REGN5067), anti-
Psl × anti-C1q scFv (REGN5409) and anti-CD20 × anti-C1q (REGN5177) bispecifc mAbs at 25° C.

| hC1q binding at 25° C. | | | | | | | | |
|---|---|---|---|---|---|---|---|---|
| REGN #/ | | Pairing | | | kₐ | k_{d} | K_{D} | t_{1/2} |
| Ab PID | C1q Clone | Clone | Ab type | Ag pair | (l/mol*s) | (1/s) | (mol/l) | (min) |
| REGN6093 | mAb17736 | mAb11829- | Influenza | Influenza | 4.80E+06 | 2.16E03− | 4.51E−10 | 5.3 |
| | | | HA × | HA | | | | |
| | | | C1q | | | | | |
| REGN6545 | mAb17736P | mAb14536 | anti- | GITR | 3.07E+06 | 1.62E−03 | 5.28E−10 | 7.1 |
| | | | GITR × | | | | | |
| | | | C1q | | | | | |
| REGN5177 | mAb17736 | mAb14303 | anti- | CD20 | 3.26E+06 | 2.24E−03 | 6.88E−10 | 5.2 |
| | | | CD20 × | | | | | |
| | | | anti-C1q | | | | | |
| | | | mAb | | | | | |
| REGN5409 | 17736 ScFv | COMP2882 | anti- | Psl | 3.51E+06 | 2.62E−03 | 7.47E−10 | 4.4 |
| | (VK-VH) | (anti-Psl) | Psl × C1q | | | | | |
| | | | ScFv | | | | | |
| REGN6546 | mAb18395 | mAb14536 | anti- | GITR | 5.43E+06 | 4.09E−03 | 7.53E−10 | 2.8 |
| | | | GITR × | | | | | |
| | | | C1q | | | | | |
| REGN5066 | mAb17736 | mAb20295 | IsdB × | IsdB | 2.51E+06 | 2.11E−03 | 8.42E−10 | 5.5 |
| | | | C1q | | | | | |
| REGN5067 | mAb18395 | mAb20295 | IsdB × | IsdB | 5.78E+06 | 4.35E−03 | 7.54E−10 | 2.7 |
| | | | C1q | | | | | |
| REGN1932 | | | Anti- | isotype | NB | NB | NB | NB |
| | | | FelD1 | control | | | | |

TABLE 25
Biacore kinetics of hC1q binding to anti-HA × anti-C1q (REGN6093), anti-GITR ×
anti-C1q (REGN6545, REGN6546), anti-IsdB × anti-C1q (REGN5066, REGN5067), anti-
Psl × anti-C1q scFv (REGN5409) and anti-CD20 × anti-C1q (REGN5177) bispecifc mAbs at 37° C.

| hC1q binding at 37° C | | | | | | | | |
|---|---|---|---|---|---|---|---|---|
| REGN #/ | | Pairing | | | kₐ | k_{d} | K_{D} | t_{1/2} |
| Ab PID | C1q Clone | Clone | Ab type | Ag pair | (l/mol*s) | (1/s) | (mol/l) | (min) |
| REGN6093 | mAb17736 | mAb11829 | Influenza | Influenza | 3.61E+06 | 4.01E−03 | 1.11E−09 | 2.9 |
| | | | HA × | HA | | | | |
| | | | C1q | | | | | |
| REGN5066 | mAb17736 | mAb20295 | IsdB × | IsdB | 3.22E+06 | 5.02E−03 | 1.56E−09 | 2.3 |
| | | | C1q | | | | | |
| REGN6545 | mAb17736 | mAb14536 | anti- | GITR | 3.34E+06 | 5.82E−03 | 1.74E−09 | 2 |
| | | | GITR × | | | | | |
| | | | C1q | | | | | |
| REGN5067 | mAb18395 | mAb20295 | IsdB × | IsdB | 6.52E+06 | 1.34E−02 | 2.05E−09 | 0.9 |
| | | | C1q | | | | | |
| REGN5409 | 17736 | COMP2882 | anti- | Psl | 4.80E+06 | 1.16E−02 | 2.41E−09 | 1 |
| | ScFv | (anti-Psl) | Psl × C1q | | | | | |
| | (VK-VH) | | ScFv | | | | | |
| REGN5177 | mAb17736 | mAb14303 | anti- | CD20 | 1.04E+07 | 4.99E−03 | 4.81E−10 | 2.3 |
| | | | CD20 × | | | | | |
| | | | anti-C1q | | | | | |
| | | | mAb | | | | | |
| REGN6546 | mAb18395 | mAb14536 | anti- | GITR | 4.90E+06 | 2.27E−02 | 4.63E−09 | 0.5 |
| | | | GITR × | | | | | |
| | | | C1q | | | | | |
| REGN1932 | | | Anti- | isotype | NB | NB | NB | NB |
| | | | FelD1 | control | | | | |

TABLE 26
Biacore kinetics of IsdB-6 × His binding to IsdB
arm of anti-Isd × C1q bispecific antibodies

| Antibody | Ag | Temp. | kₐ | k_{d} | K_{D} | t_{1/2} |
|---|---|---|---|---|---|---|
| ID | Pair | ° C. | (Ms⁻¹) | (s⁻¹) | (Molar) | (min) |
| REGN5066 | IsdB | 25 | 1.15E+05 | 2.70E−04 | 2.36E−09 | 42.8 |
| REGN5067 | IsdB | 25 | 1.20E+05 | 2.04E−04 | 1.70E−09 | 56.6 |
| REGN1932 | FelD1 | 25 | NB | NB | NB | NB |
| REGN5066 | IsdB | 37 | 1.63E+05 | 5.57E−04 | 3.43E−09 | 20.7 |
| REGN5067 | IsdB | 37 | 1.56E+05 | 5.20E−04 | 3.33E−09 | 22.2 |
| REGN1932 | FelD1 | 37 | NB | NB | NB | NB |

NB—No binding observed under test conditions described above

TABLE 27
Biacore kinetics of hGITR binding to GITR arm
of anti-GITR × anti-C1q bispecific antibodies

| Antibody | Ag | Temp. | kₐ | k_{d} | K_{D} | t_{1/2} |
|---|---|---|---|---|---|---|
| ID | Pair | ° C. | (Ms⁻¹) | (s⁻¹) | (Molar) | (min) |
| REGN6545 | GITR | 25 | 3.30E+05 | 3.26E−03 | 9.86E−09 | 3.5 |
| REGN6546 | GITR | 25 | 4.53E+05 | 3.40E−03 | 7.52E−09 | 3.4 |
| REGN1932 | FelD1 | 25 | NB | NB | NB | NB |
| REGN6545 | GITR | 37 | 2.74E+05 | 1.35E−02 | 4.92E−08 | 0.9 |
| REGN6546 | GITR | 37 | 5.43E+05 | 1.14E−02 | 2.09E−08 | 1 |
| REGN1932 | FelD1 | 37 | NB | NB | NB | NB |

TABLE 28
Biacore kinetics of Influenza A group 1 HA Foldon.6 × His binding
to HA arm of anti-HA × anti-C1q bispecific antibodies

| Antibody | Ag | TEMP | kₐ | k_{d} | K_{D} | t_{1/2} |
|---|---|---|---|---|---|---|
| ID | Pair | ° C. | (Ms⁻¹) | (s⁻¹) | (Molar) | (min) |
| REGN6093 | HA | 25 | IC | IC | IC | IC |
| REGN1932 | FelD1 | 25 | NB | NB | NB | NB |
| REGN6093 | HA | 37 | NB | NB | NB | NB |
| REGN1932 | FelD1 | 25 | NB | NB | NB | NB |

NB—No binding observed under test conditions described above;
IC—Binding was inconclusive

### Example 16. Kinetics of Human C1q (Quidel) and Paired Ag-6×his or Mmh Binding to Anti-IsdB×Anti-C1q, Anti-Influenza HA×Anti-C1q (1), Anti-GITR×Anti-C1q (2), Anti-CD20×Anti-C1q and Anti-Psi×Anti-C1q Bispecific mAbs at 25° C. and 37° C.

Equilibrium dissociation constants (K_{D} values) for the C1q arm of purified anti-IsdB×anti-C1q (2), anti-Influenza HA×anti-C1q (1), anti-GITR×anti-C1q (2), anti-CD20×anti-C1q (1), and anti-Psl×anti-C1q (1) bispecific antibodies binding to neutravidin captured biotinylated (bt) human C1q (purchased from Quidel biotinylated in-house) were determined using a real-time surface plasmon resonance biosensor (Biacore 8k). K_{D} values were also obtained for the respective paired arm of purified anti-IsdB×anti-C1q, anti-Influenza HA×anti-C1q and anti-GITR×anti-C1q bispecific antibodies binding to anti-His antibody (GE Healthcare, Lot 10266715) captured IsdB-6×His, Influenza A group HA Foldon-6×His and hGITR-mmh using a real-time surface plasmon resonance biosensor (Biacore 8k).

The CM5 Biacore sensor surface was derivatized by amine coupling with Neutravidin (Thermo Fisher catalog nr 31000) to bind biotinylated hC1q (bt-hC1q). All Biacore binding studies were performed in a buffer composed of 0.01M HEPES pH 7.4, 0.15M NaCl, 3 mM EDTA, 0.05% v/v Surfactant P20 (running buffer). Bispecific mAbs were prepared in concentrations ranging from 7.8 nM to 500 nM for bt-hC1q binding and 12.5 nM and 50 nM concentrations for His-captured paired Ag binding. Solutions were injected over the bispecific antibody-captured surface at a flow rate of 30 μL/minute. Antibody-reagent association was monitored for 2.5 minutes while dissociation was monitored for 8-10 minutes. All binding kinetic experiments were performed at 25° C. or 37° C. Kinetic association (kₐ) and dissociation (k_{d}) rate constants were determined by fitting the real-time sensorgrams to a 1:1 binding model using GE Healthcare Insight curve fitting software. Binding dissociation equilibrium constants (K_{D}) and dissociative half-lives (t %₂) were calculated from the kinetic rate constants as:

$${K}_{D} ( M ) = \frac{k d}{k a} , and t 1 / 2 ( min ) = \frac{ln ( 2 )}{60 * k d}$$

### Results and Conclusions

As shown in Table 29, at 25° C., all of the 7 anti-C1q bispecific antibodies of the disclosure bound to Neutravidin captured biotinylated human C1q (Quidel) with K_{D} values ranging from 327 nM-2.18 uM. As shown in Table 30 at 37° C., 6 of the anti-C1q antibodies of the disclosure tested bound to human bt-C1q with K_{D} values ranging from 343 nM to 8.54 uM. As shown in Table 31, at 25° C., the IsdB arm of the two anti-IsdB×anti-C1q bispecific antibodies of the disclosure bound to IsdB-6×His with K_{D} values of 2.3 nM and 3.01 nM, the HA arm of the one anti-HA×anti-C1q bispecific antibody of the disclosure bound to HA Foldon-6×His with a K_{D} value of 1.11 nM and the hGITR arm of the two anti-GITR×anti-C1q bispecific antibodies of the disclosure bound to hGITR.mmh with K_{D} values of 3.3 nM and 4.61 nM. As shown in Table 32, at 37° C., the IsdB arm of the anti-IsdB×anti-C1q bispecific antibody of the disclosure tested bound to IsdB-6×His with a K_{D} value of 7.52 nM, the HA arm of the one anti-HA×anti-C1q bispecific antibody of the disclosure bound to HA Foldon.6×His with a K_{D} value of 519 pM and the hGITR arm of the two anti-GITR×anti-C1q bispecific antibodies of the disclosure tested had K_{D} values of 7.38 nM and 16 nM. Binding of isotype control antibody (REGN1932) to human bt-C1q, IsdB-6×His, HA Foldon.6×His and hGITR.mmh was not observed under the experimental conditions described above.

TABLE 29
Bispecific Abs binding to Biotinylated hC1q at 25° C.

| REGN nr/ | | Pairing | | | kₐ | k_{d} | K_{D} | t_{1/2} |
|---|---|---|---|---|---|---|---|---|
| Ab PID | C1q Clone | Clone | Ab type | Ag pair | (l/mol*s) | (1/s) | (mol/l) | (min) |
| REGN5066 | mab17736 | mAb20295 | IsdB × | IsdB | 9.11E+04 | 3.66E−02 | 4.02E−07 | 0.32 |
| | | | C1q | | | | | |
| REGN5067 | mAb18395 | mAb20295 | IsdB × | IsdB | 1.68E+05 | 3.65E−01 | 2.18E−06 | 0.03 |
| | | | C1q | | | | | |
| REGN6093 | mAb17736 | MAb11829 | Influenza | Influenza | 9.46E+04 | 3.09E−02 | 3.27E−07 | 0.37 |
| | | | HA × | HA | | | | |
| | | | C1q | | | | | |
| REGN6545 | mAb17736 | mAb14536P | anti- | GITR | 2.41E+05 | 4.49E−01 | 1.86E−06 | 0.03 |
| | | | GITR × | | | | | |
| | | | C1q | | | | | |
| REGN6546 | H1xH18395P | H1H14536P2 | anti- | GITR | 2.20E+05 | 4.13E−01 | 1.88E−06 | 0.03 |
| | | | GITR × | | | | | |
| | | | C1q | | | | | |
| REGN5409 | 17736P ScFv | COMP2882 | anti- | Psl | 6.40E+04 | 2.76E−02 | 4.31E−07 | 0.42 |
| | (VK-VH) | (anti-Psl) | Psl × C1q | | | | | |
| | | | ScFv | | | | | |
| REGN5177 | mAb17736 | mAb28698 | anti- | CD20 | 1.01E+05 | 4.42E−02 | 4.36E−07 | 0.3 |
| | | | CD20 × | | | | | |
| | | | anti-C1q | | | | | |
| REGN1932 | | | isotype | Feld1 | NB | NB | NB | NB |
| | | | control | | | | | |

TABLE 30
Biacore kinetics for anti-IsdB × anti-C1q (REGN5066 and REGN5067),
anti-Influenza HA × anti-C1q (REGN6093), anti-GITR × anti-C1q
(REGN6545/REGN6546) and anti- × anti-C1q (REGN5409) bispecific mAbs binding to bt-hC1q

| REGN nr/ | | Pairing | | | kₐ | k_{d} | K_{D} | t_{1/2} |
|---|---|---|---|---|---|---|---|---|
| Ab PID | C1q Clone | Clone | Ab type | Ag pair | (l/mol*s) | (1/s) | (mol/l) | (min) |
| REGN5066 | mAb17736 | mAb20295 | IsdB × | IsdB | 1.17E+05 | 7.82E−02 | 6.66E−07 | 0.15 |
| | | | C1q | | | | | |
| REGN5067 | mAb18395 | mAb20295 | IsdB × | IsdB | 2.24E+05 | 8.44E−01 | 3.77E−06 | 0.01 |
| | | | C1q | | | | | |
| REGN6093 | mAb17736 | mAb11829 | Influenza | Influenza | 1.82E+05 | 9.37E−02 | 5.16E−07 | 0.12 |
| | | | HA × | HA | | | | |
| | | | C1q | | | | | |
| REGN6545 | mAb17736 | mAb14536 | anti- | GITR | 1.98E+05 | 6.58E−01 | 3.33E−06 | 0.02 |
| | | | GITR × | | | | | |
| | | | C1q | | | | | |
| REGN6546 | mAb18395 | mAb14536 | anti- | GITR | 8.05E+04 | 6.87E−01 | 8.54E−06 | 0.02 |
| | | | GITR × | | | | | |
| | | | C1q | | | | | |
| REGN5409 | mAb17736 | COMP2882 | anti- | Psl | 4.35E+04 | 1.49E−02 | 3.43E−07 | 0.78 |
| | ScFv | (anti-Psl) | Psl × C1q | | | | | |
| | (VK-VH) | | ScFv | | | | | |
| REGN1932 | | | isotype | Feld1 | NB | NB | NB | NB |
| | | | control | | | | | |

TABLE 31
Bispecific mAbs binding to IsdB.6 × His, HA foldon.6 × His and hGITR.mmh at 25° C.

| REGN | | Pairing | | | kₐ | k_{d} | K_{D} | t_{1/2} |
|---|---|---|---|---|---|---|---|---|
| nr/Ab PID | C1q Clone | Clone | Ab type | Ag pair | (l/mol*s) | (1/s) | (mol/l) | (min) |
| REGN5066 | mAb17736 | mAb20295 | IsdB × | IsdB | 4.57E+05 | 1.05E−03 | 2.30E−09 | 11.0 |
| | | | C1q | | | | | |
| REGN5067 | mAb118395 | mAb20295 | IsdB × | IsdB | 3.28E+05 | 9.89E−04 | 3.01E−09 | 11.7 |
| | | | C1q | | | | | |
| REGN6093 | mAb17736 | mAb11829 | Influenza | Influenza | 1.58E+05 | 1.75E−04 | 1.11E−09 | 66.0 |
| | | | HA × | HA | | | | |
| | | | C1q | | | | | |
| REGN6545 | mAb17736 | mAb14536 | anti- | GITR | 6.93E+05 | 2.29E−03 | 3.30E−09 | 5.04 |
| | | | GITR × | | | | | |
| | | | C1q | | | | | |
| REGN6546 | mAb18395 | mA14536 | anti- | GITR | 6.73E+05 | 3.10E−03 | 4.61E−09 | 3.73 |
| | | | GITR × | | | | | |
| | | | C1q | | | | | |
| REGN1932 | | | MAB | Feld1 | NB | NB | NB | NB |
| | | | isotype | | | | | |
| | | | control | | | | | |

TABLE 32
Bispecific mAbs binding to IsdB.6 × His, HA foldon.6 × His and hGITR.mmh at 37° C.

| REGN nr/ | | Pairing | | | Ka | Kd | KD | t½ |
|---|---|---|---|---|---|---|---|---|
| Ab PID | C1q Clone | Clone | Ab type | Ag pair | (l/mol*s) | (1/s) | (mol/l) | (min) |
| REGN5066 | mAb17736 | mAb20295 | IsdB × | IsdB | 8.74E+05 | 6.57E−03 | 7.52E−09 | 1.8 |
| | | | C1q | | | | | |
| REGN6093 | mAb17736 | mAb11829 | Influenza | Influenza | 9.94E+04 | 5.15E−05 | 5.19E−10 | 224.3 |
| | | | HA × | HA | | | | |
| | | | C1q | | | | | |
| REGN6545 | mAb17736 | mAb14536 | anti- | GITR | 1.08E+06 | 7.95E−03 | 7.38E−09 | 1.5 |
| | | | GITR × | | | | | |
| | | | C1q | | | | | |
| REGN6546 | mAb18395 | mAb14536 | anti- | GITR | 1.19E+06 | 1.90E−02 | 1.60E−08 | 0.6 |
| | | | GITR × | | | | | |
| | | | C1q | | | | | |
| REGN1932 | | | isotype | Feld1 | NB | NE | NB | NB |
| | | | control | | | | | |

### Example 17. Determination of Membrane Complement Inhibitor Levels by Flow Cytometry

Flow cytometric analysis was used to characterize the CD55 and CD59 expression levels on Jurkat T-cells and Raji B-cells. Briefly, cells were incubated at 4° C. for 60 min with BD APC anti-CD55, BD FITC anti-CD59, or isotype matched control antibodies according to the manufacturer's instructions. After incubation, the cells were washed with cold PBS containing 2% FBS, re-suspended in PBS, and analyzed by flow cytometry on an Accuri C6 flow cytometer. Unstained controls were also tested for both cell lines. The results were analyzed using FlowJo (TreeStar Inc.) software and geometric mean fluorescence for viable cells were determined. As shown in Table 33, Raji and Jurkat cells express different levels of the complement inhibitors CD55 and CD59.

TABLE 33
Relative levels of Complement Inhibitors on tested cell types

| Cell type | CD55 fold change | CD59 fold change |
|---|---|---|
| Raji | 7.7 | 6.2 |
| Jurkat | 23.4 | 53.1 |

### Example 18. Demonstrating the Ability of Anti-Psl×Anti-C1q Bispecific Antibodies to Bind Pseudomonas aeruginosa Via ELISA

Pseudomonas aeruginosa, an opportunistic human pathogen, is a significant source of disease in hospitals and among immunocompromised individuals due to antibiotic resistance and biofilm formation mediated by its surface exopolysaccharides. We investigated whether the C1q-recruiting bsAb platform was effective against this pathogen. Binding of the anti-Psl×anti-C1q bispecific antibodies of the present disclosure to Psl expressed on the surface of Pseudomonas aeruginosa was determined in an ELISA based format. Most Pseudomonas aeruginosa clinical isolates (˜85%) express the exopolysaccharide Psl protein and a bivalent monoclonal antibody (mAb) to this exopolysaccharide has shown efficacy in mouse models of infection (DiGiandomenico A., et al. J. Exp. Med. 209, 1273-1287 (2012)). Because the exopolysaccharide coats the surface of the bacterium, it can be used as a targeting arm for bispecific antibodies. A bispecific antibody using anti-Psl as one arm and anti-C1q as the second arm of the antibody has been generated. Comparison of the binding of the bivalent parent anti-Psl mAb versus the anti-Psl×C1q bispecific antibody to P. aeruginosa was tested.

### Experimental Procedure

An anti-Psl×anti-C1q bispecific antibody (REGN 5409) of this disclosure was assessed for binding to wild-type P. aeruginosa strain PAO1 (American Type Culture Collection) compared to its anti-Psl bivalent parent monoclonal antibody (mAb) (REGN2882). An overnight P. aeruginosa culture was grown in LB (Teknova), diluted 1:50 in fresh LB and grown to OD₆₀₀=˜1 at 37° C. with shaking. The culture was washed once with PBS (Gibco) and diluted to OD₆₀₀=0.25. MicroSorp 96-well plates (ThermoFisher) were coated with 100 ul per well of the P. aeruginosa suspension and incubated overnight at 4° C. The following morning, plates were washed once with wash buffer (Imidazole buffered saline with Tween-20) and blocked for two hours at 25° C. with 200 ul of blocking buffer (3% BSA in PBS). After blocking, plates were washed once, and titrations of the bispecific, bivalent and isotype-matched control antibodies (ranging from 333 nM-5.64 pM with 1:3 serial dilutions in 0.5% BSA/0.05% Tween-20/PBS) were added to the bacteria-containing wells and incubated for one hour at 25° C. Wells were washed three times and then incubated with 100 ng/ml anti-human HRP secondary antibody (Donkey anti-human IgG HRP from Jackson ImmunoResearch) per well for one hour at 25° C. Then 100 μl of SuperSignal ELISA Pico Chemiluminescent Substrate was added to each well and signal was detected using a SpectraMax i3×plate reader (Molecular Devices). Luminescence values were analyzed by a four-parameter logistic equation over an 11-point response curve (GraphPad Prism).

A bispecific antibody using anti-Psl as one arm and anti-C1q as the second arm of the antibody was generated as described in Example 11 above and assessed for its ability to deposit complement component C1q onto the surface of wild-type P. aeruginosa strain PAO1 (American Type Culture Collection) compared to its anti-Psl bivalent parent monoclonal antibody (mAb) (see REGN2882).

An overnight P. aeruginosa culture was grown in TSB (Tryptic soy broth from Teknova), diluted 1:50 in fresh TSB and grown to OD₆₀₀=˜1 at 37° C. with shaking. The culture was washed once with PBS and diluted to OD₆₀₀=0.25. MicroSorp 96-well plates (ThermoFisher) were coated with 100 ul per well of the P. aeruginosa suspension and incubated overnight at 4° C. The following morning, plates were washed three times with wash buffer (Imidazole buffered saline with Tween-20) and blocked for two hours at 25° C. with 200 ul of blocking buffer (3% BSA in PBS). Following blocking, plates were washed six times and titrations of the bispecific (REGN5409), bivalent (REGN2882), and isotype-matched control antibodies (ranging from 333 nM-5.64 pM with 1:3 serial dilutions in 0.5% BSA/10 mM HEPES/RPMI) were added to the bacteria-containing wells and incubated for one hour at 25° C. Wells were washed six times and then incubated for 1.5 hours at 37° C. with 5% complement preserved human serum (diluted in 0.5% BSA/10 mM HEPES/RPMI) that had been adsorbed with Protein A/G agarose to remove any anti-P. aeruginosa antibodies present in the serum. Wells were washed six times and C1q deposition was detected by adding 1 ug/ml goat anti-human C1q antibody (Genway) followed by 100 ng/ml anti-goat HRP secondary antibody (Jackson ImmunoResearch) (in 0.5% BSA/0.05% Tween-20/PBS) per well for one hour at 25° C. Then 100 μl of SuperSignal ELISA Pico Chemiluminescent Substrate was added to each well and signal was detected using a SpectraMax i3×plate reader (Molecular Devices). Luminescence values were analyzed by a four-parameter logistic equation over a 10-point response curve (GraphPad Prism).

Anti-Psl×anti-C1q bispecific (REGN5409) and the anti-Psl bivalent (REGN2882) hIgG1 antibodies were assessed for bactericidal activity against P. aeruginosa H103 in a whole blood survival assay. A culture of P. aeruginosa was grown in TSB overnight, washed in PBS, and resuspended to a concentration of 1×10⁵ colony forming units (CFU)/mL in PBS. In duplicates, 10³ CFU P. aeruginosa suspension in 10 ul was mixed with 80, 800 and 4000 uM antibodies in 10 ul volume and was added to 100 uL of whole human blood (in sodium citrate as anti-coagulant with additional 500 nM dabigatran to reduce clot formation). The samples were incubated in 96 well plates at 37° C. with shaking (100 rpm) for twenty-four hours. After incubation, 100 ul of agglutination lysis buffer (PBS supplemented with 200 U Streptokinase, 2 ug/mL RNase, 10 ug/mL DNase, 0.5% saponin, 100 ug trypsin per ml of PBS) was added to the samples and vigorously vortexed until the pellet disappeared. A total of 50 uL from each sample was serially diluted in PBS and plated onto LB agar plates for enumeration of CFUs.

### Results and Conclusion

As shown in Table 34, bivalent anti-Psl mAb (REGN2882) demonstrated higher binding (EC₅₀ binding of 2.699E-10) to P. aeruginosa PAO1 than the anti-Psl×anti-C1q bispecific antibody (REGN5409) that contains only one Psl-binding arm (EC₅₀ binding of 4.326E-09). Notably, however, the anti-Psl×anti-C1q bispecific antibody (REGN5409) demonstrated the ability to bind P. aeruginosa. The isotype-matched control antibodies did not bind to the bacteria. Table 35 shows the EC₅₀ binding concentration for a bivalent anti-Psl mAb (REGN2882) and anti-Psl×anti-C1q bispecific antibody (REGN5409) along with two isotope-matched control antibodies.

TABLE 34
Antibody binding to P. aeruginosa PAO1

| Antibody | Maximum binding RLUs | EC₅₀ Binding (M) |
|---|---|---|
| Bs anti-Psl × anti-C1q | 3.9 × 10⁵ | 4.326E−09 |
| Bivalent anti-Psl | 4.77 × 10⁵ | 3.45E−10 |
| Control I | | no binding |
| Control II | | no binding |

As shown in Table 35, increased levels of C1q deposition was detected on the surface of P. aeruginosa PAO1 in the presence of the anti-Psl×anti-C1q bispecific antibody (REGN5409) compared to the bivalent anti-Psl mAb (REGN2882). Isotyped-matched control antibodies did not bind to the bacteria. Table 36 shows the ability of an anti-Psl×anti-C1q bispecific antibody (REGN5409) to increase the component C1q deposition (EC₅₀ binding=3.167E-09) on the surface of P. aeruginosa PAO1 compared to bivalent anti-Psl mAb (REGN2882; EC₅₀ binding=2.529E-08).

TABLE 35
Complement component C1q deposition on surface of live P. aeruginosa
PAO1 using anti-Psl and anti-Psl × anti-C1q antibodies

| Antibody | EC₅₀ Binding (M) |
|---|---|
| REGN5409 | 3.167E−09 |
| REGN2882 | 2.529E−08 |
| Control I | no binding |
| Control II | no binding |

P. aeruginosa survival in human whole blood can be assessed in an ex vivo assay to explore the role of complement and immune effector cells to modulate bacterial growth similar to that described with S. aureus (J Exp Med. 2009 Oct. 26; 206(11):2417-27). The activity of an anti-Psl×anti-C1q bispecific antibodies in modulating P. aeruginosa survival is measured in this assay where the bispecific antibodies or bivalent parental antibodies are added into whole blood and survival is assessed after twenty-four hours.

Results are presented as percent survival of P. aeruginosa after treatment with the anti-Psl×anti-C1q bispecific (REGN5409) or the anti-Psl bivalent (REGN2882) hIgG1 antibodies as summarized in Table 36. The overall growth in whole human blood in the absence of test antibody is normalized to 100%.

Table 36 shows the ability of anti-Psl×anti-C1q bispecific antibody (REGN5409) to effectively prevent P. aeruginosa survival in human whole blood at three different concentrations (80 uM, 800 uM, and 4000 uM) compared to no treatment and treatment with an anti-Psl bivalent antibody (REGN2882). Survival of P. aeruginosa when treated with the anti-Psl bivalent antibody was unaffected with low antibody concentrations of 80 uM. At higher anti-Psl bivalent antibody concentrations of 800 and 4000 uM, increase in growth of P. aeruginosa was observed, ranging from 116-1560%. In contrast, the treatment with anti-Psl×anti-C1q bispecific antibody reduced growth by 96-99%. Addition of anti-Psl×anti-C1q bsAb to P. aeruginosa in human blood resulted in over a 1-log reduction in bacteria after 24 h compared to control or anti-Psl bivalent antibody. This result demonstrated that the C1q-recruiting bsAb platform is effective against both Gram-positive and Gram-negative bacteria and further showed that adding a C1q-recruiting arm to an antibody that lacks intrinsic CDC activity can result in a molecule that leads to significant complement activation.

TABLE 36
P. aeruginosa H103 survival in human whole blood after
treatment with anti-Psl × anti-C1q bispecific antibody

| | 80 uM | 800 uM | 4000 uM |
|---|---|---|---|
| | % survival of P. aeruginosa | | |
| | compared to whole blood (no treatment, | | |
| Antibody | normalized to 100%) | | |
| Blood + P. aeruginosa (no | 100 | 100 | 100 |
| treatment) | | | |
| REGN2882 (anti-Psl bivalent) | 99 | 116 | 1560 |
| REGN5409 (anti-Psl × anti- | 4.1 | 3.24 | 1.1 |
| C1q) | | | |

### Example 19: Demonstrating the Ability of Anti-Influenza HA×Anti-C1q Bispecific Antibodies to Bind Influenza HA Via ELISA and Mediate CDC of MDCK Infected Cells

Influenza infection is initiated by the surface glycoprotein hemagglutinin (HA) through binding to sialic acid residues at the surface of the host cell and fusion of viral and endosomal membranes. Current vaccine strategies rely on the production of influenza HA-specific antibodies to protect against viral infection (Y Q Chen, et al., Cell. 173, 417-429 (2018)). However, influenza infections can also lead to the production of antibodies that bind to infected cells and induce the lysis of infected cells through activation of complement (M. Terajima et al., Journal of Virology. 85, 13463-13467 (2011); J P. Jayasekera et al., Journal of Virology. 81, 3487-3494 (2007); J P M. Melis et al., Molecular Immunology. 67, 117-130 (2015).

Bispecific antibodies represent an emerging strategy to direct the complement system against cells infected with an infectious agent in a targeted manner. IgG antibodies have been shown to activate the classical pathway of the complement system through binding to C1q via their Fc region (M. Terajima et al., Journal of Virology. 85, 13463-13467 (2011); J P. Jayasekera et al., Journal of Virology. 81, 3487-3494 (2007); J P M. Melis et al., Molecular Immunology. 67, 117-130 (2015). Targeting the C1q component of complement in conjunction with the HA protein on the surface of influenza-infected cells and maybe even virions could further enhance the protective effect of antibodies specific for HA.

In this example, broadly neutralizing anti-HA×anti-C1q bispecific antibodies were tested for their binding to influenza A (group 1) infected cells.

### Experimental Procedure

Madin-Darby Canine Kidney (MDCK) cells were infected with Influenza A virus A/Puerto Rico/08/1934 (H1N1) at a Multiplicity Of Infection (MOI) of 0.05 for 24 hours. Infected cells were washed and fixed with 4% paraformaldehyde. Fixed cells were then blocked with a 3% Bovine Serum Albumin (BSA) solution before adding broadly-neutralizing anti-Influenza A group 1 HA×anti-C1q bispecific antibody (REGN6093), parental anti-HA specific bivalent antibody (mAb11829), parental anti-HA specific antibody with only a single arm (mAb27250), and anti-Influenza A group 1 HA×anti-FcγRIII bispecific antibody (mAb20067) or control antibodies, which consisted of an anti-IsdB×anti-C1q bispecific antibody (REGN5066), negative control #2 (a bispecific antibody comprising an anti-C1q arm and an arm targeting a target antigen other than HA), an anti-IsdB×anti C1q ScFv bispecific antibody (REGN5408), negative control #3 (a bispecific antibody comprising an anti-C1q arm and an arm targeting a target antigen other than HA), and an anti-IgG1 isotope control (REGN1932). All the antibodies were added at starting concentration of 166.67 nM and 1/4 serial dilution for each antibody. Bound antibodies to fixed cells were detected with an a-human IgG (H+L) HRP conjugated antibody (Jackson Immunoresearch, 1/5000) and chemiluminescent substrate (Life Technologies). Relative Fluorescent Units (RLUs) were measured with a Spectramax i3 plate reader (Molecular Devices).

MDCK cells were infected with Influenza A virus A/Puerto Rico/08/1934 (H1N1) at MOI 5 for 24 hours. Infected cells were harvested and combined with antibodies (0.89 μM starting concentration and 1/2 serial dilution for each antibody) and normal human serum complement (5% final concentration). After 2 hours of incubation, complement dependent cell lysis was evaluated with the CytoTox-Glo reagent (Promega). Relative Fluorescent Units (RLUs) were measured with a Spectramax i3 plate reader (Molecular Devices). The percent specific lysis is expressed relative to minimum and maximum lysis controls.

### Results and Conclusion

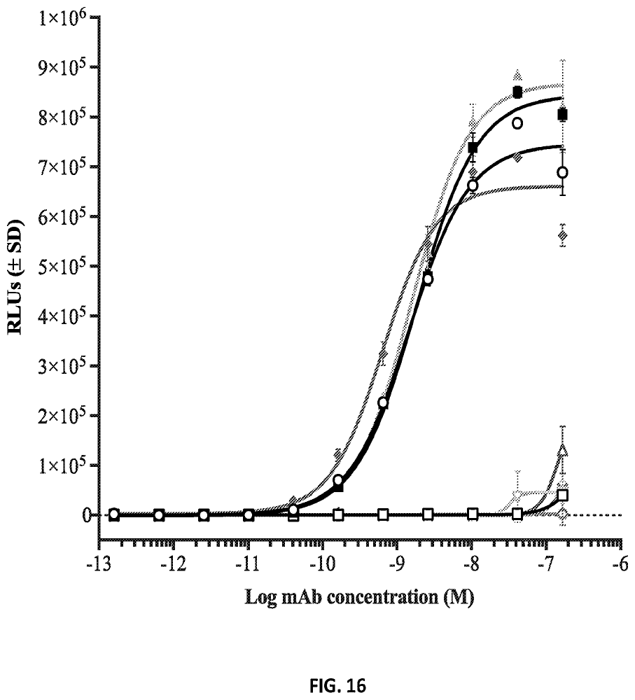

The broadly-neutralizing anti-influenza A group 1 HA×anti-C1q bispecific antibody (REGN6093) bound to HA with a similar EC50 as the parental anti-HA specific bivalent antibody (mAb11829) and parental anti-HA specific antibody with only a single arm (mAb27250; 1.85E-09 M and 1.46E-09 respectively) as shown in Table 37 and FIG. 16. All bispecific and bivalent control antibodies did not show significant binding.

As shown in Table 37 the broadly-neutralizing anti-influenza A group 1 HA×anti-C1q bispecific antibody (REGN6093) and parental control antibodies (mAb11829; mAb27250) show similar EC50s and maximum binding while control antibodies show no significant binding to A/Puerto Rico/08/1934 (H1N1)-infected cells.

TABLE 37

| | | Maximum | |
|---|---|---|---|
| | AbPID or REGN number | binding | |
| | (common name) | (RLUs) | EC₅₀ (M) |
| Influenza HA | mAb11829 (Flu A, group 1 HA) | 7.88 × 10⁵ | 1.46E−09 |
| | mAb27250 (Flu A, group 1, one | 7.19 × 10⁵ | 6.48E−10 |
| | arm) | | |
| Influenza HA × | REGN6093 (Flu A, group 1 | 8.49 × 10⁵ | 1.84E−09 |
| C1q | HA × C1q) | | |
| HA × FcγRIIIa | mAb20067 (Flu A, group 1 | 8.84 × 10⁵ | 1.62E−09 |
| | HA × FcγRIII) | | |
| Control | REGN5066 (IsdB × C1q) | 2505 | 1.67E−07 |
| bispecific | Negative control # 2 | 3.67 × 10⁴ | 1.67E−07 |
| mAbs | REGN5408 (IsdB × C1q scFv) | 2849 | 1.67E−07 |
| | Negative control #3 | 205 | 1.67E−07 |
| Isotype control | REGN1932 (hIgG1 control) | 2586 | 1.67E−07 |
| mAb | | | |

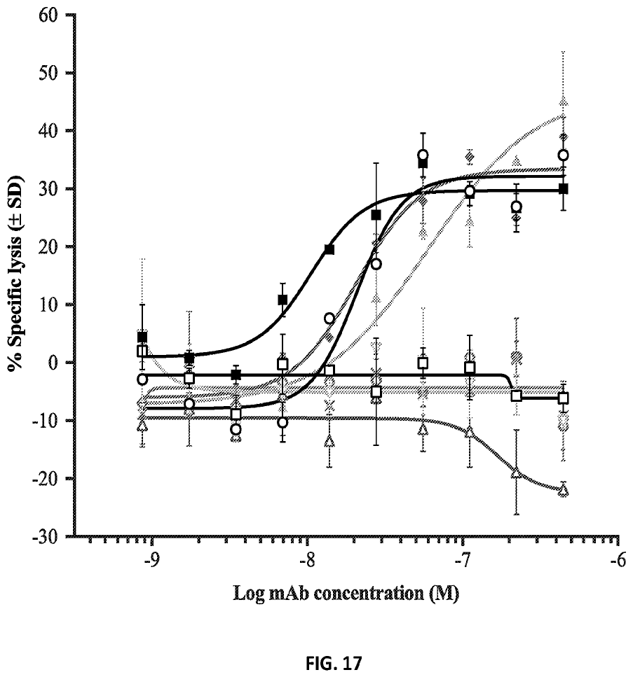

Broadly neutralizing anti-FluA group 1 HA×anti-C1q bispecific antibodies were tested for their ability to induce complement-dependent lysis of H1N1-infected cells. Bispecific antibodies targeting Influenza and the complement system may direct the complement system to the influenza-infected cells in a targeted manner. The broadly-neutralizing anti-Influenza A group 1 HA×anti-C1q bispecific antibody (REGN6093) showed increased specific lysis of A/Puerto Rico/08/134 (H1N1)-infected MDCK cells with 5% normal human serum complement compared to the parental monospecific antibody (mAb11829), 1.05E-08 M and 2.08E-08 M, respectively, as shown in Table 38. However, the anti-Influenza A group 1 HA×C1q bispecific antibody had slightly lower maximum percent lysis (30.0%) compared to the parental control (mAb11829) (35.8%) as shown in Table 38 and FIG. 17.

As shown in Table 38, anti-Influenza A group 1 HA×anti-C1q bispecific antibody (REGN6093) shows increased specific lysis of A/Puerto Rico/08/1934 (H1N1)-infected cells and slightly lower maximum specific lysis compared to the parental control antibody.

TABLE 38

| | | Maximum | |
|---|---|---|---|
| | | percent | |
| | AbPID or REGN number | specific | |
| | (common name) | lysis | EC₅₀ (nM) |
| Influenza HA | mAb11829 (Flu A, group 1 | 35.82 | 2.08E−08 |
| | HA) | | |
| | mAb27250 (Flu A, group 1, | 38.97 | 2.07E−08 |
| | one arm) | | |
| Influenza HA × | REGN6093 (Flu A, group 1 | 29.99 | 1.05E−08 |
| C1q | HA × C1q) | | |
| Influenza HA × | mAb20067 (Flu A, group 1 | 45.29 | 6.02E−08 |
| FcγRIIIa | HA × FcγRIII) | | |
| Control | REGN5066 (IsdB × C1q) | −6.15 | >8.89E−07 |
| bispecific | Negative control #2 | −10.10 | >8.89E−07 |
| mAbs | REGN5408 (IsdB × C1q | −21.85 | >8.89E−07 |
| | scFv) | | |
| | Negative control #3 | −9.08 | >8.89E−07 |
| Isotype control | REGN1932 (hIgG1 control) | −10.96 | >8.89E−07 |
| mAb | | | |

### Example 20: Anti-CD20×Anti-C1q Bispecific Antibody Binds Raji Cells Expressing CD20

Anti-CD20 antibodies (e.g., rituximab, ofatumumab) are known to activate complement and induce complement dependent lysis of lymphoma cells. In this Example, a CD20 antibody was selected (mAb14303) that has no inherent CDC activity on its own. We tested if recruiting C1q directly in a bispecific format using the mAb14303 CD20 targeting arm in an anti-CD20×anti-C1q bispecific antibody (REGN5177) can lead to robust C1q deposition and complement mediated lysis of Raji cells.

### Experimental Procedure

To determine the binding of the anti-CD20×anti-C1q bispecific antibody (REGN5177) to CD20 expressed on Raji cells, cells at a density of 1×10⁷/mL were fixed in 2% PFA. The fixed cells were blocked with 3% BSA for 1 hour at room temperature. After washing the cells with wash buffer, 100 ul of the cells were aliquoted into NUNC untreated plates. A first anti-CD20 bivalent antibody (mAb14303N2), a second anti-CD20 bivalent antibody (Rituximab), an anti-CD20×anti-C1q bispecific antibody ((REGN5177), and a parental anti-C1q bivalent antibody (REGN5201) were serially diluted 1:2 in assay dilution buffer (ADB: 0.5% BSA, 0.05% Tween-20 in 1×PBS), starting from 250 nM to 0.015 nM and added to the Raji cells in a 100 ul volume and incubated for 1 hour at room temperature. After washing the cells three times, 100 ul of 1:4000 diluted anti-human IgG HRP conjugated detection antibody in ADB was added to the cells and incubated at room temperature for 1 hour. The cells were washed three times, transferred to a NUNC maxisorp black plate, and incubated for a further 10-15 minutes with the chemiluminescent substrate before reading the plate using the Spectramax 13 plate reader (Molecular Devices).

For the complement deposition, Raji cells were resuspended in PBS at a density of 1×10⁷ cell/mL and fixed with 2% PFA for 30 minutes at room temperature. Cells were washed three times with 1× wash buffer and centrifuged between this and the below subsequent steps. The fixed cells were blocked with 3% BSA for 2 hrs at room temperature and then incubated with the antibodies from Example 21 in 0.5% BSA, 10 mM HEPES in RPMI for 1 hour at room temperature. The same antibodies from Example 21 were used and included REGN5177 (anti-CD20×anti-C1q hIgG1 antibody), Rituximab (commercially available anti-CD20 hIgG1 antibody), mAb14303 (the parental bivalent anti-CD20 hIgG1 antibody) and REGN5201 (the parental bivalent anti-C1q hIgG1 antibody). Each antibody was serially diluted 1:8 from 100 nM to 0.004 nM. After antibody binding, 5% protein A/G depleted pooled normal human serum was added for 1.5 hours at room temperature. Cells were washed 6× with wash buffer and incubated with lug/mL anti-C1q antibody for 1 hour at room temperature. After washing, a 1:4000 dilution of an anti-goat HRP detection antibody was added to the cells and incubated for an additional hour at room temperature. Finally, after washing, the chemiluminescent substrate was added to quantify the HRP conjugated secondary antibody associated with the cells. Nunc black maxisorp plates were read for luminescence by a Victor X instrument (Perkin Elmer) 10 minutes following the addition of the chemiluminescent substrate.

For the CDC bioassay, Raji cells were seeded onto a 96-well assay plates with clear bottom at 10,000 cells/well in 1% BSA containing RPMI 1640. Anti-CD20×anti-C1q bispecific antibody, anti-CD20 parental bivalent hIgG1 antibody, Rituximab, and the anti-C1q parental bivalent hIgG1 antibody were serially diluted 1:8 from 100 nM to 0.004 nM and added to the cells, along with human serum at a final concentration of 5%. Cytotoxicity was measured after 1 hour of incubation at 37° C. and in 5% C02, followed by a 30-minute incubation at 25° C., and addition of CytoTox-Glo™ reagent (Promega, #G9291). CytoTox-Glo™ is a luminescence-based reagent that measures cell killing such that increased luminescence is observed with increased cytotoxicity (measured in relative light units, RLUs). Untreated cells in control wells were lysed by treatment with digitonin immediately after addition of CytoTox-Glo™ reagent to determine maximal killing of cells. Plates were read for luminescence using a Spectramax 13 (Molecular Devices) 10-15 minutes following the addition of CytoTox-Glo™. Where calculated, the percentage of cytotoxicity was calculated with the RLU values by using the following equation:

$$\% Cytotoxicity = 100 \times \frac{( Experimental Cell Lysis - Background Cell Lysis )}{( Maximum Cell Lysis - Background Cell Lysis )}$$

In this equation “background cell lysis” is the luminescence from the cells treated with media and serum alone without any anti-CD20 antibody and the “maximum cell lysis” is the luminescence from the cells treated with digitonin. The results, expressed as % cytotoxicity or RLUs, were analyzed using nonlinear regression (4-parameter logistics) with Prism 7 software (GraphPad).

### Results Summary and Conclusion

The results were plotted as RLUs of binding on the y axis vs. log (M) antibody concentration on the x axis and were analyzed using nonlinear regression (4-parameter logistics) with Prism 7 software (GraphPad) to determine an EC₅₀ value of antibody binding. In two independent experiments, the first anti-CD20 bivalent antibody (mAb14303, the second anti-CD20 bivalent antibody (Rituximab), and the anti-CD20×anti-C1q bispecific antibody (REGN5177) bound Raji cells while no binding was observed with the parental anti-C1q bivalent antibody (REGN5201). Rituximab and mAb14303 bound with similar EC₅₀ values, 3.14×10−⁰⁹ and 6.031×10−⁰⁹ M, respectively. The EC₅₀ of anti-CD20×anti-C1q bispecific antibody binding to Raji cells was slightly lower, 3.566×10⁻⁰⁸ M. See Table 39. In summary, anti-CD20×anti-C1q bispecific antibody (REGN5177) bound Raji cells with similar EC₅₀ values as both the first anti-CD20 bivalent antibody (mAb14303) the second anti-CD20 bivalent antibody (Rituximab).

TABLE 39

| AbPID or REGN number | |
|---|---|
| (common name) | EC₅₀ (M) |
| mAb14303 (anti-CD20 bivalent) | 6.03 × 10⁻⁹ M |
| Rituximab (anti-CD20 bivalent) | 3.14 × 10⁻⁹ |
| REGN5177 (anti-CD20 × anti- | 3.56 × 10⁻⁸ M |
| C1q bispecific antibody) | |
| REGN5201 (parental anti-C1q | No binding |
| bivalent; control) | |

A bioassay to test the deposition of C1q using an anti-CD20×anti-C1q bispecific antibody on the target cell surface was developed. The assay used Raji cells, a human B cell line expressing CD20, complement preserved serum, and the test or control antibodies to assess the how much C1q is deposited on the cells.

The results, plotted as RLUs of binding on the y axis vs. log(M) antibody concentration on the x axis, were analyzed using nonlinear regression (4-parameter logistics) with Prism 7 software (GraphPad) to determine an EC50 value of C1q deposition. In two independent experiments, at the concentrations tested, C1q deposition was not detectable using this method with both Rituximab and the anti-CD20 hIgG1 parental antibody. With the anti-CD20×anti-C1q antibody bound to Raji cells, robust C1q deposition was observed, and had an average EC₅₀ value of 2.605×10⁻⁸ M. See Table 40.

TABLE 40

| AbPID or REGN number | |
|---|---|
| (common name) | C1q deposition (EC₅₀ (M)) |
| mAb14303 (anti-CD20 bivalent) | No deposition |
| Rituximab (anti-CD20 bivalent) | No deposition |
| REGN5177 (anti-CD20 × anti-C1q | 2.605 × 10⁻⁰⁸ M |
| bispecific antibody) | |

A complement dependent cytotoxicity bioassay to test the efficacy of anti-CD20×anti-C1q antibody in eliciting complement mediated killing of target cells was used. Table 41 shows the compiled results, including the EC₅₀ values of binding, C1q deposition of CDC on Raji cells, the EC₅₀ values of CDC on Raji cells, and % maximum complement dependent lysis for each antibody tested. The binding and deposition values are the average of two independent experiments and the CDC data is the average of 3 independent experiments.

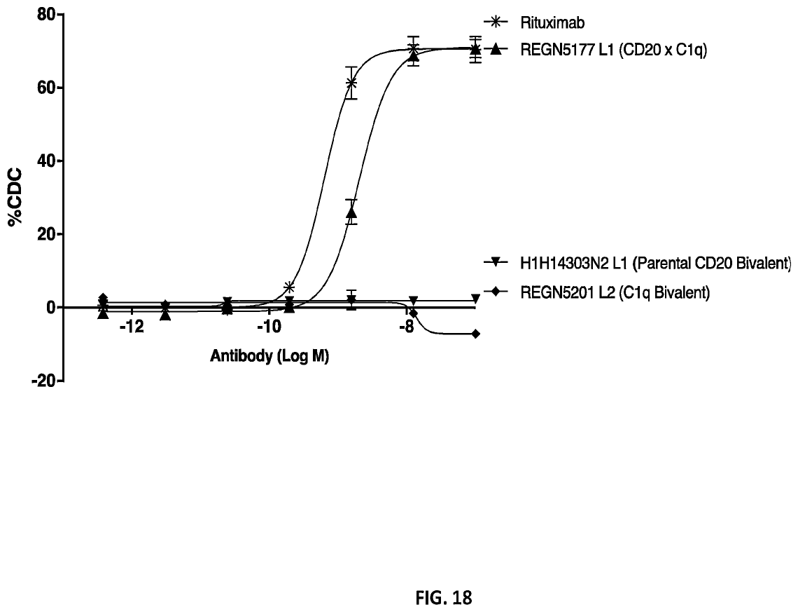

As shown in Table 41 and FIG. 18, the maximum lysis of Raji cells measured with 100 nM for the anti-CD20×anti-C1q bispecific antibody (REGN5177) was 71% compared to 62% with Rituximab. No complement mediated lysis was observed with either the anti-CD20 (mAb14303) or the anti-C1q (REGN5201) parental antibodies. The EC₅₀ values of CDC induced by Rituximab on Raji cells, 7.885×10⁻¹⁰ M, was higher than the EC₅₀ of CDC induced by the anti-CD20×anti-C1q bispecific antibody, 2.378×10˜9 M. By recruiting C1q with the bispecific arm, the mAb14303 targeting arm was able to induce similar maximum lysis of Raji cells as Rituximab with a lower EC₅₀.

TABLE 41

| | | | | | % |
|---|---|---|---|---|---|
| | | | C1q | | maximum |
| | | Binding to | deposition on | CDC on | complement |
| Antibody | | Raji cells | Raji cells | Raji cells | dependent |
| ID | Description | EC₅₀ (M) | EC₅₀ (M) | EC₅₀ (M) | lysis |
| REGN5177 | anti-CD20 × | 3.566 × 10⁻⁰⁸ | 2.605 × 10⁻⁸ | 2.378 × 10⁻⁹ | 71 |
| | anti-C1q | | | | |
| | bispecific hIgG1 | | | | |
| mAb14303 | anti-CD20 | 6.031 × 10⁻⁰⁹ | No deposition | No CDC | No CDC |
| | bivalent hIgG1 | | | | |
| Rituximab | anti-CD20 | 3.14 × 10⁻⁰⁹ | No deposition | 7.885 × 10⁻¹⁰ | 62 |
| | bivalent hIgG1 | | | | |
| REGN5201 | anti-C1q | No binding | No deposition | No CDC | No CDC |
| | bivalent hIgG1 | | | | |

### Example 21: Anti-GITR×Anti-C1q Bispecific Antibodies Target Cells Expressing Human GITR

Flow cytometric analysis was used to investigate binding of anti-GITR×anti-C1q bispecific antibodies (REGN6545; REGN6546) and the anti-GITR bivalent antibody (REGN5372) to target cells expressing human GITR. Binding was investigated using an engineered cell line, Jurkat/GITR/CD20.

### Experimental Procedure

Cells (300,000 cells/well) were incubated for 30 minutes at 4° C. with LIVE/DEAD Fixable Green Dead Cell Stain according to manufacturer's instructions to discriminate between live and dead cells. The cells were then washed twice with cold PBS containing 2% FBS, and incubated for 30 minutes at 4° C. with serial dilutions (1 pM to 1 μM) of and first and second anti-GITR×anti-C1q bispecific antibody (REGN6545, REGN6546), anti-GITR bivalent antibody (REGN5372), or anti-IgG1 isotype control antibody (REGN1932). After incubation, the cells were washed twice with cold PBS containing 2% FBS, and then fixed in BD Cytofix Buffer according to manufacturer's instructions, washed, re-suspended in PBS, and analyzed by flow cytometry on an iQue Screener flow cytometer. Unstained and secondary antibody alone controls were also tested for both cell lines. The results were analyzed using FlowJo (TreeStar Inc.) software and geometric mean fluorescence for viable cells were determined. The results were analyzed using nonlinear regression (4-parameter logistics) with Prism 5 software (GraphPad) to obtain EC₅₀ values for binding to cells.

NHS was added at a final serum concentration of 5% to target cells seeded at 5×10³ cells/well in triplicate rows of a 96-well plate. Serial dilutions of anti-GITR×anti-C1q antibodies (REGN6545; REGN6546), anti-GITR bivalent antibody (REGN5372), and control antibody (REGN1932) were then added to target cells. Final concentrations of each antibody in the assay ranged from 0.5 pM to 500 nM. To assess spontaneously generated signal (background), a buffer control sample containing all assay components except antibodies was evaluated. The cells were incubated for 3.5 hours at 37° C. and 5% CO₂. The plates were then equilibrated to room temperature for 30 minutes and CytoTox-Glo reagent was added to the wells. To determine the maximal target cell lysis signal, untreated target cells alone were lysed with digitonin prior to addition of CytoTox-Glo reagent. The plates were incubated briefly at room temperature and luminescence was measured from each well. The cytotoxic response was calculated as follows:

$$\begin{matrix}\begin{matrix}Cytotoxicity \\ =\end{matrix} \\ ( \% )\end{matrix} \frac{\begin{matrix}Experimental Signal - \\ Spontaneous Background {Signal}_{( target cells + effector cells )}\end{matrix}}{\begin{matrix}Max {Signal}_{( target cells w / digitonin )} - \\ Spontaneous Background {Signal}_{( target cells )}\end{matrix}} \times 100$$

Data was analyzed using a 4-parameter logistic equation over an 11-point dose-response curve (GraphPad Prism Version 7.0). For test articles with curves that cannot be fitted, the maximum cytotoxicity observed at any concentration of test article was reported.

### Results and Conclusion

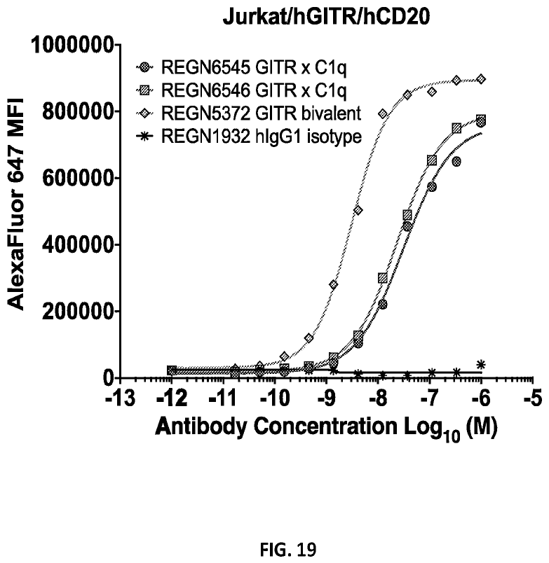

Flow cytometric analysis of anti-GITR×anti-C1q bispecific antibodies and anti-GITR bivalent antibodies bound to Jurkat/hGITR/hCD20 cells is disclosed in Table 42 and FIG. 19. As shown in Table 42, a first anti-GITR×anti-C1q bispecific antibody (REGN6545), a second anti-GITR×anti-C1q bispecific antibody (REGN6546), and anti-GITR bivalent antibody (REGN5372) bound in a dose dependent manner to Jurkat/hGITR/hCD20 cells with EC₅₀ values of 31, 24, and 3.1 nM, respectively. In contrast, an anti-IgG1 isotype control antibody (REGN1932) did not bind to Jurkat/hGITR/hCD20 cells.

TABLE 42

| Antibody | | Jurkat/hGITR/hCD20 | |
|---|---|---|---|
| ID | Description | Maximum MFI | EC₅₀ (M) |
| REGN6545 | Anti-GITR × anti-C1q, | 7.67 × 10⁵ | 3.10E−08 |
| | hIgG1 | | |
| REGN6546 | Anti-GITR × anti-C1q, | 7.75 × 10⁵ | 2.35E−08 |
| | hIgG1 | | |
| REGN5372 | Anti-GITR bivalent, | 8.97 × 10⁵ | 3.07E−09 |
| | hIgG1 | | |
| REGN1932 | hIgG1 isotype (Control) | 3.99 × 10⁴ | ND |

The ability of anti-GITR×anti-C1q antibodies (REGN6545; REGN6546) to mediate CDC in the presence of NHS was evaluated in engineered Jurkat/hGITR/hCD20 target cells.

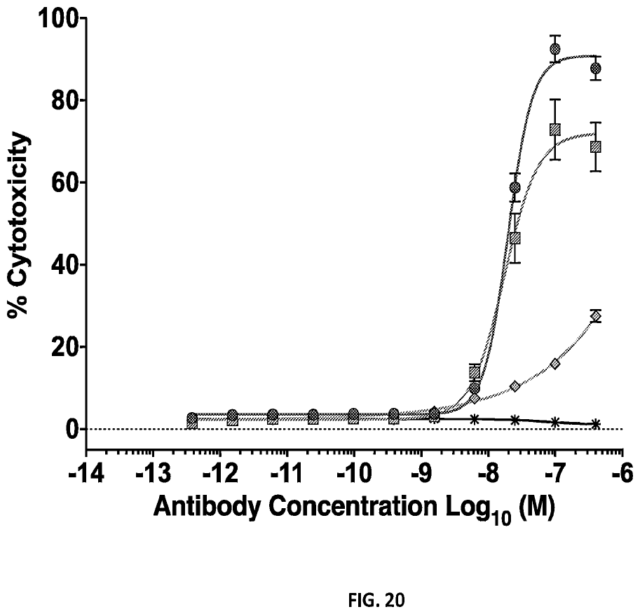

As shown in Table 43 and FIG. 20, the CDC activity of both anti-GITR×anti-C1q antibodies, REGN6545 and REGN6546, was evaluated as compared to a parental bivalent GITR antibody, REGN5372, using Jurkat/GITR/CD20 target cells in the presence of Normal Human Serum (NHS). In contrast to REGN5372, which induced weak CDC activity (28% maximum cytotoxicity against Jurkat/GITR/CD20 cells), and despite the increased expression levels of complement inhibitors on the surface of Jurkat cells compared to Raji cells (see Table 33), both anti-GITR×anti-C1q antibodies (REGN6545 and REGN6546) induced 93% and 73% maximum cytotoxicity, with EC₅₀ values of 19.7 nM and 17.4 nM, respectively. No activity was observed with any of the antibodies in the absence of Normal Human serum.

In summary, anti-GITR×anti-C1q bispecific antibodies induced complement dependent lysis, with REGN6545 having a 93% maximum cytotoxicity and an EC₅₀ of 1.97E-08 M and REGN6546 having a 73% maximum cytotoxicity and an EC₅₀ of 1.74E-08. These results, and results shown elsewhere herein, demonstrate that a C1q-recruiting platform can result in potent, targeted killing of diverse cell types by either imparting novel CDC activity or enhancing pre-existing CDC activity of a given antibody.

TABLE 43

| | | Jurkat/hGITR/hCD20 | |
|---|---|---|---|
| | | Maximum | |
| Antibody ID | Description | Cytotoxicity | EC₅₀ (M) |
| REGN6545 | Anti-GITR × anti-C1q | 93% | 1.97e−08 |
| | bispecific, hIgG1 | | |
| REGN6546 | Anti-GITR × anti-C1q | 73% | 1.74e−08 |
| | bispecific, hIgG1 | | |
| REGN5372 | Anti-GITR bivalent, hIgG1 | 28% | Not determined |
| REGN1932 | hIgG1 isotype (control) | 3% | Not determined |

### Summary

The wide-applicability of the C1q-bispecific platform approach was shown by demonstrating targeted killing of both bacterial and mammalian cells. Additionally, this approach overcomes the dependence on the antigen density and orientation of antibody binding to antigen in order to engage multiple C1q heads. The C1q-recruiting bsAb can activate complement when targeted to many different antigens including multiple S. aureus antigens, as well as antigens on P. aeruginosa, influenza-infected mammalian cells, B-cells, and T-cells. The platform should also be widely applicable to patients of different ages, genders, and immune status: the classical complement pathway and its protein components are abundant in circulation and stably expressed, whereas innate immune effector cells such as neutrophils, macrophages and T-cells have impaired function with age. Therefore a C1q-based therapeutic has the potential to be used in immune compromised patients including those on immunosuppressive therapies. Furthermore, the platform may have the potential to enhance the efficacy of existing treatments, such as antibiotics and tumor targeting agents.

Immunomodulatory therapeutics, including bsAbs, are currently being investigated as powerful ways to eliminate a variety of pathogenic cell types. The C1q-targeting platform disclosed herein represents a novel approach to leverage the cytotoxic effector functions of the immune system. The bsAbs described herein increased complement-mediated killing of bacterial and mammalian cells when targeted to antigens with diverse characteristics. The novel mechanism of this C1q-targeting platform has the possibility to be effective despite resistance mechanisms that can confound existing treatment modalities.

### Example 22: Reduced C1q Depletion with Bispecific Antibodies Having an IgG4 or IgG1 Uber Stealth Fe

In this example, the effect of the Fc region on the levels of C1q in circulation was tested using anti-IsdB×anti-C1q bispecific antibodies differing in their Fc region. REGN5066 is an anti-IsdB×anti-C1q bispecific antibody having an IgG1 Fc region, and is described in Example 11 above. REGN5331 is an anti-IsdB×anti-C1q bispecific antibody having the same V_{H} and V_{L} sequences as REGN5066 (i.e., the anti-IsdB heavy chain variable region of SEQ ID NO:26, the anti-C1q heavy chain variable region of SEQ ID NO:2, and the universal light chain variable region of SEQ ID NO:10) and having an IgG4 Fc region. REGN7017 is an anti-IsdB×anti-C1q bispecific antibody having the same V_{H} and V_{L} sequences as REGN5066 (i.e., the anti-IsdB heavy chain variable region of SEQ ID NO:26, the anti-C1q heavy chain variable region of SEQ ID NO:2, and the universal light chain variable region of SEQ ID NO:10) and having an IgG1 “uber stealth” Fc region comprising the mutations GGG- at positions 233-236 (by EU numbering) in the hinge region.

### Experimental Procedure

Humanized C1q (C1q^{Hu/Hu}) mice in a mixed background (MAID1615) were infected intraperitoneally with 200 μl volume of S. aureus Newman (1.5×10⁸ colony forming units (CFUs) per mouse). All three S. aureus strains were grown to log phase, OD₆₀₀≤1, in TSB at 37° C. and washed 3 times in PBS and adjusted to the desired density for infection. Both infected and uninfected mice were dosed subcutaneously with 100 μg of the anti-IsdB×anti-C1q IgG1 bispecific antibody (REGN5066), the anti-IsdB×anti-C1q IgG1 uber stealth bispecific antibody (REGN7017), the anti-IsdB×anti-C1q IgG4 bispecific antibody (REGN5331) or a control antibody (REGN1932) in a 100 μl volume. Blood was collected prior to infection and at 1 hr, 24 h, 48 h, and 72 h post antibody treatment (or 25 h, 48 h, 72 h, and 96 h post infection) and serum was prepared using gold-capped serum separator tubes (BD). Serum was diluted 1:2000 and analyzed by ELISA to determine C1q levels.

### Results Summary and Conclusion

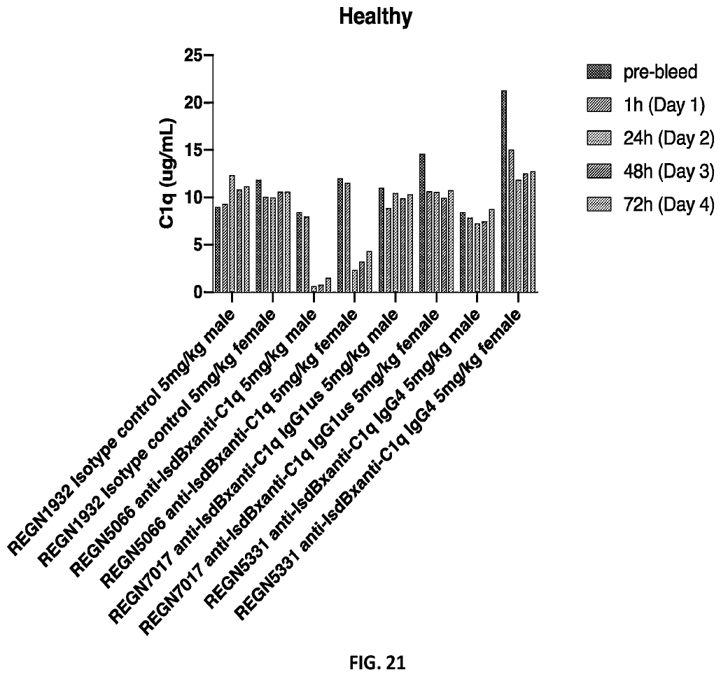

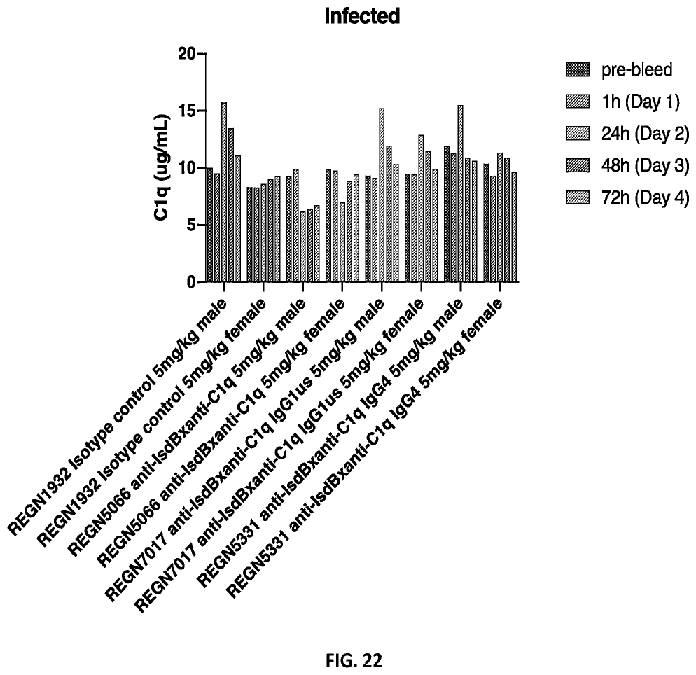

Bispecific anti-IsdB×anti-C1q antibody, REGN5066, was tested alongside IgG4 and IgG1 uber stealth versions and a control antibody to determine their effects on levels of C1q in circulation using male and female MAID1615 mice (FIGS. 21 and 22). In the uninfected animals, a 4- or 10-fold decrease in circulating C1q concentrations was observed when female or male animals, respectively, were dosed with the bispecific anti-IsdB×anti-C1q IgG1 antibody (as indicated by the arrows in FIG. 21). In contrast, C1q levels were stable throughout the course of the 4 days for uninfected mice administered an anti-IsdB×anti-C1q bispecific antibody comprising an Fc that is an IgG4 Fc or an IgG1 “uber stealth” Fc with reduced or no FcgR interactions (FIG. 21). In infected animals, where the IsdB target is present, there was a 10-35% reduction in circulating C1q concentrations in mice administered the bispecific anti-IsdB×anti-C1q IgG1 antibody, REGN5066 (FIG. 22). Similar to the uninfected animals, for female and male MAID1615 mice administered the IgG4 or IgG1 uber stealth versions of the anti-IsdB×anti-C1q bispecific antibody (REGN5331 or REGN7017, respectively), C1q levels were stable throughout the course of the infection over 4 days (FIG. 22). These experiments show that an IgG4 Fc and an IgG1 uber stealth Fc reduced C1q depletion that was observed with an anti-IsdB×anti-C1q IgG1 bispecific antibody both in the absence and presence of S. aureus target.

The present disclosure is not to be limited in scope by the specific embodiments described herein. Indeed, various modifications of the disclosure in addition to those described herein will become apparent to those skilled in the art from the foregoing description. Such modifications are intended to fall within the scope of the appended claims. All publications, patents, and patents applications referred to herein are hereby incorporated by reference.

What is claimed is:

1. An isolated bispecific antigen binding molecule comprising:
(a) a first antigen-binding domain that binds a target antigen; and
(b) a second antigen-binding domain that binds a complement component.

2. The isolated bispecific antigen binding molecule of claim 1, wherein the target antigen:
(a) is expressed on the surface of a cell or organism; or
(b) is a soluble antigen.

3. An isolated bispecific antigen binding molecule comprising:
(a) a first antigen-binding domain that binds a target antigen on the surface of a cell or organism, wherein the target antigen is a marker of a disease state of a subject; and
(b) a second antigen-binding domain that binds a complement component.

4. The isolated bispecific antigen binding molecule of any one of claims 1 to 3, wherein the target antigen comprises an antigen found on the surface of a bacteria, on the surface of a virus, on the surface of a virus infected cell, on the surface of a cancer cell, or on the surface of an immune cell.

5. The isolated bispecific antigen binding molecule of claim 3 or claim 4, wherein the disease state of the subject comprises a bacterial infection, a viral infection, cancer, or an immune dysfunction.

6. The isolated bispecific antigen binding molecule of any one of claims 1 to 5, wherein the target antigen comprises:
(a) a Staphylococcus species target antigen;
(b) an Influenza virus target antigen;
(c) a Pseudomonas species target antigen;
(d) a cancer cell antigen;
(e) a B-cell surface antigen; or
(f) a T-cell surface antigen.

7. The isolated bispecific antigen binding molecule of any one of claims 1 to 6, wherein the bispecific antigen binding molecule binds to the target antigen with an EC₅₀ of about 10 nM or less.

8. The isolated bispecific antigen binding molecule of any one of claims 1 to 7, wherein the bispecific antigen binding molecule promotes human C1q deposition on an organism or cell comprising the target antigen with an EC₅₀ of about 10 nM or less.

9. The isolated bispecific antigen binding molecule of any one of claims 1 to 8, wherein the first antigen binding domain binds to a Staphylococcus aureus target antigen.

10. The isolated bispecific antigen binding molecule of claim 9, wherein the first antigen-binding domain binds a Staphylococcus aureus IsdB antigen, a Staphylococcus aureus protein A antigen, a Staphylococcus aureus IsdA antigen, a Staphylococcus aureus Poly-N-acetyl glucosamine (PNAG) antigen, or a Staphylococcus aureus Clumping factor A (ClfA) antigen.

11. The isolated bispecific antigen binding molecule of any one of claims 1 to 8, wherein the first antigen binding domain binds a Pseudomonas aeruginosa target antigen.

12. The isolated bispecific antigen binding molecule of claim 11, wherein the first antigen-binding domain binds a Pseudomonas aeruginosa Psl antigen.

13. The isolated bispecific antigen binding molecule of any one of claims 1 to 8, wherein the first antigen binding domain binds an influenza virus target antigen.

14. The isolated bispecific antigen binding molecule of claim 13, wherein the first antigen binding domain binds an influenza hemagglutinin target antigen.

15. The isolated bispecific antigen binding molecule of claim 14, wherein the influenza hemagglutinin target protein comprises a H1 type hemagglutinin.

16. The isolated bispecific antigen binding molecule of any one of claims 1 to 8, wherein the first antigen binding domain binds a glucocorticoid-induced tumor necrosis receptor (GITR).

17. The isolated bispecific antigen binding molecule of any one of claims 1 to 8, wherein the first antigen binding domain binds a glycosylated phosphoprotein target antigen on the surface of B-cells.

18. The isolated bispecific antigen binding molecule of claim 17, wherein the first antigen binding domain binds a B-lymphocyte CD20 antigen.

19. The isolated bispecific antigen binding molecule of any one of claims 1 to 18, wherein the second antigen-binding domain binds complement component C1q.

20. The isolated bispecific antigen binding molecule of claim 19, wherein the complement component C1q is a human C1q.

21. The isolated bispecific antigen binding molecule of claim 19 or claim 20, wherein the second antigen binding domain binds to complement component C1q with a K_{D} of less than about 25 nM, less than about 20 nM, less than about 15 nM, less than about 10 nM, less than about 5 nM, less than about 1 nM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured in a surface plasmon resonance assay.

22. The isolated bispecific antigen binding molecule of any one of claims 1 to 8 or 19 to 21, wherein the first antigen-binding domain specifically binds IsdB and comprises:
(a) three heavy chain complementarity determining regions (HCDRs) contained within a heavy chain variable region (HCVR) comprising an amino acid sequence of SEQ ID NO:26; and
(b) three light chain complementarity determining regions (LCDRs) contained within a light chain variable region (LCVR) comprising an amino acid sequence of SEQ ID NO: 10.

23. The isolated bispecific antigen binding molecule of claim 22, wherein the HCDRs comprise a heavy chain CDR1 (HCDR1) comprising an amino acid sequence of SEQ ID NO: 28, a heavy chain CDR2 (HCDR2) comprising an amino acid sequence of SEQ ID NO: 30, and a heavy chain CDR3 (HCDR3) comprising an amino acid sequence of SEQ ID NO: 32.

24. The isolated bispecific antigen binding molecule of claim 22 or claim 23, wherein the LCDRs comprise a light chain CDR1 (LCDR) comprising an amino acid sequence of SEQ ID NO:12, a light chain CDR2 (LCDR2) comprising an amino acid sequence of SEQ ID NO:14, and a light chain CDR3 (LCDR3) comprising an amino acid sequence of SEQ ID NO:16.

25. The isolated bispecific antigen binding molecule of any one of claims 22 to 24, wherein the first antigen binding domain comprises a heavy chain (HC)/light chain (LC) pair comprising the amino acid sequences of SEQ ID NOs:84/85.

26. The isolated bispecific antigen binding molecule of any one of claims 1 to 8 or 19 to 21, wherein the first antigen-binding domain specifically binds CD20 and comprises:
(a) three heavy chain complementarity determining regions (HCDRs) contained within a heavy chain variable region (HCVR) comprising an amino acid sequence of SEQ ID NO: 34; and
(b) three light chain complementarity determining regions (LCDRs) contained within a light chain variable region (LCVR) comprising an amino acid sequence of SEQ ID NO: 10.

27. The isolated bispecific antigen binding molecule of claim 26, wherein the HCDRs comprise a heavy chain CDR1 (HCDR1) comprising an amino acid sequence of SEQ ID NO: 36, a heavy chain CDR2 (HCDR2) comprising an amino acid sequence of SEQ ID NO: 38, and a heavy chain CDR3 (HCDR3) comprising an amino acid sequence of SEQ ID NO: 40.

28. The isolated bispecific antigen binding molecule of claim 26 or claim 27, wherein the LCDRs comprise a light chain CDR1 (LCDR) comprising an amino acid sequence of SEQ ID NO:12, a light chain CDR2 (LCDR2) comprising an amino acid sequence of SEQ ID NO:14, and a light chain CDR3 (LCDR3) comprising an amino acid sequence of SEQ ID NO:16.

29. The isolated bispecific antigen binding molecule of any one of claims 26 to 28, wherein the first antigen binding domain comprises a HC/LC pair comprising the amino acid sequences of SEQ ID NOs:86/85.

30. The isolated bispecific antigen binding molecule of any one of claims 1 to 8 or 19 to 21, wherein the first antigen-binding domain specifically binds HA and comprises:
(a) three heavy chain complementarity determining regions (HCDRs) contained within a heavy chain variable region (HCVR) comprising an amino acid sequence of SEQ ID NO: 42; and
(b) three light chain complementarity determining regions (LCDRs) contained within a light chain variable region (LCVR) comprising an amino acid sequence of SEQ ID NO: 10.

31. The isolated bispecific antigen binding molecule of claim 30, wherein the HCDRs comprise a heavy chain CDR1 (HCDR1) comprising an amino acid sequence of SEQ ID NO: 44, a heavy chain CDR2 (HCDR2) comprising an amino acid sequence of SEQ ID NO: 46, and a heavy chain CDR3 (HCDR3) comprising an amino acid sequence of SEQ ID NO: 48.

32. The isolated bispecific antigen binding molecule of claim 30 or claim 31, wherein the LCDRs comprise a light chain CDR1 (LCDR) comprising an amino acid sequence of SEQ ID NO:12, a light chain CDR2 (LCDR2) comprising an amino acid sequence of SEQ ID NO:14, and a light chain CDR3 (LCDR3) comprising an amino acid sequence of SEQ ID NO:16.

33. The isolated bispecific antigen binding molecule of any one of claims 30 to 32, wherein the first antigen binding domain comprises a HC/LC pair comprising the amino acid sequences of SEQ ID NOs:87/85.

34. The isolated bispecific antigen binding molecule of any one of claims 1 to 8 or 19 to 21, wherein the first antigen-binding domain specifically binds GITR and comprises:
(a) three heavy chain complementarity determining regions (HCDRs) contained within a heavy chain variable region (HCVR) comprising an amino acid sequence of SEQ ID NO: 50; and
(b) three light chain complementarity determining regions (LCDRs) contained within a light chain variable region (LCVR) comprising an amino acid sequence of SEQ ID NO: 10.

35. The isolated bispecific antigen binding molecule of claim 34, wherein the HCDRs comprise heavy chain CDR1 (HCDR1) comprising an amino acid sequence of SEQ ID NO: 52, a heavy chain CDR2 (HCDR2) comprising an amino acid sequence of SEQ ID NO: 54, and a heavy chain CDR3 (HCDR3) comprising an amino acid sequence of SEQ ID NO: 56.

36. The isolated bispecific antigen binding molecule of claim 34 or claim 35, wherein the LCDRs comprise a light chain CDR1 (LCDR) comprising an amino acid sequence of SEQ ID NO:12, a light chain CDR2 (LCDR2) comprising an amino acid sequence of SEQ ID NO:14, and a light chain CDR3 (LCDR3) comprising an amino acid sequence of SEQ ID NO:16.

37. The isolated bispecific antigen binding molecule of any one of claims 34 to 36, wherein the first antigen binding domain comprises a HC/LC pair comprising the amino acid sequences of SEQ ID NOs:88/85.

38. The isolated bispecific antigen binding molecule of any one of claims 1 to 8 or 19 to 21, wherein the first antigen-binding domain specifically binds protein A and comprises:
(a) three heavy chain complementarity determining regions (HCDRs) contained within a heavy chain variable region (HCVR) comprising an amino acid sequence of SEQ ID NO: 58; and
(b) three light chain complementarity determining regions (LCDRs) contained within a light chain variable region (LCVR) comprising an amino acid sequence of SEQ ID NO: 10.

39. The isolated bispecific antigen binding molecule of claim 38, wherein the HCDRs comprise a heavy chain CDR1 (HCDR1) comprising an amino acid sequence of SEQ ID NO: 60, a heavy chain CDR2 (HCDR2) comprising an amino acid sequence of SEQ ID NO: 62, and a heavy chain CDR3 (HCDR3) comprising an amino acid sequence of SEQ ID NO: 64.

40. The isolated bispecific antigen binding molecule of claim 38 or claim 39, wherein the LCDRs comprise a light chain CDR1 (LCDR) comprising an amino acid sequence of SEQ ID NO:12, a light chain CDR2 (LCDR2) comprising an amino acid sequence of SEQ ID NO:14, and a light chain CDR3 (LCDR3) comprising an amino acid sequence of SEQ ID NO:16.

41. The isolated bispecific antigen binding molecule of any one of claims 38 to 40, wherein the first antigen binding domain comprises a HC/LC pair comprising the amino acid sequences of SEQ ID NOs:90/85.

42. The isolated bispecific antigen binding molecule of any one of claims 1 to 8 or 19 to 21, wherein the first antigen-binding domain specifically binds IsdA and comprises:
(a) three heavy chain complementarity determining regions (HCDRs) contained within a heavy chain variable region (HCVR) comprising an amino acid sequence of SEQ ID NO: 66; and
(b) three light chain complementarity determining regions (LCDRs) contained within a light chain variable region (LCVR) comprising an amino acid sequence of SEQ ID NO: 10.

43. The isolated bispecific antigen binding molecule of claim 42, wherein the HCDRs comprise a heavy chain CDR1 (HCDR1) comprising an amino acid sequence of SEQ ID NO: 68, a heavy chain CDR2 (HCDR2) comprising an amino acid sequence of SEQ ID NO: 70, and a heavy chain CDR3 (HCDR3) comprising an amino acid sequence of SEQ ID NO: 72.

44. The isolated bispecific antigen binding molecule of claim 42 or claim 43, wherein the LCDRs comprise a light chain CDR1 (LCDR) comprising an amino acid sequence of SEQ ID NO:12, a light chain CDR2 (LCDR2) comprising an amino acid sequence of SEQ ID NO:14, and a light chain CDR3 (LCDR3) comprising an amino acid sequence of SEQ ID NO:16.

45. The isolated bispecific antigen binding molecule of any one of claims 42 to 44, wherein the first antigen binding domain comprises a HC/LC pair comprising the amino acid sequences of SEQ ID NOs:91/85.

46. The isolated bispecific antigen binding molecule of any one of claims 1 to 8 or 19 to 21, wherein the first antigen binding domain specifically binds ClfA and comprises a HC/LC pair comprising the amino acid sequences of SEQ ID NOs:73/74.

47. The isolated bispecific antigen binding molecule of any one of claims 1 to 8 or 19 to 21, wherein the first antigen binding domain specifically binds PNAG and comprises a HC/LC pair comprising the amino acid sequences of SEQ ID NOs:76/77.

48. The isolated bispecific antigen binding molecule of any one of claims 1 to 8 or 19 to 21, wherein the first antigen binding domain specifically binds Psl and comprises a HC/LC pair comprising the amino acid sequences of SEQ ID NOs:78/79.

49. An isolated bispecific antigen binding molecule comprising:
(a) a first antigen-binding domain that binds a target antigen; and
(b) a second antigen-binding domain that binds human C1q.

50. The isolated bispecific antigen binding molecule of claim 49, wherein the target antigen:
(a) is expressed on the surface of a cell or organism; or
(b) is a soluble antigen.

51. The isolated bispecific antigen binding molecule of claim 49 or claim 50, wherein the second antigen-binding domain comprises a HCDR1 comprising an amino acid sequence selected from the group consisting of SEQ ID NO:4 and SEQ ID NO:20, a HCDR2 comprising an amino acid sequence selected from the group consisting of SEQ ID NO:6 and SEQ ID NO:22, and a HCDR3 comprising an amino acid sequence selected from the group consisting of SEQ ID NO:8 and SEQ ID NO:24.

52. The isolated bispecific antigen binding molecule of claim 51, wherein the second antigen-binding domain comprises:
a HCDR1 comprising the amino acid sequence of SEQ ID NO:4, a HCDR2 comprising the amino acid sequence of SEQ ID NO:6, and a HCDR3 comprising the amino acid sequence of SEQ ID NO:8; or
a HCDR1 comprising the amino acid sequence of SEQ ID NO:20, a HCDR2 comprising the amino acid sequence of SEQ ID NO:22, and a HCDR3 comprising the amino acid sequence of SEQ ID NO:24.

53. The isolated bispecific antigen binding molecule of claim 51 or claim 52, wherein the second antigen-binding domain comprises a HCVR comprising the amino acid sequence of SEQ ID NO:2 or SEQ ID NO:18.

54. The isolated bispecific antigen binding molecule of any one of claims 49 to 53, wherein the first antigen-binding domain comprises a HCVR comprising:
a HCDR1 comprising the amino acid sequence of SEQ ID NO:28, a HCDR2 comprising the amino acid sequence of SEQ ID NO:30, and a HCDR3 comprising the amino acid sequence of SEQ ID NO:32; or
a HCDR1 comprising the amino acid sequence of SEQ ID NO:36, a HCDR2 comprising the amino acid sequence of SEQ ID NO:38, and a HCDR3 comprising the amino acid sequence of SEQ ID NO:40; or
a HCDR1 comprising the amino acid sequence of SEQ ID NO:44, a HCDR2 comprising the amino acid sequence of SEQ ID NO:46, and a HCDR3 comprising the amino acid sequence of SEQ ID NO:48; or
a HCDR1 comprising the amino acid sequence of SEQ ID NO:52, a HCDR2 comprising the amino acid sequence of SEQ ID NO:54, and a HCDR3 comprising the amino acid sequence of SEQ ID NO:56; or
a HCDR1 comprising the amino acid sequence of SEQ ID NO:60, a HCDR2 comprising the amino acid sequence of SEQ ID NO:62, and a HCDR3 comprising the amino acid sequence of SEQ ID NO:64; or
a HCDR1 comprising the amino acid sequence of SEQ ID NO:68, a HCDR2 comprising the amino acid sequence of SEQ ID NO:70, and a HCDR3 comprising the amino acid sequence of SEQ ID NO:72; or
a HCDR1 comprising amino acids 26-33 of SEQ ID NO:73, a HCDR2 comprising amino acids 51-58 of SEQ ID NO:73, and a HCDR3 comprising amino acids 96-106 of SEQ ID NO:73; or
a HCDR1 comprising amino acids 27-32 of SEQ ID NO:76, a HCDR2 comprising amino acids 51-58 of SEQ ID NO:76, and a HCDR3 comprising amino acids 96-106 of SEQ ID NO:76; or
a HCDR1 comprising amino acids 31-35 of SEQ ID NO:78, a HCDR2 comprising amino acids 50-65 of SEQ ID NO:78, and a HCDR3 comprising amino acids 98-109 of SEQ ID NO:78.

55. The isolated bispecific antigen binding molecule of any one of claims 49 to 54, further comprising a LCVR comprising:
a LCDR1 comprising the amino acid sequence of SEQ ID NO:12, a LCDR2 comprising the amino acid sequence of SEQ ID NO:14, and a LCDR3 comprising the amino acid sequence of SEQ ID NO:16; or
a LCDR1 comprising amino acids 27-32 of SEQ ID NO:74, a LCDR2 comprising amino acids 51-55 of SEQ ID NO:74, and a LCDR3 comprising amino acids 89-98 of SEQ ID NO:74; or
a LCDR1 comprising amino acids 27-32 of SEQ ID NO:77, a LCDR2 comprising amino acids 50-52 of SEQ ID NO:77, and a LCDR3 comprising amino acids 98-101 of SEQ ID NO:77; or
a LCDR1 comprising amino acids 23-33 of SEQ ID NO:79, a LCDR2 comprising amino acids 49-55 of SEQ ID NO:79, and a LCDR3 comprising amino acids 88-99 of SEQ ID NO:79.

56. The isolated bispecific antigen binding molecule of claim 55, wherein the LCVR comprises a LCDR1 comprising the amino acid sequence of SEQ ID NO:12, a LCDR2 comprising the amino acid sequence of SEQ ID NO:14, and a LCDR3 comprising the amino acid sequence of SEQ ID NO:16.

57. The isolated bispecific antigen binding molecule of claim 56, comprising a LCVR comprising the amino acid sequence of SEQ ID NO:10.

58. A pharmaceutical composition comprising the isolated bispecific antigen binding molecule of any one of claims 1 to 57 and a pharmaceutically acceptable carrier.

59. A method of activating or enhancing cell killing, the method comprising contacting the cell with the isolated bispecific antigen binding molecule of any one of claims 1 to 57 or the pharmaceutical composition of claim 58.

60. A method of treating cancer comprising: administering the bispecific antigen binding molecule of any one of claims 1 to 8, 16 to 21, 26 to 29, 34 to 37, or 49 to 57, or the pharmaceutical composition of claim 58, to a subject having cancer.

61. The method of claim 60, wherein the bispecific antigen binding molecule comprises a first antigen binding domain that binds CD20 or GITR.

62. The method of claim 61, wherein the bispecific antigen binding molecule comprises a first antigen binding domain that binds CD20.

63. The method of claim 62, wherein the bispecific antigen binding molecule comprises a first antigen binding domain of any one of claims 26 to 29.

64. The method of claim 61, wherein the bispecific antigen binding molecule comprises a first antigen binding domain that binds GITR.

65. The method of claim 64, wherein the bispecific antigen binding molecule comprises a first antigen binding domain of any one of claims 34 to 37.

66. A method of treating a bacterial infection comprising: administering the bispecific antigen binding molecule of any one of claims 1 to 12, 19 to 25, or 38 to 57, or the pharmaceutical composition of claim 58, to a subject having a bacterial infection.

67. The method of claim 66, wherein the bispecific antigen binding molecule comprises a first antigen binding domain that binds Pseudomonas aeruginosa.

68. The method of claim 67, wherein the bispecific antigen binding molecule comprises a first antigen binding domain that binds the Psl antigen of Pseudomonas aeruginosa.

69. The method of claim 68, wherein the bispecific antigen binding molecule comprises a first antigen binding domain of claim 48.

70. A method of treating a viral infection comprising: administering the bispecific antigen binding molecule of any one of claims 1 to 8, 13 to 15, 19 to 21, 30 to 33, or 49 to 57, or the pharmaceutical composition of claim 58, to a subject having a viral infection.

71. The method of claim 70, wherein the bispecific antigen binding molecule comprises a first antigen binding domain that specifically binds Influenza or an influenza infected cell.

72. The method of claim 71, wherein the bispecific antigen binding molecule comprises a first antigen binding domain that specifically binds the hemagglutinin antigen of a H1N1 strain of influenza.

73. The method of claim 71, wherein the bispecific antigen binding molecule comprises a first antigen binding domain of any one of claims 30 to 33.
